(19) United States
(12) Patent Application Publication
(10) Pub. No.: US 2024/0390475 A1
(43) Pub. Date: Nov. 28, 2024

(54) HUMAN PAPILLOMAVIRUS VACCINES AND USES OF THE SAME

(71) Applicant: PRECIGEN, INC., Germantown, MD (US)

(72) Inventors: Douglas E. BROUGH, Germantown, MD (US); Cheryl BOLINGER, Germantown, MD (US); Ramya YARLAGADDA, Germantown, MD (US); Vinodhbabu KURELLA, Germantown, MD (US); Prabakaran PONRAJ, Germantown, MD (US); Simon METENOU, Germantown, MD (US); Kuan-fu DING, Germantown, MD (US)

(73) Assignee: PRECIGEN, INC., Germantown, MD (US)

(21) Appl. No.: 18/671,275

(22) Filed: May 22, 2024

Related U.S. Application Data
(60) Provisional application No. 63/468,119, filed on May 22, 2023.

(51) Int. Cl. A61K 39/12; A61K 39/00; A61K 45/06; A61P 35/00

(52) U.S. Cl. CPC A61K 39/12; A61K 45/06; A61P 35/00; A61K 2039/53; A61K 2039/585; A61K 2039/892

(57) ABSTRACT

Multi-antigenic human papilloma virus (HPV) molecular vaccine constructs for use and treatment of HPV-associated disorders and pathologies, such as HPV molecular vaccines targeting HPV16-, HPV18-, and HPV-45-associated pathologies.

### SEQUENCE LISTING

The instant application contains a Sequence Listing which has been submitted electronically in XML file format and is hereby incorporated by reference in its entirety. Said XML copy, created on May 15, 2024, is named 75594-408670_SL.xml and is 577,560 bytes in size.

### BACKGROUND OF THE INVENTION

The human papillomavirus (HPV) infects millions of individuals and causes over 600,000 cases of HPV-associated malignancies worldwide each year. Int J Cancer. 2017; 141 (4): 664-670. There are approximately 47,000 new cases of HPV-associated malignancies diagnosed in the United States annually. Although many papillomavirus infections are benign and will resolve on their own, some persistent infections will evolve into epithelial cell dysplasia and may result in cancer of the cervix, vulva, penis, oropharyngeal cavity, head and neck, and anal cavity. Int J Oncol. 2018; 52 (3): 637-655. In general, HPV is thought to be responsible for more than 90% of anal and cervical cancers, about 70% of vaginal and vulvar cancers, and more than 60% of penile cancers. Oropharyngeal cancers traditionally have been caused by tobacco and alcohol, but recent studies show that about 70% of cancers of the oropharynx may be linked to HPV. https://www.cdc.gov/cancer/hpv/statistics/index.htm.

Over 200 types of HPV are categorized into high-risk and low-risk groups depending on their oncogenic potential. Among the high-risk HPV types, HPV types 16 and 18 are the most prevalent and carcinogenic. J Gynecol Oncol. 2016; 27 (2): e21-e21; N Engl J Med. 2003; 348 (6): 518-527. Together, HPV 16 and 18 are responsible for approximately 70% of cervical cancer cases. J Biomed Sci. 2016; 23 (1): 75. Early (E) HPV genes (E1-E8) regulate viral expression and replication, and late (L) genes control viral protein coding. Virology. 1977; 76 (2): 569-580; J Virol. 1977; 24 (1): 108-120; Virology. 1979; 96 (2): 547-552.

Several prophylactic HPV vaccines developed to target the major capsid protein L1 of the HPV viral particle have been shown to be effective and represent a major breakthrough in the reduction of HPV malignancies in developed countries. J Biomed Sci. 2016; 23 (1): 75. Prophylactic vaccines prevent healthy individuals from acquiring HPV infections, and they prevent previously infected individuals from being reinfected. Unfortunately, prophylactic vaccines are not effective against established HPV infections and the resultant HPV-associated malignant or premalignant lesions. Discov Med. 2010; 10 (50): 7-17; Expert Opin Emerg Drugs. 2012; 17 (4): 469-492. Unlike prophylactic HPV vaccines, which are used to generate neutralizing antibodies against the viral capsid protein L1 to prevent infection, therapeutic HPV vaccines are used to stimulate cell-mediated immune responses to specifically target and kill infected cells. Clin Diagn Lab Immunol. 2001; 8 (2): 209-220.

The E6 and E7 proteins of HPV 16 and 18 represent potential targets for therapeutic vaccines because they are responsible for maintenance of the malignant phenotype and are constitutively expressed in the tumors. Expert Opin Emerg Drugs. 2012; 17 (4): 469-492. Moreover, they are not endogenously expressed on any human tissues, so there is very low risk of inducing autoimmune events with a vaccine targeting these proteins. Several CD8+ T cell epitopes of E6 and E7 capable of eliciting cytotoxic T lymphocyte (CTL) responses have previously been identified, and clinical studies employing diverse vaccine platforms have demonstrated various degrees of effectiveness in terms of eliciting HPV-specific responses and clinical benefits. These include live vector, peptide or protein, cell-based, and nucleic acid vaccines. J Immunol. 1994; 152 (8): 3904-3912; Virus Res. 1998; 54 (1): 23-29; Clin Cancer Res. 2001; 7 (3 Suppl): 788s-795s; Expert Opin Emerg Drugs. 2012; 17 (4): 469-492; Clin Exp Immunol. 2015; 181 (1): 164-178; Cancers (Basel). 2011; 3 (3): 3461-3495; Int J Cancer. 2000; 86 (5): 725-730; J Formos Med Assoc. 2010; 109 (1): 4-24; Oncologist. 2005; 10 (7): 528-538; BMC Cancer. 2014; 14:748; Expert Rev Vaccines. 2007; 6 (2): 227-239; Expert Rev Vaccines. 2016; 15 (8): 989-1007.

The naturally existing sequence variations on numerous HPV strains present a significant hurdle to the development of effective, broad spectrum HPV vaccines. For example, recurrent/metastatic cervical cancer linked to HPV16/18 has a poor prognosis, with responses to treatment with chemotherapy±bevacizumab+pembrolizumab around 70% with approximately 10.4 months overall survival. Keynote-826; N Engl J Med 2021; 385:1856-1867.

As a solution to this problem, the present vaccine design approach utilizes advanced bioinformatics and protein engineering approaches to select and design HPV 16/18 antigen sequences with broad coverage of T cell epitopes, novel mutations, and enhancer agonist peptides. Drawing on information available of extended coverage of antigen regions with CTL-specific epitopes and in silica prediction results, the designed HPV vaccine antigens of the present invention are targeted to induce robust HPV-16 and HPV-18 specific responses and provide therapeutic benefit to persons at risk of HPV-derived cancers.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF INVENTION

Provided herein is a non-naturally occurring polynucleotide encoding a polypeptide construct comprising an immune response-inducing human papilloma virus (HPV) peptide.

In some embodiments, the non-naturally occurring polynucleotide encodes a polypeptide construct comprising two or more HPV peptides. In some embodiments, the two or more HPV peptides comprise one or more HPV-16 immune response-inducing peptide sequences (HPV-16 peptides).

In some embodiments, the polypeptide construct comprises an HPV-16 E5 peptide, an HPV-16 E6 peptide and/or an HPV-16 E7 peptide. In some embodiments, the polypeptide construct comprises an HPV-16 E5 peptide having the sequence of SEQ ID NO: 47. In some embodiments, the polypeptide construct comprises an HPV-16 E6 peptide having the sequence of SEQ ID NO: 45. In some embodiments, the polypeptide construct comprises an HPV-16 E7 peptide having the sequence of SEQ ID NO: 46.

In some embodiments, the polypeptide construct comprises an HPV-18 peptide. In some embodiments, the polypeptide construct comprises an HPV-18 E5 peptide, an HPV-18 E6 peptide, and/or an HPV-18 E7 peptide. In some embodiments, the polypeptide construct comprises an HPV-18 E5 peptide having the sequence of SEQ ID NO: 50. In some embodiments, the polypeptide construct comprises an HPV-16 E6 peptide having the sequence of SEQ ID NO: 48. In some embodiments, the polypeptide construct comprises an HPV-18 E7 peptide having the sequence of SEQ ID NO: 49.

In some embodiments, the polypeptide has the sequence of SEQ ID NO: 51.

In some embodiments, at least one of the one or more HPV peptides is connected to an agonist peptide (also referred to as an enhancer agonist peptide). In some embodiments, the agonist peptide has a sequence as shown in Table 5. In some embodiments, the polypeptide has the sequence of SEQ ID NO: 53.

Further provided herein is a vector comprising any of the polynucleotides provided herein. In some embodiments, the vector is an adenoviral vector. In some embodiments, the adenoviral vector is a gorilla adenoviral vector.

Provided herein is an E6 peptide, wherein the E6 peptide comprises an E18A amino acid substitution and at least one of an L50G, E148A, T149A, Q150A, or L151A amino acid substitution as compared to a wildtype E6 peptide. In some embodiments, the E6 peptide comprises E18A, L50G, E148A, T149A, Q150A, and L151A amino acid substitutions. In some embodiments, the E6 peptide has the sequence of SEQ ID NO: 45. In some embodiments, said E6 peptide is fused to an agonist peptide. In some embodiments, said agonist peptide is fused to at least one of a C-terminus and an N-terminus of said E6 peptide. In some embodiments, the wildtype E6 peptide is from HPV-16.

Provided herein is an E6 peptide, wherein the E6 peptide comprises a deletion as compared to a wildtype E6 peptide, wherein the deletion comprises a C-terminus of the wildtype E6 peptide. In some embodiments, the deletion is that of the amino acids from residue position 121 to the C-terminus of the wildtype E6 peptide. In some embodiments, the E6 peptide comprises at least one of an E18A and an L50G substitution as compared to wildtype E6 peptide. In some embodiments, the wildtype E6 peptide is from HPV-18. In some embodiments, the E6 peptide has the sequence of SEQ ID NO: 48.

Provided herein is an E7 peptide, wherein the E7 peptide comprises a deletion of the N-terminus of wildtype E7 peptide. In some embodiments, the deletion is that of the amino acids at residue positions 1-39 of wildtype E7 peptide. In some embodiments, the E7 peptide comprises at least one of an E55A and an L74R substitution as compared to the wildtype E7 peptide. In some embodiments, the wildtype E7 peptide is from HPV-18. In some embodiments, the E7 peptide has the sequence of SEQ ID NO: 49.

Provided herein is an E5 peptide, wherein the E5 peptide comprises a deletion of amino acids at residue positions 41-57 of wildtype E5 peptide. In some embodiments, the E5 peptide has the sequence of SEQ ID NO: 47. In some embodiments, the wildtype E5 peptide is from HPV-16.

Provided herein is an E5 peptide, wherein the E5 peptide comprises a deletion of amino acid at residue positions 27-40 and/or amino acids at residue positions 54-57 of wildtype E5 peptide. In some embodiments, the E5 peptide has the sequence of SEQ ID NO: 50. In some embodiments, the wildtype E5 peptide is from HPV-18.

Provided herein is a polypeptide construct comprising any one of the presently described E5, E6, and E7 peptides.

Provided herein is a polypeptide construct comprising an HPV-16 E6 peptide comprising an E18A amino acid substitution and at least one of an L50G, E148A, T149A, Q150A, or L151A amino acid substitution as compared to a wildtype HPV-16 E6 peptide. In some embodiments, the HPV-16 E6 peptide comprises E18A, L50G, E148A, T149A, Q150A, and L151A amino acid substitutions. In some embodiments, the HPV-16 E6 peptide has the sequence of SEQ ID NO: 45. In some embodiments, the polypeptide construct further comprises an HPV-16 E7 peptide comprising at least one of an H2P, C24G, E46A, or L67R amino acid substitution as compared to a wildtype HPV-16 E7 peptide. In some embodiments, the HPV-16 E7 peptide comprises H2P, C24G, E46A, and L67R amino acid substitutions. In some embodiments, the HPV-16 E7 peptide has the sequence of SEQ ID NO: 46. In some embodiments, the polypeptide construct further comprises an HPV-16 E5 peptide. In some embodiments, the HPV-16 E5 peptide comprises a deletion of one or more amino acids as compared to a wildtype HPV-16 E5 peptide. In some embodiments, the deletion comprises amino acids at residue positions 41-57 of the wildtype HPV-16 E5 peptide. In some embodiments, the HPV-16 E5 peptide has the sequence of SEQ ID NO: 47.

In some embodiments, the polypeptide construct comprises: (a) an HPV-16 E6 peptide comprising an E18A amino acid substitution and at least one of an L50G, E148A, T149A, Q150A, or L151A amino acid substitution as compared to a wildtype HPV-16 E6 peptide; and (b) an HPV-18 E6 peptide. In some embodiments, the HPV-18 E6 peptide comprises an E18A and an L50G substitution as compared to a wildtype HPV-18 E6 peptide. In some embodiments, the HPV-18 E6 peptide comprises a deletion of at least one C-terminus amino acid relative to said wildtype HPV-18 E6 peptide. In some embodiments, the deletion comprises the amino acids from amino acid at residue position 121 to the C-terminus of the wildtype HPV-18 E6 peptide. In some embodiments, the HPV-18 E6 peptide has the sequence of SEQ ID NO: 48. In some embodiments, the polypeptide construct further comprises an HPV-18 E7 peptide. In some embodiments, the HPV-18 E7 peptide comprises an E55A and an L74R substitution as compared to a wildtype HPV-18 E7 peptide. In some embodiments, the HPV-18 E7 peptide comprises a deletion of at least one amino acid from an N-terminus of the HPV-18 E7 peptide. In some embodiments, the deletion comprises the amino acids at residue positions 1-40 of the wildtype HPV-18 E7 peptide. In some embodiments, the HPV-18 E7 peptide has the sequence of SEQ ID NO: 49. In some embodiments, said polypeptide construct further comprises an HPV-18 E5 peptide. In some embodiments, the HPV-18 E5 peptide comprises a deletion of at least one amino acid as compared to a wildtype HPV-18 E5 peptide. In some embodiments, the deletion comprises the amino acids at residue positions 27-40 or the amino acids at residue positions 54-57 of the wildtype HPV-18 E5 peptide. In some embodiments, the HPV-18 E5 peptide has the sequence of SEQ ID NO: 50. In some embodiments, the polypeptide construct has the sequence of SEQ ID NO: 51. In some embodiments, the polypeptide construct further comprises at least one agonist peptide. In some embodiments, the at least one agonist peptide has an agonist peptide sequence as shown in Table 5. In some embodiments, the polypeptide construct has the sequence of SEQ ID NO: 53.

Provided herein is a polypeptide construct comprising an ankyrin-like repeat domain and an HPV peptide. In some embodiments, the ankyrin-like repeat protein is a human ankyrin-like repeat protein. In some embodiments, the HPV peptide is linked to the ankyrin-like repeat protein by a linker. In some embodiments, the polypeptide construct comprises an HPV-16 peptide and/or an HPV-18 peptide. In some embodiments, the polypeptide construct comprises an HPV-16 E5 peptide, an HPV-16 E6 peptide, and/or an HPV-16 E7 peptide. In some embodiments, the polypeptide construct comprises an HPV-18 E6 peptide and/or an HPV-18 E7 peptide. In some embodiments, the polypeptide construct comprises an HPV-16 E5 sequence, an HPV-16 E6 sequence, an HPV-16 E7 sequence, an HPV-18 E6 sequence and/or an HPV-18 E7 sequence as shown in Table 5. In some embodiments, the polypeptide construct has the sequence of SEQ ID NO: 52. In some embodiments, the polypeptide construct further comprises at least one agonist peptide. In some embodiments, the polypeptide construct comprises three agonist peptides. In some embodiments, the polypeptide construct has the sequence of SEQ ID NO: 54.

Provided herein is a polypeptide construct comprising at least two HPV peptides shown in Table 5 connected by a KK linker. In some embodiments, the polypeptide construct comprises at least one of an HPV-16 peptide or an HPV-18 peptide as shown in Table 5. In some embodiments, the polypeptide construct comprises an HPV-16 E5 peptide, an HPV-16 E6 peptide, and/or an HPV-16 E7 peptide as shown in Table 5. In some embodiments, the polypeptide construct comprises an HPV-18 E6 peptide and/or an HPV-18 E7 peptide as shown in Table 5. In some embodiments, the polypeptide construct comprises each of the peptides shown in Table 5. In some embodiments, each of such peptides is connected to another of such peptides by a KK linker. In some embodiments, the polypeptide construct has the sequence of SEQ ID NO: 55.

Provided herein is a polynucleotide encoding any of the presently described polypeptide constructs. Also provided herein is a vector comprising the polynucleotide. In some embodiments, the vector is an adenoviral vector. In some embodiments, said adenoviral vector is a gorilla adenoviral vector.

Provided herein is a vector, wherein the vector is an adenoviral vector that comprises a polynucleotide that encodes at least one HPV peptide.

Provided herein is a vector, wherein the vector is a gorilla adenoviral vector comprising a polynucleotide that encodes at least one HPV peptide.

In some embodiments, any of the polynucleotides and polypeptide constructs described herein is for use in a vaccine. Also provided herein is a vector comprising the polynucleotide. In some embodiments, the vector is an adenoviral vector. In some embodiments, the adenoviral vector is a gorilla adenoviral vector.

In some embodiments, the compositions and methods of the present invention can be combined with at least one additional therapy. In some embodiments, combination therapy involves administering the composition or vector expressing the novel HPV antigen designs disclosed herein before, during, or after the administration at least one other distinct therapeutic agent to enhance the treatment efficacy of various medical conditions, such as cancer. Such additional therapies include radiation therapy, surgery (e.g., debulking), chemotherapy, gene therapy, DNA therapy, virus therapy, RNA therapy, immunotherapy, bone marrow transplantation, nanotherapy, monoclonal antibody therapy, or a combination of the aforementioned therapies. The additional therapy may be in the form of an adjuvant or neoadjuvant therapy.

In some embodiments, the additional therapy involves the administration of an anti-inflammatory agent, analgesic, biological response modifier, cytokine, interferon, interleukin, CAR-T cell, bifunctional protein, colony stimulating factor, tumor necrosis factor, surface active agent, small molecule enzyme inhibitor, or anti-metastatic agent.

In some embodiments, the HPV vaccine antigens provided herein are delivered in combination with at least one interleukin. In one embodiment, the interleukin is IL-12. In some embodiments, IL-12 is delivered intratumorally.

In some embodiments, the HPV vaccine antigens provided herein are delivered in combination with a bifunctional fusion protein. In one embodiment, the bifunctional fusion protein is bintrafusp alfa (i.e., a bifunctional fusion protein composed of the extracellular domain of transforming growth factor (TGF)-βRII (a TGF-β “trap”) fused to a human IgG1 mAb blocking programmed cell death ligand 1).

In some embodiments, the HPV vaccine antigens provided herein are delivered in combination with at least one histone deacetylase inhibitor.

In some embodiments, the HPV vaccine antigens provided herein are delivered in combination with pembrolizumab.

In some embodiments, the HPV vaccine antigens provided herein are delivered in combination with docetaxel.

In some embodiments, the HPV vaccine antigens provided herein are delivered in combination with cisplatin.

In some embodiments, the HPV vaccine antigens provided herein are delivered in combination with docetaxel and cisplatin.

Provided herein is a polynucleotide encoding a fusion protein comprising (a) an HPV-16 E5 peptide or an HPV-18 E5 peptide; and (b) an HPV-16 E6 peptide, an HPV-16 E7 peptide, an HPV-18 E6 peptide, or an HPV-18 E7 peptide.

In some embodiments, any of the fusion proteins described herein comprises an HPV-16 E5 peptide comprising the amino acid sequence of SEQ ID NO: 130. In some embodiments, any of the fusion proteins described herein comprises an HPV-16 E6 peptide comprising the amino acid sequence of any one of SEQ ID NOs: 113-121. In some embodiments, any of the fusion proteins described herein comprises an HPV-18 E6 peptide comprising the amino acid sequence of any one of SEQ ID NOs: 131-138. In some embodiments, any of the fusion proteins described herein an HPV-16 E7 peptide comprising the amino acid sequence of any one of SEQ ID NOS: 122-129. In some embodiments, any of the fusion proteins described herein comprises an HPV-18 E7 peptide comprising the amino acid sequence of any one of SEQ ID NOs: 139-144.

In some embodiments, any of the fusion proteins described herein further comprises an agonist peptide. In some embodiments, the agonist peptide comprises an amino acid sequence of any one of SEQ ID NOs: 145-147.

In some embodiments, any of the fusion proteins described herein further comprises an ankyrin-like repeat domain. In some embodiments, the ankyrin-like repeat domain is located between two of the HPV peptides.

In some embodiments, any of the fusion proteins described herein comprises three or more HPV peptides and an ankyrin-like repeat domain between each of the HPV peptides. In some embodiments, any of the fusion proteins described herein comprises an amino acid sequence having at least 90% identity with SEQ ID NO: 243. In some embodiments, any of the fusion proteins described herein comprises an amino acid sequence having at least 95% identity with SEQ ID NO: 243. In some embodiments, any of the fusion proteins described herein comprises an amino acid sequence having at least 97% identity with SEQ ID NO: 243. In some embodiments, any of the fusion proteins described herein comprises an amino acid sequence having at least 98% identity with SEQ ID NO: 243. In some embodiments, any of the fusion proteins described herein comprises an amino acid sequence having at least 99% identity with SEQ ID NO: 243. In some embodiments, any of the fusion proteins described herein comprises the amino acid sequence of SEQ ID NO: 243 or a conservatively-substituted variant thereof. In some embodiments, tie fusion protein comprises the amino acid sequence of SEQ ID NO: 243.

In some embodiments, any of the fusion proteins described herein comprises an amino acid sequence having at least 90% identity with SEQ ID NO: 51. In some embodiments, any of the fusion proteins described herein comprises an amino acid sequence having at least 90% identity with SEQ ID NO: 52. In some embodiments, any of the fusion proteins described herein comprises an amino acid sequence having at least 90% identity with SEQ ID NO: 53.

In some embodiments, any of the fusion proteins described herein can be operably linked to at least one of: a promoter; a 5′ untranslated region (UTR); a transcription start site (TSS); a 3′ UTR; a tetracycline responsive element; and a kozak region. In some embodiments, the promoter is operably linked to a promoter enhancer region.

Provided herein is a vector comprising any one of the polynucleotides described herein. In some embodiments, the vector is a plasmid, a viral vector, or a non-viral vector. In some embodiments, the viral vector is an adenoviral vector. In some embodiments, the adenoviral vector is deficient in one or more elements selected from an E1-E4 region and an L1-L5 region. In some embodiments, the adenoviral vector comprises one or more elements selected from E2B, L1, L2, L3, E2A, L4, E3, L5, inverted terminal repeat (ITR), poly(a) site, and a spacer. In some embodiments, the adenoviral vector is a gorilla adenoviral vector. In some embodiments, the adenoviral vector is a GC46 gorilla adenoviral vector.

In some embodiments, any of the vectors described herein comprise a nucleic acid sequence having at least 90% identity with SEQ ID NO: 244.

Provided herein is a method of inducing an anti-HPV immune response in a subject in need thereof, the method comprising administering a therapeutically effective amount of any of the polynucleotides described herein to the subject. In some embodiments, the therapeutically effective amount comprises about 0.1×10⁹ to about 10×10¹² particle units of the vector.

Provided herein is a method of treating an HPV-associated disease or disorder in a subject in need thereof, the method comprising administering to the subject a therapeutically effective amount of any of the polynucleotides described herein. In some embodiments, the HPV-associated disease or disorder is a HPV-16 associated disease or disorder, an HPV-18 associated disease or disorder, or an HPV-45 associated disease or disorder. In some embodiments, the HPV-associated disease or disorder is a HPV-associated cancer. In some embodiments, the HPV-associated cancer is a lower genital tract neoplasia, a cervical cancer, a vulvar cancer, an anal cancer, a penile cancer, a head and neck cancer.

In any of the methods of treating an HPV-associated disease or disorder provided herein, the therapeutically effective amount comprises about 0.1×10⁹ to about 10×10¹² particle units of the vector.

In any of the methods of treating an HPV-associated disease or disorder provided herein, the method further comprises administering an additional therapy. In some embodiments, the additional therapy comprises the administration of at least one of the following: an anti-inflammatory agent; an analgesic; a biological response modifier; a cytokine; an interferon; an interleukin; a CAR-T cell; a bifunctional protein; a colony stimulating factor; a tumor necrosis factor; a surface active agent; a small molecule enzyme inhibitor; a chemotherapy agent; and an anti-metastatic agent. In some embodiments, the additional therapy comprises the administration of a biological response modifier. In some embodiments, the biological response modifier is an immune checkpoint inhibitor. In some embodiments, the immune checkpoint inhibitor is pembrolizumab. In some embodiments, the additional therapy comprises the administration of a chemotherapy agent. In some embodiments, the chemotherapy agent is a histone decetylase (HDAC) inhibitor.

Provided herein is a fusion protein encoded by any of the polynucleotides provided herein.

Provided herein is a composition comprising any of the polynucleotides provided herein.

Any of the compositions provided herein, including any of the polynucleotides, polypeptide constructs, and vectors is for use in treating a disease or disorder in a subject in need thereof.

Any of the compositions provided herein, including any of the polynucleotides, polypeptide constructs, and vectors is for use in the manufacture of a medicament for use in treating a disease or disorder in a subject in need thereof.

Provided herein is a kit comprising any of the polynucleotides, polypeptide constructs, or vectors provided herein.

Provided herein is a vaccine comprising any of the polynucleotides, polypeptide constructs, or vectors provided herein.

In some embodiments, any of the vaccines provided herein is for use in treating a disease or disorder in a subject in need thereof.

### BRIEF DESCRIPTION OF THE DRAWINGS

FIG. 1 is a schematic diagram of HPV genome. The HPV genome includes seven early genes (E1 to E7) and two late genes (L1 and L2), and each gene has specific functions. E5, E6, and E7 genes are associated with cancer development.

FIG. 2 is a schematic overall workflow implemented for designing HPV vaccine antigens.

FIG. 3 are schematic diagrams of HPV16 and HPV18 antigenic designs for HPV design 1 and HPV design 3. Consensus sequence information was utilized to select HPV16/HPV18 reference sequences for the design which included all major variants. The vaccine composition comprising different E6, E7 and E5 protein components with domain boundaries and mutation information is shown. These different domains contain most prevalent peptides deduced from Immune Epitope Database (IEDB) predictions for MHC-I binding. HPV design 3 is similar to HPV design 1 with the addition of enhancer agonist peptides.

FIG. 4A and FIG. 4B depict homology models of HPV design 2 and HPV design 4, respectively. A homology model is used to assess the overall structural feature and to compare the HPV design against native ankyrin repeats. HPV designs are shown in the same orientation and suggest different structural conformations due to shuffled peptides although maintaining the same overall fold.

FIG. 5A shows HPV design 5 (subject) (SEQ ID NO: 322) mapped onto HPV design 4 (Query) (SEQ ID NO: 321) using protein blast. Strong and weak binders were identified using netMHC. FIG. 5B shows density plots of HPV designs 4 and 5 which were extracted based on the mapped positions. Similar patterns in predicted strong/weak binding peptides were observed. The binding affinity predictions on the matched regions of HPV designs 4 and 5 were similar.

FIG. 6 is a schematic illustration showing short and long primer and probe sets generated for RNA qPCR relative expression assay. Specific primers were designed for each HPV antigen design.

FIG. 7A shows NetMHC 4.0 antigenicity predictions. Predicted strong and weak binding peptide indices are plotted against peptide locations. FIG. 7B shows NetMHC 4.0 antigenicity prediction density plot. First/second order differentials are employed in order to identify peaks. FIG. 7C shows amino acid sequences aligned against consensus sequences in order to determine coverage across HPV subtypes. FIG. 7C discloses SEQ ID NOS 288-314, respectively, in order of appearance.

FIG. 8 shows a comparison of HPV design 1 (SEQ ID NO: 323) and HPV design 3 (SEQ ID NO: 324).

FIG. 9A shows HPV16 E6 (SEQ ID NO: 45) sequence alignment to wildtype HPV16 E6 from UP P03126 (SEQ ID NO: 315). FIG. 9B shows HPV16 E7 (SEQ ID NO: 46) sequence alignment to wildtype HPV16 E7 from UP P03129 (SEQ ID NO: 316). FIG. 9C shows HPV16 E5 (SEQ ID NO: 47) sequence alignment to wildtype HPV16 E5 from UP P06927 (SEQ ID NO: 317).

FIG. 10A shows HPV18 E6 (SEQ ID NO: 48) sequence alignment to wildtype HPV18E6 from UP P06463 (SEQ ID NO: 318). FIG. 10B shows HPV18 E7 (SEQ ID NO: 49) sequence alignment to wildtype HPV18E7 from UP P06788 (SEQ ID NO: 319). FIG. 10C shows HPV18 E5 (SEQ ID NO: 50) sequence alignment to wildtype HPV18E5 from UP P06792 (SEQ ID NO: 320).

FIGS. 11A-B show the design of the recombinant gorilla adenovirus vaccine AdV-HPV16/18 (comprising the antigen construct of SEQ ID NO: 243), where FIG. 11A is a schematic representation of the multigene-deleted gorilla adenovector GC46, and FIG. 11B is a schematic structural representations of the HPV antigen constructs.

FIG. 12 depicts a GC46 vector engineer with E1 and E4 region deletions and the CMV HPV16/18 expression cassette (antigen) in the E1 region.

FIGS. 13A-B are AdV-HPV16/18 antigen schematics where FIG. 13A shows a predicted structure of the AdV-HPV16/18 antigen structure and FIG. 13B shows an AdV-HPV16/18 antigen with HPV epitopes/peptides and ankyrin-like linker/scaffold noted. FIG. 13B discloses SEQ ID NO: 243.

FIGS. 14A-G depict data collected from antitumor studies of AdV-HPV16/18 in a humanized mouse model. In FIGS. 14A and 14B, HPV-specific IFN-γ production from healthy-donor PBMCs were stimulated by autologous dendritic cells infected with HPV vaccine constructs, nos. 1-5, for 2 IVS cycles and restimulated twice with overlapping HPV16 E6/E7 15-mer peptides. IFN-γ levels in supernatants 24 and 48 hours after the second restimulation are shown for HPV16 E6 (FIG. 14A) and HPV16 E7 (FIG. 14B). In FIGS. 14C and 14D, NSG-β2m−/− mice bearing HPV+ cervical cancer (SiHa) were reconstituted with human PBMCs (day 7, blue arrow) and treated twice with weekly PBS (100 μL), empty vector (1×10⁹ VP), or HPV vaccine constructs (nos. 1, 3, and 4; 1×10⁹ VP) s.c. (red arrows). FIG. 14C shows tumor volumes, and FIG. 14D shows tumor weights on day 31. In FIGS. 14E and 14F, humanized NSG-β2m−/− mice bearing SiHa tumors were vaccinated 4 times with AdV-HPV16/18 (construct no. 4). FIG. 14E show tumor volumes, and FIG. 14F shows tumor weights on day 43. In FIG. 14G, tumors from FIG. 14C were dissected and stained using the Perkin Elmer Opal IHC kit. Central tumor sections are shown for a representative mouse from the empty vector and AdV-HPV16/18 groups. Red, CD8+ T cells; green, CD4+ T cells; blue, DAPI. Medians are shown. Kruskal-Wallis and repeated measures ANOVA. *P<0.05, **P<0.01, ****P<0.0001. EV, empty vector; IVS, in vitro stimulation; PBMCs, peripheral blood mononuclear cells; PBS, phosphate buffered saline; VP, virus particles. Scale bars: 100 μm.

In FIGS. 15A-15E, C57BL/6 mice bearing s.c. TC-1 HPV16⁺ tumors were treated with 3 weekly s.c. injections of PBS control (100 μL) or AdV-HPV16/18 (1×10⁹ VP), starting on day 4 after tumor implantation (red arrows). FIG. 15A shows average tumor volumes of AdV-HPV16/18 versus control-treated mice; P<0.0001. FIG. 15B shows tumor weights (medians) at the end of study; P<0.05. Mice nos. 10 and 13 were defined as Best Responders, with tumor weights below the group median, and mice nos. 11 and 14 were defined as Nonresponders, with tumor weights above the group median. FIGS. 15C and 15D show flow cytometry of single-cell suspensions of tumors. In FIG. 15C, CD8⁺ and CD4⁺ T cell tumor infiltration are shown from a representative mouse from each group. FIG. 15D shows multifunctional (IFN-γ⁺GzmB⁺) CD8⁺ T cell infiltration from 1 representative mouse from each group. In FIG. 15E, splenocytes from 6 mice from each group were stimulated with overlapping HPV16 E6 15-mer peptides in an ELIspot assay. FIG. 15E shows the number of SFCs per 2.5×10⁵ splenocytes of AdV-HPV16/18-versus control-treated mice after deducting the negative control. Mann-Whitney U and repeated measures ANOVA tests were used. *P<0.05, **P<0.01, ****P<0.0001. SFCs, spot-forming cells; VP, virus particles.

In FIGS. 16A-F, C57BL/6 mice (n=7-8 per group) bearing s.c. TC-1 HPV16⁺ murine tumors were treated in 2 separate studies with empty vector control (1×10⁹ VP, s.c.), PBS (100 μL, s.c.), or AdV-HPV16/18 (1×10⁹ VP, s.c.) on days 7 and 14 after tumor implantation. FIG. 16A shows study 1, end-of-study tumor weights for individual mice. FIG. 16B shows study 2, end-of-study tumor weights for individual mice. FIG. 16C is a meta-analysis of both studies, showing end-of-study tumor weights. FIGS. 16D-18F show flow cytometry of single-cell suspensions of tumor tissue. T cell subsets are shown per mg tumor. The Ki67 stain was only performed in 1 study. FIGS. 16A-F show the medians. Mann-Whitney U and Kruskal-Wallis tests were used. *P<0.05, **P<0.01, ***P<0.001. VP, virus particles.

In FIGS. 17A-I, C57BL/6 mice (n=6-7 per group) bearing s.c. TC-1 HPV16 tumors were treated with empty vector control (1×10⁹ VP, s.c.) or AdV-HPV16/18 (1×10⁹ VP, s.c.) on days 7 and 14 after tumor implantation. At the end of study (day 23), tumors were harvested. CD45⁺ TILs were isolated and stimulated overnight in triplicate wells with combined overlapping HPV16 E6/E7 15-mer peptides or DMSO (negative control), and they were then evaluated by flow cytometry. FIGS. 17A-F show the number of cells/mg tumor for total CD8 (FIG. 17A), IFN-γ-producing CD8 (FIG. 17B), IFN-γ plus GzmB-producing CD8 (FIG. 17C), total CD4 (FIG. 17D), IFN-γ-producing CD4 (FIG. 17E), and IFN-γ plus GzmB-producing CD4 (FIG. 17F). In FIG. 17G, unstimulated TILs were also evaluated for CD8 and HPV16 E7 tetramer. FIGS. 17H and 17I show an ELIspot assay that was performed on splenocytes using overlapping 15-mer peptides for HPV16 E6 (FIG. 17H) and HPV18 E6 (FIG. 17I). Medians are shown. Mann-Whitney U test was used. *P<0.05, **P<0.01. TILs, tumor-infiltrating lymphocytes; TME, tumor microenvironment; VP, virus particles.

FIG. 18 shows tumor volumes where C57BL/6 mice bearing TC-1 tumors were treated with AdV-HPV16/18 alone or in combination with bintrafusp alfa, an anti-PD-L1/TGF-β trap protein, and assessed for primary tumor growth.

FIG. 19 shows a schema for administration of AdV-HPV16/18 alone or in combination with bintrafusp alfa, an anti-PD-L1/TGF-β trap protein, in in subjects with HPV associated cancers.

FIG. 20 is a bar graph showing time to response and duration of response to treatment following administration of AdV-HPV16/18 alone and in combination with bintrafusp alfa in patients with recurrent/metastatic HPV-associated cancers.

FIG. 21A is a dot plot graph showing neutralizing anti-GC46 antibody titers relative to the number of AdV-HPV16/18 vaccinations administered during following administration of AdV-HPV16/18 alone. FIG. 21B is a dot plot graph showing neutralizing anti-GC46 antibody titers relative to the number of AdV-HPV16/18 vaccinations following administration of AdV-HPV16/18 and bintrafusp alfa.

FIG. 22A shows a CT scan of the cervix of an individual cervical cancer patient who had a complete response to combination therapy with AdV-HPV16/18 and bintrafusp alfa. FIG. 22B is a bar graph showing the same patient's T-cell activity relative to the number of AdV-HPV16/18 vaccinations administered.

FIG. 23 shows a schema for administration of AdV-HPV16/18 alone or in combination with bintrafusp alfa, an anti-PD-L1/TGF-β trap protein, in subjects with HPV associated cancers./18 vaccinations following administration of AdV-HPV16/18 and bintrafusp alfa.

FIG. 24 shows development of HPV-specific T cell development of HPV-specific T cell responses in patients where no such pre-existing responses were detected for (A) HPV16 and (B) HPV18. The timepoint (in days) evaluated for each patient is indicated along the x-axis, and the CD4+ or CD8+ phenotype is indicated in the legend.

FIG. 25 shows the absolute number of T cells producing cytokines and/or CD107a (left y-axis) in response to (A) HPV16 and (B) HPV18 peptides in the patient experiencing a complete response with treatment through treatment and beyond one year. Vector neutralizing antibody titer at available timepoints plotted on right y-axis (black square). The production of anti-GC46 vector-neutralizing antibodies did not abrogate development of HPV specific T cell responses. The timepoint (in days) is indicated along the x-axes, and the CD4+ or CD8+ phenotype readout is indicated in the legend.

FIG. 26 shows an increase in both activated CD8+ memory T cells and mature NK cells as early as 2 weeks after first vaccine dose of AdV-HPV16/18 only seen in patient achieving complete response (CR). Percent change in (A) total CD8+ T cell frequencies and (B) total NK cell frequencies at D15 compared to baseline.

FIG. 27 shows levels of (A) IL-8 and (B) TNFα during the first 8 weeks on study. IL-8 and TNFα levels significantly increased two weeks after first treatment with AdV-HPV16/18 and bintrafusp alfa.

FIG. 28 shows Kaplan-Meier survival curves by Mantel-Cox log-rank tests for (A) progression-free survival (PFS) and (B) overall survival (OS) for patients treated with AdV-HPV16/18 in combination with bintrafusp alfa and shows that lower serum concentrations of IL-8 prior to start of treatment associated with prolonged survival. The cutoff was set at 23.0 pg/ml.

FIG. 29 shows changes in HPV circulating tumor DNA (ctDNA) during treatment with AdV-HPV16/18. (A) Absolute copies of HPV16 or HPV18 ctDNA per ml of plasma. (B) Percent changes in ctDNA levels during treatment compared to baseline. Best observed response (BOR) and HPV genotype indicated in legend.

FIG. 30 shows a sequence alignment comparing HPV45 E6 protein (SEQ ID NO: 325) with the following HPV16 E6 or HPV18 E6 epitopes: HPV16 E6 peptide 5 (pep5) (SEQ ID NO: 117), HPV16 E6 peptide 7 (SEQ ID NO: 119), HPV16 E6 peptide 8 (SEQ ID NO: 120), HPV18 E6 peptide 1 (SEQ ID NO: 131), HPV18 E6 peptide 2 (SEQ ID NO: 132), HPV18 E6 peptide 4 (SEQ ID NO: 184), HPV18 E6 peptide 5 (SEQ ID NO: 135), and Agonist peptide 1 (SEQ ID NO: 176).

FIG. 31 shows a sequence alignment comparing HPV45 E7 protein (SEQ ID NO: 326) with the following HPV18 E7 epitopes: HPV18 E7 peptide 1 (pep1) (SEQ ID NO: 226), HPV18 E7 peptide 2 (SEQ ID NO: 194), HPV18 E7 peptide 3 (SEQ ID NO: 192), HPV18 E7 peptide 4 (SEQ ID NO: 186), HPV18 E7 peptide 5 (SEQ ID NO: 240), and HPV18 E7 peptide 6 (SEQ ID NO: 182).

FIG. 32 shows a sequence alignment comparing HPV45 E7 protein (SEQ ID NO: 326) with the following HPV18 E7 epitopes: HPV18 E7 peptide 1 (SEQ ID NO: 226), HPV18 E7 peptide 4 (SEQ ID NO: 186), and HPV18 E7 peptide 6 (SEQ ID NO: 182).

### DETAILED DESCRIPTION OF THE INVENTION

It is to be understood that the present disclosure is not limited to the particular embodiments described herein and as such can vary. Although various features of the disclosure can be described in the context of a single embodiment, the features can also be provided separately or in any suitable combination. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

### Definitions

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Use of the term “including” as well as other forms, such as “include,” “includes,” and “included,” is not limiting; i.e., “including” does not mean “limited to.”

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional possible components, elements, or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The term “isolated” and its grammatical equivalents as used herein refer to the removal of a nucleic acid, protein, polypeptide, cell, or other material from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” It is to be understood, however, that nucleic acids and proteins can be formulated with diluents or adjuvants and still for practical purposes be isolated. For example, nucleic acids typically are mixed with an acceptable carrier or diluent when used for introduction into cells. The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

“Nucleic acid,” “nucleic acid molecule,” “polynucleotide,” “polynucleotide construct,” “oligonucleotide,” and their grammatical equivalents as used herein refer to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA It also includes modified, for example, by methylation and/or by capping, and unmodified forms of the polynucleotide. The term is also meant to include molecules that include non-naturally occurring, synthetic, and semi-synthetic nucleotides and polynucleotides as well as nucleotide analogs. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant polynucleotide” is a polynucleotide that has undergone a molecular biological manipulation. The polynucleotide sequences and vectors disclosed or contemplated herein can be introduced into a cell by, for example, transfection, transformation, or transduction.

The term “fragment,” as applied to a polynucleotide or nucleic acid sequence, refers to a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 6, 8, 9, 10, 12, 15, 18, 20, 21, 22, 23, 24, 25, 30, 39, 40, 42, 45, 48, 50, 51, 54, 57, 60, 63, 66, 70, 75, 78, 80, 90, 100, 105, 120, 135, 150, 200, 300, 500, 720, 900, 1000, 1500, 2000, 3000, 4000, 5000, or more consecutive nucleotides of a nucleic acid according to the invention.

As used herein, an “isolated polynucleotide” or “isolated nucleic acid fragment” refers to a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

The term “gene” and its grammatical equivalents refers to a polynucleotide comprising nucleotides that encode a functional molecule, including functional molecules produced by transcription only (e.g., a bioactive RNA species) or by transcription and translation (e.g., a polypeptide). The term “gene” encompasses cDNA and genomic DNA nucleic acids. “Gene” also refers to a nucleic acid fragment that expresses a specific RNA, protein or polypeptide, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “genome” includes chromosomal as well as mitochondrial, chloroplast and viral DNA or RNA. The term “probe” refers to a single-stranded nucleic acid molecule that can base pair with a complementary single stranded target nucleic acid to form a double-stranded molecule.

“Heterologous DNA” refers to DNA not naturally located in the cell, or in a chromosomal site of the cell. The heterologous DNA may include an exogenous gene. “Exogenous gene” means a gene foreign to the subject, that is, a gene which is introduced into the subject through a transformation process, an unmutated version of an endogenous mutated gene or a mutated version of an endogenous unmutated gene. Exogenous genes can be either natural or synthetic genes which are introduced into the subject in the form of DNA or RNA which may function through a DNA intermediate such as by reverse transcriptase. Such genes can be introduced into target cells, directly introduced into the subject, or indirectly introduced by the transfer of transformed cells into the subject.

A “primer” refers to an oligonucleotide that hybridizes to a target nucleic acid sequence to create a double stranded nucleic acid region that can serve as an initiation point for DNA synthesis under suitable conditions. Such primers may be used in a polymerase chain reaction or for DNA sequencing.

A DNA “coding sequence” or “coding region” refers to a double-stranded DNA sequence that encodes a polypeptide and can be transcribed and translated into a polypeptide in a cell, ex vivo, in vitro or in vivo when placed under the control of suitable regulatory sequences. “Suitable regulatory sequences” refers to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures. The boundaries of the coding sequence are determined by a start codon at the 5′ (amino) terminus and a translation stop codon at the 3′ (carboxy) terminus. A coding sequence can include, but is not limited to, prokaryotic sequences, cDNA from mRNA, genomic DNA sequences, and even synthetic DNA sequences. If the coding sequence is intended for expression in an eukaryotic cell, a polyadenylation signal and transcription termination sequence will usually be located 3′ to the coding sequence.

“Open reading frame” is abbreviated ORF and refers to a length of nucleic acid sequence, either DNA, cDNA or RNA, that comprises a translation start signal or initiation codon, such as an ATG or AUG, and a termination codon and can be potentially translated into a polypeptide sequence.

The term “downstream” refers to a nucleotide sequence that is located 3′ to a reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to a reference nucleotide sequence. In particular, upstream nucleotide sequences generally relate to sequences that are located on the 5′ side of a coding sequence or starting point of transcription. For example, most promoters are located upstream of the start site of transcription.

The term “response element” refers to one or more cis-acting DNA elements which confer responsiveness on a promoter mediated through interaction with the DNA-binding domains of a transcription factor. This DNA element may be either palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element is incorporated. The DNA binding domain of the transcription factor binds, in the presence or absence of a ligand, to the DNA sequence of a response element to initiate or suppress transcription of downstream gene(s) under the regulation of this response element. Examples of DNA sequences for response elements of the natural ecdysone receptor include: RRGG/TTCANTGAC/ACYY (SEQ ID NO: 275) (see Cherbas et. al., Genes Dev. 1991); AGGTCAN₍ₙ₎ AGGTCA, where N₍ₙ₎ can be one or more spacer nucleotides (SEQ ID NO: 276) (see D'Avino et al., Mol. Cell. Endocrinol. 113:1 1995); and GGGTTGAATGAATTT (SEQ ID NO: 277) (see Antoniewski et al., Mol. Cell Biol. 14:4465 1994).

The term “operably linked” as used herein refers to refers to the physical and/or functional linkage of a DNA segment to another DNA segment in such a way as to allow the segments to function in their intended manners. A DNA sequence encoding a gene product is operably linked to a regulatory sequence when it is linked to the regulatory sequence, such as, for example, promoters, enhancers and/or silencers, in a manner which allows modulation of transcription of the DNA sequence, directly or indirectly. For example, a DNA sequence is operably linked to a promoter when it is ligated to the promoter downstream with respect to the transcription initiation site of the promoter, in the correct reading frame with respect to the transcription initiation site and allows transcription elongation to proceed through the DNA sequence. An enhancer or silencer is operably linked to a DNA sequence coding for a gene product when it is ligated to the DNA sequence in such a manner as to increase or decrease, respectively, the transcription of the DNA sequence. Enhancers and silencers can be located upstream, downstream or embedded within the coding regions of the DNA sequence. A DNA for a signal sequence is operably linked to DNA coding for a polypeptide if the signal sequence is expressed as a pre-protein that participates in the secretion of the polypeptide. Linkage of DNA sequences to regulatory sequences is typically accomplished by ligation at suitable restriction sites or via adapters or linkers inserted in the sequence using restriction endonucleases known to one of skill in the art.

As used herein, the term “codon degenerate variant” refers to a modified nucleic acid sequence that encodes the same amino acid sequence as the original sequence but differs in the specific nucleotides comprising the codons. The genetic code is degenerate, meaning that multiple codons can code for the same amino acid. For example, the amino acid leucine can be encoded by six different codons: CTG, CTT, CTC, CTA, TTG, and TTA. A codon degeneracy table, also known as a genetic code table or codon table, is a chart that provides information about the relationship between codons (sequences of three nucleotides) and the corresponding amino acids they encode. The table lists the 64 possible codons and indicates which amino acid each codon represents. Table 1 is an example of a codon degeneracy table:

TABLE 1
Codon Degeneracy Table
Nucleotide Position in Codon - Amino Acid Encoded

| First | Second nucleotide | | | | Third |
|---|---|---|---|---|---|
| nucleotide | U | C | A | G | nucleotide |
| U | UUU - Phe | UCU - Ser | UAU - Tyr | UGU - Cys | U |
| | UUC - Phe | UCC - Ser | UAC - Tyr | UGC - Cys | C |
| | UUA - Leu | UCA - Ser | UAA - * | UGA - * | A |
| | UUG - Leu | UCG - Ser | UAG - * | UGG - Trp | G |
| C | CUU - Leu | CCU - Pro | CAU - His | CGU - Arg | U |
| | CUC - Leu | CCC - Pro | CAC - His | CGC - Arg | C |
| | CUA - Leu | CCA - Pro | CAA - Gln | CGA - Arg | A |
| | CUG - Leu | CCG - Pro | CAG - Gln | CGG - Arg | G |
| A | AUU - Ile | ACU - Thr | AAU - Asn | AGU - Ser | U |
| | AUC - Ile | ACC - Thr | AAC - Asn | AGC - Ser | C |
| | AUA - Ile | ACA - Thr | AAA - Lys | AGA - Arg | A |
| | AUG - Met | ACG - Thr | AAG - Lys | AGG - Arg | G |
| G | GUU - Val | GCU - Ala | GAU - Asp | GGU - Gly | U |
| | GUC - Val | GCC - Ala | GAC - Asp | GGC - Gly | C |
| | GUA - Val | GCA - Ala | GAA - Glu | GGA - Gly | A |
| | GUG - Val | GCG - Ala | GAG - Glu | GGG - Gly | G |

(U indicating “Uracil” as incorporated into mRNA in place of T (Thymine) as found in DNA; both of which form complementary base-pairs with “A” (Adenine); “*” indicates stop codons.)

The following definitions supplement those in the art and are directed to the current application and are not to be imputed to any related or unrelated case, e.g., to any commonly owned patent or application. Accordingly, the terminology used herein is for the purpose of describing particular embodiments only, and is not intended to be limiting.

Furthermore, publicly available software resources are readily available for computer-generated “reverse-translation,” also known as, “back translation” of polypeptide sequences, i.e., converting polypeptide sequences into nucleotide sequences encoding same). See, e.g., Madeira, F., et al., Nucleic Acids Res, 47 (W1), W636-W641 (2019); Madeira, F., et al., Curr Protoc in Bioinformatics, 66 (1): c74 (2019); Chojnacki, S, et al., Nucleic Acids Res. 2017 Jul. 3; 45 (W1): W550-W553 (2017); Athey, J., et al., BMC Bioinformatics 18:391 (2017).

As used herein, a codon degenerate variant may be utilized to optimize gene expression or enhance protein production. By modifying the codons within a nucleic acid sequence, it is possible to utilize codons that are more frequently used or preferred by the host organism's translational machinery. This can lead to increased efficiency in protein expression or improved compatibility with a specific host organism.

The term “expression” as used herein refers to the transcription and stable accumulation of sense (mRNA) or antisense RNA derived from a nucleic acid or polynucleotide. Expression may also refer to translation of mRNA into a protein or polypeptide.

The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like. The expression cassettes described herein may comprise a total length of between about 500 to about 10,000 bp, about 1,000 to about 5,000 bp, about 1,500 to about 4,500 bp, about 1,800 to about 4,400 bp, about 2,000 to about 4,500 bp, about 2,100 to about 4,400 bp, about 2,200 to about 4,300 bp, about 2,300 to about 4,200 bp, about 2,400 to about 4,100 bp, about 2,500 to about 4,000 bp, about 2,600 to about 3,900 bp, about 2700 to about 3800 bp, about 2,800 to about 3,800 bp, about 2,900 to about 3,700 bp, about 3,000 to about 3,600 bp, about 3,100 to about 3,500 bp, about 3,150 to about 3,450 bp, about 3,200 to about 3,400 bp, about 3,250 to about 3,350 bp, or about 3300 bp. Alternatively, the expression cassette may comprise any number of base pairs falling within these ranges. For instance, the expression cassette may comprise about 500 bp, about 750 bp, about 1,000 bp, about 1,250 bp, about 1,500 bp, about 1,750 bp, about 2,000 bp, about 2,250 bp, about 2,500 bp, about 2,550 bp, about 2,600 bp, about 2,650 bp, about 2,700 bp, about 2,750 bp, about 2,800 bp, about 2,850 bp, about 2,900 bp, about 2,950 bp, about 3000 bp, about 3,050 bp, about 3,100 bp, about 3,150 bp, about 3,200 bp, about 3,250 bp, about 3,300 bp, about 3,350 bp, about 3,400 bp, about 3450 bp, about 3,500 bp, about 3,550 bp, about 3,600 bp, about 3,650 bp, about 3,700 bp, about 3,750 bp, about 3,800 bp, about 3,850 bp, about 3,900 bp, about 3,950 bp, about 4,000 bp, about 4,050 bp, about 4,100 bp, about 4,150 bp, about 4,200 bp, about 4,250 bp, about 4,300 bp, about 4,350 bp, about 4,400 bp, about 4,450 bp, or about 4,500 bp. In one aspect, the expression cassette comprises about 2,800 bp. In another aspect, the expression comprises 2,825 bp.

As used herein, the term “vector” refers to any vehicle for the cloning of and/or transfer of a nucleic acid into a host cell. A vector may be a replicon to which another DNA segment may be attached so as to bring about the replication of the attached segment. A “replicon” refers to any genetic element (e.g., plasmid, phage, cosmid, chromosome, virus) that functions as an autonomous unit of DNA replication in vivo, i.e., capable of replication under its own control. The term “vector” includes both, viral and nonviral vehicles for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as pBR322 or pUC plasmid derivatives, or the Bluescript vector. Another example of vectors that are useful in the invention is the ULTRAVECTOR® Production System (Intrexon Corp., Blacksburg, VA) as described in WO 2007/038276. For example, the insertion of the DN fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

As used herein, the term “plasmid” refers to an extra-chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the terms “cloning vector” and “replicon” refer to a unit length of a nucleic acid, preferably DNA, that replicates sequentially and which comprises an origin of replication, such as a plasmid, phage or cosmid, to which another nucleic acid segment may be attached so as to bring about the replication of the attached segment. Cloning vectors may be capable of replication in one cell type and expression in another (“shuttle vector”). Cloning vectors may comprise one or more sequences that can be used for selection of cells comprising the vector and/or one or more multiple cloning sites for insertion of sequences of interest.

As used herein, the term “viral vector” as used herein refers to a virus, viral particle, or derivative thereof, capable of transferring a nucleic acid into a cell or to the transferred nucleic acid itself. Viral vectors and transfer plasmids contain structural and/or functional genetic elements that are primarily derived from a virus. Viral vectors, and particularly retroviral vectors, have been used in a wide variety of gene delivery applications in cells, as well as living animal subjects. Viral vectors that can be used include, but are not limited to, retrovirus, adeno-associated virus, pox, baculovirus, vaccinia, herpes simplex, Epstein-Barr, adenovirus, geminivirus, and caulimovirus vectors. Non-viral vectors include plasmids, liposomes, electrically charged lipids (cytofectins), DNA-protein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

As used herein, the term “adenovirus” and “adenoviral vector” as used herein, refers to an adenovirus that retains the ability to participate in the adenovirus life cycle and/or which has been physically inactivated by, for example, disruption (e.g., sonication), denaturing (e.g., using heat or solvents), or cross-linkage (e.g., via formalin cross-linking). The “adenovirus life cycle” includes (1) virus binding and entry into cells, (2) transcription of the adenoviral genome and translation of adenovirus proteins, (3) replication of the adenoviral genome, and (4) viral particle assembly (see, e.g., Fields Virology, 5^{th} ed., Knipe et al. (eds.), Lippincott Williams & Wilkins, Philadelphia, PA (2006)). Adenoviruses, as used and described herein may also be rendered replication deficient (i.e., do not retain ability to participate in the adenovirus life cycle) by deletion of one or more parts of the naturally occurring viral genome. “Adenoviruses” and “Adenoviral vector,” as used and described herein, may include an adenovirus in which the adenoviral genome has been manipulated to accommodate a nucleic acid sequence that is non-native with respect to the adenoviral genome. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

As used herein, “AdV-HPV16/18” refers to a vector comprising the antigen construct of SEQ ID NO: 243.

As used herein, the terms “MOI” or “Multiplicity of Infection” refer to the average number of virus particles that infect a single cell in a specific experiment (e.g., recombinant virus or control virus).

As used herein, the term “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, the term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, the term “electroporation” refers to the use of a transmembrane electric field pulse to transiently increase the permeability of a cell membrane, allowing the introduction of exogenous biological materials, such as DNA, RNA, peptides, polypeptides, proteins, enzymes, or ribonucleoproteins (RNPs), into the cell. The electric field pulse creates transient pores in the cell membrane, facilitating the uptake of the biological material. Electroporation can be performed using specialized buffers and devices that control the pH, conductivity, osmolality, and other parameters to optimize the process and enhance transfection efficiency while minimizing cell damage. Electroporation can be used to introduce exogenous materials (e.g., biomolecules, plasmids, oligonucleotides, expression cassettes, siRNA, drugs, and ions) into various cell types, including primary human blood cells, immune cells, pluripotent precursor cells, fibroblasts, and endothelial cells, for applications in gene therapy, cell therapy, and biotechnology research.

As used herein, the term terms “induce,” “induction” and their grammatical equivalents as used herein refer to an increase in nucleic acid sequence transcription, promoter activity and/or expression brought about by a transcriptional regulator, relative to some basal level of transcription.

As used herein, the terms “promoter” and “promoter sequence” are used interchangeably and refer to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths may have identical promoter activity.

The promoter sequence is typically bounded at its 3′ terminus by the transcription initiation site and extends upstream (5′ direction) to include the minimum number of bases or elements necessary to initiate transcription at levels detectable above background. Within the promoter sequence is found a transcription initiation site (conveniently defined for example, by mapping with nuclease SI), as well as protein binding domains (consensus sequences) responsible for the binding of RNA polymerase.

The source of the promoter can be natural or synthetic, and the source of the promoter should not limit the scope of the invention described herein. In other words, the promoter may be directly cloned from cells, or the promoter may have been previously cloned from a different source, or the promoter may have been synthesized.

As used herein, the term “transcriptional regulator” refers to a biochemical element that acts to prevent or inhibit the transcription of a promoter-driven DNA sequence under certain environmental conditions (e.g., a repressor or nuclear inhibitory protein), or to permit or stimulate the transcription of the promoter-driven DNA sequence under certain environmental conditions (e.g., an inducer or an enhancer).

As used herein, the term “enhancer” refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

As used herein, the term “therapeutic switch promoter” (“TSP”) refers to a promoter that controls expression of a gene switch component. Gene switches and their various components are described in detail elsewhere herein. In certain embodiments a TSP is constitutive, i.e., continuously active. A constitutive TSP may be either constitutive-ubiquitous (i.e., generally functions, without the need for additional factors or regulators, in any tissue or cell) or constitutive-tissue or cell specific (i.e., generally functions, without the need for additional factors or regulators, in a specific tissue type or cell type). In certain embodiments a TSP of the invention is activated under conditions associated with a disease, disorder, or condition. In certain embodiments of the invention where two or more TSPs are involved the promoters may be a combination of constitutive and activatable promoters. As used herein, a “promoter activated under conditions associated with a disease, disorder, or condition” includes, without limitation, disease-specific promoters, promoters responsive to particular physiological, developmental, differentiation, or pathological conditions, promoters responsive to specific biological molecules, and promoters specific for a particular tissue or cell type associated with the disease, disorder, or condition, e.g. tumor tissue or malignant cells. TSPs can comprise the sequence of naturally occurring promoters, modified sequences derived from naturally occurring promoters, or synthetic sequences (e.g., insertion of a response element into a minimal promoter sequence to alter the responsiveness of the promoter).

Therapeutic switch promoters useful in the invention may include any promoter that is useful for treating, ameliorating, or preventing a specific disease, disorder, or condition. Examples include, without limitation, promoters of genes that exhibit increased expression only during a specific disease, disorder, or condition and promoters of genes that exhibit increased expression under specific cell conditions (e.g., proliferation, apoptosis, change in pH, oxidation state, oxygen level). In some embodiments where the gene switch comprises more than one transcription factor sequence, the specificity of the therapeutic methods can be increased by combining a disease- or condition-specific promoter with a tissue- or cell type-specific promoter to limit the tissues in which the therapeutic product is expressed. Thus, tissue- or cell type-specific promoters are encompassed within the definition of therapeutic switch promoter.

The term “ecdysone receptor-based,” with respect to a gene switch, refers to a gene switch comprising at least a functional part of a naturally occurring or synthetic ecdysone receptor ligand binding domain and which regulates gene expression in response to a ligand that binds to the ecdysone receptor ligand binding domain. Examples of ecdysone-responsive systems are described in U.S. Pat. Nos. 7,091,038 and 6,258,603. In one embodiment, the system is the RheoSwitch® Therapeutic System (RTS), which contains two fusion proteins, the DEF domains of a mutagenized ecdysone receptor (EcR) fused with a Gal4 DNA binding domain and the EF domains of a chimeric RXR fused with a VP16 transcription activation domain, expressed under a constitutive promoter.

A coding sequence is “under the control” of transcriptional and translational control sequences in a cell when RNA polymerase transcribes the coding sequence into mRNA, which is then trans-RNA spliced (if the coding sequence contains introns) and translated into the protein encoded by the coding sequence.

“Transcriptional and translational control sequences” refer to DNA regulatory sequences, such as promoters, enhancers, terminators, and the like, that provide for the expression of a coding sequence in a host cell. In eukaryotic cells, polyadenylation signals are control sequences. Enhancers that may be used in embodiments of the invention include but are not limited to: an SV40 enhancer, a cytomegalovirus (CMV) enhancer, an elongation factor 1 (EF 1) enhancer, yeast enhancers, viral gene enhancers, and the like.

The terms “3′ non-coding sequences” and “3′ untranslated region (UTR)” refer to DNA sequences located downstream (3′) of a coding sequence and may comprise polyadenylation [poly(A)] recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

As used herein, the term “regulatory region” refers to a nucleic acid sequence that regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include origins of replication, RNA splice sites, promoters, enhancers, transcriptional termination sequences, and signal sequences which direct the polypeptide into the secretory pathways of the target cell.

As used herein, the term “modulate” means to induce, reduce or inhibit nucleic acid or gene expression, resulting in the respective induction, reduction or inhibition of protein or polypeptide production.

As used herein, the term “inducible promoter” refers to a promoter which is induced into activity by the presence or absence of transcriptional regulators, e.g., biotic or abiotic factors. Inducible promoters are useful because the expression of genes operably linked to them can be turned on or off at certain stages of development of an organism or in a particular tissue. Non-limiting examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. The inducible promoter can be part of a gene switch or genetic switch. The inducible promoter can be a gene switch ligand inducible promoter. In some cases, an inducible promoter can be a small molecule ligand-inducible two polypeptide ecdysone receptor-based gene switch. In some cases, a gene switch can be selected from ecdysone-based receptor components as described in, but without limitation to, any of the systems described in: International Patent Applications WO 2001/070816; WO 2002/029075; WO 2002/066613; WO 2002/066614; WO 2002/066612; WO 2002/066615; WO 2003/027266; WO 2003/027289; WO 2005/108617; WO 2009/045370; WO 2009/048560; WO 2010/042189; WO 2010/042189; WO 2011/119773; and WO 2012/122025; and U.S. Pat. Nos. 7,091,038; 7,776,587; 7,807,417; 8,202,718; 8,105,825; 8,168,426; 7,531,326; 8,236,556; 8,598,409; 8,715,959; 7,601,508; 7,829,676; 7,919,269; 8,030,067; 7,563,879; 8,021,878; 8,497,093; 7,935,510; 8,076,454; 9,402,919; 9,493,540; 9,249,207; and 9,492,482, each of which is incorporated by reference in its entirety.

As used herein, two or more individually operable gene regulation systems are said to be “orthogonal” when: a) modulation of each of the given systems by its respective ligand, at a chosen concentration, results in a measurable change in the magnitude of expression of the gene of that system, and b) the change is statistically significantly different than the change in expression of all other systems simultaneously operable in the cell, tissue, or organism, regardless of the simultaneity or sequentiality of the actual modulation. Preferably, modulation of each individually operable gene regulation system effects a change in gene expression at least 2-fold greater than all other operable systems in the cell, tissue, or organism, e.g., at least 5-fold, 10-fold, 100-fold, or 500-fold greater. Ideally, modulation of each of the given systems by its respective ligand at a chosen concentration results in a measurable change in the magnitude of expression of the gene of that system and no measurable change in expression of all other systems operable in the cell, tissue, or organism. In such cases the multiple inducible gene regulation system is said to be “fully orthogonal.” Useful orthogonal ligands and orthogonal receptor-based gene expression systems are described in US 2002/0110861 A1.

As used herein, the term “gene switch” as used herein refers to the combination of a response element associated with a promoter, and a ligand-dependent transcription factor-based system which, in the presence of one or more ligands, modulates the expression of a gene into which the response element and promoter are incorporated. The term “a polynucleotide encoding a gene switch” refers to the combination of a response element associated with a promoter, and a polynucleotide encoding a ligand-dependent transcription factor-based system which, in the presence of one or more ligands, modulates the expression of a gene into which the response element and promoter are incorporated. Tightly regulated inducible gene expression systems or gene switches, such as EcR based systems, are useful for various applications such as gene therapy, large scale production of proteins in cells, cell based high throughput screening assays, functional genomics and regulation of traits in transgenic plants and animals. Such inducible gene expression systems can include ligand inducible heterologous gene expression systems.

As used herein, the term “CAP” or “cap” refers to a modified nucleotide, generally a 7-methyl guanosine, linked 3′ to 5′ (7meG-ppp-G), to the 5′ end of a eukaryotic mRNA, that serves as a required element in the normal translation initiation pathway during expression of protein from that mRNA.

As used herein, the term “Sleeping Beauty (SB) Transposon System” refers a synthetic DNA transposon system for to introducing DNA sequences into the chromosomes of vertebrates. Some exemplary embodiments of the system are described, for example, in U.S. Pat. Nos. 6,489,458, 8,227,432, 9,228,180 and WO/2016/145146. The Sleeping Beauty transposon system is composed of a Sleeping Beauty (SB) transposase and a SB transposon. In embodiments, the Sleeping Beauty transposon system can include the SB11 transposon system, the SB100X transposon system, or the SB110 transposon system.

As used herein, the term “transposon” or “transposable element” (TE) refers to a vector DNA sequence that can change its position within the genome, sometimes creating or reversing mutations and altering the cell's genome size. Transposition often results in duplication of the TE. Class I TEs are copied in two stages: first they are transcribed from DNA to RNA, and the RNA produced is then reverse transcribed to DNA This copied DNA is then inserted at a new position into the genome. The reverse transcription step is catalyzed by a reverse transcriptase, which can be encoded by the TE itself The characteristics of retrotransposons are similar to retroviruses, such as HIV. The cut-and-paste transposition mechanism of class II TEs does not involve an RNA intermediate. The transpositions are catalyzed by several transposase enzymes. Some transposases non-specifically bind to any target site in DNA, whereas others bind to specific DNA sequence targets. The transposase makes a staggered cut at the target site resulting in single-strand 5′ or 3′ DNA overhangs (sticky ends). This step cuts out the DNA transposon, which is then ligated into a new target site; this process involves activity of a DNA polymerase that fills in gaps and of a DNA ligase that closes the sugar-phosphate backbone. This results in duplication of the target site. The insertion sites of DNA transposons can be identified by short direct repeats which can be created by the staggered cut in the target DNA and filling in by DNA polymerase, followed by a series of inverted repeats important for the TE excision by transposase. Cut-and-paste TEs can be duplicated if their transposition takes place during S phase of the cell cycle when a donor site has already been replicated, but a target site has not yet been replicated. Transposition can be classified as either autonomous or non-autonomous in both Class I and Class II TEs. Autonomous TEs can move by themselves while non-autonomous TEs require the presence of another TE to move. This is often because non-autonomous TEs lack transposase (for class II) or reverse transcriptase (for class I).

As used herein, the term “transposase” refers to an enzyme that binds to the end of a transposon and catalyzes the movement of the transposon to another part of the genome by a cut and paste mechanism or a replicative transposition mechanism.

As used herein, the terms “polypeptide,” “peptide,” “polypeptide construct,” and “peptide construct” and their grammatical equivalents, refer to a polymeric compound comprised of covalently linked amino acid residues. A “mature protein” is a protein which is full-length and which, optionally, includes glycosylation or other modifications typical for the protein in a given cellular environment. As disclosed herein, embodiments of the invention include HPV antigens/antigenic polypeptides, peptides, and mature proteins described herein and also polynucleotides (DNA or RNA) that encode the same. Polypeptides and proteins disclosed herein (including functional fragments and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, omithine, α-aminocyclopentane carboxylic acid, α-aminocyclohexane carboxylic acid, α-aminocycloheptane carboxylic acid, α-(2-amino-2-norbomane)-carboxylic acid, α, γ-diaminobutyric acid, α,β-diaminopropionic acid, homophenylalanine, and α-tert-butylglycine.

As used herein, the term “polypeptide fragment” refers to a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of at least 2, 3, 4, 5, 6, 8, 10, 13, 14, 15, 16, 17, 18, 19, 20, 21, 22, 25, 26, 30, 35, 40, 45, 50, 100, 200, 240, or 300 or more amino acids.

As used herein, the terms “isolated polypeptide,” “isolated peptide,” or “isolated protein” refer to a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be, for example, due to incomplete purification, addition of stabilizers, or compounding into a pharmaceutically acceptable preparation.

As used herein, the term “identical” or “sequence identity” in the context of two nucleic acid sequences or amino acid sequences of polypeptides refers to the residues in the two sequences which are the same when aligned for maximum correspondence over a specified comparison window. A “comparison window,” as used herein, refers to a segment of at least about 20 contiguous positions, usually about 50 to about 200, more usually about 100 to about 150 in which a sequence can be compared to a reference sequence of the same number of contiguous positions after the two sequences are aligned optimally. Methods of alignment of sequences for comparison are well-known in the art. Optimal alignment of sequences for comparison can be conducted by the local homology algorithm of Smith and Waterman, Adv. Appl. Math., 2:482 (1981); by the alignment algorithm of Needleman and Wunsch, J Mal. Biol., 48:443 (1970); by the search for similarity method of Pearson and Lipman, Proc. Nat. Acad Sci U.S.A., 85:2444 (1988); by computerized implementations of these algorithms (including, but not limited to CLUSTAL in the PC/Gene program by Intelligentics, Mountain View Calif, GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment.

In one class of embodiments, the polypeptides herein are at least 80%, 85%, 90%, 98%, 99%, 99.1%, 99.5%, 99.9%, 99.99%, or 100% identical to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can be 50%, 60%, 70%, 75%, 80%, 85%, 90%, 98%, 99%, 99%, 99.1%, 99.5%, 99.9%, 99.99%, or 100% identical to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

As used herein, the term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including but not limited to those described above or in, e.g., Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Methods to determine identity and similarity are codified in publicly available computer programs. Sequence alignments and percent identity calculations may be performed using sequence analysis software such as the MegAlign (or more recently MegAlign Pro) program of the LASERGENE bioinformatics computing suite (DNASTAR Inc., Madison, Wis.). Multiple alignment of the sequences may be performed using a Clustal method of alignment (Higgins et al., CABIOS. 5:151 1989) with the default parameters (GAP PENALTY=10, GAP LENGTH PENALTY=10). Default parameters for pairwise alignments using a Clustal method may be selected: KTUPLE 1, GAP PENALTY=3, WINDOW=5 and DIAGONALS SAVED=5.

As used herein, the term “substantially similar” and its grammatical equivalents as applied to nucleic acid or amino acid sequences mean that a nucleic acid or amino acid sequence comprises a sequence that has at least 90% sequence identity or more, such as at least 95%, at least 98%, at least 99%, at least 99.1%, at least 99.2%, at least 99.3%, at least 99.4%, at least 99.5%, at least 99.6%, at least 99.7%, at least 99.8%, at least 99.9%, and at least 99.99% sequence identity to a reference sequence using the comparison programs described above, e.g., BLAST, using standard parameters. The term “substantially identical” and its grammatical equivalents as applied to nucleic acid or amino acid sequences mean that a nucleic acid or amino acid sequence comprises a sequence that has at least 99%, such as at least at least 99.1%, at least 99.2%, at least 99.3%, at least 99.4%, at least 99.5%, at least 99.6%, at least 99.7%, at least 99.8%, at least 99.9%, and at least 99.99% sequence identity to a reference sequence using the comparison programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (scc Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

As used herein, the term “functional fragment,” or its grammatical equivalents, is used herein to mean a portion, fragment, or segment of a biological molecule that retains the essential functional characteristics or activities of the original biological molecule. The term “functional variant,” or its grammatical equivalents, is used herein to mean a modified form of a biological molecule that retains the essential functional characteristics or activities of the original molecule while exhibiting some degree of variation. It includes a biological molecule that has been altered, such as through genetic engineering or mutagenesis techniques, to introduce specific changes while preserving the biological molecule's overall functionality. A functional variant may have one or more amino acid substitutions, insertions, or deletions compared to the original molecule, while still maintaining the desired biological activity or function. The techniques for obtaining these variants, including genetic (suppressions, deletions, mutations, etc.), chemical, and enzymatic techniques, are known to persons having ordinary skill in the art. In one embodiment, a variant biological comprises at least about 14 monomers (e.g., nucleotides or amino acids).

As used herein, the term “homology” in all its grammatical forms and spelling variations refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

The term “substitution,” when used in the context of an amino acid sequence refers to a variation wherein one amino acid in the amino acid sequence is replaced by another. The nomenclature used to denote amino acid substitutions follows a standardized format. Taking “L50G” as an example, “L” represents the amino acid leucine (abbreviated as “L”) at the original position, “50” signifies the position of the amino acid in the amino acid sequence in relation to the N-terminus thereof (in this case, the amino acid is the 50^{th} amino acid from the N-terminus of the sequence), and “G” indicates the substituted amino acid, in this instance, glycine (abbreviated as “G”). Therefore, an “L50G” denotes a substitution where leucine at position 50 of the amino acid sequence (relative to the N-terminus thereof) has been replaced by glycine.

As used herein, the term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (see Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Examples of conservative mutations include amino acid substitutions of amino acids within the sub-groups above, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free-NH₂ can be maintained. Exemplary conservative amino acid substitutions are shown in the following chart:

TABLE 2
Exemplary conservative amino acid substitutions

| Type of Amino Acid | Substitutable Amino Acids |
|---|---|
| Hydrophilic | Ala, Pro, Gly, Glu, Asp, Gln, Asn, Ser, Thr |
| Sulphydryl | Cys |
| Aliphatic | Val, Ile, Leu, Met |
| Basic | Lys, Arg, His |
| Aromatic | Phe, Tyr, Trp |

An amino acid sequence that differs from a reference amino acid sequence by only conservative amino acid substitutions will be referred to herein as a “conservatively-substituted variant” of the reference sequence. Given the established knowledge and well-known techniques in protein science, it is well within the skill of a person of ordinary skill in the art to determine the functional impact of a “conservatively-substituted variant” as compared to the reference amino acid sequence.

In some embodiments, the functional variant may be a conservatively-substituted variant of the reference sequence. In some embodiments, the conservatively-substituted variant may differ from the amino acid sequence of the reference sequence by 100 or fewer conservative amino acid substitutions. In some embodiments, the conservatively-substituted variant may differ from the amino acid sequence of the reference sequence by 90 or fewer amino acid substitutions. In some embodiments, the conservatively-substituted variant may differ from the amino acid sequence of the reference sequence by 80 or fewer amino acid substitutions. In some embodiments, the conservatively-substituted variant may differ from the amino acid sequence of the reference sequence by 70 or fewer conservative amino acid substitutions. In some embodiments, the conservatively-substituted variant may differ from the amino acid sequence of the reference sequence by 60 or fewer conservative amino acid substitutions. In some embodiments, the conservatively-substituted variant may differ from the amino acid sequence of the reference sequence by 50 or fewer conservative amino acid substitutions. In some embodiments, the conservatively-substituted variant may differ from the amino acid sequence of the reference protein by 40 or fewer conservative amino acid substitutions. In some embodiments, the conservatively-substituted variant may differ from the amino acid sequence of the reference sequence by 30 or fewer conservative amino acid substitutions. In some embodiments, the conservatively-substituted variant may differ from the amino acid sequence of the reference sequence by 20 or fewer conservative amino acid substitutions. In some embodiments, the conservatively-substituted variant may differ from the amino acid sequence of the reference sequence by 10 or fewer conservative amino acid substitutions. In some embodiments, the conservatively-substitute variant may differ from the reference sequence by 9 or fewer, 8 or fewer, 7 or fewer, 6 or fewer, 5 or fewer, 4 or fewer, 3 or fewer, 2 or fewer, or 1 conservative amino acid substitutions. In some embodiments, the conservatively-substituted variant may differ from the amino acid sequence of the reference sequence by at least 100 and 150 conservative amino acid substitutions. In some embodiments, the conservatively-substituted variant may differ from the amino acid sequence of the reference sequence by at least 150 conservative amino acid substitutions.

An amino acid sequence that differs from a reference amino acid sequence by at least one non-conservative amino acid substitution will be referred to herein as a “non-conservatively-substituted variant” of the reference sequence. As used herein, the term “non-conservative amino acid substitution” refers to an amino acid substitution between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the homologous parent protein. Amino acid substitutability is discussed in more detail, for example, in L. Y. Yampolsky and A. Stoltzfus, “The Exchangeability of Amino acids in Proteins,” Genetics 2005 August; 170 (4): 1459-1472. Given the established knowledge and well-known techniques in protein science, it is well within the skill of a person of ordinary skill in the art to determine the functional impact of a non-conservative amino acid substitution in a functional variant as compared to the reference amino acid sequence.

In some embodiments, the functional variant may differ from the amino acid sequence of the reference sequence by at least one non-conservative amino acid substitution. In some embodiments, the functional variant may differ from the amino acid sequence of the reference sequence by at least two, at least three, at least four, at least five, at least six, at least seven, at least eight, at least nine, or at least ten non-conservative amino acid substitutions. In some embodiments, the functional variant may differ from the amino acid sequence of the reference sequence by between ten and 20 non-conservative amino acid substitutions. In some embodiments, the functional variant may differ from the amino acid sequence of the reference sequence by between 21 and 30 non-conservative amino acid substitutions. In some embodiments, the functional variant may differ from the amino acid sequence of the reference sequence by between 31 and 40 non-conservative amino acid substitutions. In some embodiments, the functional variant may differ from the amino acid sequence of the reference sequence by between 41 and 50 non-conservative amino acid substitutions. In some embodiments, the functional variant may differ from the amino acid sequence of the reference sequence by between 51 and 60 non-conservative amino acid substitutions. In some embodiments, the functional variant may differ from the amino acid sequence of the reference sequence by between 61 and 70 non-conservative amino acid substitutions. In some embodiments, the functional variant may differ from the amino acid sequence of the reference sequence by between 71 and 80 non-conservative amino acid substitutions. In some embodiments, the functional variant may differ from the amino acid sequence of the reference sequence by between 81 and 90 non-conservative amino acid substitutions. In some embodiments, the functional variant may differ from the amino acid sequence of the reference sequence by between 91 and 100 non-conservative amino acid substitutions. In some embodiments, the functional variant may differ from the amino acid sequence of the reference sequence by at least 100 non-conservative amino acid substitutions.

As used herein, the term “antibody” refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CH1, CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDRI, CDR2, and CDR3, form the “hypervariable region” of an antibody, which is responsible for antigen binding.

As used herein, the term “functional antibody fragment” and “functional fragment of an antibody,” or their grammatical equivalents, are used interchangeably to mean a portion, fragment, or segment of the antibody that retains the essential functional characteristics or activities of the original antibody. In one embodiment, that activity is the ability to specifically bind to an antigen. (See, generally, Holliger et al., Nat. Biotech., 23 (9): 1126-1129 (2005)). The functional antibody fragment may comprise, for example, one or more CDRs, the variable region (or portions thereof), the constant region (or portions thereof), or combinations thereof. Non-limiting examples of functional antibody fragments include: (i) an antigen-binding fragment (Fab), which is a monovalent fragment consisting of the VL, VH, CL, and CH1 domains; (ii) a F(ab′)2 fragment, which is a bivalent fragment comprising two Fab fragments linked by a disulfide bridge at the stalk region; (iii) a variable fragment (“Fv”) consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al., Science, 242:423-426 (1988); Huston et al., Proc. Natl. Acad Sci. USA, 85:5879-5883 (1988); and Osbourn et al., Nat. Biotechnol., 16:778 (1998)) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Functional antibody fragments are known in the art and are described in more detail in, e.g., U.S. Pat. No. 8,603,950.

As used herein, the term “antibody-like molecules” can be for example proteins that are members of the Ig-superfamily which are able to selectively bind a partner. MHC molecules and T cell receptors are such molecules. In one embodiment, the antibody-like molecule is a TCR. In one embodiment, the TCR has been modified to increase its MHC binding affinity.

As used herein, the term “antigen recognition moiety” or “antigen recognition domain” refers to a molecule or portion of a molecule that specifically binds to an antigen. In one embodiment, the antigen recognition moiety is an antibody, antibody-like molecule or fragment thereof and the antigen is a tumor antigen.

As used herein, the term “immune cells” includes dendritic cells, macrophages, neutrophils, mast cells, cosinophils, basophils, natural killer cells and lymphocytes (e.g., B and T cells).

As used herein, the terms “T cell” or “T lymphocyte” refer to a type of lymphocyte that plays a central role in cell-mediated immunity. They can be distinguished from other lymphocytes, such as B cells and natural killer cells (NK cells), by the presence of a T-cell receptor (TCR) on the cell surface. “TCRs” are protein molecules found on the surface of T cells, which are a type of white blood cell involved in the adaptive immune response. A TCR's variable domain contains the highly polymorphic loops referred to as complementarity determining regions (CDRs), which are responsible for binding to the peptide-presenting MHC. There are two major forms of TCRs: αβ TCR and γδ TCR. Both forms consist of two protein chains, known as alpha (α) and beta (β) chains for αβ TCRs and gamma (γ) and delta (δ) chains for γδ TCRs. These chains come together to form a heterodimeric structure. The majority of T cells in the human immune system express αβ TCRs. The α chain and β chain of αβ TCRs are encoded by separate gene segments, which undergo recombination during T cell development to generate diverse TCR specificities. The α and β chains each contain variable (V), diversity (D), and joining (J) gene segments, similar to the antibody gene rearrangement process. The combination of V, D, and J gene segments contributes to the unique antigen-binding specificity of the αβ TCR. The αβ TCR recognizes antigenic peptides presented in the context of major histocompatibility complex (MHC) molecules on the surface of antigen-presenting cells. In contrast to αβ TCRs, γδ TCRs are less prevalent in the immune system but still play important roles. The γ and δ chains of γδ TCRs are also encoded by separate gene segments and undergo recombination during T cell development. The γδ TCR gene rearrangement process is distinct from that of αβ TCRs. γδ T cells often exhibit a tissue-specific distribution and are found in epithelial tissues, such as the skin and gut. γδ TCRs can recognize a variety of antigens, including certain peptides and non-peptide molecules, independently of MHC presentation. Both αβ TCRs and γδ TCRs participate in immune surveillance and response, but they have different functions and specificities. The αβ TCRs are predominantly involved in recognizing peptides presented by major histocompatibility complex (MHC) molecules, while γδ TCRs can have more diverse antigen recognition capabilities.

TCRs, and constructs encoding TCRs, that recognize MHC-antigen complexes, can be generated and introduced into T cells (known as TCR T cells), and the ensuing TCR-peptide-MHC interaction can be exploited to trigger an immune response. Greenbaum et al., Cancer Immunol Res 1 Nov. 2021; 9 (11): 1252-1261. There is an interest in using TCRs with higher than normal range of affinity for peptide-MHC antigens (type I), referred to as high affinity TCRs, to: 1) driving the activity of CD4 helper T cells (which do not have a CD8 co-receptor), or 2) developing soluble TCRs that can be used to directly target cells by attaching “effector” molecules (e.g., antibody Fc regions, toxic drugs, or antibody scFvs such as anti-CD 3 antibodies to form bispecific proteins) (Ashfield and Jakobsen, IDrugs, 9,554-9 (2006); Foote and Eisen Proc Natl Acad Sci USA, 97:10679-81 (2000); Holler et al., Proc Natl Acad Sci USA, 97:5387-92 (2000); Molloy et al., Curr Opin Pharmacol, 5:438-43 (2005); Richman and Kranz, Biomol Eng, 24:361-73 (2007)). This approach may also overcome the problem faced by some cancer patients whereby their T cells do not express TCRs with sufficient specificity and binding affinity for the underlying tumor antigen. For example, over 300 MHC restricted T cell defined tumor antigens (Cheever et al., Clin Cancer Res. 2009; 15 (17): 5323-5337) have been identified. These tumor antigens include mutated peptides, differentiation antigens, and over-expressed antigens, all of which serve as targets for therapy. Since most cancer antigens described to date are derived from intracellular proteins that can only be targeted at the cell surface in the context of MHC molecules, TCRs are ideal candidates for therapy as they have evolved to recognize this class of antigens. Similarly, TCRs can detect peptides derived from viral proteins that have been naturally processed in infected cells and displayed on the cell surface by MHC molecules. However, patients with these diseases may not have an optimized TCR that binds and destroys infected cells. Finally, in methods with high specificity, TCRs may be used as receptor antagonists for autoimmune targets, or as delivery agents to immunosuppress local immune cell responses, thereby avoiding general immunosuppression.

As used herein, the term “T helper cells” (TH or Th cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surfaces. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including THI, TH2, TH3, TH9, THI 7, TH22 or TFH (T follicular helper cells), which secrete different cytokines to facilitate different types of immune responses. Signaling from the APCs directs T cells into particular subtypes.

As used herein, the term “cytotoxic T cells” (TC cells, or CTLs) or “cytotoxic T lymphocytes” destroy virus-infected cells and tumor cells, and are also implicated in transplant rejection. These cells are also known as CD8+ T cells since they express the CD8 glycoprotein at their surfaces. These cells recognize their targets by binding to antigen associated with MHC class I molecules, which are present on the surface of all nucleated cells. Through IL-10, adenosine, and other molecules secreted by regulatory T cells, the CD8+ cells can be inactivated to an anergic state, which prevents autoimmune diseases.

As used herein, the term “memory T cells” refers to a subset of antigen-specific T cells that persist long-term after an infection has resolved. They quickly expand to large numbers of effector T cells upon re-exposure to their cognate antigen, thus providing the immune system with memory against past infections. Memory T cells comprise three subtypes: central memory T cells (TcM cells) and two types of effector memory T cells (TEM cells and TEMRA cells). Memory cells can be either CD4+ or CD8+. Memory T cells typically express the cell surface proteins CD45RO, CD45RA and/or CCR7.

As used herein, the term “regulatory T cells” (Treg cells), formerly known as suppressor T cells, refer to T cells that play a role in the maintenance of immunological tolerance. Their major role is to shut down T cell-mediated immunity toward the end of an immune reaction and to suppress autoreactive T cells that escaped the process of negative selection in the thymus.

As used herein, the term “Natural killer T cells” (NKT cells—not to be confused with natural killer cells of the innate immune system) refer to those cells that bridge the adaptive immune system with the innate immune system. Unlike conventional T cells that recognize peptide antigens presented by major histocompatibility complex (MHC) molecules, NKT cells recognize glycolipid antigen presented by a molecule called CD Id. Once activated, these cells can perform functions ascribed to both T helper (TH) and cytotoxic T (TC) cells (i.e., cytokine production and release of cytolytic/cell killing molecules). They are also able to recognize and eliminate some tumor cells and cells infected with herpes viruses.

As used herein, the term “proliferative disease” refers to a unifying concept in which excessive proliferation of cells and/or turnover of cellular matrix contributes significantly to the pathogenesis of the disease, including cancer.

“Patient” or “subject” as used herein refers to a mammalian subject diagnosed with or suspected of having or developing a disease or disorder such as cancer. In some embodiments, the term “patient” refers to a mammalian subject with a higher than average likelihood of developing a proliferative disorder such as cancer. Exemplary patients can be humans, apes, dogs, pigs, cattle, cats, horses, goats, sheep, rodents and other mammalians that can benefit from the therapies disclosed herein. Exemplary human patients can be male and/or female. “Patient in need thereof” or “subject in need thereof” is referred to herein as a patient diagnosed with or suspected of having a disease or disorder, for instance, but not restricted to human papilloma virus (HPV) infection.

“Administering” is referred to herein as providing one or more compositions described herein to a patient or a subject. By way of example and not limitation, composition administration, e.g., injection, can be performed by intravenous injection, subcutaneous injection, intradermal injection, intraperitoneal injection, or intramuscular injection. One or more such routes can be employed. Parenteral administration can be, for example, by bolus injection or by gradual perfusion over time. Alternatively, or concurrently, administration can be by the oral route. Additionally, administration can also be by surgical deposition, or positioning of a medical device. A pharmaceutical composition can comprise a composition of the invention as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

As used herein, the term “therapeutic product” refers to a therapeutic polypeptide or therapeutic polynucleotide which imparts a beneficial function to the host cell in which such product is expressed. Therapeutic polypeptides may include, without limitation, peptides as small as three amino acids in length, single- or multiple-chain proteins, and fusion proteins. Therapeutic polynucleotides may include, without limitation, antisense oligonucleotides, small interfering RNAs, ribozymes, and RNA external guide sequences. The therapeutic product may comprise a naturally occurring sequence, a synthetic sequence or a combination of natural and synthetic sequences.

As used herein, the term “treatment,” “treating,” or its grammatical equivalents refers to obtaining a desired pharmacologic and/or physiologic effect. In embodiments, the effect is therapeutic, i.e., the effect partially or completely cures a disease and/or adverse symptom or pathological manifestation attributable to the disease. To this end, the inventive method comprises administering a therapeutically effective amount of a composition of the invention expressing the inventive nucleic acid sequence, or a vector comprising the inventive nucleic acid sequences.

As used herein, a “treatment interval” refers to a treatment cycle, for example, a course of administration of a therapeutic agent that may be repeated, e.g., on a regular schedule. In some embodiments, a dosage regimen may have one or more periods of no administration of the therapeutic agent in between treatment intervals.

As used herein, a “dosage regimen” or “dosing regimen” includes a treatment regimen based on a determined set of doses. The terms “dose” and “dosing” as used herein refers to the administration of a substance to achieve a therapeutic objective (e.g., the treatment of a tumor).

The terms “administered in combination,” “co-administration,” or “co-administering,” or “co-providing” as used herein means that two (or more) different treatments are delivered to the subject during the course of the subject's affliction with a disease or disorder, for example, the two or more treatments are delivered after the subject has been diagnosed with the disease or disorder and before the disease or disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments may be partially additive, wholly additive, or greater than additive. The delivery may be such that an effect of the first treatment delivered is still detectable when the second is delivered.

In some embodiments of the present invention, a first treatment and a second treatment may be administered simultaneously (e.g., at the same time), in the same or in separate compositions, or sequentially. Sequential administration refers to administration of one treatment before (e.g., immediately before; less than 5, 10, 15, 30, 45, or 60 minutes before; 1, 2, 3, 4, 6, 8, 10, 12, 16, 20, 24, 48, 72, 96 or more hours before; 4, 5, 6, 7, 8, 9 or more days before; or 1, 2, 3, 4, 5, 6, 7, 8 or more weeks before) administration of an additional (e.g., secondary) treatment. The order of administration of the first and secondary treatment may also be reversed.

The term “therapeutically effective amount,” “therapeutic amount,” “immunologically effective amount,” “anti-tumor effective amount,” “tumor-inhibiting effective amount” or its grammatical equivalents refers to an amount effective, at dosages and for periods of time necessary, to achieve a desired therapeutic result. The therapeutically effective amount can vary according to factors such as the disease state, age, sex, and weight of the individual, and the ability of a composition described herein to elicit a desired response in one or more subjects.

Alternatively, the pharmacologic and/or physiologic effect of administration of one or more compositions described herein to a patient or a subject of can be “prophylactic,” i.e., the effect completely or partially prevents a disease or symptom thereof. A “prophylactically effective amount” refers to an amount effective, at dosages and for periods of time necessary, to achieve a desired prophylactic result (e.g., prevention of disease or prevention of manifestation of a target pathology).

### I. Vectors

Genetic therapy involving the introduction of a transgene (e.g., via vaccination) expressing an exogenous protein to a subject has proven useful in the treatment of diseases and disorders in subjects in need thereof. For such introduction, a vector, for example, a viral vector, comprising a transgene encoding such a protein is typically used.

The present invention is directed in part to a vector comprising an expression cassette, the expression cassette comprising a transgene encoding an HPV antigen design.

In certain embodiments, the vector is a plasmid.

Another suitable vector is an integrating expression vector. Such vectors are able to randomly integrate into the host cell's DNA, or can include a recombination site to enable the specific recombination between the expression vector and the host cell's chromosome. Such integrating expression vectors can utilize the endogenous expression control sequences of the host cell's chromosomes to effect expression of the desired protein. Examples of vectors that integrate in a site-specific manner include, for example, components of the flp-in system from Invitrogen (Carlsbad, Calif.) (e.g., pcDNATM5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) FLEXI™ from Promega (Madison, Wis.).

A. Methods for Introducing Nucleic Acids into Cells

Methods of introducing and expressing genes into a cell are well known. In the context of an expression vector, the vector can be readily introduced into a host cell, e.g., mammalian, bacterial, yeast, or insect cell by any method in the art. For example, the expression vector can be transferred into a host cell by physical, chemical, or biological means. Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### 1. Physical Methods

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. Sec, for example, Sambrook et al., Molecular Cloning: A Laboratory Manual, Cold Spring Harbor Laboratory, New York (2001). In embodiments, a method for the introduction of a polynucleotide into a host cell is calcium phosphate transfection or polyethylenimine (PEI) Transfection.

In some embodiments, a method for introduction of a polynucleotide into a host cell is electroporation. Electroporation is a technique that uses electrical pulses to temporarily increase the permeability of cell membranes, allowing the uptake of nucleic acid molecules into the cells. This process enhances the delivery and expression of a biological material (e.g., peptide or nucleic acid) in the subject's cells, potentially improving the immune response against HPV. In some embodiments, the biological material is an HPV antigen. In other embodiments, the biological material is an HPV antigen-encoding nucleic acid.

Electroporation buffers may contain water, sugars, sugar alcohols, chloride salts, and buffering agents. The pH, conductivity, and osmolality of the buffer are carefully controlled. The buffer may be used with an UltraPorator™ electroporation apparatus and cartridge. The UltraPorator™ electroporation apparatus is designed for rapid manufacturing of gene and cell therapies and may be used as a scale-up and commercialization solution for decentralized cell manufacturing. Sec, e.g., PCT/US20/59984 (filed Nov. 11, 2020) and U.S. patent application Ser. No. 17/095,028 (filed Nov. 11, 2020).

In some embodiments, a suspension is formed by combining cells obtained from a human with an exogenous biological material in the buffer, and then an electric current is applied to the suspension to facilitate the introduction of the biological material into the cells. The voltage pulse may have a field strength of 1-10 kV/cm, a duration of 5-250 μs, and a current density of at least 2 A/cm². The method can be used to introduce biological materials, such as nucleic acids, peptides, polypeptides, proteins, enzymes, or RNPs, into primary human blood cells, pluripotent precursor cells, fibroblasts, and endothelial cells. In some embodiments, the method is used to introduce biologically active material into primary human blood cells, pluripotent precursor cells of human blood, as well as primary human fibroblasts and endothelial cells. In some embodiments, the cells are human blood cells, for example immune cells. In certain embodiments, the immune cells are neutrophils, cosinophils, basophils, mast cells, monocytes, macrophages, dendritic cells, natural killer cells, and lymphocytes (B cells and T cells), or some combination thereof. In some embodiments, the lymphocytes are T-cells. In certain embodiments, the cells are obtained from a patient.

In some embodiments, the transfection yield and transfected cell recovery yield using the electroporation buffer may be significantly higher than those obtained using control buffers. In some embodiments, the transfection yield with a buffer of the invention is at least about 1.1 times that of the transfection yield with a control buffer, for example, 1.1, 1.2, 1.3, 1.4, 1.5, 1.6, 1.7, 2.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 times higher than that of a control buffer.

In some of the methods described herein, HPV antigens are administered to a subject. In some aspects, these methods include introducing the nucleic acid molecules of the invention into the subject, followed by electroporation. In a specific embodiment, a nucleic acid molecule (e.g., a plasmid encoding an antigen or therapeutic protein of interest) is injected into the target tissue of a subject, such as the skin or muscle, using a conventional needle or a needle-free injection device. Shortly after the injection, a handheld electroporation device is applied to the injection site, such as by contacting the skin or tissue. The device delivers brief, controlled electrical pulses to the tissue, creating transient pores in the cell membranes, making them more permeable to the nucleic acid molecule, which then enters the cells through the pores created by electroporation. Once inside the cells, the nucleic acid molecule is translated into the desired antigen or therapeutic protein. The produced antigen stimulates an immune response, which can protect against the targeted pathogen. In the case of therapeutic proteins, they can exert their intended effects within the cells or tissues.

In some embodiments, the methods may involve administering multiple copies of a single nucleic acid molecule, such as a single plasmid, or multiple copies of two or more different nucleic acid molecules, such as two or more different plasmids. The number of different nucleic acid molecules administered can vary depending on the specific application and may include two, three, four, five, six, seven, eight, nine, ten, or more distinct nucleic acid sequences. This approach allows for the delivery of multiple HPV antigens or the co-delivery of additional immunostimulatory factors to enhance the immune response.

Genetic constructs containing the HPV antigen-encoding nucleic acids can be administered using various methods, including electroporation devices, traditional syringes, standard needles, side port needles (as described in U.S. Publ. No. 2023/0017972, incorporated herein by reference), needleless injection devices, or “microprojectile bombardment gene guns.” Each of these methods has its advantages and can be selected based on factors such as the target tissue, the desired level of gene expression, and the specific application.

Several minimally invasive electroporation devices and methods have been described in the literature and are incorporated herein by reference. These include the devices and methods disclosed in published U.S. patent application No. 20080234655; U.S. Pat. Nos. 6,520,950; 7,171,264; 6,208,893; 6,009,347; 6,120,493; 7,245,963; 7,328,064; 6,763,264; and 20,240,123052. These devices and methods are designed to efficiently deliver nucleic acid molecules into cells while minimizing tissue damage and discomfort to the subject. By using these minimally invasive electroporation techniques, the HPV antigen-encoding nucleic acids can be effectively introduced into the subject's cells, leading to the production of the HPV antigen and the stimulation of an immune response.

### 2. Chemical Methods

Chemical methods for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

### B. Viral-Based Delivery Systems

Also provided herein are viral-based delivery systems, such as viral vectors for delivering nucleic acids. Representative viral vectors include adeno-associated viral vectors, adenoviral vectors, retroviral vectors, and herpes virus-based vectors. Viral vectors may be used as delivery vehicles for nucleic acids encoding a therapeutic molecule, for example, an anti-inflammatory agent, while also avoiding immune-surveillance by host cells. Retrovirus, adenovirus, adeno-associated virus (AAV), and herpes simplex virus have all been adapted for viral vector applications. Robbins et al., Pharmacology & Therapeutics, 80:35-47 (1998). In particular, recombinant adenoviral vectors offer high levels of transgene expression in which the vector remains as episomal DNA without integration into the host genome. The high transduction efficiency and high levels of short-term gene expression make adenoviral vectors ideal for gene therapy and vaccine applications. Furthermore, these viral vectors may be made replication-defective by the deletion of essential viral genes and the replacement thereof with expressions cassettes comprising a foreign therapeutic gene.

Viral vectors that are not infectious in humans or engineered to remove or inactivate their infectious properties are desired for use in genetic therapy as they are efficient at delivering transgenes and can deliver a high payload of nucleic acids to dendritic cells.

The efficacy of treatment using viral vectors, however, is limited by their immunogenicity. For example, human adenoviral vectors are commonly used in genetic therapy; however, as a majority of the U.S. population has been exposed to wild-type forms of such viruses, much of the population has pre-existing immunity thereto. As a result, such vectors and the transgenes carried thereon are quickly cleared from the bloodstream. Further, the immunogenicity of such vectors limit their efficacy in cases of repeat dosing.

### 1. Retroviral Vectors

In certain embodiments, the viral vector is a retroviral vector, such as a lentivirus vector. Vectors derived from retroviruses are suitable tools for achieving long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

### 2. Adeno-Associated Viral Vectors

In certain embodiments, the viral vector is an adeno-associated viral vector. Such vectors are derived from adeno-associated viruses. An advantage to the use of such vectors is that they are low immunogenicity in humans. Another advantage of using such vectors is that they are small and compact and thus can be efficiently used in delivering genes to a cell. However, their size also limits their payload capacity as compared with the use of an adenoviral vector. They are also more difficult to produce as compared with adenoviral vectors. In addition, they have narrow tissue tropism.

### 3. Herpes Virus-Based Vectors

In certain embodiments, the viral vector is a herpes virus-based vector. Such vectors are derived from the herpes simplex virus (HSV). Such vectors are known to be able to infect a wide variety of cell types and have a long persistence in the host. However, they are more difficult to engineer as compared with adenoviral vectors.

### 4. Adenoviral-Based Vectors

In certain embodiments, the viral vector is an adenoviral vector. Such vectors are derived from the adenovirus, for example, a human adenovirus (e.g. human Ad5 type adenovirus), an avian adenovirus, or a gorilla adenovirus. Adenoviruses are generally associated with benign pathologies in humans, and the genomes of adenoviruses isolated from a variety of species, including humans, have been extensively studied. Adenoviral vectors are advantageous as they are capable of infecting a wide variety of cell types. They are also capable of being relatively easily engineered and can carry a high payload.

The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the adenoviral serotypes 1 through 51, which are currently available from the American Type Culture Collection (ATCC, Manassas, Va.), or from any other serotype of adenovirus available from any other source. For instance, an adenovirus can be of subgroup A (e.g., serotypes 12, 18, and 31), subgroup B (e.g., serotypes 3, 7, 11, 14, 16, 21, 34, and 35), subgroup C (e.g., serotypes 1, 2, 5, and 6), subgroup D (e.g., serotypes 8, 9, 10, 13, 15, 17, 19, 20, 22-30, 32, 33, 36-39, and 42-47), subgroup E (serotype 4), subgroup F (serotypes 40 and 41), or any other adenoviral serotype.

The adenoviral vector can be any adenoviral vector capable of growth in a cell, which is in some significant part (although not necessarily substantially) derived from or based upon the genome of an adenovirus. The adenoviral vector can be based on the genome of any suitable wild-type adenovirus. In certain embodiments, the adenoviral vector is derived from the genome of a wild-type adenovirus of group C, especially of serotype 2 or 5. Adenoviral vectors are well known in the art and are described in, for example, U.S. Pat. Nos. 5,559,099, 5,712,136, 5,731,190, 5,837,511, 5,846,782, 5,851,806, 5,962,311, 5,965,541, 5,981,225, 5,994,106, 6,020,191, and 6,113,913, International Patent Applications WO 95/34671, WO 97/21826, and WO 00/00628, and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996).

Adenovirus is a medium-sized (90-100 nm), non-enveloped icosahedral virus containing approximately 36 kb of double-stranded DNA. The adenovirus capsid mediates the key interactions of the early stages of the infection of a cell by the virus, and is required for packaging adenovirus genomes at the end of the adenovirus life cycle. The capsid comprises 252 capsomeres, which includes 240 hexons, 12 penton base proteins, and 12 fibers. Ginsberg et al., Virology, 28:782-783 (1966). The hexon comprises three identical proteins, namely polypeptide II. Roberts et al., Science, 232:1148-1151 (1986). The penton base comprises five identical proteins and the fiber comprises three identical proteins. Proteins IIIa, VI, and IX are present in the adenoviral coat and are believed to stabilize the viral capsid. Stewart et al., Cell, 67:145-54 (1991) and Stewart et al., EMBO J., 12 (7): 2589-99 (1993). The expression of the capsid proteins, with the exception of pIX, is dependent on the adenovirus polymerase protein. Therefore, major components of an adenovirus particle are expressed from the genome only when the polymerase protein gene is present and expressed.

Several features of adenoviruses make them ideal vehicles for transferring genetic material to cells for therapeutic applications. For example, adenoviruses can be produced in high titers (e.g., about 10¹³ particle units (PU)), and can transfer genetic material to non-replicating and replicating cells. In addition, the adenoviral genome can be manipulated to carry a large amount of exogenous DNA (up to about 8 kb), and the adenoviral capsid can potentiate the transfer of even longer sequences. Curiel et al., Hum. Gene Ther., 3:147-154 (1992). Additionally, adenoviruses generally do not integrate into the host cell chromosome, but rather are maintained as a linear episome, thereby minimizing the likelihood that a recombinant adenovirus will interfere with normal cell function.

The adenovirus may be modified, for example, using methods known in the art, to be used as an adenoviral vector, e.g., a gene delivery vehicle. The adenovirus and adenoviral vector may be replication-competent, conditionally replication-competent, or replication-deficient.

A replication-competent adenovirus or adenoviral vector can replicate in typical host cells, i.e., cells typically capable of being infected by an adenovirus. A replication-competent adenovirus or adenoviral vector can have one or more mutations as compared to the wild-type adenovirus (e.g., one or more deletions, insertions, and/or substitutions) in the adenoviral genome that do not inhibit viral replication in host cells. For example, the adenovirus or adenoviral vector can have a partial or entire deletion of the adenoviral early region known as the E3 region, which is not essential for propagation of the adenovirus or adenoviral genome.

A conditionally-replicating adenovirus or adenoviral vector is an adenovirus or adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., promoter. In such an embodiment, replication requires the presence or absence of specific factors that interact with the transcription control sequence. Conditionally-replicating adenoviral vectors are further described in U.S. Pat. No. 5,998,205.

A replication-deficient adenovirus or adenoviral vector is an adenovirus or adenoviral vector that requires complementation of one or more gene functions or regions of the adenoviral genome that are required for replication as a result of, for example, a deficiency in one or more replication-essential gene function or regions, such that the adenovirus or adenoviral vector does not replicate in typical host cells, especially those in a human to be infected by the adenovirus or adenoviral vector.

A deficiency in a gene function or genomic region, as used herein, is defined as a disruption (e.g., deletion) of sufficient genetic material of the adenoviral genome to obliterate or impair the function of the gene (e.g., such that the function of the gene product is reduced by at least about 2-fold, 5-fold, 10-fold, 20-fold, 30-fold, or 50-fold) whose nucleic acid sequence was disrupted (e.g., deleted) in whole or in part. Deletion of an entire gene region often is not required for disruption of a replication-essential gene function. However, for the purpose of providing sufficient space in the adenoviral genome for one or more transgenes, removal of a majority of one or more gene regions may be desirable. While deletion of genetic material is preferred, mutation of genetic material by addition or substitution also is appropriate for disrupting gene function. Replication-essential gene functions are those gene functions that are required for adenovirus replication (e.g., propagation) and are encoded by, for example, the adenoviral early regions (e.g., the E1, E2, and E4 regions), late regions (e.g., the L1, L2, L3, L4, and L5 regions), genes involved in viral packaging (e.g., the IVa2 gene), and virus-associated RNAs (e.g., VA-RNA-1 and/or VA-RNA-2).

Whether the adenovirus or adenoviral vector is replication-competent or replication-deficient, the adenovirus or adenoviral vector typically retains at least a portion of the adenoviral genome. The adenovirus or adenoviral vector can comprise any portion of the adenoviral genome, including protein coding and/or non-protein coding regions. The adenovirus or adenoviral vector may comprise, for example, at least one nucleic acid sequence that encodes an adenovirus protein. The adenovirus or adenoviral vector can comprise a nucleic acid sequence that encodes any suitable adenovirus protein, for example, a protein encoded by any one of the early region genes (i.e., E1A, E1B, E2A, E2B, E3, and/or E4 regions), or a protein encoded by any one of the late region genes, which encode the virus structural proteins (i.e., L1, L2, L3, L4, and L5 regions).

It should be appreciated that the deletion of different regions of the adenoviral vector can alter the immune response of the mammal. In particular, the deletion of different regions can reduce the inflammatory response generated by the adenoviral vector. Furthermore, the adenoviral vector's coat protein can be modified to decrease the adenoviral vector's ability or inability to be recognized by a neutralizing antibody directed against the wild-type coat protein, as described in International Patent Application WO 98/40509.

In certain embodiments, the adenovirus or adenoviral vector comprises one or more nucleic acid sequences that encode the pIX protein, the DNA polymerase protein, the penton protein, the hexon protein, and/or the fiber protein. The adenovirus or adenoviral vector can comprise a full-length nucleic acid sequence that encodes a full-length amino acid sequence of an adenovirus protein. Alternatively, the adenovirus or adenoviral vector can comprise a portion of a full-length nucleic acid sequence that encodes a portion of a full-length amino acid sequence of an adenovirus protein. A “portion” of an amino acid sequence comprises at least three amino acids (e.g., about 3 to about 1,200 amino acids). Preferably, a “portion” of an amino acid sequence comprises 3 or more (e.g., 5 or more, 10 or more, 15 or more, 20 or more, 25 or more, 30 or more, 40 or more, or 50 or more) amino acids, but less than 1,200 (e.g., 1,000 or less, 800 or less, 700 or less, 600 or less, 500 or less, 400 or less, 300 or less, 200 or less, or 100 or less) amino acids. Preferably, a portion of an amino acid sequence is about 3 to about 500 amino acids (e.g., about 10, 100, 200, 300, 400, or 500 amino acids), about 3 to about 300 amino acids (e.g., about 20, 50, 75, 95, 150, 175, or 200 amino acids), or about 3 to about 100 amino acids (e.g., about 15, 25, 35, 40, 45, 60, 65, 70, 80, 85, 90, 95, or 99 amino acids), or a range defined by any two of the foregoing values. More preferably, a “portion” of an amino acid sequence comprises no more than about 500 amino acids (e.g., about 3 to about 400 amino acids, about 10 to about 250 amino acids, or about 50 to about 100 amino acids, or a range defined by any two of the foregoing values).

The adenovirus pIX protein is present in the adenovirus capsid, has been shown to strengthen hexon nonamer interactions, and is essential for the packaging of full-length genomes. See, e.g., Boulanger et al., J Gen. Virol., 44:783-800 (1979); Horwitz M. S., “Adenoviridae and their replication” in Virology, 2^{nd} ed., B. N. Fields et al. (eds.), Raven Press, Ltd., New York, pp. 1679-1721 (1990), Ghosh-Choudhury et al., EMBO J., 6:1733-1739 (1987) and van Oostrum et al., J. Virol., 56:439-448 (1985). In addition to its contribution to adenovirus structure, pIX also has been shown to exhibit transcriptional properties, such as stimulation of adenovirus major late promoter (MLP) activity. See, e.g., Lutz et al., J. Virol., 71 (7): 5102-5109 (1997). Nucleic acid sequences that encode all or a portion of an adenovirus pIX protein have been described, for example, in WO 2019/173465 and WO 2022/115470.

The adenovirus DNA polymerase protein is essential for viral DNA replication both in vitro and in vivo. The polymerase co-purifies in a complex with the precursor (pTP) of the terminal protein (TP), which is covalently attached to the 5′ ends of adenovirus DNA. Field et al., J. Biol. Chem., 259:9487-9495 (1984). Both the adenovirus DNA polymerase and pTP are encoded by the E2 region. The polymerase protein is required for the expression of all the structural proteins except for pIX. Without the gene sequence for polymerase protein, polymerase protein is not produced. As a result, the viral genome is not replicated, the Major Late Promoter is not activated, and the capsid proteins are not expressed. Nucleic acid sequences that encode all or a portion of an adenovirus DNA polymerase protein have been described, for example, in WO 2019/173465 and WO 2022/115470. Nucleic acid sequences that encode all or a portion of an adenovirus DNA polymerase protein include, for example, SEQ ID NO: 7 and SEQ ID NO: 2. Amino acid sequences that comprise a full-length adenovirus DNA polymerase, or a portion thereof, include, for example, SEQ ID NO: 17 and SEQ ID NO: 12.

The adenovirus hexon protein is the largest and most abundant protein in the adenovirus capsid. The hexon protein is essential for virus capsid assembly, determination of the icosahedral symmetry of the capsid (which in turn defines the limits on capsid volume and DNA packaging size), and integrity of the capsid. In addition, hexon is a primary target for modification in order to reduce neutralization of adenoviral vectors. See, e.g., Gall et al., J. Virol., 72:10260-264 (1998), and Rux et al., J. Virol., 77 (17): 9553-9566 (2003). The major structural features of the hexon protein are shared by adenoviruses across serotypes, but the hexon protein differs in size and immunological properties between serotypes. Jornvall et al., J. Biol. Chem., 256 (12): 6181-6186 (1981). A comparison of 15 adenovirus hexon proteins revealed that the predominant antigenic and serotype-specific regions of the hexon appear to be in loops 1 and 2 (i.e., LI or l1, and LII or l2, respectively), within which are seven discrete hypervariable regions (HVR 1 to HVR7) varying in length and sequence between adenoviral serotypes. Crawford-Miksza et al., J. Virol., 70 (3): 1836-1844 (1996). Nucleic acid sequences that encode all or a portion of an adenovirus hexon protein have been described, for example, in WO 2019/173465 and WO 2022/115470. Nucleic acid sequences that encode all or a portion of an adenovirus hexon protein include, for example, SEQ ID NO: 9 and SEQ ID NO: 4. Amino acid sequences that comprise a full-length adenovirus hexon protein, or a portion thereof, include, for example, SEQ ID NO: 19 and SEQ ID NO: 14.

The adenovirus fiber protein is a homotrimer of the adenoviral polypeptide IV that has three domains: the tail, shaft, and knob. Devaux et al., J. Molec. Biol., 215:567-88 (1990), Yeh et al., Virus Res., 33:179-98 (1991). The fiber protein mediates primary viral binding to receptors on the cell surface via the knob and the shaft domains. Henry et al., J. Virol., 68 (8): 5239-46 (1994). The amino acid sequences for trimerization are located in the knob, which appears necessary for the amino terminus of the fiber (the tail) to properly associate with the penton base. Novelli et al., Virology, 185:365-76 (1991). In addition to recognizing cell receptors and binding the penton base, the fiber contributes to serotype identity. Fiber proteins from different adenoviral serotypes differ considerably. See, e.g., Green et al., EMBO J., 2:1357-65 (1983), Chroboczek et al., Virology, 186:280-85 (1992), and Signas et al., J. Virol., 53:672-78 (1985). Thus, the fiber protein has multiple functions key to the life cycle of adenovirus. Nucleic acid sequences that encode all, or a portion of, an adenovirus fiber protein have been described, for example, in WO 2019/173465 and WO 2022/115470. Nucleic acid sequences that encode all, or a portion of, an adenovirus fiber protein include, for example, SEQ ID NO: 10 and SEQ ID NO: 5. Amino acid sequences that comprise a full-length adenovirus fiber protein, or a portion thereof, include, for example, SEQ ID NO: 20 and SEQ ID NO: 15.

The adenovirus penton base protein is located at the vertices of the icosahedral capsid and comprises five identical monomers. The penton base protein provides a structure for bridging the hexon proteins on multiple facets of the icosahedral capsid, and provides the essential interface for the fiber protein to be incorporated in the capsid. Each monomer of the penton base contains an RGD tripeptide motif. Neumann et al., Gene, 69:153-157 (1988). The RGD tripeptide mediates binding to av integrins and adenoviruses that have point mutations in the RGD sequence of the penton base are restricted in their ability to infect cells. Bai et al., J. Virol., 67:5198-5205 (1993). Thus, the penton base protein is essential for the architecture of the capsid and for maximum efficiency of virus-cell interaction. Nucleic acid sequences that encode all, or a portion of, an adenovirus penton base protein have been described, for example, in WO 2019/173465 and WO 2022/115470. Nucleic acid sequences that encode all, or a portion of, an adenovirus penton base protein include, for example, SEQ ID NO: 8 and SEQ ID NO: 3. Amino acid sequences that comprise a full-length adenovirus penton base protein, or a portion thereof, include, for example, SEQ ID NO: 18 and SEQ ID NO: 13.

The adenovirus or adenoviral vector can comprise one, two, three, four, or all five of the aforementioned sequences alone or in any combination. In this respect, the adenovirus or adenoviral vector may comprise any combination of any two of the aforementioned sequences, any combination of any three of the aforementioned sequences, any combination of any four of the aforementioned sequences, or all five of the aforementioned sequences.

In certain embodiments, the adenovirus or adenoviral vector is replication-deficient, such that the replication-deficient adenovirus or adenoviral vector requires complementation of at least one replication-essential gene function of one or more regions of the adenoviral genome for propagation (e.g., to form adenoviral vector particles).

The replication-deficient adenovirus or adenoviral vector can be modified in any suitable manner to cause the deficiencies in the one or more replication-essential gene functions in one or more regions of the adenoviral genome for propagation. The complementation of the deficiencies in the one or more replication-essential gene functions of one or more regions of the adenoviral genome refers to the use of exogenous means to provide the deficient replication-essential gene functions. Such complementation can be effected in any suitable manner, for example, by using complementing cells and/or exogenous DNA (e.g., helper adenovirus) encoding the disrupted replication-essential gene functions.

In some embodiments, the adenovirus or adenoviral vector is deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenoviral vector (HC-Ad). See Morsy et al., Proc. Natl. Acad. Sci. USA, 95:965-976 (1998); Chen et al., Proc. Natl. Acad. Sci. USA, 94:1645-1650 (1997); and Kochanek et al., Hum. Gene Ther., 10:2451-2459 (1999). The adenoviral vector also can have essentially the entire adenoviral genome removed, in which case at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large insert.

In certain embodiments, the adenoviral vector is “multiply-deficient,” meaning that the adenoviral vector is deficient in one or more gene functions required for viral replication in each of two or more regions of the adenoviral genome. For example, the aforementioned E1-deficient or E1/E3-deficient adenoviral vector can be further deficient in at least one replication-essential gene function of the E4 region (denoted an E1/E4-deficient adenoviral vector). An adenoviral vector deleted of the entire E4 region can elicit a lower host immune response.

Examples of replication-deficient adenoviral vectors are disclosed in U.S. Pat. Nos. 5,837,511; 5,851,806; 5,994,106; 6,127,175; 6,482,616; and 7,195,896, and International Patent Application Publications WO 1994/028152, WO 1995/002697, WO 1995/016772, WO 1995/034671, WO 1996/022378, WO 1997/012986, WO 1997/021826, and WO 2003/022311.

The early regions of the adenoviral genome include the E1, E2, E3, and E4 regions. The E1 region comprises the E1A and E1B subregions, and one or more deficiencies in replication-essential gene functions in the E1 region can include one or more deficiencies in replication-essential gene functions in either or both of the E1A and E1B subregions, thereby requiring complementation of the E1A subregion and/or the ElB subregion of the adenoviral genome for the adenovirus or adenoviral vector to propagate (e.g., to form adenoviral vector particles). The E2 region comprises the E2A and E2B subregions, and one or more deficiencies in replication-essential gene functions in the E2 region can include one or more deficiencies in replication-essential gene functions in either or both of the E2A and E2B subregions, thereby requiring complementation of the E2A subregion and/or the E2B subregion of the adenoviral genome for the adenovirus or adenoviral vector to propagate (e.g., to form adenoviral vector particles).

The E3 region does not include any replication-essential gene functions, such that a deletion of the E3 region in part or in whole does not require complementation of any gene functions in the E3 region for the adenovirus or adenoviral vector to propagate (e.g., to form adenoviral vector particles). In the context of the present disclosure, the E3 region is defined as the region that initiates with the open reading frame that encodes a protein with high homology to the 12.5K protein from the E3 region of human adenovirus 5 (NCBI reference sequence AP_000218) and ends with the open reading frame that encodes a protein with high homology to the 14.7K protein from the E3 region of human adenovirus 5 (NCBI reference sequence AP_000224.1). The E3 region can be deleted in whole or in part, or retained in whole or in part. The size of the deletion can be tailored so as to retain an adenovirus or adenoviral vector whose genome closely matches the optimum genome packaging size. A larger deletion will accommodate the insertion of larger heterologous nucleic acid sequences in the adenovirus or adenoviral genome. In some embodiments of the present disclosure, the L4 polyadenylation signal sequences, which reside in the E3 region, are retained.

The E4 region comprises multiple open reading frames (ORFs). An adenovirus or adenoviral vector with a deletion of all of the open reading frames of the E4 region except ORF6, and in some cases ORF3, does not require complementation of any gene functions in the E4 region for the adenovirus or adenoviral vector to propagate (e.g., to form adenoviral vector particles). Conversely, an adenovirus or adenoviral vector with a disruption or deletion of ORF6, and in some cases ORF3, of the E4 region (e.g., with a deficiency in a replication-essential gene function based in ORF6 and/or ORF3 of the E4 region), with or without a disruption or deletion of any of the other open reading frames of the E4 region or the native E4 promoter, polyadenylation sequence, and/or the right-side inverted terminal repeat (ITR), requires complementation of the E4 region (specifically, of ORF6 and/or ORF3 of the E4 region) for the adenovirus or adenoviral vector to propagate (e.g., to form adenoviral vector particles).

The late regions of the adenoviral genome include the L1, L2, L3, L4, and L5 regions. The adenovirus or adenoviral vector also can have a mutation in the major late promoter (MLP), as discussed in International Patent Application Publication WO 2000/000628, which can render the adenovirus or adenoviral vector replication-deficient if desired.

The one or more regions of the adenoviral genome that contain one or more deficiencies in replication-essential gene functions desirably are one or more early regions of the adenoviral genome, i.e., the E1, E2, and/or E4 regions. Thus, in certain embodiments, the adenoviral vector lacks all or part of such regions.

The replication-deficient adenovirus or adenoviral vector also can have one or more mutations as compared to the wild-type adenovirus (e.g., one or more deletions, insertions, and/or substitutions) in the adenoviral genome that do not inhibit viral replication in host cells. Thus, in addition to one or more deficiencies in replication-essential gene functions, the adenovirus or adenoviral vector can be deficient in other respects that are not replication-essential. For example, the adenovirus or adenoviral vector can have a partial or entire deletion of the adenoviral early region known as the E3 region, which is not essential for propagation of the adenovirus or adenoviral genome.

In some embodiments, the adenovirus or adenoviral vector is replication-deficient and requires, at most, complementation of the E1 region or the E4 region of the adenoviral genome, for propagation (e.g., to form adenoviral vector particles). In some such embodiments, the adenoviral vector may lack all or a portion of the E1 and/or E4 region. Thus, the replication-deficient adenovirus or adenoviral vector requires complementation of at least one replication-essential gene function of the E1A subregion and/or the E1B region of the adenoviral genome (denoted an E1-deficient adenoviral vector), or the E4 region of the adenoviral genome (denoted an E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, and at least one gene function of the nonessential E3 region of the adenoviral genome (denoted an E1/E3-deficient adenoviral vector). The adenovirus or adenoviral vector can be deficient in at least one replication-essential gene function (desirably all replication-essential gene functions) of the E4 region of the adenoviral genome, and at least one gene function of the nonessential E3 region of the adenoviral genome (denoted an E3/E4-deficient adenoviral vector).

In some embodiments, the adenovirus or adenoviral vector is replication-deficient and requires, at most, complementation of the E2 region, preferably the E2A subregion, of the adenoviral genome, for propagation (e.g., to form adenoviral vector particles). Thus, the replication-deficient adenovirus or adenoviral vector requires complementation of at least one replication-essential gene function of the E2A subregion of the adenoviral genome (denoted an E2A-deficient adenoviral vector) for propagation (e.g., to form adenoviral vector particles). The adenovirus or adenoviral vector can be deficient in at least one replication-essential gene function (desirably all replication-essential gene functions) of the E2A region of the adenoviral genome and at least one gene function of the nonessential E3 region of the adenoviral genome (denoted an E2A/E3-deficient adenoviral vector).

In some embodiments, the adenovirus or adenoviral vector is replication-deficient and requires, at most, complementation of the E1 and E4 regions of the adenoviral genome for propagation (e.g., to form adenoviral vector particles). In some such embodiments, the adenoviral vector may lack all or a portion of the E1 and/or E4 region. Thus, the replication-deficient adenovirus or adenoviral vector requires complementation of at least one replication-essential gene function of both the E1 and E4 regions of the adenoviral genome (denoted an E1/E4-deficient adenoviral vector) for propagation (e.g., to form adenoviral vector particles). The adenovirus or adenoviral vector can be deficient in at least one replication-essential gene function (desirably all replication-essential gene functions) of the E1 region of the adenoviral genome, at least one replication-essential gene function of the E4 region of the adenoviral genome, and at least one gene function of the nonessential E3 region of the adenoviral genome (denoted an E1/E3/E4-deficient adenoviral vector). The adenovirus or adenoviral vector preferably requires, at most, complementation of the E1 region of the adenoviral genome for propagation, and does not require complementation of any other deficiency of the adenoviral genome for propagation. More preferably, the adenovirus or adenoviral vector requires, at most, complementation of the E1 and E4 regions of the adenoviral genome for propagation, and does not require complementation of any other deficiency of the adenoviral genome for propagation.

The adenovirus or adenoviral vector, when deficient in multiple replication-essential gene functions of the adenoviral genome (e.g., an E1/E4-deficient adenoviral vector), can include a spacer sequence to provide viral growth in a complementing cell line similar to that achieved by adenoviruses or adenoviral vectors deficient in a single replication-essential gene function (e.g., an E1-deficient adenoviral vector). The spacer sequence can contain any nucleotide sequence or sequences which are of a desired length, such as sequences at least about 15 base pairs (e.g., between about 15 nucleotides and about 12,000 nucleotides), preferably about 100 nucleotides to about 10,000 nucleotides, more preferably about 500 nucleotides to about 8,000 nucleotides, even more preferably about 1,500 nucleotides to about 6,000 nucleotides, and most preferably about 2,000 to about 3,000 nucleotides in length, or a range defined by any two of the foregoing values. The spacer sequence can be coding or non-coding and native or non-native with respect to the adenoviral genome, but does not restore the replication-essential function to the deficient region. The spacer also can contain an expression cassette. More preferably, the spacer comprises a polyadenylation sequence and/or a gene that is non-native with respect to the adenovirus or adenoviral vector. The use of a spacer in an adenoviral vector is further described in, for example, U.S. Pat. No. 5,851,806 and International Patent Application Publication WO 1997/021826.

By removing all or part of the adenoviral genome, for example, the E1, E3, and E4 regions of the adenoviral genome, the resulting adenovirus or adenoviral vector is able to accept inserts of exogenous nucleic acid sequences while retaining the ability to be packaged into adenoviral capsids. An exogenous nucleic acid sequence can be inserted at any position in the adenoviral genome so long as insertion in the position allows for the formation of adenovirus or the adenoviral vector particle. The exogenous nucleic acid sequence preferably is positioned in the E1 region, the E3 region, or the E4 region of the adenoviral genome.

The replication-deficient adenovirus or adenoviral vector of the present disclosure can be produced in complementing cell lines that provide gene functions not present in the replication-deficient adenovirus or adenoviral vector, but required for viral propagation, at appropriate levels in order to generate high titers of viral vector stock. Such complementing cell lines are known and include Human Embryonic Kidney (HEK) 293 cells (described in, e.g., Graham et al., J. Gen. Virol., 36:59-72 (1977)), PER.C6 cells (described in, e.g., International Patent Application Publication WO 1997/000326, and U.S. Pat. Nos. 5,994,128 and 6,033,908), and 293-ORF6 cells (described in, e.g., International Patent Application Publication WO 95/34671 and Brough et al., J. Virol., 71:9206-9213 (1997)). Other suitable complementing cell lines to produce the replication-deficient adenovirus or adenoviral vector of the present disclosure include complementing cells that have been generated to propagate adenoviral vectors encoding transgenes whose expression inhibits viral growth in host cells (see, e.g., U.S. Patent Application Publication No. 2008/0233650). Additional suitable complementing cells are described in, for example, U.S. Pat. Nos. 6,677,156 and 6,682,929, and International Patent Application Publication WO 2003/020879.

In some instances, the cellular genome need not comprise nucleic acid sequences, the gene products of which complement for all of the deficiencies of a replication-deficient adenoviral vector. One or more replication-essential gene functions lacking in a replication-deficient adenoviral vector can be supplied by a helper virus, e.g., an adenoviral vector that supplies in trans one or more essential gene functions required for replication of the replication-deficient adenovirus or adenoviral vector. Alternatively, the inventive adenovirus or adenoviral vector can comprise a non-native replication-essential gene that complements for the one or more replication-essential gene functions lacking in the inventive replication-deficient adenovirus or adenoviral vector. For example, an E1/E4-deficient adenoviral vector can be engineered to contain a nucleic acid sequence encoding E4 ORF 6 that is obtained or derived from a different adenovirus (e.g., an adenovirus of a different serotype than the inventive adenovirus or adenoviral vector, or an adenovirus of a different species than the inventive adenovirus or adenoviral vector).

a. Gorilla-Based Adenovirus Vectors

In some embodiments, the adenovirus described herein is isolated from a gorilla. The Western Gorilla species includes the subspecies Western Lowland Gorilla (Gorilla gorilla gorilla) and Cross River Gorilla (Gorilla gorilla diehli) and the Eastern Gorilla species includes the subspecies Mountain Gorilla (Gorilla beringei beringei) and Eastern Lowland Gorilla (Gorilla beringei graueri). See, e.g., Wilson and Reeder, eds., Mammalian Species of the World, 3^{rd} ed., Johns Hopkins University Press, Baltimore, Md. (2005). In some embodiments, the adenovirus of the present disclosure is isolated from Mountain Gorilla (Gorilla beringei beringei). Previous research has characterized numerous gorilla adenoviruses and their genomic sequences (see, e.g., WO 2013/052832, WO 2013/052811, WO 2013/052799; WO 2019/173465, WO 2022/115470).

Gorilla adenoviruses share similarities with human adenoviruses in terms of vector design and safety, offering benefits like efficient transgene delivery and replication incompetence through targeted deletions. Importantly, compared to human adenoviruses, pre-existing human immunity to gorilla adenoviruses is minimal. This lack of recognition by human immune systems minimizes potential pre-existing immunity hurdles in gene therapy and vaccine applications.

In certain embodiments, the adenoviral vector is derived from a gorilla adenovirus type 40 (GAd40), such as GC44, GC45, or GC46. In certain embodiments, the adenoviral vector represents a functional adaptation of the aforementioned. These adaptations may encompass sequences encoding functional variants of their constituent components, such as the E2B, E2A, E3, and L1-L5 regions, as well as inverted terminal repeats. Also envisaged are functional adaptations of such vectors featuring codon degenerate variants of the sequences encoding the E2B, E2A, E3, and L1-L5 regions.

In particularly preferred embodiments, the adenoviral vector is derived from GC46, a newly-isolated and unique gorilla adenovirus strain, isolated from a healthy African gorilla stool specimen. This adenovirus is closely related to and clusters phylogenetically with the human species C adenoviruses based on hexon, DNA polymerase and Exon 4 ORF6 protein sequence comparison. Duncan et al., Virology, 444:119-123 (2013). The sero-prevalence of gorilla adenovirus type GC46, is less than about 6% in the United States. In comparison, the sero-prevalence of Ad5 type is about 57%, with most of the seropositive individuals having high titers (above 200 IC90). Johnson et al., Molecular Therapy, 22:196-205 (2014). Therefore, compared to traditional adenovirus therapies based on the Ad5 serotype, pre-existing neutralizing activity to gorilla adenovirus type GC46 is rare and weak in the United States. In addition, comparative studies from human sera samples from Sub-Saharan Africa confirmed the rare and weak pre-existing neutralizing activity in the human population. These data suggest that pre-existing neutralizing activity to GC46 will not significantly interfere with molecular vaccines and therapeutics built on this platform, which makes the gorilla adenovirus GC46 well suited as a back bone viral vector.

In some embodiments, the gorilla adenovirus vaccine may encode between about 1 and about 200 non-HLA-restricted epitopes from HPV 16/18. For example, the gorilla adenovirus vaccine may encode about 1, about 5, about 10, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 non-HLA-restricted epitopes from HPV 16/18.

In a specific embodiment of the present invention, the gorilla adenovirus vaccine encodes about 35 non-HLA-restricted epitopes from HPV 16/18.

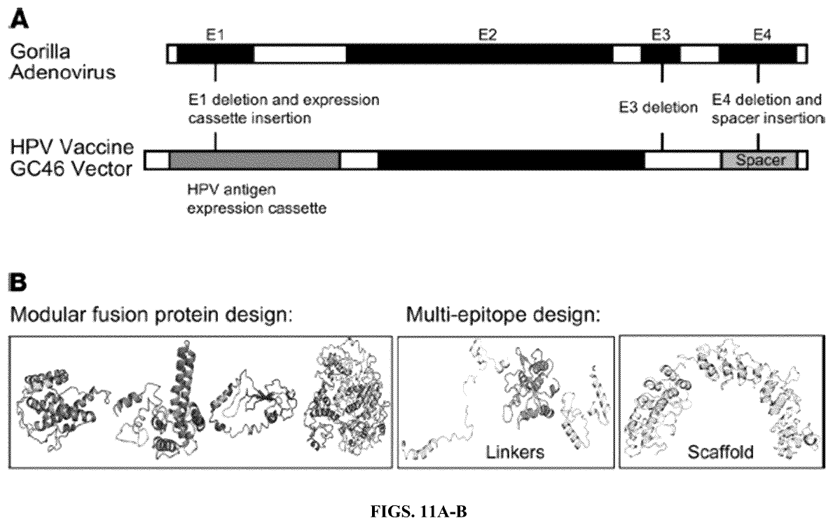

In certain embodiments, the adenovirus vector is a gorilla adenovirus vector engineered to delete portions of or the entire E1 and/or E4 regions. The deletion in the E1 region may, for example, render the adenovirus vector replication-deficient (FIG. 11A) and include bases 459 through 3411, resulting in deletion of the E1A and E1B promoters and open reading frames. The deletion in the E4 region may, for example, be inclusive of bases 34144 to 36824 and remove all the E4 open reading frames (ORFs), therefore eliminating essential elements for gorilla adenovirus replication. (The gorilla adenovirus coordinates provided herein are based on a wild-type adenovirus genome size of 37,213 base pairs.)

The modified gorilla adenovirus vector with the deletions of and/or in the E1 and/or E4 regions may provide one or more advantages over unmodified vector backbones. For example, the extended deletions of the adenoviral genome may provide enhanced payload capacity to the adenovirus vector. A second potential advantage is reduced risk of Replication Competent Adenovirus (RCA) generation during adenovirus vector production. A third advantage is that the elimination of E1 and E4 expression products may work to further silence other regions of the viral genome.

Thus, in one aspect, the gorilla adenovirus vectors described herein have the E1 region, or portions thereof, deleted. In another aspect, the gorilla adenovirus vectors described herein have the E4 region, or portions thereof, deleted. In another aspect the gorilla adenoviral vectors described herein have both the E1 and E4 regions, or portions thereof, deleted. In one aspect, the deletion(s) in the E1 and/or E4 regions comprise from about 100 to about 5,000 base pairs (bp) in length as compared to the wild-type. For example, the deletion(s) in the E1 and/or E4 regions may comprise about 100 bp, about 500 bp, about 1,000 bp, about 1,500 bp, about 2,000 bp, about 2,500 bp, about 3,000 bp, about 3,500 bp, about 4,000 bp, about 4,500 bp, or about 5,000 bp in length as compared to the wild-type. In some embodiments, the deletion(s) in the E1 and/or E4 regions comprise from about 100 bp to about 5,000 bp, or about 500 bp to about 4,500 bp, about 750 bp to about 4,000 bp, or about 1,000 bp to about 3,750 bp, or about 1,250 bp to about 3,500 bp, or about 1,500 bp to about 3,500 bp, or about 1,750 bp to about 3,500 bp, or about 2,000 bp to about 3,500 bp, or about 2,000 bp to about 3,000 bp. In specific embodiments, the deletion(s) in the E1 and/or E4 regions comprise about 3,000 bp in length as compared to the wild-type.

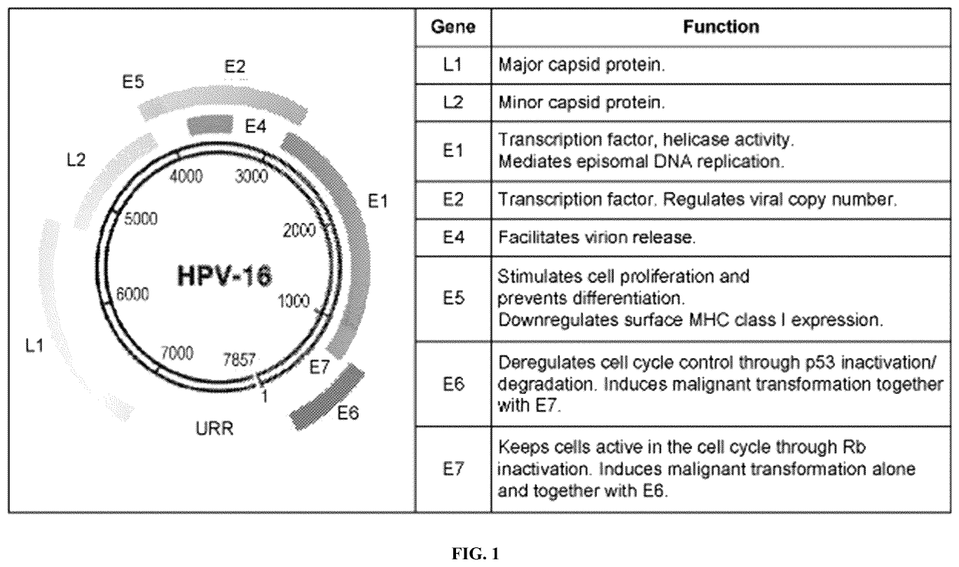

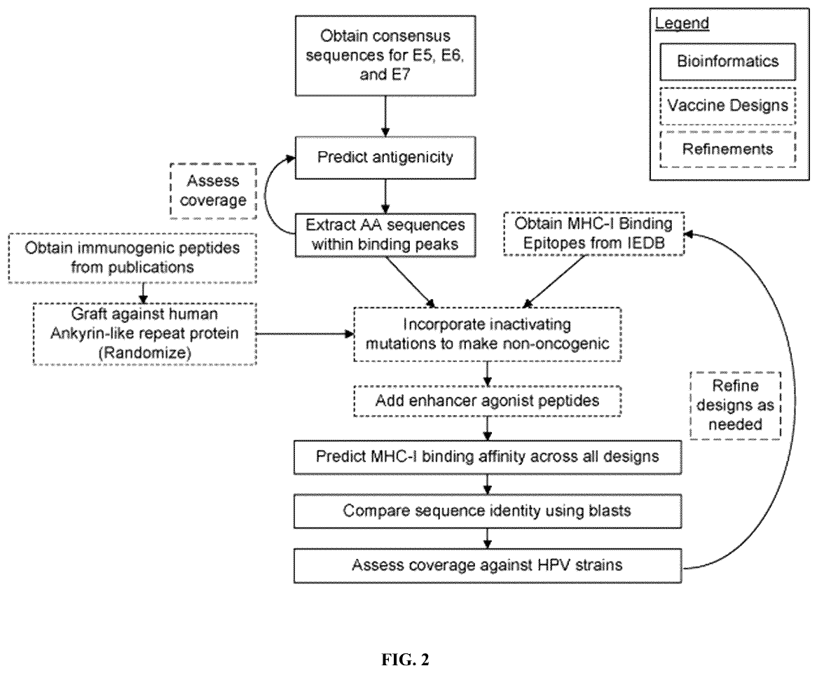

In certain embodiments, deletion of the E4 region removes all predicted open reading frames (ORFs) therein. To avoid the potential for low levels of production of adenoviral vectors with E4 deletions, spacer sequences may be inserted within the E4 deleted region, as depicted in FIG. 1, to stop any potential transcription initiated from the retained E4 promoter. In one aspect, the gorilla adenoviral vector described herein comprises a spacer sequence inserted in place of the deleted portion of the E4 region. In one aspect, the spacer sequence comprises a Bovine Growth Hormone polyadenylation (BGH polyA) signal sequence that is inserted in place of the deleted E4 ORFs, but any suitable spacer sequence may be used. In some aspects, the spacer sequence is about 10 to about 500 base pairs (bp) in length. For example, the spacer sequence may be about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, or about 500 bp in length. Alternatively, the spacer sequence may be about 50 bp to about 100 bp, about 100 bp to about 150 bp, about 150 bp to about 200 bp, about 200 bp to about 250 bp, about 250 bp to about 300 bp, about 300 bp to about 350 bp, about 350 bp to about 400 bp, about 400 bp to about 450 bp, or about 450 bp to about 500 bp, as compared to the wild-type. The spacer may also be any length within these ranges. For instance, the spacer may be about 250 bp to about 350 bp, about 260 bp to about 340 bp, about 270 bp to about 330 bp, about 280 bp to about 320 bp, or about 290 bp to about 310 bp in length. In one aspect, the spacer sequence is about 300 base pairs in length. In yet another aspect, the spacer sequence is 278 bp.

In another aspect, the location of the spacer sequence is at about 34,000 thru 34,500 base pairs of the vector genome as compared to the wild-type. In yet another aspect, the location of the spacer sequence is at 34,040 thru 34,317 base pairs of the vector genome as compared to the wild-type. In another aspect the spacer sequence comprises a nucleic acid sequence of SEQ ID NO: 157.

In certain embodiments, the vector has all of the E1 and E4 regions deleted. In some such embodiments, the E1 or E4 region is replaced with an expression cassette comprising a transgene or a spacer. In some such embodiments, the E1 region is replaced with the expression cassette and the E4 region is replaced with a spacer.

In one embodiment, an E1/E4-deficient GC46 adenoviral vector can be produced in any complementing cell line that provides for the functions of E1 and E4 ORF6, for example, an engineered 293 cell. Such cells may be cultured, for example, in a serum-free suspension in a shaker flask and infected with master virus bank at a multiplicity of infection (MOI) of 100 PU/cell. The culture harvest may be downstream processed and purified using three rounds of cesium chloride density gradient ultracentrifugation to yield a highly purified material. This material may then be frozen and later thawed and sterile-filtered and filled into vials which can be stored in a freezer at about −60 to about −90° C.

In some embodiments, the vector of the present invention may be made by isolating the GC46 gorilla adenovector from nonhuman primate sources, cloning the isolated GC46 genome, deleting the E1 and E4 regions of GC46, and inserting an expression cassette in the E1 region that expresses a human papilloma virus (HPV) 16/18 antigen design (e.g., any antigen design construct described herein, including, but not limited to SEQ ID NO: 243) and is under the control of a cytomegalovirus (CMV) immediate early promoter. In one embodiment described below, the CMV-HPV 16/18 antigen design contains 35 non-HLA-restricted epitopes of HPV 16 and 18. The overall workflow of the presently disclosed HPV vaccine designs is shown in FIG. 2 and is further detailed in the examples.

In some embodiments, the vector comprises a nucleic acid sequence encoding a fusion protein comprising an amino acid sequence having at least 80%, 85%, 90%, 95%, 96%, 97%, 98%, 99%, 99.5%, 99.6%, 99.7%, 99.8%, or 99.9% identity with SEQ ID NO: 243. In some embodiments, the fusion protein comprises an amino acid sequence of SEQ ID NO: 243 or a conservatively-substituted variant thereof.

In some embodiments, the vector comprises a nucleic acid sequence having at least 80% (e.g., 80%, 85%, 90%, 95%, 96%, 97%, 98%, 99%, 99.5%, 99.9% or more) identity with SEQ ID NO: 244. In some embodiments, the vector comprises a nucleic acid sequence having at least 90% identity with SEQ ID NO: 244. In some embodiments, the vector comprises a nucleic acid sequence having at least 95% identity with SEQ ID NO: 244. In some embodiments, the vector comprises a nucleic acid sequence having at least 96% identity with SEQ ID NO: 244. In some embodiments, the vector comprises a nucleic acid sequence having at least 97% identity with SEQ ID NO: 244. In some embodiments, the vector comprises a nucleic acid sequence having at least 98% identity with SEQ ID NO: 244. In some embodiments, the vector comprises a nucleic acid sequence having at least 99% identity with SEQ ID NO: 244. In some embodiments, the vector comprises a nucleic acid sequence having at least 99.5% identity with SEQ ID NO: 244. In some embodiments, the vector comprises a nucleic acid sequence having at least 99.9% identity with SEQ ID NO: 244. In some embodiments, the vector comprises a nucleic acid sequence having from 1 to about 500 nucleotide substitutions (e.g., from 1 to about 450, from 1 to about 450, from 1 to about 350, from 1 to about 300, from 1 to about 250, from 1 to about 200, from 1 to about 150, from 1 to about 100, from 1 to about 90, from 1 to about 80, from 1 to about 70, from 1 to about 60, from 1 to about 50, from 1 to about 40, from 1 to about 30, from 1 to about 25, from 1 to about 20, from 1 to about 15, or from 1 to about 10) compared to SEQ ID NO: 244. In some embodiments, the vector comprises a nucleic acid sequence having SEQ ID NO: 244.

### C. Non-Viral Based Delivery Systems

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

The use of lipid formulations is contemplated for the introduction of the nucleic acids into a host cell (in vitro, ex vivo or in vivo). In another aspect, the nucleic acid can be associated with a lipid. The nucleic acid associated with a lipid can be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they can be present in a bilayer structure, as micelles, or with a “collapsed” structure. They can also simply be interspersed in a solution, possibly forming aggregates that are not uniform in size or shape. Lipids are fatty substances which can be naturally-occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids can be obtained from Avanti Polar Lipids, Inc. (Birmingham, Ala.). Stock solutions of lipids in chloroform or chloroform/methanol can be stored at about −200 C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5:505-10 (1991)). However, compositions that have different structures in solution than the normal vesicular structure are also encompassed. For example, the lipids can assume a micellar structure or merely exist as nonuniform aggregates of lipid molecules. Also contemplated are lipofectamine-nucleic acid complexes.

In some instances, polynucleotides encoding polypeptides can also be introduced into cells using non-viral based delivery systems, such as the “Sleeping Beauty (SB) Transposon System.” In embodiments, a modified effector cell described herein and other genetic elements are delivered to a cell using the SB11 transposon system, the SB100X transposon system, the SB110 transposon system, the piggyBac transposon system (see, e.g., Wilson et al., “PiggyBac Transposon-mediated Gene Transfer in Human Cells,” Molecular Therapy 15:139-145 (2007), incorporated herein by reference in its entirety) and/or the piggyBac transposon system (see, e.g., Mitra et al., “Functional characterization of piggy Bac from the bat Myotis lucifugus unveils an active mammalian DNA transposon,” Proc. Natl. Acad. Sci USA 110:234-239 (2013). Additional transposases and transposon systems are provided in U.S. Pat. Nos. 6,489,458; 6,613,752, 7,148,203; 7,985,739; 8,227,432; 9,228,180; U.S. Patent Pub. No. 2011/0117072; Mates et al., Nat Genet, 41 (6): 753-61 (2009). doi: 10.1038/ng.343. Epub 2009 May 3, Gene Ther., 18 (9): 849-56 (2011). doi: 10.1038/gt.2011.40. Epub 2011 Mar. 31 and in Ivies et al., Cell, 91 (4): 501-10, (1997), each of which is incorporated herein by reference in their entirety.

Additional suitable non-viral systems can include integrating expression vectors, which can randomly integrate into the host cell's DNA, or can include a recombination site to enable the specific recombination between the expression vector and the host cell's chromosome. Targeted integration of transgenes into predefined genetic loci is a desirable goal for many applications. First, a first recombination site for a site-specific recombinase is inserted at a genomic site, either at a random or at a predetermined location. Subsequently, the cells are transfected with a plasmid carrying the gene or DNA of interest and the second recombination site and a source for recombinase (expression plasmid, RNA, protein, or virus-expressing recombinase). Recombination between the first and second recombination sites leads to integration of plasmid DNA. Such integrating expression vectors can utilize the endogenous expression control sequences of the host cell's chromosomes to effect expression of the desired protein.

In some embodiments, targeted integration is promoted by the presence of sequences on the donor polynucleotide that are homologous to sequences flanking the integration site. For example, targeted integration using the donor polynucleotides described herein can be achieved following conventional transfection techniques, e.g. techniques used to create gene knockouts or knockins by homologous recombination. In other embodiments, targeted integration is promoted both by the presence of sequences on the donor polynucleotide that are homologous to sequences flanking the integration site, and by contacting the cells with donor polynucleotide in the presence of a site-specific recombinase. By a site-specific recombinase, or simply a recombinase, it is meant is a polypeptide that catalyzes conservative site-specific recombination between its compatible recombination sites. As used herein, a site-specific recombinase includes native polypeptides as well as derivatives, variants and/or fragments that retain activity, and native polynucleotides, derivatives, variants, and/or fragments that encode a recombinase that retains activity.

Also provided herein is a system for integrating heterologous genes in a host cell, said system comprising one or more gene expression cassettes. In some instances, the system includes a first gene expression cassette comprising a first polynucleotide encoding a first polypeptide construct. In other instances, the system can include a second gene expression cassette comprising a second polynucleotide encoding a second polypeptide construct. In yet other instances, the system can include a third gene expression cassette. In one embodiment, one of the gene expression cassettes can comprise a gene switch polynucleotide encoding one or more of: (i) a transactivation domain; (ii) nuclear receptor ligand binding domain; (iii) a DNA-binding domain; and (iv) ecdysone receptor binding domain. In another embodiment, the system further includes recombinant attachment sites; and a serine recombinase; such that upon contacting said host cell with at least said first gene expression cassette, in the presence of said serine recombinase, said heterologous genes are integrated in said host cell.

In some instances, the system further comprises a ligand; such that upon contacting said host cell, in the presence of said ligand, said heterologous gene are expressed in said host cell. In one instance, the system also includes recombinant attachment sites. In some instances, one recombination attachment site is a phage genomic recombination attachment site (attP) or a bacterial genomic recombination attachment site (attB). In one instance, the host cell is an eukaryotic cell. In another instance, the host cell is a human cell. In further instances, the host cell is a T cell or NK cell.

### II. Expression Cassettes

Any of the expression cassettes described herein or polynucleotides comprising any of the expression cassettes described herein can be used as a component in a vaccine, e.g., an HPV vaccine.

### A. Transgenes

The vector of the present invention may comprise an expression cassette for expressing a transgene. A “transgene” comprises a non-native nucleic acid sequence that is operably linked to appropriate regulatory elements (e.g., a promoter), such that the non-native nucleic acid sequence can be expressed to produce a protein (e.g., peptide or polypeptide). The regulatory elements (e.g., promoter) can be native or non-native to the adenovirus or adenoviral vector.

A “non-native” nucleic acid sequence is any nucleic acid sequence (e.g., DNA, RNA, or cDNA sequence) that is not a naturally occurring nucleic acid sequence of an adenovirus in a naturally occurring position. Thus, the non-native nucleic acid sequence can be naturally found in an adenovirus, but located at a non-native position within the adenoviral genome and/or operably linked to a non-native promoter. The terms “non-native nucleic acid sequence,” “heterologous nucleic acid sequence,” and “exogenous nucleic acid sequence” are synonymous and can be used interchangeably in the context of the present disclosure. The non-native nucleic acid sequence preferably is DNA and preferably encodes a protein (i.e., one or more nucleic acid sequences encoding one or more proteins).

The non-native nucleic acid sequence can encode a therapeutic protein that can be used to prophylactically or therapeutically treat a mammal for a disease. Examples of suitable therapeutic proteins include anti-inflammatory agents such as cytokines, toxins, tumor suppressor proteins, growth factors, hormones, receptors, mitogens, immunoglobulins, neuropeptides, neurotransmitters, and enzymes. Alternatively, the non-native nucleic acid sequence can encode an antigen of a pathogen (e.g., a bacterium or a virus), and the adenovirus or adenoviral vector can be used as a vaccine.

### B. Promoters

Genetic regulatory components of the therapeutic expression cassette are selected to confer a high level expression of the transgene. Thus, another aspect of the present disclosure is an expression cassette that further comprises a promoter. A promoter is a region of a polynucleotide that initiates transcription of a coding sequence. Promoters are located near the transcription start sites of genes, on the same strand and upstream on the DNA (towards the 5′ region of the sense strand). Some promoters are constitutive as they are active in all circumstances in the cell, while others are regulated becoming active in response to specific stimuli, e.g., an inducible promoter. Yet other promoters are tissue specific or activated promoters, including but not limited to T-cell specific promoters.

The term “promoter activity” and its grammatical equivalents as used herein refer to the extent of expression of nucleotide sequence that is operably linked to the promoter whose activity is being measured. Promoter activity can be measured directly by determining the amount of RNA transcript produced, for example, by Northern blot analysis or indirectly by determining the amount of product coded for by the linked nucleic acid sequence, such as a reporter nucleic acid sequence linked to the promoter.

### 1. Inducible Promoters

In certain embodiments, the promoter is an inducible promoter. An inducible promoter is a promoter that is induced into activity by the presence or absence of transcriptional regulators, e.g., biotic or abiotic factors. Inducible promoters are useful because the expression of genes operably linked to them can be turned on or off at certain stages of development of an organism or in a particular tissue. Examples of inducible promoters include alcohol-regulated promoters, tetracycline-regulated promoters, steroid-regulated promoters, metal-regulated promoters, pathogenesis-regulated promoters, temperature-regulated promoters and light-regulated promoters. In some embodiments, the inducible promoter is part of a genetic switch. The inducible promoter can be a gene switch ligand inducible promoter. In some cases, an inducible promoter can be a small molecule ligand-inducible two polypeptide ecdysone receptor-based gene switch, such as RHEOSWITCH® gene switch, such as the system described in WO 2018/132494. In some cases, a gene switch can be selected from ecdysone-based receptor components as described in, but without limitation to, any of the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. Pat. No. 8,715,959 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. Pat. No. 9,402,919).

An inducible promoter typically utilizes a ligand for dose-regulated control of expression of said at least two genes. In some cases, the ligand can be selected from a group consisting of ecdysteroid, 9-cis-retinoic acid, synthetic analogs of retinoic acid, N,N′-diacylhydrazines, oxadiazolines, dibenzoylalkyl cyanohydrazines, N-alkyl-N,N′-diaroylhydrazines, N-acyl-N-alkylcarbonylhydrazines, N-aroyl-N-alkyl-N′-aroylhydrazines, arnidoketones, 3,5-di-tert-butyl-4-hydroxy-N-isobutyl-benzamide, 8-O-acetylharpagide, oxysterols, 22(R) hydroxycholesterol, 24 (S) hydroxycholesterol, 25-epoxycholesterol, T0901317, 5-alpha-6-alpha-epoxycholesterol-3-sulfate (ECHS), 7-ketocholesterol-3-sulfate, framesol, bile acids, 1,1-biphosphonate esters, juvenile hormone III, RG-115819 (3,5-Dimethyl-benzoic acid N-(1-ethyl-2,2-dimethyl-propyl)-N′-(2-methyl-3-methoxy-benzoyl)-hydrazide-), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide), and any combination thereof.

### 2. Non-Inducible Promoters

In certain embodiments, the promoter is a non-inducible promoter, including, e.g., tissue-specific, strong constitutive, or minimal promoters known in the art. Suitable non-inducible promoters may include, for example, an CMV promoter, a SV40 promoter, a CAG promoter, or others. In certain embodiments, the promoter is a CMV promoter.

### 3. Tissue-Specific Promoters

In certain embodiments, the promoter may be a tissue-specific promoter. Herein “tissue-specific” refers to regulated expression of a gene in a subset of tissues or cell types. In some cases, the tissue-specific promoter can be regulated spatially such that the promoter drives expression only in certain tissues or cell types of an organism. In other cases, the tissue-specific promoter can be regulated temporally such that the promoter drives expression in a cell type or tissue differently across time, including during development of an organism. In some cases, the tissue-specific promoter is regulated both spatially and temporally. In certain embodiments, the tissue-specific promoter is activated in certain cell types either constitutively or intermittently at particular times or stages of the cell type. For example, the tissue-specific promoter can be a promoter that is activated when a specific cell such as a T cell or a NK cell is activated. T cells can be activated in a variety of ways, for example, when presented with peptide antigens by MHC class II molecules.

### 4. Synthetic and Engineered Promoters

Synthetic promoters are also contemplated for use in the expression cassette described herein, and may be engineered to improve expression characteristics. Synthetic promoters may include a variety of sub-components including, but not limited to, blocking sequences, enhancers, and various responsive elements.

In certain embodiments, the promoter is an engineered promoter or variant thereof. As described herein, the promoter can incorporate minimal promoter sequences from IL-2 and one or more of the following: nuclear factor of activated T-cells (NFAT) response element(s); NFIL2D response element, NF-κB/TCF response element, NFAT/NFIL2B response element or NFIL2A/OCT response element. NFAT transcription factors are key modulators of effector T-cell states. NFATs are early transcriptional checkpoint progressively driving exhaustion. NFATs are quickly activated in T cells following TCR stimulation and form a protein complex with AP-1 induced by appropriate co-stimulation signaling and regulate effector genes and T-cell functions. NFAT response element(s) can be fused with other minimal promoter sequences (e.g. IL2 minimal promoter) to drive expression of transgenes in response to T cell activation. Further examples of response elements are described in Mattila et al., EMBO J., 9 (13): 4425-33 (1990).

### 5. Activation-Specific Promoters

In certain embodiments, the promoter is an activation-specific promoter, for example, interleukin-2 (IL2) promoter and Programmed Death (PD)-1 (CD279) promoter. Gene switch components can also be conditionally expressed upon immune cell activation by fusing binding sites for other nuclear factors like NF-KB of proinflammatory signaling pathway to minimal promoter sequence (e.g. IL2).

In certain embodiments, the promoter comprises IL-2 core promoter (SEQ ID NO: 26). In some embodiments, at least one promoter comprises IL-2 minimal promoter (SEQ ID NO: 27). In another embodiment, at least one promoter comprises IL-2 enhancer and promoter variant (SEQ ID NOs: 26-28). In yet another embodiment, at least one promoter comprises NF-κB binding site (SEQ ID NOs: 30-32). In some embodiments, at least one promoter comprises (NF-κB)₁-IL2 promoter variant (SEQ ID NO: 30). In some embodiments, at least one promoter comprises (NF-κB)₃-IL2 promoter variant (SEQ ID NO: 31). In some embodiments, at least one promoter comprises (NF-κB)₆-IL2 promoter variant (SEQ ID NO: 32). In some embodiments, at least one promoter comprises 1× nuclear factor of activated T-cells (NFAT) response elements-IL2 promoter variant (SEQ ID NO: 33). In another embodiment, at least one promoter comprises 3×NFAT response element (SEQ ID NOs: 34-35). In yet another embodiment, at least one promoter comprises 6×NFAT response elements-IL2 promoter variant (SEQ ID NOs: 36-39). In some embodiments, at least one promoter comprises human EF1A1 promoter variant (SEQ ID NO: 40-41). In some embodiment, at least one promoter comprises human EF1A1 promoter and enhancer (SEQ ID NO: 42). In some embodiments, at least one promoter comprises human UBC promoter (SEQ ID NO: 43). In some embodiments, at least one promoter comprises 6 site GAL4-inducible proximal factor binding element (PFB). In some embodiment, at least one promoter comprises synthetic minimal promoter 1 (inducible promoter) (SEQ ID NO: 44). Sequences for such promoters are described in, for example, for example, in WO 2019/173465 and WO 2022/115470.

In certain embodiments, the promoter can be any one or more of: IL-2 core promoter, IL-2 minimal promoter, IL-2 enhancer and promoter variant, (NF-κB)₁-IL2 promoter variant, (NF-κB)₃-IL2 promoter variant, (NF-κB)₆-IL2 promoter variant, 1×NFAT response elements-IL2 promoter variant, 3×NFAT response elements-IL2 promoter variant, 6×NFAT response elements-IL2 promoter variant, human EEF1A1 promoter variant, human EEF1A1 promoter and enhancer, human UBC promoter and synthetic minimal promoter 1. In certain embodiments, the promoter nucleotides can comprise SEQ ID NOS: 26-44.

### 6. Constitutive Promoters

In certain embodiments, the promoter is a constitutive promoter. Examples of such promoters include the simian virus 40 (SV40) early promoter, mouse mammary tumor virus (MMTV), human immunodeficiency virus (HIV) long terminal repeat (LTR) promoter, MoMuL V promoter, an avian leukemia virus promoter, an Epstein-Barr virus immediate early promoter, a Rous sarcoma virus promoter, as well as human gene promoters such as the actin promoter, the myosin promoter, the hemoglobin promoter, and the creatine kinase promoter.

### 7. Viral Promoters

Exemplary promoters used in the vectors described herein include viral promoters in operable combination with a heterologous nucleic acid sequence encoding the cytokine. Exemplary viral promoters may be derived from multiple known viruses, including but not limited to retroviral vectors, lentiviral vectors, adenovirus vectors, adeno-associated virus vectors (AAV), alphavirus vectors and the like. Non-limiting examples of potentially useful viral vectors include Human Immunodeficiency Virus (HIV), Respiratory Syncytial Virus (RSV), Cytomegalovirus (CMV), Simian virus 40, Herpes Simplex Virus (HSV), Adenovirus (AV), Adeno-Associated Virus (AAV), or Lentivirus (LV). For example, specific viral promoters contemplated herein include cytomegalovirus (CMV) immediate early promoter, CAG promoter (which is a combination of the CMV early enhancer element and chicken beta-actin promoter), simian virus 40 (SV40) promoter, the 35S RNA and 19S RNA promoters of cauliflower mosaic virus (CaMV), the coat protein promoter to tobacco mosaic virus (TMV), and any variants thereof. Examples of mammalian promoters include human elongation factor 1α-subunit (EF1-1α) promoter, human ubiquitin C (UCB) promoter, murine phosphoglycerate kinase-1 (PGK) promoter, and any variants thereof.

### 8. Other Promoter Elements

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

Synthetic promoters useful in the present invention may include enhancer sequences. In one aspect, the enhancer may be an mCMV enhancer sequence. In another aspect, the mCMV enhancer sequence is about 200 to about 1,000 bp in length. In another aspect, the enhancer sequence is about 365 bp in length. In yet another aspect, the enhancer sequence comprises the nucleic acid sequence of SEQ ID NO: 148 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 148, or a conservatively-substituted variant of SEQ ID NO: 148, or a non-conservatively-substituted variant of SEQ ID NO: 148.

In another aspect, the mCMV enhancer comprises transcription factor binding sites. In yet another aspect, the transcription factor binding sites comprise Sp1, Ebox, ETS, TRE, CREB, and GATA binding sites.

Responsive elements may also be included in the promoters described herein. Various responsive elements are known in the art. For example, to reduce expression of the transgene during adenovirus production, which can sometimes negatively impact overall production titer, a Tetracyline Responsive Element (TRE, 2× TetO) may be positioned between the TATA box and the transcription initiation site within a promoter element of the promoter. Thus, when the vector is produced in cell lines that express the Tetracycline (Tet) repressor, expression of the transgene driven by a promoter containing a TRE is reduced. Gall et al., Molecular Biotechnology, 35:263-273 (2007). In the absence of tetracycline, the Tet repressor interacts with the TRE element and blocks the initiation of transcription. Upon infection of producer cells that do not express the Tet repressor, normal expression levels are observed. Thus, in one aspect described herein, the synthetic promoter comprises a TRE. The TRE comprises about 10 to about 100 bp in length. In another aspect, the TRE ranges from 40 bp to 50 bp in length. In yet another aspect, the TRE comprises a nucleic acid sequence of SEQ ID NO: 150 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 150, or a conservatively-substituted variant of SEQ ID NO: 150, or a non-conservatively-substituted variant of SEQ ID NO: 150.

In certain embodiments, the promoter comprises a transcription blocker, an enhancer sequence and a responsive element. In some such embodiments, the promoter comprises an mCMV enhancer and a TRE.

### B. Untranslated Regions

In some embodiments, the expression cassette may include an untranslated region (UTR) to regulate or enhance transgene expression. In one aspect, the expression cassette may include an artificial untranslated region. A 5′UTR with a splice unit has been demonstrated to enhance expression of a transgene cassette. Thus, in an embodiment, the cassette comprises a 5′UTR with a splice unit. In certain embodiments, the 5′UTR is engineered to include a synthetic splice site sequence spanning a canine ATP2A2 intron 2 followed by the 5′ UTR of bovine CSN2 gene. In another aspect, the 5′ UTR comprises a nucleic acid sequence of SEQ ID NO: 151 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 151, or a conservatively-substituted variant of SEQ ID NO: 151, or a non-conservatively-substituted variant of SEQ ID NO: 151.

### C. Termination Sequences

In another aspect, the expression cassette further comprises a termination sequence. The open reading frame of the cytokine transgene may be followed by a termination sequence. Like the promoter sequence, the termination sequence may also include various regulatory elements to ensure proper 3′ transcript end processing. In one aspect, the termination sequence comprises a partial human growth hormone (HGH) 3′ untranslated region. In another aspect, the termination sequence comprises a polyadenylation signal, including but not limited to a SV40 polyadenylation signal and/or a LTR polyadenylation signal. In yet another aspect, the termination sequence comprises a human beta actin (ACTb) transcriptional termination signal sequence. In another aspect, the termination sequence comprises a HGH 3′ untranslated region, a polyadenylation signal, and a human beta actin transcriptional termination sequence. In another aspect, the termination sequence comprises the nucleic acid sequence of SEQ ID NO: 156 or a functional variant thereof, e.g., a nucleic acid having at least 80%, 85%, 90%, 95%, 96%, 97%, 98%, 99%, 99.1%, 99.5%, 99.9%, or 99.99% sequence identity with SEQ ID NO: 156, or a conservatively-substituted variant of SEQ ID NO: 156, or a non-conservatively-substituted variant of SEQ ID NO: 156.

### D. Polynucleotide Linkers

Also contemplated herein are expression cassettes and constructs comprising a polynucleotide linker to facilitate the expression of the polynucleotides and functionality of the polypeptides described herein.

In some cases, the linker may be a cleavable linker. The polynucleotide linker can be an oligomer. The polynucleotide linker can be a DNA double strand, single strand, or a combination thereof. In some cases, the linker can be RNA. A polynucleotide linker can be a double-stranded segment of DNA containing desired restriction sites that can be added to create end structures that are compatible with a vector comprising a polynucleotide described herein.

In some cases, a polynucleotide linker can be useful for modifying vectors comprising polynucleotides described herein. For example, a vector modification comprising a polynucleotide linker can be a change in a multiple cloning site, or the addition of a poly-histidine tail. Polynucleotide linkers can also be used to adapt the ends of blunt insert DNA for cloning into a vector cleaved with a restriction enzyme with cohesive end termini. The use of polynucleotide linkers can be more efficient than a blunt ligation into a vector and can provide a method of releasing an insert from a vector in downstream applications. The insert may be a polynucleotide sequence encoding polypeptides useful for therapeutic applications.

In some embodiments, the polynucleotide linker may be ligated into a vector comprising a polynucleotide described herein by a T4 ligase in some cases. To facilitate a ligation an excess of polynucleotide linkers can be added to a composition comprising an insert and a vector. In some cases, an insert and vector are pre-treated before a linker is introduced. For example, pre-treatment with a methylase can prevent unwanted cleavage of insert DNA.

In some cases, the polynucleotides or genes described herein may be separated by 1, 2, 3, 4, 5, 6, 7, 8, 9, or up to 10 linkers.

### 1. IRES Sequences

In some embodiments, the polynucleotide(s) described herein may be linked by an “internal ribosome entry site,” or “IRES” element. An IRES can allow simultaneous expression of multiple genes. For example, an IRES sequence can permit production of multiple proteins from a single mRNA transcript. A ribosome can bind to an IRES in a 5′-cap independent manner and initiate translation.

In an expression cassette comprising an IRES sequence, a first gene can be translated by a cap-dependent, ribosome scanning, mechanism with its own 5′-UTR, whereas translation of a subsequent gene can be accomplished by direct recruitment of a ribosome to an IRES in a cap-independent manner. An IRES sequence can allow eukaryotic ribosomes to bind and begin translation without binding to a 5′ capped end. An IRES sequence can allow expression of multiple genes from one transcript (Mountford and Smith, Trends Genet. 11 (5): 179-84 (1995)).

In certain cases, an IRES region can be derived from a virus, such as picornavirus, encephalomyocarditis virus, hepatitis C virus IRES sequence. In other cases, an IRES sequence can be derived from an encephalomyocarditis virus. The term “EMCV” or “encephalomyocarditis virus” as used herein, refers to any member isolate or strain of the encephalomyocarditis virus species of the genus of the family Picornaviridae. Examples are: EMCV-R (Rueckert) strain virus and Columbia-SK virus. In some cases, a cellular IRES element, such as eukaryotic initiation factor 4G, immunoglobulin heavy chain binding protein, c-myc proto-oncogene, vascular endothelial growth factor, fibroblast growth factor-I IRES, or any combination or modification thereof can be used. In some cases, a cellular IRES can have increased gene expression when compared to a viral IRES.

An IRES sequence of viral, cellular, or a combination thereof can be utilized in the vector. An IRES can be from encephalomyocarditis (EMCV) or poliovirus (PV). In some cases, an IRES element is selected from a group consisting of Poliovirus (PV), Encephalomyelitis virus (EMCV), Foot-and-mouth disease virus (FMDV), Porcine teschovirus-1 (PTV-1), Aichivirus (AiV), Sencca Valley virus (SVV), Hepatitis C virus (HCV), Classical swine fever virus (CSFV), Human immunodeficiency virus-2 (HIV-2), Human immunodeficiency virus-I (HIV-I), Moloney murine leukemia virus (MoMLV), Feline immunodeficiency virus (FIV), Mouse mammary tumor virus (MMTV), Human cytomegalovirus latency (pUL138), Epstein-Barr virus (EBNA-1), Herpes virus Marek's disease (MDV RLORF9), SV40 polycistronic 19S (SV40 19S), Rhopalosiphum padi virus (RhPV), Cricket paralysis virus (CrPV), Ectropis obliqua picorna-like virus (EoPV), Plautia stali intestine virus (PSIV), Triatoma virus (TrV), Bee paralysis dicistrovirus (IAPV, KBV), Black currant reversion virus (BRV), Pelargonium flower break virus (PFBV), Hibiscus chlorotic ringspot virus (HCRSV), Crucifer-infecting tobamovirus (CrTMV), Potato leaf roll polcrovirus (PLRV), Tobacco etch virus (TEV), Giardiavirus (GLV), Leishmania RNA virus-I (LRV-1), and combinations or modifications thereof.

In some cases, an IRES is selected from a group consisting of Apaf-1, XIAP, HIAP2/c-IAP1, DAP5, Bcl-2, c-myc, CAT-I, INR, Differentiation LEF-1, PDGF2, HIF-1a, VEGF, FGF2, BiP, BAG-I, CIRP, p53, SHMTI, PITSLREp58, CDKI, Rpr, hid, hsp70, grim, skl, Antennapedia, dFoxO, dinR, Adh-Adhr, HSPI0I, ADH, URE-2, GPRI, NCE102, YMR181a, MSNI, BOil, FLO8, GICI, and any combination or modification thereof. When an IRES element is included between two open reading frames (ORFs), initiation of translation can occur by a canonical 5′-m7GpppN cap-dependent mechanism in a first ORF and a cap-independent mechanism in a second ORF downstream of the IRES element.

In some cases, an IRES sequence can be from about 9 to about 1,000 base pairs. For example, an IRES sequence can be from about 9 to about 150 base pairs, or from about 150 to about 400 base pairs, from about 400 to about 600 base pairs, or from about 600 to 1,000 base pairs. In some embodiments, the IRES sequence is about 9, about 25, about 50, about 75, about 100, about 125, about 150, about 175, about 200, about 225, about 250, about 275, about 300, about 325, about 350, about 275, about 400, about 425, about 450, about 475, about 500, about 525, about 550, about 575, about 600, about 625, about 650, about 675, about 700, about 750, about 800, about 850, about 900, about 950, or about 1,000 base pairs.

In some cases, expression of a downstream gene within a vector comprising an IRES sequence can be reduced. For example, a gene following an IRES sequence can have reduced expression over a gene preceding an IRES sequence. Reduced expression can be from 1% to 99.9% reduction over a preceding gene, including, e.g., a 1%, 5%, 10%, 15%, 20%, 25%, 30%, 35%, 40%, 45%, 50%, 55%, 60%, 65%, 70%, 75%, 80%, 85%, 90%, 95%, 99%, 99.5%, or 99.9% reduction over a preceding gene.

### 2. Viral 2A Sequences

In some embodiments, the polynucleotide(s) described herein may be linked by a viral 2A element or sequence. 2A elements can be shorter than IRES, having from 5 to 100 base pairs. In some cases, a 2A sequence may comprise 5, 10, 15, 20, 25, 30, 35, 40, 45, 50, 55, 60, 65, 70, 75, 80, 85, 90, or 100 base pairs. 2A linked genes can be expressed in one single open reading frame and “self-cleavage” can occur co-translationally between the last two amino acids, GP, at the C-terminus of the 2A polypeptide, giving rise to equal amounts of co-expressed proteins.

A viral 2A sequence can be about 20 amino acids. In some cases, a viral 2A sequence can contain a consensus motif Asp-Val/Ile-Glu-X-Asn-Pro-Gly-Pro (SEQ ID NO: 278). A consensus motif sequence can act co-translationally. For example, formation of a normal peptide bond between a glycine and praline residue can be prevented, which can result in ribosomal skipping and cleavage of a nascent polypeptide. This effect can produce multiple genes at equimolar levels.

A 2A peptide can allow translation of multiple proteins in a single open reading frame into a polypeptide that can be subsequently cleaved into individual polypeptide through a ribosome-skipping mechanism (Funston et al., J Gen. Viral. 89 (Pt 2): 389-96 (2008)). In some embodiments, a 2A sequence can include: F/T2A, T2A, p2A, 2A, T2A, E2A, F2A, and BmCPV2A, BmIFV2A, and any combination thereof.

In some cases, a vector can comprise an IRES sequence and a 2A linker sequence. In other cases, expression of multiple genes linked with 2A peptides can be facilitated by a spacer sequence (GSG) ahead of the 2A peptides. In some cases, constructs can combine a spacers, linkers, adaptors, promoters, or combinations thereof. For example, a linker can have a spacer (SGSG (SEQ ID NO: 84) or GSG or Whitlow linker) and furin linker (R-A-K-R (SEQ ID NO: 86)) cleavage site with different 2A peptides. A spacer can be an I-Ceui. In some cases, a linker can be engineered. For example, a linker can be designed to comprise chemical characteristics such as hydrophobicity. In some cases, at least two linker sequences can produce the same protein. In other cases, multiple linkers can be used in a vector. For example, genes of interest can be separated by at least two linkers.

### 3. Polypeptide Linkers Encoded by the Polynucleotide(s)

In certain embodiments, the polynucleotides described herein may encode two or more polypeptides. In some of those embodiments, the polynucleotides may be separated by an intervening sequence encoding an intervening linker polypeptide. As used herein, the term “intervening linker polypeptide” means an amino acid sequence separating two or more polypeptides encoded by a polynucleotide, and is distinguishable from the term “peptide linker” which refers to the sequence of amino acids, which is optionally included in a polypeptide construct disclosed herein, to connect the transmembrane domain to the cell surface polypeptide (e.g., comprising a truncated variant of a natural polypeptide).

In certain cases, the intervening linker polypeptide is a cleavage-susceptible intervening linker polypeptide. In some embodiments, the intervening linker polypeptide is a cleavable or ribosome skipping linker. In some embodiments, the cleavable linker or ribosome skipping linker sequence is selected from the group consisting of 2A, GSG-2A, GSG linker, SGSG linker (SEQ ID NO: 84), furinlink variants and derivatives thereof. In some embodiments, the 2A linker is a p2A linker, a T2A linker, F2A linker or E2A linker. In some embodiments, polypeptides of interest are expressed as fusion proteins linked by a cleavage-susceptible intervening linker polypeptide. In certain embodiments, cleavage-susceptible intervening linker polypeptide(s) can be any one or more of: F/T2A, T2A, p2A, 2A, GSG-p2A, GSG linker, and furinlink variants. Linkers (polynucleotide and polypeptide sequences), such as those disclosed in PCT/US2016/061668 (WO2017083750) published 18 May 2017, which is incorporated by reference herein. In certain cases, a furin intervening linker polypeptide may be encoded by a polynucleotide sequence polynucleotide sequence comprising “CGTGCAAAGCGT (SEQ ID NO: 69)” or “AGAGCTAAGAGG (SEQ ID NO: 112)”.

In some embodiments, an intervening linker polypeptide comprises an amino acid sequence having at least 70%, 75%, 80%, 85%, 90%, 95%, 99%, 99.1%, 99.5%, 99.9%, 99.99%, or 100% identity with the amino acid sequence of a Whitlow linker (SEQ ID NO: 81), a linker of SEQ ID NO: 82, GSG linker, SGSG linker (SEQ ID NO: 84), (G4S) 3 linker (SEQ ID NO: 85), Furin cleavage site/Furlink 1 (SEQ ID NO: 86), Fmdv linker (SEQ ID NO: 87), Thosea asigna virus 2A region (T2A) (SEQ ID NO: 88), Furin-GSG-T2A (SEQ ID NO: 89), Furin-SGSG-T2A (SEQ ID NO: 90), porcine teschovirus-1 2A region (P2A) (SEQ ID NO: 91), GSG-P2A (SEQ ID NO: 92), equine rhinitis A virus 2A region (E2A) (SEQ ID NO: 93), foot-and-mouth disease virus 2A region (F2A) (SEQ ID NO: 94), FP2A (SEQ ID NO: 95), Linker-GSG (SEQ ID NO: 96), or a linker of SEQ ID NO: 97. In some cases, an intervening linker polypeptide comprises an amino acid sequence having at least 70%, 75%, 80%, 85%, 90%, 95%, 99%, 99.1%, 99.5%, 99.9%, 99.99%, or 100% identity with the amino acid sequence of SEQ ID NOS: 82, 96, and/or 97.

In some embodiments, the intervening linker polypeptide comprises a conservatively-substituted or a non-conservatively-substituted variant of a Whitlow linker (SEQ ID NO: 81), a linker of SEQ ID NO: 82, GSG linker, SGSG linker (SEQ ID NO: 84), (G4S) 3 linker (SEQ ID NO: 85), Furin cleavage site/Furlink1 (SEQ ID NO: 86), Fmdv linker (SEQ ID NO: 87), Thosea asigna virus 2A region (T2A) (SEQ ID NO: 88), Furin-GSG-T2A (SEQ ID NO: 89), Furin-SGSG-T2A (SEQ ID NO: 90), porcine teschovirus-1 2A region (P2A) (SEQ ID NO: 91), GSG-P2A (SEQ ID NO: 92), equine rhinitis A virus 2A region (E2A) (SEQ ID NO: 93), foot-and-mouth disease virus 2A region (F2A) (SEQ ID NO: 94), FP2A (SEQ ID NO: 95), Linker-GSG (SEQ ID NO: 96), or a linker of SEQ ID NO: 97. In some cases, an intervening linker polypeptide comprises an amino acid sequence having at least 70%, 75%, 80%, 85%, 90%, 95%, 99%, 99.1%, 99.5%, 99.9%, 99.99%, or 100% identity with the amino acid sequence of SEQ ID NOS: 82, 96, and/or 97.

In some embodiments, the intervening linker polypeptide comprises a furin polypeptide and a 2A polypeptide connected by a polypeptide linker comprising at least three hydrophobic amino acids. In some cases, at least three hydrophobic amino acids are selected from the list consisting of glycine (Gly)(G), alanine (Ala)(A), valine (Val)(V), leucine (Leu)(L), isoleucine (Ile)(I), praline (Pro)(P), phenylalanine (Phe)(F), methionine (Met)(M), tryptophan (Trp)(W). In some cases, a polypeptide linker can also include one or more GS linker sequences, for instance (GS)n (SEQ ID NO: 279), (SG)n (SEQ ID NO: 280), (GSG)n (SEQ ID NO: 281), and (SGSG)n (SEQ ID NO: 282), wherein n can be any number from zero to fifteen.

The linkers described herein can, in certain cases, improve biological activity, increase expression yield, and achieving desirable pharmacokinetic profiles. A linker can also comprise hydrazone, peptide, disulfide, or thioester.

In some cases, the intervening linker polypeptide described herein is a flexible linker. Flexible linkers can be applied when a joined domain requires a certain degree of movement or interaction. Flexible linkers can be composed of small, non-polar (e.g., Gly) or polar (e.g., Ser or Thr) amino acids. A flexible linker can have sequences consisting primarily of stretches of Gly and Ser residues (“GS” linker). An example of a flexible linker can have the sequence of (Gly-Gly-Gly-Gly-Ser)n (SEQ ID NO: 283). By adjusting the copy number “n”, the length of this exemplary GS linker can be optimized to achieve appropriate separation of functional domains, or to maintain necessary inter-domain interactions. Besides GS linkers, other flexible linkers can be utilized for recombinant fusion proteins. In some cases, flexible linkers can also be rich in small or polar amino acids such as Gly and Ser, but can contain additional amino acids such as Thr and Ala to maintain flexibility. In other cases, polar amino acids such as Lys and Glu can be used to improve solubility.

Flexible linkers useful in the present invention may be rich in small or polar amino acids such as Gly and Ser to provide good flexibility and solubility. Flexible linkers can be suitable choices when certain movements or interactions are desired for fusion protein domains. In addition, although flexible linkers cannot have rigid structures, they can serve as a passive linker to keep a distance between functional domains. The length of flexible linkers can be adjusted to allow for proper folding or to achieve optimal biological activity of the fusion proteins.

In some cases, the intervening linker polypeptide described herein is a rigid linker. A rigid linker can be utilized to maintain a fixed distance between domains of a polypeptide. Examples of rigid linkers can be: Alpha helix-forming linkers, Pro-rich sequence, (XP) n, X-Pro backbone, A (EAAAK) nA (n=2-5) (SEQ ID NO: 284), to name a few. Rigid linkers can exhibit relatively stiff structures by adopting a-helical structures or by containing multiple Pro residues in some cases.

In some embodiments, the intervening linker polypeptide may be non-cleavable. Non-cleavable linkers can covalently join functional domains together to act as one molecule throughout an in vivo processes or an ex vivo process.

In other embodiments, the intervening linker polypeptide may be cleavable. A cleavable linker can be introduced to release free functional domains in vivo. A cleavable linker can be cleaved by the presence of reducing reagents, proteases, to name a few. For example, a reduction of a disulfide bond can be utilized to produce a cleavable linker. In the case of a disulfide linker, a cleavage event through disulfide exchange with a thiol, such as glutathione, could produce a cleavage. In other cases, an in vivo cleavage of a linker in a recombinant fusion protein can also be carried out by proteases that can be expressed in vivo under pathological conditions (e.g. cancer or inflammation), in specific cells or tissues, or constrained within certain cellular compartments. In some cases, a cleavable linker can allow for targeted cleavage. For example, the specificity of many proteases can offer slower cleavage of a linker in constrained compartments. A cleavable linker can also comprise hydrazone, peptides, disulfide, or thioester. For example, a hydrazone can confer serum stability. In other cases, a hydrazone can allow for cleavage in an acidic compartment. An acidic compartment can have a pH up to 7. A linker can also include a thioether. A thioether can be nonreducible A thioether can be designed for intracellular proteolytic degradation.

### 4. Engineered and Designed Linkers

In some embodiments, the polynucleotide linker may be engineered or designed. Methods of designing linkers can be computational. In some cases, computational methods can include graphic techniques. Computation methods can be used to search for suitable peptides from libraries of three-dimensional peptide structures derived from databases. For example, a Brookhaven Protein Data Bank (PDB) can be used to span the distance in space between selected amino acids of a linker.

### C. Adenoviral Expression Cassettes

In cases where an adenoviral vector is used, the expression cassette may be located at the E1 region deletion junction or the E4 deletion junction. In certain embodiments, the expression cassette is located in the E1 region deletion junction.

In certain embodiments, the expression cassette is cloned in the right-to-left orientation with respect to the adenovirus viral genome.

In certain embodiments, the expression cassette, as cloned in the right-to-left orientation within the adenovirus viral genome, comprises a nucleic acid sequence of SEQ ID NO: 242 or a functional variant thereof (e.g., a nucleic acid having at least 80%, 85%, 90%, 95%, 96%, 97%, 98%, 99%, 99.1%, 99.5%, 99.9%, or 99.99% sequence identity with SEQ ID NO: 242 or a codon degenerate variant of SEQ ID NO: 242, or a conservatively-substituted variant of SEQ ID NO: 242, or a non-conservatively-substituted variant of SEQ ID NO: 242).

### D. Packaging Sequences

In addition to the expression cassette, the vector may further comprise a packaging sequence. As used herein, the term “packaging sequence” refers to sequences located within the gorilla adenoviral genome which are required for insertion of the viral DNA into the viral capsid or particle. See Ostapchuk et al., Curr. Topics in Microbiology and Immunology, 272:165-185 (1995) and Ahi et al., Frontiers in Microbiology, 7:150 (2016).

### E. HPV Early Region Proteins

Constitutive or induced expression of HPV early (E) region proteins provide targets for an effective HPV vaccine. See FIG. 1.

HPV genes (E1-E8) regulate viral expression and replication, and late (L) genes control viral protein coding. HPV early region protein functions include the following: E1, E2 have functions in viral replication/transcription (e.g., E2 regulates expression of E6 and E7; and, E1/E2 interaction is essential for viral replication); E4, E5 have increased expression during late stage of viral replication cycle; and, E6, E7 act co-operatively during replication (E6 is required for episomal genome maintenance, E7 expands compartment of epithelial cells active in DNA replication).

The E6 and E7 proteins of HPV 16 and 18 represent potential targets for therapeutic vaccines because they are responsible for maintenance of the malignant phenotype and are constitutively expressed in the tumors. Moreover, they are not endogenously expressed on any human tissues, so there is very low risk of inducing autoimmune events with a vaccine targeting these proteins. Several CD8+ T cell epitopes of E6 and E7 capable of eliciting cytotoxic T lymphocyte (CTL) responses have previously been identified, and clinical studies employing diverse vaccine platforms have demonstrated various degrees of effectiveness in terms of eliciting HPV-specific responses and clinical benefits. These include live vector, peptide or protein, cell-based, and nucleic acid vaccines. The majority of these vaccines target HPV oncoproteins E6 and E7 with the goal to activate HPV antigen-specific CD8+ cytotoxic T cells or CD4⁺ helper T cells. These therapeutic vaccines also differ by their routes of administration.

An exemplary embodiment of the present invention is an HPV16/18 vaccine that delivers an multi-epitope antigen design containing 35 non-HLA-restricted epitopes of HPV 16 and 18-namely, 32 key immunogenic (CTL specific) peptides from E6 (HPV16/18), E7 (HPV-16/18), and E5 (HPV16) and 3 unique agonist peptides.

### III. Antigenicity Bioinformatics Workflow for HPV Vaccine Designs

### A. Identification of New HPV Antigenic Components

Based on the important role that HPV E2 and E4 genetic components play in HPV essential functions, the location of the corresponding proteins, as well as in-silico prediction, E2- and E4-derived antigens were identified for an HPV therapeutic vaccine. Non-oncogenic and viral inactivation genetic modifications were also applied to eliminate viral and oncogenic biological activity from HPV proteins, such as in HPV E2 and E6 proteins.

Genetic manipulation was also applied to achieve production of protein sequences reordered in such manner as to retain immunogenic features of the peptides, but eliminate oncogenic and viral amplification functions of E7 and E4, respectively.

As such, some of the innovative aspects of the designs exemplified in this specification include: (1) use of gene constructs encoding fusion proteins comprising four or more different HPV proteins; (2) combining amino acid point mutations and overlapping polypeptide sequence shuffling techniques to inactivate oncogenic and essential viral functions; (3) incorporation of HPV proteins comprising multiple antigenic components from HPV proteins which are highly expressed in host infected cells; (4) first known hybrid antigen designs; (5) combining epitopes from high cancer risk and low cancer risk HPV strains; (6) use of mixed and regularly repeating linkers; (7) use of rigid linkers to stabilize polypeptide subunits and prevent undesirable intra-molecular interactions; (8) use of cleavable linkers between epitopes; and/or (9) dual use of linker sequence to provide both protein-protein linker (-) function as well as antigens and epitopes, per se (i.e., antigenicity conferred by the linker sequences).

Antigenicity is the capacity to stimulate the production of antibodies or cell-mediated immune responses. The antigenicity of the final design sequences was predicted by the Vaxjen software, which is an alignment-independent model for antigen recognition based on main chemical properties of amino acid sequences. The results indicate that the five antigen sequences are antigenic. See, Table 4 (Antigenicity Virus & Tumor).

TABLE 4
In-silico Evaluation

| | Antigenicity | | | |
|---|---|---|---|---|
| Design No. | Virus | Tumor | Allergenicity | Host Cross-Reactivity |
| 1 | 0.5288 | 0.6134 | Non-allergenic | No hits |
| 2 | 0.5252 | 0.5814 | Non-allergenic | No hits |
| 3 | 0.5587 | 0.6322 | Non-allergenic | No hits |
| 4 | 0.5451 | 0.5809 | Non-allergenic | No hits |
| 5 | 0.4964 | 0.5997 | Non-allergenic | No hits |

Allergens are small antigens that commonly provoke an antibody response. Allergenicity, whether the antigen is an allergen or non-allergen was predicted by ALLERTOP, a bioinformatics-based allergen prediction software with machine learning methods for classification. It includes logistic regression (LR), decision tree (DT), naive Bayes (NB), random forest (RF), multilayer perceptron (MLP), and k nearest neighbors (kNN). The results indicate that the five antigen sequences are non-allergenic. See, Table 4 (Allergenicity).

Cross-reactivity or invocation of autoimmune side effects in various tissues has important safety implications in adoptive immunotherapy. Sequence homology analyses were performed to assess if those novel antigens have cross-reactivity with human proteome with blast search, basic local alignment search tool. No host cross reactivity was identified in these five antigen sequences. See, Table 4 (Host Cross-Reactivity).

### B. Software/Tools

Software tools utilized in performance of the designs described herein include, but are not limited to:
ALLERTOP (See, AllerTOP v.2—a server for in silico prediction of allergens; J Mol Model. 2014 June; 20 (6): 2278. doi: 10.1007/s00894-014-2278-5. Epub 2014 May 31.)
ANN (See, Reliable prediction of T-cell epitopes using neural networks with novel sequence representations; Protein Sci. 2003 May; 12 (5): 1007-17.)
BLAST (Basic Local Alignment Search Tool; NCBI, National Center for Biotechnology Information, U.S. National Library of Medicine 8600 Rockville Pike, Bethesda MD, 20894 USA.)
CLUSTALW2 (EMBL-EBI, Wellcome Genome Campus, Hinxton, Cambridgeshire, CB10 1SD, UK. +44 (0) 1223 49 44 44.)
GENEIOUS VI 1.1.5 (See, geneious.com/biopharma/)
IEDB CONSENSUS (See, A consensus epitope prediction approach identifies the breadth of murine T (CD8+)-cell responses to vaccinia virus; Nat Biotechnol. 2006 July; 24 (7): 817-9. Epub 2006 Jun. 11)
NETMHCPAN 4.0 (See, Gapped sequence alignment using artificial neural networks: application to the MHC class I system; Bioinformatics. 2016 Feb. 15; 32 (4): 511-7. doi: 10.1093/bioinformatics/btv639. Epub 2015 Oct. 29.)
PHYRE2 (See, The Phyre2 web portal for protein modeling, prediction and analysis; 7 May 2015; nature.com/articles/nprot.2015.053; doi.org/10.1038/nprot.2015.053.)
PYMOL MOLECULAR GRAPHICS SYSTEM V2. 1. 1 (See, pymol.org/2/; sourceforge.net/projects/pymol/support)
VAXJEN (See, VaxiJen: a server for prediction of protective antigens, tumour antigens and subunit vaccines; BMC Bioinformatics. 2007 Jan. 5; 8:4 . . . )

### III. Variants of Exemplary HPV Antigen Design

In an exemplary embodiment, the polypeptide construct of the present invention has a sequence of SEQ ID NO: 243 or a functional variant thereof (e.g., an amino acid sequence having at least 80%, 85%, 90%, 95%, 96%, 97%, 98%, 99%, 99.1%, 99.5%, 99.9%, or 99.99% sequence identity with SEQ ID NO: 243, or a conservatively-substituted variant of SEQ ID NO: 243, or a non-conservatively-substituted variant of SEQ ID NO: 243).

In certain embodiments, the polypeptide construct of the present invention comprises a functional variant of SEQ ID NO: 243 that, when compared to SEQ ID NO: 243, has similar or enhanced binding affinity to HLA proteins associated with HPV16/18 and/or effects a similar or enhanced immunogenic response. Such a variant can be readily determined by sequence alignment software such as ClustalW (see also infra Example 1).

In certain embodiments, the variant has the same ankyrin scaffold as SEQ ID NO: 243 where various HPV and agonist peptides of SEQ ID NO: 243 are shuffled in an order different from those of SEQ ID NO: 243. In one such embodiment, the variant has a sequence of any one of SEQ ID NOs 250-261.

In certain embodiments, the variant has the same ankyrin scaffold as SEQ ID NO: 243 and fewer HPV and agonist peptides than SEQ ID NO: 243. In some embodiments, the variant has 1, 2, 3, 4, 5, 6, 7, 8, 9, or 10 fewer HPV/agonist peptides compared to SEQ ID NO: 243. In some embodiments, the variant has a sequence of SEQ ID NO: 262, SEQ ID NO: 265, SEQ ID NO: 266, or SEQ ID NO: 267.

In certain embodiments, the variant has the same ankyrin scaffold as SEQ ID NO: 243 and more HPV and agonist peptides than SEQ ID NO: 243. In some embodiments, the variant has 1, 2, 3, 4, 5, 6, 7, 8, 9, or 10 more HPV/agonist peptides compared to SEQ ID NO: 243. In some embodiments, the variant has a sequence of SEQ ID NO: 263, SEQ ID NO: 268, SEQ ID NO: 269, or SEQ ID NO: 270.

In certain embodiments, the variant differs from SEQ ID NO: 243 in that the variant has conservative amino acid substitutions within the ankyrin-like repeat domains of SEQ ID NO: 243. In some embodiments, one or more conservative amino acid substitutions within the ankyrin-like repeat domains of SEQ ID NO: 243 correspond to position(s) selected from 1-5, 15-21, 31-33, 43-45, 55-63, 73-79, 100-102, 112-121, 137-141, 151-153, 163-173, 184-187, 197-199, 209-220, 231-233, 249-251, 262-264, 275-295, 304-311, 322-324, 335-337, 349-357, 373-379, 389-391, 402-411, 422-426, 437-439, 450-459, 475-478, 488-490, 500-511, 521-523, 534-536, 546-548, 559-561, or 571-572. In one such embodiment, the variant has a sequence of any one of SEQ ID NOS: 245-249.

In certain embodiments, the variant differs from SEQ ID NO: 243 in that the variant has conservative amino acid substitutions within the ankyrin-like repeat domains of SEQ ID NO: 243, as well as more HPV and agonist peptides than SEQ ID NO: 243. In some embodiments, the variant has a sequence of SEQ ID NO: 271.

In certain embodiments, the variant differs from SEQ ID NO: 243 in that the variant has conservative amino acid substitutions within the ankyrin-like repeat domains of SEQ ID NO: 243, as well as fewer HPV and agonist peptides than SEQ ID NO: 243. In some embodiments, the variant has a sequence of SEQ ID NO: 272.

In certain embodiments, the variant differs from SEQ ID NO: 243 in that the variant has conservative amino acid substitutions within the ankyrin-like repeat domains of SEQ ID NO: 243, as well as various HPV and agonist peptides of SEQ ID NO: 243 shuffled in a different order from those of SEQ ID NO: 243. In some embodiments, the variant has a sequence of SEQ ID NO: 264.

In certain embodiments, the variant is identical to SEQ ID NO: 243 except the variant has conservative amino acid substitutions within the ankyrin-like repeat domains of SEQ ID NO: 243, various HPV and agonist peptides of SEQ ID NO: 243 shuffled in a different order from those of SEQ ID NO: 243, and comprises more HPV and agonist peptides than SEQ ID NO: 243. In some embodiments, the variant has a sequence of SEQ ID NO: 274.

In certain embodiments, the variant is identical to SEQ ID NO: 243 except the variant has conservative amino acid substitutions within the ankyrin-like repeat domains of SEQ ID NO: 243, various HPV and agonist peptides of SEQ ID NO: 243 shuffled in a different order from those of SEQ ID NO: 243, and comprises fewer HPV and agonist peptides than SEQ ID NO: 243. In some embodiments, the variant has a sequence of SEQ ID NO: 273.

Any of the variant HPV antigen designs or polypeptide constructs thereof can be used as a component in a vaccine, e.g., a HPV vaccine.

### IV. Methods of Treatment

The present invention relates in part to a method of treating a disease or disorder in a subject in need thereof, comprising administering to the subject a polynucleotide, polypeptide, vector, composition, vaccine, or cell of the present invention. In certain embodiments, the method involves administering a polynucleotide, polypeptide, vector, composition, vaccine, or cell of the present invention to subjects with anogenital warts, lower genital tract neoplasia (e.g., cervical, vaginal, and vulvar intraepithelial neoplasia), cervical cancer, vulvar cancer, anal cancer, penile cancer, or head and neck cancers. In certain embodiments, the method involves administering a polynucleotide, polypeptide, vector, composition, vaccine, or cell of the present invention to subjects with malignancies caused by HPV 16/18.

The present invention also relates in part to a method for priming of T-cell responses against HPV-infected (e.g., HPV 16/18⁺) cells in a subject in need thereof (e.g., a subject with penile, cervical, anal, or oropharyngeal cancer), the method comprising administering to the subject the vector of the present invention. In certain embodiments, the method involves the administration of a polynucleotide, polypeptide, vector, composition, vaccine, or cell of the present invention to subjects with malignancies caused by HPV 16/18.

In certain embodiments, the method of the invention protects against disease progression with a lower PU dose than previous methods known in the art. For example, in some embodiments the method protects against disease protection with a 5e⁹ PU dose of the vector, composition, or vaccine. In other embodiments the method protects against disease protection with a 5e¹⁰ PU dose of the vector, composition, or vaccine. In some embodiments, the method of the invention protects against disease progression with fewer administrations of the therapeutic composition than previous methods known in the art. For example, in some embodiments the method protects against disease protection with only a single administration of the vector, composition, or vaccine.

In certain embodiments, the subject being treated is a mammal, for example, a primate. In some embodiments, the subject being treated is a human.

The method may involve the administration of the polynucleotide, polypeptide, vector, composition, vaccine, or cell in an amount therapeutically-effective to treat the disease or disorder, or increase the activity of T-cell responses against specific HPV proteins or antigens. The effective amount may vary depending on the subject's condition, age, gender, medical history, and/or weight. The amount may also vary depending on the condition to be treated, the anti-inflammatory agent encoded, the type of vector, cell, and/or vaccine used for administration, and the route of administration.

In certain embodiments, the vector, composition, or vaccine is administered in doses. The amount of the vector, composition, or vaccine that is administered in a dose is a therapeutically effective amount of the vector, composition, or vaccine. In certain embodiments, the dosage amount in a dose may comprise about 0.1×10⁹ to about 10×10¹² particle units, 0.1×10⁹ to about 1.0×10¹² particle units, about 0.1×10⁹ to about 10×10¹¹ particle units, about 0.1×10⁹ to about 1.0×10¹¹ particle units, about 0.5×10⁹ to about 0.5×10¹¹ particle units, about 0.5×10⁹ to about 0.1×10¹¹ particle units, about 1.0×10¹⁰ to about 10×10¹¹ particle units, about 1.0×10¹⁰ to about 0.1×10¹¹ particle units, about 0.1×10¹¹ to about 10×10¹¹ particle units, about 0.5×10¹¹ to about 9×10¹¹ particle units, about 0.5×10¹¹ to about 8×10¹¹ particle units, about 0.5×10¹¹ to about 7×10¹¹ particle units, about 0.5×10¹¹ to about 6×10¹¹ particle units, about 0.5×10¹² to about 10×10¹² particle units, about 0.5×10¹² to about 1.0×10¹² particle units, about 1.0×10¹¹ to about 0.1×10¹² particle units, about 0.1×10¹² to about 10×10¹² particle units, about 1×10¹⁰ particle units, about 5×10¹⁰ particle units, about 5×10¹¹ particle units, about 6×10¹¹ particle units, about 7×10¹¹ particle units, about 8×10¹¹ particle units, about 9×10¹¹ particle units, about 10×10¹¹ particle units, about 1×10¹² particle units, about 2×10¹² particle units, about 3×10¹² particle units, about 4×10¹² particle units, about 5×10¹² particle units, about 6×10¹² particle units, about 7×10¹² particle units, about 8×10¹² particle units, about 9×10¹² particle units, or about 10×10¹² particle units.

In certain embodiments, the dosage amount may comprise about 1.0×10⁵ to about 1.0×10¹⁰ plaque forming units (PFU), for example, about 0.5×10⁵ to about 0.5×10¹⁰ PFU, about 0.1×10⁵×0.1×10¹⁰ PFU, about 1×10⁶ to about 1×10⁹ PFU, about 0.5×10⁶ to about 0.5×10⁹ PFU, about 0.1×10⁶ to about 0.1×10⁹ PFU, about 1×10⁷ to about 1×10⁸ PFU, about 0.5×10⁷ to about 0.5×10⁸ PFU, about 0.1×10⁷ to about 0.1×10⁸ PFU, about 1.0×10⁶ to about 1.0×10⁹ PFU, about 0.5×10⁶ to about 0.5×10⁹ PFU, about 1.0×10⁷ to about 1×10⁸ PFU, about 1.0×10⁶ to about 1.0×10⁸ PFU, about 0.5×10⁶ to about 0.5×10⁸ PFU, or about 0.1×10⁶ to about 0.1×10⁸ PFU.

In certain embodiments, the dosage amount of any of the polynucleotides encoding any of the fusion proteins described herein may comprise from about 1×10⁻⁵ to about 10 micrograms (μg) of the polynucleotide. For example, the dosage amount of any of the polynucleotides encoding any of the fusion proteins described herein may comprise from about 5×10⁻⁵ to about 5 μg, from about 1×10⁻⁴ to about 1, from about 5×10⁻⁴ to about 5 μg, from about 5×10⁻³ to about 5 μg, from about 0.05 to about 5 μg, from about 0.25 to about 5 μg, from about 0.5 to about 5 μg, from about 0.75 to about 5 μg, from about 5×10⁻⁴ to about 1 μg, from about 5×10⁻⁴ to about 0.5 μg, from about 5×10⁻⁴ to about 0.05 μg, or from about 5×10⁻⁴ to about 5×10⁻³ μg of the polynucleotide.

In some embodiments, the viral vector and/or polynucleotide encoding any of the fusion proteins described herein may be quantified by Quantitative PCT Analysis (Q-PCR) or analytical HPLC.

For the treatment of HPV-associated pathologies, a dose of the vector may, for example, be about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

The dose may be adjusted during the course of treatment, for example, after the levels of expression of the transgene are monitored. If the levels are higher or lower than desired, the amount or frequency of the dose may be adjusted accordingly.

The specific initial and continuing dosage regimen for each patient will vary according to the nature and severity of the condition as determined by the attending diagnostician, the activity of the therapeutic agent, the age of the patient, the diet of the patient, time of administration, route of administration, rate of excretion of the drug, drug combinations, and the like.

The desired mode of treatment, number of doses, routes of administration, and dose schedules may be ascertained and/or adjusted in accordance with methodologies known in the art.

The method disclosed herein contemplates the use of any administration route known in the art for delivery of a polynucleotide, polypeptide, vector, composition, vaccine, or cell. For example, administration may be oral, subcutaneous, intramuscular, intravenous, intracranial, intra-articular, intradermal, or transdermal. In certain embodiments, the subcutaneous or intra-articular administration is by way of a syringe. In some such embodiments, the dosage amount of the vector is contained in a composition in the form of an injectable formulation.

In certain embodiments, the dosage amount is contained in a composition having a volume of about 0.1 to about 5 ml, about 0.1 to about 4 ml, about 0.1 to about 3 ml, about 0.1 to about 2 ml, about 0.25 to about 1.75 ml, about 0.5 to about 1.5 ml, about 0.75 to about 1.25 ml, or about 1.0 ml. In some embodiments, the dosage amount is contained in a composition having a volume of about 0.1 ml, about 0.2 ml, about 0.3 ml, about 0.4 ml, about 0.5 ml, about 0.6 ml, about 0.7 ml, about 0.8 ml, about 0.9 ml, about 1.0 ml, about 1.2 ml, about 1.3 ml, about 1.4 ml, about 1.5 ml, about 1.6 ml, about 1.7 ml, about 1.8 ml, about 1.9 ml, about 2.0 ml, about 2.1 ml, about 2.2 ml, about 2.3 ml, about 2.4 ml, about 2.5 ml, about 2.6 ml, about 2.7 ml, about 2.8 ml, about 2.9 ml, about 3.0 ml, about 3.1 ml, about 3.2 ml, about 3.3 ml, about 3.4 ml, about 3.5 ml, about 3.6 ml, about 3.7 ml, about 3.8 ml, about 3.9 ml, about 4.0 ml, about 4.1 ml, about 4.2 ml, about 4.3 ml, about 4.4 ml, about 4.5 ml, about 4.6 ml, about 4.7 ml, about 4.8 ml, about 4.9 ml, or about 5.0 ml.

Administration of the polynucleotide, polypeptide, vector, composition or vaccine may be at any suitable site on the subject. The choice of administration site will vary depending on factors such as the volume of the dose to be administered, the subject's age, the subject's sex, and the type of active agent to be administered. Subcutaneous administration may, for example, be to the subject's limbs, buttocks, or abdomen. For doses having larger volumes, intramuscular administration is preferred. Such may be, for example, to the subject's deltoid, vastus lateralis, ventrogluteal, or dorsogluteal muscle. Intravenous administration may, for example, be to the subject's arm (e.g. at the bend of the arm), the back of the subject's hand, or the top of the subject's foot. Intra-articular administration may, for example, be to the subject's knee, hip, shoulder, or ankle.

The dosing regimen will vary depending on the subject's age, the subject's sex, and the type of active agent to be administered. The dose may be administered hourly, daily, weekly, monthly, or annually.

In certain embodiments, the doses are delivered at intervals at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days apart. In certain embodiments, the doses are delivered at intervals of about twice per day, about once every day, about twice per week, about once every week, about once every two weeks, about once every three weeks, about once every four weeks, or about once every five weeks. In certain embodiments, the second dose is administered about one week after the first dose, about two weeks after the first dose, about three weeks after the first dose, about four weeks after the first dose, or about five weeks after the first dose; the third dose is administered two weeks after the second dose, about three weeks after the second dose, about four weeks after the second dose, about five weeks after the second dose, or about six weeks after the second dose; and the fourth dose is administered about three weeks after the third dose, about four weeks after the third dose, about five weeks after the third dose, about six weeks after the third dose, about seven weeks after the third dose, about eight weeks after the third dose, about nine weeks after the third dose, about ten weeks after the third dose, about eleven weeks after the third dose, or about twelve weeks after the third dose. In one embodiment, the second dose is administered about two weeks after the first dose, the third dose is administered about six weeks after the second dose, and the fourth dose is administered about twelve weeks after the third dose.

In some embodiments, the treatment involves surgical debulking, usually by means of debridement, angiolytic laser, cryotherapy, or carbon dioxide laser. In some embodiments, the surgical procedure is performed via microscopic or endoscopic rigid laryngoscopy, for example, using either a laser or microdebrider to remove papillomas.

This may be preceded and/or followed by administration of the polynucleotide, polypeptide, vector, composition or vaccine of the present invention (alone or in combination with another therapeutic agent). In some embodiments, the treatment of a patient with the polynucleotide, polypeptide, vector, vaccine, or cell described herein, or a pharmaceutical composition comprising the same, reduces and/or eliminates the need for repeated surgical debulking.

### V. Gene Switch Systems

The gene switch may be any gene switch that regulates gene expression by addition or removal of a specific ligand. In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/001471 1, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety.

### VII. Manufacture of a Medicament

The present invention also relates in part to a use of the polynucleotide, polypeptide, vector, vaccine, or cell described herein, or a composition comprising the same, in the manufacture of a medicament for use in treating a disease or disorder in a subject in need thereof. In certain embodiments, the disease or disorder may be a proliferative disease or disorder, such as cancer (e.g., HPV 16/18 malignancies).

### IX. Combination Therapies

In certain embodiments, the compositions and methods of the present invention can be combined with at least one additional active agent or therapy. Such additional therapies include radiation therapy, surgery (e.g., debulking), chemotherapy, gene therapy, DNA therapy, virus therapy, RNA therapy, immunotherapy, bone marrow transplantation, nanotherapy, monoclonal antibody therapy, or a combination of the aforementioned therapies. The additional therapy may be in the form of an adjuvant or neoadjuvant therapy.

In some embodiments, the combination therapy comprises the administration of the polynucleotide, polypeptide, vector, vaccine, or cell described herein, or a composition comprising the same, and the concomitant administration of one or more additional compounds, molecules, compositions, or agents. The present invention also relates in part to a combination therapy comprising the administration of the polynucleotide, polypeptide, vector, vaccine, or cell described herein, or a composition comprising the same, and the concomitant use of a surgical or non-surgical procedure.

### A. Administration of Combination Therapies

In certain embodiments, the compositions of the present invention may be administered before, during, after, or in various combinations with additional therapy, such as immune checkpoint therapy. The administration may be made at intervals ranging from simultaneous to minutes to days to weeks. In embodiments where the present composition is provided to the patient separately from the additional therapeutic agent, the operator may generally ensure that no significant time has elapsed between each delivery time, so that the two compositions can continue to exert a beneficial combination effect on the patient. The two therapies may therefore be provided to the patient within about 12 to 24 hours, or 48 hours, or 72 hours of each other, more specifically within about 6 to 12 hours of each other. In some situations, treatment periods are significant over several days (2, 3, 4, 5, 6 or 7 days) to weeks (1, 2, 3, 4, 5, 6, 7 or 8 weeks) between each dose It may be desirable to extend the period.

Various combinations may be used. For the following examples, the composition of the present invention is “A” and the additional therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy to a patient will follow the general protocol for administration of the compound, given the toxicity of the agents, if present. Thus, in some embodiments, there is a step of monitoring toxicity resulting from the combination therapy.

In certain embodiments, the at least one additional therapy comprises the co-administration of an additional agent. In some embodiments, the additional agent may be contained in the same composition that contains the polynucleotide, polypeptide, vector, vaccine, or cell described herein. Such combination therapies may serve to enhance the treatment of a disease or disorder (e.g., improving the subject's response, prolonging the effects of the treatment) and/or to reduce any side-effects of treatment with the anti-inflammatory agent. In some embodiments, HPV 16/18 malignancies are being treated.

Any suitable agent that may be combined with the polynucleotide, polypeptide, vector, vaccine, or cell described herein, or a composition comprising the same, may be used. For example, the agent may be a therapeutic agent, such as a chemotherapy agent, an anti-inflammatory agent, an analgesic, a biological response modifier, a vector comprising such agents, or a cell comprising the therapeutic agent or a nucleic acid encoding the same.

In certain embodiments, the additional agent is administered at or near the same location as the composition comprising the vector of the present invention is administered. In certain other embodiments, the additional agent is administered at a different location, for example, at the opposite side or extremity.

Administration of the additional agent may be simultaneous with the administration of the composition comprising the vector of the present invention. In certain embodiments, the additional agent is contained in the same formulation as that containing the vector and can be administered with the vector in one unitary dose. In certain other embodiments, the additional agent is not contained in the same formulation but is administered at the same time or within a limited time frame (e.g., a single day, hour, or fraction of an hour) from the administration of the vector.

Alternatively, administration of the additional agent may be sequential in relation to the administration of the vector of the present invention. Such may be preferred in instances where minimizing adverse reactions is desired. In such embodiments, the additional agent may be administered on a schedule in accordance with approved dosing regimens for that agent. Alternatively, the agent may be administered in accordance with a schedule that serves to better maximize the therapeutic effects of the combination therapy, while minimizing adverse reactions.

The timing of administration can be tailored to the specific mechanisms of action and pharmacokinetics of each therapy, maximizing synergistic effects and minimizing overlaps in potential toxicities. Furthermore, the treatment regimen can be adapted based on individual patient response and disease progression, offering flexibility for personalized therapeutic strategies. For example, in embodiments in which an interleukin, for example IL-12, is one of the immunotherapies, its initial administration may precede other agents to prime the immune system for enhanced response, followed by subsequent therapies to amplify and direct the activated immune response. Alternatively, concurrent administration of the interleukin with an immunotherapy can create a synergistic immediate boost in anti-tumor activity, while continued interleukin treatment supports sustained immune engagement. The sequential or concurrent administration of interleukin with other immunotherapies offers a dynamic approach to orchestrating robust and durable anti-tumor immune responses, providing greater therapeutic potential compared to administration of individual immunotherapies or agents alone.

In some embodiments, more than one doses of a first therapy is administered to the subject. In certain embodiments, more than one doses of a second therapy is administered to the subject. In still further embodiments, more than one doses of a third therapy is administered to the subject.

In some embodiments, subsequent doses of the first therapy are administered once every one, two, three or four weeks after the initial dose of the first therapy. In certain embodiments, subsequent doses of the second therapy are administered once every one, two, three or four weeks after the initial dose of the second therapy. In still further embodiments, subsequent doses of the third therapy are administered once every one, two, three or four weeks after the initial dose of the third therapy.

In some embodiments, the initial dose of the first therapy is administered at about 1, 2, 3, 4, 5, 6, 7, 8, 9, 10, 11 or 12 hours prior to the administration of the second therapy. In some embodiments, the initial dose of the first therapy is administered at about 1, 2, 3, 4, 5, 6, 7, 8, 9, 10, 11, 12, 13, or 14 days prior to the administration of the second therapy. In some embodiments, the initial dose of the first therapy is administered at about 2, 3, 4, 5, 6, 7, 8, 9, or 10 weeks prior to the administration of the second therapy. In some embodiments, the initial dose of the first therapy is administered at about 2, 3, 4, 5, or 6 months prior to the administration of the second therapy.

In some embodiments, the initial dose of the first therapy is administered at about 1, 2, 3, 4, 5, 6, 7, 8, 9, 10, 11 or 12 hours after the administration of the second therapy. In some embodiments, the initial dose of the first therapy is administered at about 1, 2, 3, 4, 5, 6, 7, 8, 9, 10, 11, 12, 13, or 14 days after the administration of the second therapy. In some embodiments, the initial dose of the first therapy is administered at about 2, 3, 4, 5, 6, 7, 8, 9, or 10 weeks after the administration of the second therapy. In some embodiments, the initial dose of the first therapy is administered at about 2, 3, 4, 5, or 6 months after the administration of the second therapy.

In some embodiments, the initial dose of the first therapy is administered at about 1, 2, 3, 4, 5, 6, 7, 8, 9, 10, 11 or 12 hours prior to the administration of the third therapy. In some embodiments, the initial dose of the first therapy is administered at about 1, 2, 3, 4, 5, 6, 7, 8, 9, 10, 11, 12, 13, or 14 days prior to the administration of the third therapy. In some embodiments, the initial dose of the first therapy is administered at about 2, 3, 4, 5, 6, 7, 8, 9, or 10 weeks prior to the administration of the third therapy. In some embodiments, the initial dose of the first therapy is administered at about 2, 3, 4, 5, or 6 months prior to the administration of the third therapy.

In some embodiments, the initial dose of the first therapy is administered at about 1, 2, 3, 4, 5, 6, 7, 8, 9, 10, 11 or 12 hours after the administration of the third therapy. In some embodiments, the initial dose of the first therapy is administered at about 1, 2, 3, 4, 5, 6, 7, 8, 9, 10, 11, 12, 13, or 14 days after the administration of the third therapy. In some embodiments, the initial dose of the first therapy is administered at about 2, 3, 4, 5, 6, 7, 8, 9, or 10 weeks after the administration of the third therapy. In some embodiments, the initial dose of the first therapy is administered at about 2, 3, 4, 5, or 6 months after the administration of the third therapy.

In some embodiments, the initial dose of the second therapy is administered at about 1, 2, 3, 4, 5, 6, 7, 8, 9, 10, 11 or 12 hours prior to the administration of the third therapy. In some embodiments, the initial dose of the second therapy is administered at about 1, 2, 3, 4, 5, 6, 7, 8, 9, 10, 11, 12, 13, or 14 days prior to the administration of the third therapy. In some embodiments, the initial dose of the second therapy is administered at about 2, 3, 4, 5, 6, 7, 8, 9, or 10 weeks prior to the administration of the third therapy. In some embodiments, the initial dose of the second therapy is administered at about 2, 3, 4, 5, or 6 months prior to the administration of the third therapy.

In some embodiments, the initial dose of the second therapy is administered at about 1, 2, 3, 4, 5, 6, 7, 8, 9, 10, 11 or 12 hours after the administration of the third therapy. In some embodiments, the initial dose of the second therapy is administered at about 1, 2, 3, 4, 5, 6, 7, 8, 9, 10, 11, 12, 13, or 14 days after the administration of the third therapy. In some embodiments, the initial dose of the second therapy is administered at about 2, 3, 4, 5, 6, 7, 8, 9, or 10 weeks after the administration of the third therapy. In some embodiments, the initial dose of the second therapy is administered at about 2, 3, 4, 5, or 6 months after the administration of the third therapy.

### B. Exemplary Combination Therapies

Anti-inflammatory agents for use in the such combination therapy include: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.

Exemplary analgesics for use in combination therapy include acetaminophen, oxycodone, tramadol or proporxyphene hydrochloride.

The HPV vaccine antigens of the present invention may be administered in combination with a second therapeutic agent, such as a biological response modifier. Exemplary biological response modifiers suitable for use in combination therapy according to the present invention include, for example, molecules directed against cell surface markers (e.g., CD4, CD5, etc.); cytokine inhibitors, such as the TNF inhibitors (e.g., etanercept (ENBREL®), adalimumab (HUMIRA®), and infliximab (REMICADE®); chemokine inhibitors; cell signaling inhibitors, such as EGFR inhibitors (e.g., Gefinitnib (IRESSA®) and Erlotinib (TARCEVA®)), nucleotide analogs (e.g., Cidofovir), angiogenesis inhibitors, such as Bevacizumab (AVASTIN®), non-steroidal anti-inflammatory compounds (NSAIDs), such as COX-2-selective drugs (e.g., Celexecob (CELEBREX®)), immune checkpoint inhibitors, such as PD-1 inhibitors (e.g., Pembrolizumab (KEYTRUDA®), Nivolumab (OPDIVO®), and Cemiplimab (LIBTAYO®) and PD-L1 inhibitors (e.g., Atezolizumab (TECENTRIQ®), Avelumab (BAVENCIO®), and Durvalumab (IMFINZI®), adhesion molecule inhibitors, and other adjuvant therapies. In some embodiments, the second therapeutic agent is an immune checkpoint inhibitor. In some embodiments, the second therapeutic agent is a PD-1 inhibitor. In some embodiments, the second therapeutic agent is Pembrolizumab (KEYTRUDA®). The biological response modifiers include monoclonal antibodies as well as recombinant forms of molecules. Exemplary disease-modifying anti-rheumatic drugs (DMARDs) include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the biological response modifier is administered at a dose ranging from about 0.1 mg/kg to about 10 mg/kg. In particular embodiments, the biological response modifier is administered at a dose of about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 5 mg/kg, or about 10 mg/kg. In a particular embodiment, the biological response modifier is administered at a dose of 2 mg/kg. In another embodiment, the biological response modifier is administered at a dose of 10 mg/kg.

In some embodiments, the biological response modifier is administered at a dose of 10 mg, 50 mg, 100 mg, 150 mg, 200 mg, 250 mg, 300 mg, 350 mg, 400 mg, 450 mg, 500 mg, 550 mg, 600 mg, 650 mg, 700, mg, 750 mg, 800 mg, 850 mg, 900 mg, 950 mg, or 1,000 mg. In a particular embodiment, the biological response modifier is administered at a dose of 200 mg. In another embodiment, the biological response modifier is administered at a dose of 400 mg.

The dosing regimen for the biological response modifier may be adjusted based on individual patient factors, such as body weight, renal function, and liver function. A person of ordinary skill in the art can determine the most appropriate dosing schedule for each patient.

The biological response modifier may be administered prior to, concurrently with, or subsequent to the administration of the HPV vaccine antigens. For example, the biological response modifier may be administered approximately 1 day, 3 days, 1 week, 2 weeks, or 1 month before or after administration of the HPV vaccine antigens.

In some embodiments, the biological response modifier may be administered multiple times, including but not limited to twice a week, once a week, once every two weeks, once every three weeks, once every four weeks, once every five weeks, once every six weeks, once every seven weeks, once every eight weeks, once every nine weeks, or once every ten weeks.

The duration of treatment with the biological response modifier may vary depending on the cancer type, response to therapy, and tolerability. In some cases, treatment may continue until disease progression or unacceptable toxicity, while in others, a fixed duration of treatment (e.g., 1-2 years) may be recommended. In some embodiments, the duration of therapy for the biological response modifier may be up to 12 months, 18 months, 24 months, 30 months, or 36 months. In a particular embodiment, the duration of therapy for the biological response modifier is up to 24 months.

The HPV vaccine antigens of the present invention may be administered in combination with a biological response modifier at the dosing ranges disclosed herein for the treatment of various cancers. Exemplary cancers that may be treated with the combination of HPV vaccine antigens and a biological response modifier include, but are not limited to, cervical cancer, vulvar cancer, vaginal cancer, anal cancer, penile cancer, oropharyngeal cancer (throat cancer), recurrent respiratory papillomatosis (RRP), melanoma, non-small cell lung cancer (NSCLC), small cell lung cancer (SCLC), head and neck squamous cell carcinoma (HNSCC), classical Hodgkin lymphoma (cHL), primary mediastinal large B-cell lymphoma (PMBCL), urothelial carcinoma, microsatellite instability-high (MSI-H) or mismatch repair deficient (dMMR) solid tumors, gastric cancer, esophageal cancer, hepatocellular carcinoma (HCC), Merkel cell carcinoma (MCC), renal cell carcinoma (RCC), endometrial carcinoma, tumor mutational burden-high (TMB-H) solid tumors, cutaneous squamous cell carcinoma (cSCC), and triple-negative breast cancer (TNBC).

In some embodiments of the present invention, the biological response modifier administered in combination with the HPV vaccine antigens is Pembrolizumab. Pembrolizumab is a humanized monoclonal antibody that blocks the interaction between PD-1 and its ligands, PD-L1 and PD-L2. Pembrolizumab may be administered at the dosing ranges disclosed herein, and may be dependent on the cancer being treated and the patient's individual characteristics. In some embodiments, the dosing regimen for Pembrolizumab may be: a) 200 mg, administered as an intravenous infusion over 30 minutes every 3 weeks (Q3W); b) 400 mg, administered as an intravenous infusion over 30 minutes every 6 weeks (Q6W); c) 2 mg/kg, administered as an intravenous infusion over 30 minutes every 3 weeks (Q3W); or d) 10 mg/kg, administered as an intravenous infusion over 30 minutes every 2 weeks (Q2W) or every 3 weeks (Q3W).

In particular embodiments, the combination of HPV vaccine antigens and a biological response modifier, such as Pembrolizumab, is administered for the treatment of cervical cancer, HPV-related carcinoma, HPV-related malignancy, and/or oropharyngeal squamous cell carcinoma. These cancers are known to be associated with HPV infection, and the combination therapy disclosed herein may provide enhanced therapeutic efficacy compared to either the HPV vaccine antigens or the biological response modifier alone.

In certain embodiments, HPV vaccine antigens provided herein are co-delivered and/or co-expressed (e.g., as part of the same HPV antigen delivery vector or via a separate vector) along with other cytokines. In certain embodiments, HPV vaccine antigens provided herein, are polynucleotides encoding gene-switch polypeptides and a cytokine, or variant or derivative thereof, and methods and systems incorporating the same. Cytokine is a category of small proteins between about 5-20 kDa that are involved in cell signaling. In some instances, cytokines include chemokines, interferons, interleukins, colony-stimulating factors or tumor necrosis factors. In some embodiments, chemokines play a role as a chemoattractant to guide the migration of cells, and is classified into four subfamilies: CXC, CC, CX3C, and XC. Exemplary chemokines include chemokines from the CC subfamily: CCLI, CCL2 (MCP-1), CCL3, CCL4, CCL5 (RANTES), CCL6, CCL7, CCL8, CCL9 (or CCLI0), CCLI 1, CCL12, CCL13, CCL14, CCL15, CCL16, CCLI 7, CCL18, CCL19, CCL20, CCL21, CCL22, CCL23, CCL24, CCL25, CCL26, CCL27, and CCL28; the CXC subfamily: CXCLI, CXCL2, CXCL3, CXCL4, CXCL5, CXCL6, CXCL7, CXCL8, CXCL9, CXCLI0, CXCLII, CXCL12, CXCL13, CXCL14, CXCL15, CXCL16, and CXCLI 7; the XC subfamily: XCLI and XCL2;
and the CX3C subfamily CX3CL1.

In certain embodiments, HPV vaccine antigens provided herein are co-delivered and/or co-expressed along with cyclin-dependent kinase inhibitors (CKIs). In some embodiments, the CKIs specifically inhibit CDK4 and CDK6 (e.g., p16INK4a). In some embodiments, the CKIs consist of one or more 21Cip1, p27Kip1, or p57Kip2. In some embodiments, the CKIs are delivered via administration of Palbociclib (Ibrance), Ribociclib (Kisqali), or Abemaciclib (Verzenio).

In some embodiments, the dose of the CKIs administered is about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, or 1000 mg.

In certain embodiments, HPV vaccine antigens provided herein are co-delivered and/or co-expressed (e.g., as part of the same HPV antigen delivery vector or via a separate vector) along with interferons. Interferons (IFNs) comprise interferon type I (e.g. IFN-α, IFN-β, IFN-ε, IFN-κ, and IFN-ω), interferon type II (e.g. IFN-γ), and interferon type 111. In some embodiments, IFN-α is further classified into about 13 subtypes including IFNAI, IFNA2, IFNA4, IFNA5, IFNA6, IFNA7, IFNA8, IFNA 10, IFNA13, IFNA14, IFNA16, IFNAI 7, and IFNA21.

In certain embodiments, HPV vaccine antigens provided herein are co-delivered and/or co-expressed (e.g., as part of the same HPV antigen delivery vector or via a separate vector) along with an interleukin. Interleukins are expressed by leukocytes or white blood cells and promote the development and differentiation of T and B lymphocytes and hematopoietic cells. Exemplary interleukins include IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and IL-36, and functional fragments or variants thereof. In some embodiments, interleukins are IL-2, IL-12, IL-15, IL-21, or functional fragment or variants thereof. In some embodiments, the interleukin is IL-15, or a functional fragment or variant thereof, and is comprised in a fusion protein comprising IL-15, or a functional variant thereof, and IL-15a, or a functional fragment or variant thereof.

In certain embodiments, the interleukin is IL-12, or a functional fragment or variant thereof.

IL-12 is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as p70), and a homodimer of p40 are formed following protein synthesis. IL-12 is the master regulator of the immune system. IL-12 induces the local and systemic production of IL-12, initiates a cytokine cascade resulting in downstream endogenous interferon-γ (IFN-γ), and via these signaling pathways activates both innate (i.e., NK cells) and adaptive (i.e., cytotoxic T lymphocytes) immunities. See FIG. 11. The adaptive immune system induces T cells to change from a naïve phenotype to an effector functional type or a memory type. The Th1/Th2 phenotype reflects the result of naïve T cell activation. IL-12 also acts to remodel the tumor microenvironment (TME) and has anti-angiogenic effects. IL-12 binds to the IL-12 receptor (IL-12R), which is a heterodimeric receptor formed by IL-12R-β1 and IL-12R-β2. The receptor complex is primarily expressed by T cells, but also other lymphocyte subpopulations have been found to be responsive to IL-12.

IL-12 is a candidate for tumor immunotherapy in humans because it functions in bridging innate and adaptive immunity. Indeed, IL-12 has proven effective in animal models of tumor therapy. However, clinically severe side effects were frequently associated with systemic administration of IL-12 in human therapeutic studies. Despite such hurdles, however, IL-12 continues to be of significant interest for use in human (clinical) oncology, particularly because its full therapeutic potential when used by itself or in combination with other onco-therapeutic compounds and methods of treatment, or in particular, via local production rather than systemic administration, has not been fully investigated, much less realized.

In certain embodiments, the IL-12 is a single chain IL-12 (scIL-12), protease sensitive IL-12, destabilized IL-12, membrane bound IL-12, or intercalated IL-12. In some instances, the IL-12 variants are as described in WO2015/095249, WO2016/048903, WO2017/062953.

In certain embodiments, HPV vaccine antigens provided herein are delivered to, and/or are expressed in a subject, in conjunction with delivery and/or expression of IL-12, or a functional fragment or variant thereof. In some embodiments an IL-12 polypeptide, or functional fragment or variant thereof, is expressed from the same HPV vaccine antigen expression vector. In other embodiments, the IL-12 polypeptide, or functional fragment or variant thereof, is expressed from a separate vector in conjunction with HPV vaccine antigen delivery or expression. In some embodiments, the vector expressing IL-12, or a functional fragment or variant thereof, is a replication-deficient adenoviral vector (e.g., a GC46 Gorilla adenovector).

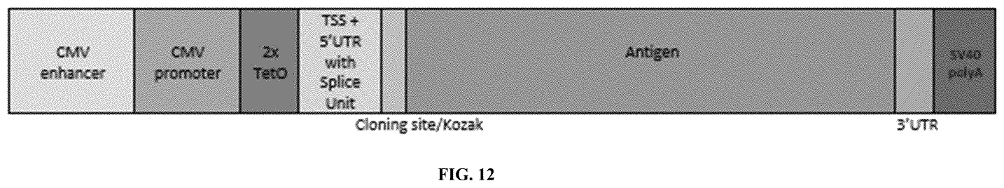

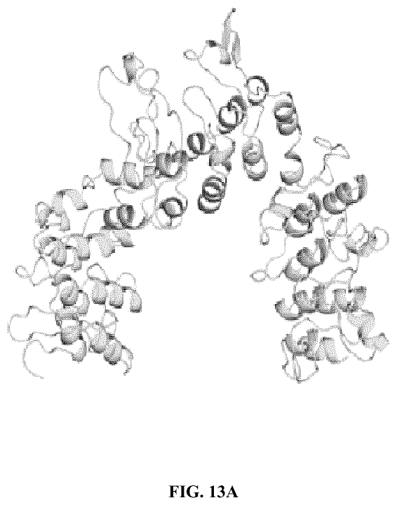

In certain embodiments, in conjunction with delivery or expression of the present invention (i.e., the novel HPV antigen designs disclosed herein), expression of an interleukin in a subject is controlled by constitutive or inducible regulation of expression. In a preferred embodiment, in conjunction with delivery or expression of the present invention, expression of the interleukin in a subject is controlled by inducible regulation of expression (also referred to as, inducibly regulated expression of interleukin). See FIG. 12.

In certain embodiments, the IL-12 is expressed in a genetic construct comprising a polynucleotide encoding IL12p40, or a functional fragment or variant thereof, linked by way of an IRES (e.g., an EMCV IRES) to a polynucleotide encoding an IL12p35, or a functional fragment or variant thereof. In certain other embodiments, the IL-12 is expressed as a fusion protein comprising an IL12p40, or a functional fragment or variant thereof, and IL12p35, or a functional fragment or variant thereof. In certain such embodiments, the IL12p40, or a functional fragment or variant thereof, is linked by way of a peptide linker with IL12p35, or a functional fragment or variant thereof.

In certain embodiments, in conjunction with delivery or expression of the present invention, IL-12 is expressed as a single chain IL12p70 built into a GC46 Gorilla adenovector (either the same, or separate from, the adenovector delivering the HPV vaccine antigen of the present invention) that has the capability to deliver dose-dependent production of bioactive IL12. In further embodiments, there is no preexisting immunity or presence of neutralizing antibodies directed against the GC46 gorilla adenovector(s) that may limit utility in treating patients with the present invention in combination with IL-12. In certain embodiments, the single chain IL-12p70 has bioactivity similar to that of natural recombinant protein and no propensity of producing the regulatory IL-12p40 homodimer.

In certain embodiments, the interleukin is delivered intratumorally in conjunction with the present invention. In other embodiments, the interleukin is delivered locally to the site of the tumor or to a lymph node associated with the tumor.

In certain embodiments, the vector expressing the interleukin is administered at a unit dose of about 1×10¹¹, 2×10¹¹, 3×10¹¹, 4×10¹¹, 5×10¹¹, 6×10¹¹, 7×10¹¹, 8×10¹¹, 9×10¹¹, or 1×10¹², or 2×10¹² viral particles (vp). In some embodiments, the vector is administered at a dose of about 2×10¹¹ vp. In other embodiments, the vector is administered at a dose of about 5×10¹¹ vp.

The initial dose of the composition or vector expressing the novel HPV antigen designs disclosed herein and the initial dose of interleukin is administered concurrently or sequentially. For example, the initial dose of the composition or vector expressing the HPV antigens may be administered at a period of time after the initial dose of interleukin. Alternatively, the initial dose of the composition or vector expressing the HPV antigens may be administered at a period of time before the initial dose of interleukin. In some embodiments, the initial dose of interleukin is administered at about 1, 2, 3, 4, 5, 6, 7 or more days prior to the administration of the composition or vector expressing the HPV antigens. In some embodiments, one or more subsequent doses of interleukin are administered after the administration of the initial dose of the composition or vector expressing the HPV antigens. In some embodiments, one or more subsequent doses of interleukin are administered within 7 to 28 days after the administration of the composition or vector expressing the HPV antigens. In some embodiments, one or more subsequent doses of interleukin are administered at least 7, 8, 9, 10, 11, 12, 13, 14, 15, 16, 17, 18, 19, 20, 21, 22, 23, 24, 25, 26, 27, 28 or more days after administration of the composition or vector expressing the HPV antigens. In some embodiments, one of the subsequent doses of interleukin is administered at 15 days after administration of the composition or vector of expressing the HPV antigens.

In other embodiments, subsequent doses of interleukin are administered once every one, two, three or four weeks after the first dose of interleukin. In further such embodiments, subsequent doses of interleukin are administered once every two weeks or once every four weeks after the first dose of interleukin.

In some embodiments, the interleukin is a membrane-bound IL-15. In certain such embodiments, the membrane-bound IL-15 (mbIL-15) comprises a full-length IL-15 (e.g., a native IL-15 polypeptide) or functional fragment or variant thereof, fused in frame with a full length IL-15Rα, or a functional fragment or variant thereof. In some cases, the IL-15 is indirectly linked to the IL-15Rα through a linker. In some instances, the mbIL-15 is as described in Hurton et al., “Tethered IL-15 augments antitumor activity and promotes a stem-cell memory subset in tumor-specific T cells,” PNAS 2016.

In certain embodiments, HPV vaccine antigens provided herein are co-delivered and/or co-expressed (e.g., as part of the same HPV antigen delivery vector or via a separate vector) along with tumor necrosis factors. Tumor necrosis factors (TNFs) are a group of cytokines that modulate apoptosis. In some instances, there are about 19 members within the TNF family, including, not limited to, TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).

In certain embodiments, HPV vaccine antigens provided herein, are co-delivered and/or co-expressed (e.g., as part of the same HPV antigen delivery vector or via a separate vector) along with colony stimulating factors. Colony-stimulating factors (CSFs) are secreted glycoproteins that interact with receptor proteins on the surface of hemopoietic stem cells, which subsequently modulates cell proliferation and differentiation into specific kind of blood cells. In some instances, a CSF comprises macrophage colony-stimulating factor, granulocyte macrophage colony-stimulating factor (GM-CSF), granulocyte colony-stimulating factor (G-CSF) or promegapoietin.

In certain embodiments, HPV vaccine antigens provided herein may be co-delivered and/or co-expressed (e.g., as part of the same HPV antigen delivery vector or via a separate vector) along with surface active agents such as immune-stimulating complexes (ISCOMS). Freunds incomplete adjuvant, LPS analog including monophosphoryl Lipid A (WL), muramyl peptides, quinone analogs and vesicles such as squalene and squalene, and hyaluronic acid may also be used administered in conjunction with the gene construct.

In certain embodiments, an additional element may be added which serves as a target for cell destruction if it is desirable to eliminate cells receiving the genetic construct for any reason. A herpes thymidine kinase (tk) gene in an expressible form can be included in the gene construct. The drug gangcyclovir can be administered to the individual and that drug will cause the selective killing of any cell producing tk, thus, providing the means for the selective destruction of cells with the genetic construct.

In certain embodiments, the additional therapy is administration of a small molecule enzyme inhibitor or anti-metastatic agent. In some embodiments, the additional therapy is the administration of a side effect limiting agent (e.g., an agent intended to lower the incidence and/or severity of side effects of treatment, such as nausea, etc.). In some embodiments, the additional therapy is radiation therapy. In some embodiments, the additional therapy is surgery. In further embodiments, the surgery is debulking surgery. In some embodiments, the additional therapy is a combination of radiation therapy and surgery. In some embodiments, the additional therapy is gamma irradiation.

### 1. Chemotherapy

In some embodiments, the additional therapy is chemotherapy. The additional therapy can be one or more chemotherapeutic agents known in the art, such as dacarbazine or temozolomide. The term “chemotherapy” refers to the use of drugs to treat cancer. “Chemotherapy agent” refers to a compound or composition administered in the treatment of cancer. These agents or drugs are classified according to their mode of activity within the cell, for example whether they affect the cell cycle and at what stage. Alternatively, the agent can be characterized based on its ability to directly cross-link DNA, insert into DNA, or influence nucleic acid synthesis to induce chromosomal and mitotic mutations.

Exemplary chemotherapy agents that may be administered in combination with the compositions of the present invention include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denilcukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 1311-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). Examples of further chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards s chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-F-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; ctoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; ctoposide (VP-16); ifosfamide; mitoxantrone; vincristine; vinorelbine; novantrone; teniposide; edatrexate; daunomycin; aminopterin; xeloda; ibandronate; irinotecan CPT-11); (e.g., topoisomerase inhibitor RFS 2000; difluoromethylornithine (DMFO); retinoids such as retinoic acid; capecitabine; and pharmaceutically acceptable salts, acids or derivatives of any of the above, and combinations thereof.

In some embodiments, the chemotherapy agent is a histone decetylase (“HDAC”) inhibitor. HDACs are enzymes involved in the regulation of gene expression by modifying chromatin structure. They remove acetyl groups from histones, leading to chromatin condensation and repression of gene transcription. In cancer, dysregulation of histone acetylation and deacetylation processes can contribute to the development and progression of the disease. HDAC inhibitors represent a promising class of anticancer agents that target epigenetic alterations and dysregulated gene expression in cancer cells. HDAC inhibitors may inhibit angiogenesis, induce apoptosis or cell cycle arrest in cancer cells, and/or promote histone acetylation, which can lead to re-expression of tumor suppressor gene and inhibition of oncogenes. While several HDAC inhibitors have been developed and are undergoing clinical trials, further research is needed to optimize their efficacy and safety profiles for the treatment of various types of cancer. Examples of HDAC inhibitors that may be administered in combination with the compositions of the present invention include, without limitation, vorinostat, romidepsin, belinostat, panobinostat, entinostat, and trichostatin A.

In some embodiments, the chemotherapy agent is a taxoid. For example, the taxoid is docetaxel. In some embodiments, the chemotherapy agent is a platinum coordination complex. For example, the platinum coordination complex is cisplatin. In some embodiments, two chemotherapy agents are used. For example, the two chemotherapy agents are a taxoid and a platinum coordination complex. In some embodiments, the two chemotherapy agents are docetaxel and cisplatin.

In some embodiments, the chemotherapy agent is administered at a dose ranging from about 0.1 mg/kg to about 10 mg/kg. In particular embodiments, the chemotherapy agent is administered at a dose of about 0.1 mg/kg, about 0.3 mg/kg, about 0.5 mg/kg, about 1 mg/kg, about 1.5 mg/kg, about 2 mg/kg, about 2.5 mg/kg, about 3 mg/kg, about 5 mg/kg, or about 10 mg/kg. In a particular embodiment, the chemotherapy agent is administered at a dose of 2 mg/kg. In another embodiment, the chemotherapy agent is administered at a dose of 3.675 mg/kg. In another embodiment, the chemotherapy agent is administered at a dose of 10 mg/kg.

In some embodiments, the chemotherapy agent is administered at a dose of 10 mg, 50 mg, 75 mg, 100 mg, 142.5 mg, 150 mg, 200 mg, 250 mg, 300 mg, 350 mg, 400 mg, 450 mg, 500 mg, 550 mg, 600 mg, 650 mg, 700, mg, 750 mg, 800 mg, 850 mg, 900 mg, 950 mg, or 1,000 mg. In a particular embodiment, the chemotherapy agent is administered at a dose of 142.5 mg. In a particular embodiment, the chemotherapy agent is administered at a dose of 200 mg. In another embodiment, the chemotherapy agent is administered at a dose of 400 mg.

The dosing regimen for the chemotherapy agent may be adjusted based on individual patient factors, such as body weight, renal function, and liver function. A person of ordinary skill in the art can determine the most appropriate dosing schedule for each patient.

The chemotherapy agent may be administered prior to, concurrently with, or subsequent to the administration of the HPV vaccine antigens. For example, the chemotherapy agent may be administered approximately 1 day, 3 days, 1 week, 2 weeks, or 1 month before or after administration of the HPV vaccine antigens.

In some embodiments, the chemotherapy agent may be administered multiple times, including but not limited to twice a week, once a week, once every two weeks, once every three weeks, once every four weeks, once every five weeks, once every six weeks, once every seven weeks, once every eight weeks, once every nine weeks, or once every ten weeks.

The duration of treatment with the chemotherapy agent may vary depending on the cancer type, response to therapy, and tolerability. In some cases, treatment may continue until disease progression or unacceptable toxicity, while in others, a fixed duration of treatment (e.g., 1-2 years) may be recommended. In some embodiments, the duration of therapy for the chemotherapy agent may be up to 12 months, 18 months, 24 months, 30 months, or 36 months. In a particular embodiment, the duration of therapy for the chemotherapy agent is up to 24 months.

The HPV vaccine antigens of the present invention may be administered in combination with one or more chemotherapy agents at the dosing ranges disclosed herein for the treatment of various cancers. Exemplary cancers that may be treated with the combination of HPV vaccine antigens and a chemotherapy agent include, but are not limited to, cervical cancer, vulvar cancer, vaginal cancer, anal cancer, penile cancer, oropharyngeal cancer (throat cancer), recurrent respiratory papillomatosis (RRP), melanoma, non-small cell lung cancer (NSCLC), small cell lung cancer (SCLC), head and neck squamous cell carcinoma (HNSCC), classical Hodgkin lymphoma (cHL), primary mediastinal large B-cell lymphoma (PMBCL), urothelial carcinoma, microsatellite instability-high (MSI-H) or mismatch repair deficient (dMMR) solid tumors, gastric cancer, esophageal cancer, hepatocellular carcinoma (HCC), Merkel cell carcinoma (MCC), renal cell carcinoma (RCC), endometrial carcinoma, tumor mutational burden-high (TMB-H) solid tumors, cutaneous squamous cell carcinoma (cSCC), and triple-negative breast cancer (TNBC).

In some embodiments of the present invention, the chemotherapy agent administered in combination with the HPV vaccine antigens is docetaxel, cisplatin, or a combination thereof. Docetaxel, cisplatin, or a combination thereof may be administered at the dosing ranges disclosed herein, and may be dependent on the cancer being treated and the patient's individual characteristics. In some embodiments, the dosing regimen for docetaxel may be 142.5 mg (or 75 mg/m²), administered as an intravenous infusion over 60 minutes every 3 weeks (Q3W). In some embodiments, the dosing regimen for cisplatin may be 142.5 mg (or 75 mg/m²), administered as an intravenous infusion over 60 minutes every 3 weeks (Q3W).

In particular embodiments, the combination of HPV vaccine antigens and one or more chemotherapy agents, such as docetaxel or cisplatin, is administered for the treatment of cervical cancer, HPV-related carcinoma, HPV-related malignancy, head and neck cancer, and/or oropharyngeal squamous cell carcinoma. These cancers are known to be associated with HPV infection, and the combination therapy disclosed herein may provide enhanced therapeutic efficacy compared to either the HPV vaccine antigens or the chemotherapy agent(s) alone.

### 2. Radiation Therapy

In certain embodiments, radiation therapy may be used in combination with any of the methods of treatment described herein. “Radiation therapy” refers to treatment for a disease or disorder (typically, cancer) where radioactive energy is used to destroy cells and their division. Modern radiation therapy systems use relatively high energy beams of radiation from radioactive isotopes or electron beam X-Ray or as γ-rays generators. Radiation therapy includes external beam radiation, intensity modulated radiation therapy (IMRT), focused radiation, and any form of radiosurgery including Gamma Knife, Cyberknife, Linac, and interstitial radiation (e.g. implanted radioactive seeds, GliaSite balloon), and/or with surgery. Other forms of DNA damage factors that may be implemented in radiation therapy include microwave, proton beam irradiation (U.S. Pat. Nos. 5,760,395 and 4,870,287) and ultraviolet irradiation The dose range of X-rays ranges from 50 to 200 rotgens per day for a long period of time (3 to 4 weeks) to 2000 to 6000 lentgens for a single dose. The range of irradiation of radioactive isotopes can vary widely, depending on the half-life of the isotope, the intensity and type of radiation emitted, and the rate of absorption of neoplastic cells.

In certain embodiments, radiation therapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

### 3. Immunotherapy

In some embodiments, the subject may also be administered an immunotherapeutic agent. “Immunotherapy” refers to a treatment that uses a subject's immune system to treat cancer, e.g., cancer vaccines, cytokines, use of cancer-specific antibodies, T cell therapy, and dendritic cell therapy. In the context of cancer therapy, immunotherapy generally relies on the use of immune effector cells and molecules to target and destroy cancer cells.

In some embodiments, the subject is also administered an immune modulator. An “immune modulator” is a type of drug (large or small molecule, including but not limited to antibodies (immunoglobulins) and other proteins), vaccine or cell therapy which induces, amplifies, attenuates or prevents change in the immune system cells, such as T cells, and some cancer cells. An immune modulator may be, for example, an immune checkpoint inhibitor, a vaccine, a molecule that stimulates T cells and/or NK cells, a cytokine, an antigen specific binder, a T cell, a NK cell, chimeric antigen receptor (CAR) or T-cell receptor (TCR), or a cell expressing a CAR or TCR. Immune modulators may be used to treat cancer, alone or in conjuction with other compounds. Immune modulators include a chemotherapy or a radiation.

Examples of immune modulators include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interleukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophase inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“HMR-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), STNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP S”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-1BB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), CAMP receptor protein (“CRP”), Human Epidermal Growth Factor Receptor 2 (“ErbB2”), FoUistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD F”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL RI”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACT”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor k B (“RANK”). In certain preferred embodiments, the subject is also administered IFN-gamma (IFN γ). In some embodiments, the subject is pretreated with IFNγ, such as with low doses of IFNγ, prior to administering the TCR-modified immune effector cells disclosed herein (e.g., the adoptive immunotherapy compositions disclosed herein comprising the TCR-T cells disclosed herein).

As noted above, in certain embodiments, the immunotherapy can be a cytokine. In some embodiments, the cytokine is a membrane-bound cytokine, which is co-expressed with a chimeric antigen receptor described herein. In some instances, the cytokine comprises a chemokine, an interferon, an interleukin, a colony-stimulating factor or a tumor necrosis factor. In some instances, one or more methods described herein further comprise administration of a cytokine selected from IL2, IL7, IL12, IL15, a fusion of IL-15 and IL-15Rα, IL21, IFNγ or TNF-α.

Non-limiting examples of immune modulators by target type are shown in Table 3.

TABLE 3
Immune Modulators by Target Type

| Category | Target | Examples | Brief Description |
|---|---|---|---|
| Immune | | | |
| Checkpoint | | | |
| Inhibitors | | | |
| | PD-1 inhibitors | Cemiplimab | Block PD-1 protein on T |
| | | Nivolumab | cells, allowing them to |
| | | Pembrolizumab | attack cancer cells more |
| | | Pidilizumab | effectively. |
| | | Spartalizumab | |
| | | AMP-224 | |
| | | AUNP-12 | |
| | | BGB-A317 | |
| | | MEDI-0680 | |
| | | STI-A1110 | |
| | PD-L1 inhibitors | Atezolizumab | Block PD-L1 protein on |
| | | Avelumab | cancer cells, preventing |
| | | Durvalumab | them from hiding from T |
| | | BMS-936559 | cells. |
| | | CK-301 | |
| | | KD033 | |
| | CTLA-4 | Ipilimumab | Block CTLA-4 protein, |
| | inhibitors | Tremelimumab | boosting the immune |
| | | | system's response to |
| | | | cancer. |
| | Negative | VISTA inhibitors | Target VISTA protein, a |
| | checkpoint | | negative regulator of |
| | regulators | | immune response. |
| | Co-inhibitory | Tim-3 inhibitors | Target various co- |
| | receptor targets | Tim-3 ligands | inhibitory receptors and |
| | | Galectin 9 antagonists | their ligands, enhancing |
| | | CNC225 | immune response. |
| | | HMGB1 | |
| | | VB4-845 | |
| | | Phosphatidyl serine | |
| | | CECAM-1 | |
| | | LAG-3 | |
| | | IMP321 | |
| | | TIGIT | |
| | | BMS986207 | |
| | CD25 (IL2RA) | Daclizumab | Targets CD25 receptor, |
| | | | reducing regulatory T |
| | | | cells that suppress |
| | | | immune response. |
| | Toll-like | Anti-TLR2 antibodies | Blocks TLR2 signaling, |
| | Receptors | TLR2 blockade drugs | potentially modulating |
| | (TLRs) | CU-CPT22 | the immune response. |
| | | C29 | |
| | | MMG-11 | |
| Intrinsic | | | |
| Pathways | | | |
| | IDO1 | Indoximod | Inhibits IDO1 enzyme, |
| | | | preventing cancer cells |
| | | | from suppressing |
| | | | immune response. |
| | TDO | CRD1152 | Inhibits TDO enzyme, |
| | | | another player in immune |
| | | | suppression by cancer cells. |
| | Ectonucleotidase | CD39 inhibitors | Block CD39 and CD73 |
| | inhibitors and | CD73 inhibitors | enzymes, involved in |
| | antibodies | OREG-103/BY40 | regulating immune |
| | | BMS-986179 | response. |
| | B7-H3 | MGC018 | Targets B7-H3 protein, |
| | | | potentially blocking its |
| | | | immune-suppressive |
| | | | effects. |
| | Bispecifics | Beta-TRAP (TGF-beta & PD-L1) | Dual-targeting drugs |
| | | BMS-936559 (PD-L1 & B7.1) | hitting two immune |
| | | | checkpoints |
| | | | simultaneously. |
| Co-Stimulatory | | | |
| Receptor Targets | | | |
| | Tumor necrosis | GITR agonists | Target various members |
| | factor (TNF) | (INCAGN01876, JNJ- | of the TNF superfamily |
| | superfamily | 68284767, JNJ-7424) | involved in immune |
| | members | GITR Ligands (AMG594, | activation. |
| | | ALT-836) | |
| | | CD27 agonists (Varlimumab, | |
| | | CDX-1127, SHR-1210) | |
| | | CD137 (4-1BB) agonists | |
| | | (Urelumab, BMS-663516, | |
| | | Utomilumab, PF-05080825) | |
| | | TNF-alpha Fusion Proteins | |
| | | (AMG 592, REGN3783) | |
| | Co-Stimulatory | OX40 (CD134) agonists | Stimulate co-stimulatory |
| | receptor targets | MEDI6469 (OX40) | receptors on T cells, |
| | | CD40 agonists | boosting their activity. |
| | | Dacetuzumab (CD40) | |
| | | ICP-870,893 (CD40) | |
| | Toll-like | VTX-2337 (TLR8) | Activate TLR8, a |
| | receptor (TLR) | | receptor triggering |
| | agonists | | immune response. |
| | CD28 | BTLA antagonists | Block BTLA, a co- |
| | superfamily | | inhibitory molecule. |
| | costimulatory | | |
| | molecules | | |
| | | ICOS agonists | Activates ICOS, a |
| | | Vopratelimab (GSK335) | molecule on immune |
| | | STI-A1110 | cells that plays a crucial |
| | | AT9283 | role in their activation |
| | | A-419 | and survival. |
| Tumor-Specific | | | |
| Antigens | | | |
| | | MAGE-A antigens | Play a crucial role in the |
| | | NY-ESO-1 | development of cancer |
| | | CEA (carcinoembryonic | immunotherapies, |
| | | antigen) | offering potential targets |
| | | HER2/neu | for the immune system to |
| | | EGFR (epidermal growth factor | recognize and destroy |
| | | receptor) | cancer cells |
| | | Cetuximab (Erbitux) | |
| | | Blinatumomab | |
| | | Catumaxomab | |
| | | Ertumaxomab | |
| Antibody-Drug | | | |
| Conjugates | | | |
| (ADCs) | | | |
| | | Daratumumab (CD38) | Combine antibody |
| | | Isatuximab (CD38) | targeting with potent |
| | | Lirilumab (KIR) | drugs for targeted |
| | | Elotuzumab (SLAMF-7) | delivery |
| | | IPH2201 (DNAM-1) | |
| | | Arabinoxylan (NKG2A/D) | |
| | | MGN-3 (Nectin-½) | |
| Cancer | | | |
| Vaccines | | | |
| | | Dendritic cell vaccine | Train immune system to |
| | | TUMAP vaccine | recognize and attack |
| | | Oncofetal antigen vaccine | specific cancer cells |
| | | Autologous tumor cell lysate | |
| | | vaccine | |
| Viral | | | |
| Vaccines | | | |
| | | HPV (e.g., HPV16 peptide | Target specific viruses |
| | | vaccines, like E6/E7) | associated with cancer |
| | | HBV | development |
| | | Shingles | |
| | | CMV | |
| | | EBV | |
| Immunostimulant | | | |
| Adjuvants | | | |
| | | TLR9 and STING ligands | Enhance immune |
| | | | response to vaccines or |
| | | | other therapies |
| Cellular | | | |
| Therapies | | | |
| | Lymphokine- | Metastatic melanoma | Utilize modified immune |
| | activated killer | Renal cell carcinoma | cells to directly attack |
| | (LAK) cells | Other cancers | cancer cells |
| | Tumor | Melanoma | TILs are immune cells |
| | infiltrating | Glioblastoma | found within tumors and |
| | lymphocytes | | offer a promising |
| | (TILs) | | personalized approach to |
| | | | cancer immunotherapy |
| | Cytokine- | Interleukin-2 (IL-2) | Stimulate and direct the |
| | activated killer | Interferon-alpha (IFN-α) | immune system against |
| | cells | Granulocyte-macrophage | cancer |
| | | colony-stimulating factor | |
| | | (GM-CSF) | |
| | | Cytokine-induced Killer (CIK) | |
| | | cell | |
| | Chimeric antigen | Approved targets (e.g., CD19, | Uses genetically |
| | receptor (CAR) | BCMA) | modified T cells to target |
| | | B-cell targets (e.g., CD20, | and destroy cancer cells |
| | | CD22, CD33, CD38, CD44, | |
| | | CD79b, CD123, FLT3) | |
| | | T-cell targets (CD25, CD4, | |
| | | CD5, CD7, CTLA-4, PD-1) | |
| | | Myeloid targets (CD33, CD123) | |
| | | Solid tumor targets (e.g., HER2, | |
| | | EGFR, mesothelin, ROR1, | |
| | | CEA, MAGE-A, GD2, PSMA, | |
| | | mesothelin, CD22, EGFR, | |
| | | Folate receptor α, Mucins, etc.) | |
| | T-Cell Receptor | MAGE-A3 | Uses a patient's naturally |
| | (TCR) | NY-ESO-1 | occurring TCRs, which |
| | | HPV-specific | receptors recognize |
| | | | specific antigens on the |
| | | | surface of cells. |

An “immune checkpoint inhibitor” is a type of drug (large or small molecule, including but not limited to antibodies (immunoglobulins) and other proteins) which block certain proteins made by some types of immune system cells, such as T cells, and some cancer cells. These proteins help keep immune responses in check and limit or prevent T cells from killing cancer cells. When these proteins are blocked, the molecular “brakes” on the immune system are released and T cells can better (i.e., more effectively) kill cancer cells. Examples of checkpoint proteins found on T cells or cancer cells include PD-1/PD-L1 and CTLA-4/B7-1/B7-2. Immune checkpoint inhibitors may be used to treat cancer; alone or in conjunction with other compounds.

In some of the embodiments of the methods described herein, the immune checkpoint inhibitor is for example, a PD-1 binder, a PD-L1 binder, a CTLA-4 binder, a V-domain immunoglobulin suppressor of T cell activation (VISTA) binder, a TIM-3 binder, a TIM-3 ligand binder, a LAG-3 binder, a T-cell immunoreceptor with Ig and ITIM domains (TIGIT) binder, a B- and T-cell attenuator (BTLA) binder, a B7-H3 binder, a TGFbeta and PD-L1 bispecific binder or a PD-L1 and B7.1 bispecific binder.

In some embodiments, the PD-1 binder is an antibody that specifically binds PD-1. In some embodiments, the PD-1 binder is an antagonist. In some embodiments, the antibody that binds PD-1 is pembrolizumab (KEYTRUDA, MK-3475; CAS #1374853-91-4) developed by Merck, pidilizumab (CT-011; CAS #1036730-42-3) developed by Curetech Ltd., nivolumab (OPDIVO, BMS-936558, MDX-1106; CAS #946414-94-4) developed by Bristol Myer Squibb, MEDI0680 (AMP-514); developed by AstraZenenca/MedImmune, cemiplimab-rwlc (REGN2810, LIBTAYO®□□ CAS #1801342-60-8) developed by Regeneron Pharmaceuticals, BGB-A317 developed by BeiGene Ltd., spartalizumab (PDR-001; CAS #1935694-88-4) developed by Novartis, or STI-A1110 developed by Sorrento Therapeutics. In some embodiments, the antibody that binds PD-1 is described in PCT Publication WO2014/179664, for example, an antibody identified as APE2058, APE1922, APE1923, APE1924, APE 1950, or APE 1963 developed by Anaptysbio, or an antibody containing the CDR regions of any of these antibodies. In other embodiments, the PD-1 binder is a fusion protein that includes the extracellular domain of PD-L1 or PD-L2, for example, AMP-224 (AstraZeneca/MedImmune). In other embodiments, the PD-1 binder is a peptide inhibitor, for example, AUNP-12 developed by Aurigene.

In some embodiments, the PD-L1 binder is an antibody that specifically binds PD-L1. In some embodiments, the PD-L1 binder is an antagonist. In some embodiments, the antibody that binds PD-L1 is atezolizumab (RG7446, MPDL3280A; Tecentriq; CAS #1380723-44-3) developed by Genentech, durvalumab (MEDI4736, IMFINZI®□□ CAS #1428935-60-7) developed by AstraZeneca/MedImmune, BMS-936559 (MDX-1105) developed by Bristol Myers Squibb, avelumab (MSB0010718C; Merck KGaA; Bavencio; CAS #1537032-82-8), KD033 (Kadmon), the antibody portion of KD033, STI-A 1014 (Sorrento Therapeutics) or CK-301 (Checkpoint Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies.

In some embodiments, the CTLA-4 binder is an antibody that specifically binds CTLA-4. In some embodiments, the CTLA-4 binder is an antagonist. In some embodiments, the antibody that binds CTLA-4 is ipilimumab (YERVOY) developed by Bristol Myer Squibb or tremelimumab (CP-675,206) developed by MedImmune/AtraZenica then Pfizer. In some embodiments, the CTLA-4 binder is an antagonistic CTLA-4 fusion protein or soluble CTLA-4 receptor, for example, KAHR-102 developed by Kahr Medical Ltd.

In some embodiments, the 4-1BB (CD137) binder is a binding molecule, such as an anticalin. In some embodiments, the 4-1BB binder is an agonist. In some embodiments, the anticalin is PRS-343 (Pieris AG). In some embodiments, the 4-1BB binder is an agonistic antibody that specifically binds 4-1BB. In some embodiments, antibody that binds 4-1BB is PF-2566 (PF-05082566) developed by Pfizer or urelumab (BMS-663513) developed by Bristol Myer Squibb.

In some embodiments, the LAG3 binder is an antibody that specifically binds LAG3. In some embodiments, the LAG3 binder is an antagonist. In some embodiments, the antibody that binds LAG3 is IMP701 developed by Prima BioMed, IMP731 developed by Prima BioMed/GlaxoSmithKline, BMS-986016 developed by Bristol Myer Squibb, LAG525 developed by Novartis, and GSK2831781 developed by Glaxo SmithKline. In some embodiments, the LAG-3 antagonist includes a soluble LAG-3 receptor, for example, IMP321 developed by Prima BioMed.

In some embodiments, the KIR binder is an antibody that specifically binds KIR. In some embodiments, the KIR binder is an antagonist. In some embodiments, the antibody that binds KIR is lirilumab developed by Bristol Myer Squibb/Innate Pharma.

In some embodiments, a combination of controlled expression of IL-12 with a check point inhibitor, such as but not limited to, a PD-1-specific antibody (e.g., nivolumab) provides improved cancer treatment, such as but not limited to brain cancer (e.g., gliomas/glioblastomas) wherein IL-12 provides therapeutically effective recruitment and infiltration of T cells (such as killer T-cells) into the tumor while the check point inhibitor (e.g., anti-PD-1 antibody) provides for enhanced and/or improved immune cell function and activity within the tumor (i.e., improved anti-tumor immune cell activity).

### 4. Hormone Therapy

In some embodiments, a therapeutic agent for use in combination with a composition of the present invention as described herein may be a hormonal regulating agent (e.g., hormone therapy), such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogen (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

### 5. Surgery

In some embodiments, the compositions of the present invention are administered conjointly with surgery. Therapeutic surgery includes resection in which all or part of a cancer tissue is physically removed, dissected and/or destroyed, and the treatment, chemotherapy, radiotherapy, hormone therapy, gene therapy, immunotherapy and/or alternative therapy of this embodiment. It can be used in conjunction with other therapies. Tumorectomy refers to the physical removal of at least a portion of a tumor. In addition to tumor resection, surgical treatment may include laser surgery, cold surgery, electrosurgery and microscopically controlled surgery. Debulking refers to the reduction of as much of the volume (i.e., bulk) of a tumor without the intention of a complete eradication. Debulking is usually achieved by surgical removal.

Upon incision of some or all cancer cells, tissues or tumors, cavities may form in the body. Treatment can be by perfusion, direct injection, or topical application to the affected area using additional anti-cancer therapies. Such treatment may be, for example, every 1, 2, 3, 4, 5, 6 or 7 days, every 1, 2, 3, 4 or 5 weeks, or every 1, 2, 3, 4, 5, 6, 7, 8, 9, 10, 11 or 12 months. Such treatment can also be achieved in various dosages.

### 6. Other Combination Therapies

It is also contemplated that other agents may be used in combination with certain aspects of the present embodiments to improve the therapeutic efficacy of the treatment. These additional agents include agents that affect up-regulation of cell surface receptors and GAP junctions, cell proliferation inhibitors and differentiators, cell adhesion inhibitors, agents that increase the sensitivity of hyperproliferative cells to apoptosis inducers, or other biological agents. An increase in intercellular signaling due to an increase in the number of GAP junctions will enhance the anti-hyperproliferative effect on adjacent hyperproliferative cell populations.

In other embodiments, cell proliferation inhibition or differentiation agents can be used in combination with certain aspects of this embodiment to improve the anti-hyperproliferative efficacy of the treatment. It is believed that cell adhesion inhibitors can improve the efficacy of this embodiment. Examples of cell adhesion inhibitors include topical adhesion kinase (FAK) inhibitors and lovastatin. Additionally, it is contemplated that other agents, such as antibody c225, that increase the sensitivity of the hyperproliferative cells to apoptosis, may be used in combination with certain aspects of this embodiment to improve therapeutic efficacy.

### 7. Combination Adjuvant Therapies for HPV 16/18 Malignancies

The present invention also relates to a method for treating an HPV16/18-associated cancer, the method comprising administering to a patient in need thereof a vaccine adenoviral vector as described herein combined with a bifunctional fusion protein (or fragment or a variant thereof). In some embodiments, the fusion protein promotes natural killer cell-mediated killing of tumor cells. In some embodiments, the fusion protein comprises: (a) a cytokine trap; and (b) a PD-1 inhibitor or anti-PD-1 antibody or a functional fragment or a variant thereof. In some embodiments, the cytokine trap (e.g., TGF-beta trap) is fused to a PD-1 inhibitor optionally via a cleavable or non-cleavable linker. In some embodiments, the cytokine trap (e.g., TGF-beta trap) is a cytokine receptor (e.g., TGFβRII) In some embodiments, the cytokine receptor sequence comprises an extracellular domain (ECD) of the receptor or a functional fragment or variant thereof.

In some embodiments, the fusion protein comprising an anti-PD-1 antibody or a functional fragment or a variant thereof fused to a TGF-beta trap, can elicit a synergistic anti-tumor effect due to the simultaneous blockade of the interaction between PD-L1 on tumor cells and PD-1 on immune cells, and the neutralization of TGF-beta in the tumor microenvironment. In some embodiments, the TGF-beta trap (e.g., TGFβRII) is fused to a variable region of heavy chain (V_{H}) of PD-1 antibody or a fragment/variant thereof. In other embodiments, the TGF-beta trap is fused to IgG of a PD-1 antibody. In certain aspects, the IgG is IgG1, IgG2, IgG3, or IgG4. In an embodiment, the IgG is IgG4.

In some embodiments, the TGF-beta trap (e.g., TGFβRII) is fused to a variable region of heavy chain (V_{H}) of PD-1 antibody or a fragment/variant thereof via a linker. In some embodiments, the TGF-beta trap (e.g., TGFβRII) is fused to a constant region of the V_{H} of PD-1 antibody or a fragment/variant thereof via a linker.

In some embodiments, the TGF-beta trap (e.g., TGFβRII) is fused a variable region of light chain (V_{L}) of PD-1 antibody or a fragment/variant thereof. In some embodiments, the TGF-beta trap (e.g., TGFβRII) is fused to a constant region of the V_{L} of PD-1 antibody or a fragment/variant thereof via a linker. In some embodiments, the TGF-beta trap (e.g., TGFβRII) is fused a variable region of light chain (V_{L}) of PD-1 antibody or a fragment/variant thereof via a linker. In one aspect, the TGF-beta trap (e.g., TGFβRII) is fused to either the N- or C-terminus of the VL or VH chain or a fragment/variant thereof via a linker.

Examples of bifunctional fusion proteins that may be used in combination with the present invention include those described in PCT/US2019/041085 and PCT/US2021/041082. In an exemplary embodiment, the bifunctional fusion protein is bintrafusp alfa (“BA”). BA is a first-in-class bifunctional fusion protein composed of TGFβRII (a type II receptor for TGFβ) fused to a human IgG1 mAb blocking programmed cell death ligand 1 (PD-L1).

In some embodiments, the dose of the bifunctional fusion protein (e.g., bintrafusp alfa) administered is at least 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.2 mg, 2.4 mg, 2.6 mg, 2.8 mg, 3 mg, 3.2 mg, 3.4 mg, 3.6 mg, 3.8 mg, 4 mg, 4.2 mg, 4.4 mg, 4.6 mg, 4.8 mg, 5 mg, 5.5 mg, 6 mg, 6.5 mg, 7 mg, 7.5 mg, 8 mg, 8.5 mg, 9 mg, 9.5 mg, 10 mg, 11 mg, 12 mg, 13 mg, 14 mg, 15 mg, 16 mg, 17 mg, 18 mg, 19 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 550 mg, 600 mg, 650 mg, 700 mg, 750 mg, 800 mg, 850 mg, 900 mg, 950 mg, 1000 mg, 1200 mg, 1400 mg, 1600 mg, 1800 mg, 2000 mg, 2500 mg, 3000 mg, 3500 mg, 4000 mg, 4500 mg, or 5000 mg.

In some embodiments, the dose of the bifunctional fusion protein (e.g., bintrafusp alfa) administered is about 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1105 mg, 1110 mg, 1115 mg, 1120 mg, 1125 mg, 1130 mg, 1135 mg, 1140 mg, 1145 mg, 1150 mg, 1155 mg, 1160 mg, 1165 mg, 1170 mg, 1175 mg, 1180 mg, 1185 mg, 1190 mg, 1195 mg, 1200 mg, 1205 mg, 1210 mg, 1215 mg, 1220 mg, 1225 mg, 1230 mg, 1235 mg, 1240 mg, 1245 mg, 1250 mg, 1255 mg, 1260 mg, 1265 mg, 1270 mg, 1275 mg, 1280 mg, 1285 mg, 1290 mg, 1295 mg, 1300 mg, 1305 mg, 1310 mg, 1315 mg, 1320 mg, 1325 mg, 1330 mg, 1335 mg, 1340 mg, 1345 mg, 1350 mg, 1355 mg, 1360 mg, 1365 mg, 1370 mg, 1375 mg, 1380 mg, 1385 mg, 1390 mg, 1395 mg, 1400 mg, 1405 mg, 1410 mg, 1415 mg, 1420 mg, 1425 mg, 1430 mg, 1435 mg, 1440 mg, 1445 mg, 1450 mg, 1455 mg, 1460 mg, 1465 mg, 1470 mg, 1475 mg, 1480 mg, 1485 mg, 1490 mg, 1495 mg, 1500 mg, 1505 mg, 1510 mg, 1515 mg, 1520 mg, 1525 mg, 1530 mg, 1535 mg, 1540 mg, 1545 mg, 1550 mg, 1555 mg, 1560 mg, 1565 mg, 1570 mg, 1575 mg, 1580 mg, 1585 mg, 1590 mg, 1595 mg, 1600 mg, 1605 mg, 1610 mg, 1615 mg, 1620 mg, 1625 mg, 1630 mg, 1635 mg, 1640 mg, 1645 mg, 1650 mg, 1655 mg, 1660 mg, 1665 mg, 1670 mg, 1675 mg, 1680 mg, 1685 mg, 1690 mg, 1695 mg, 1700 mg, 1705 mg, 1710 mg, 1715 mg, 1720 mg, 1725 mg, 1730 mg, 1735 mg, 1740 mg, 1745 mg, 1750 mg, 1755 mg, 1760 mg, 1765 mg, 1770 mg, 1775 mg, 1780 mg, 1785 mg, 1790 mg, 1795 mg, 1800 mg, 1805 mg, 1810 mg, 1815 mg, 1820 mg, 1825 mg, 1830 mg, 1835 mg, 1840 mg, 1845 mg, 1850 mg, 1855 mg, 1860 mg, 1865 mg, 1870 mg, 1875 mg, 1880 mg, 1885 mg, 1890 mg, 1895 mg, 1900 mg, 1905 mg, 1910 mg, 1915 mg, 1920 mg, 1925 mg, 1930 mg, 1935 mg, 1940 mg, 1945 mg, 1950 mg, 1955 mg, 1960 mg, 1965 mg, 1970 mg, 1975 mg, 1980 mg, 1985 mg, 1990 mg, 1995 mg, or 2000 mg.

In certain embodiments, the dose of the bifunctional fusion protein (e.g., bintrafusp alfa) administered is about 1200 mg.

In some embodiments, the bifunctional fusion protein is administered as a single dose. In other embodiments, the bifunctional fusion protein is administered every other week, every two weeks (i.e., biweekly), every three weeks, every four weeks, once weekly, twice weekly, or three times a week. In an exemplary embodiment, the bifunctional fusion protein (e.g., bintrafusp alfa) is administered biweekly.

In some embodiments, the initial dose of the composition or vector expressing the HPV antigens of the present invention and the initial dose of the bifunctional fusion protein (e.g., bintrafusp alfa) is administered concurrently or sequentially. For example, the initial dose of the composition or vector expressing the HPV antigens is administered at a period of time after the initial dose of the bifunctional fusion protein. Alternatively, the initial dose of the composition or vector expressing the HPV antigens is administered at a period of time before the initial dose of the bifunctional fusion protein. In some embodiments the initial dose of the bifunctional fusion protein is administered at about 1, 2, 3, 4, 5, 6, 7 or more days prior to the administration of the composition or vector expressing the HPV antigens. In some embodiments one or more subsequent doses of the bifunctional fusion protein are administered after the administration of the initial dose of the composition or vector expressing the HPV antigens. In some embodiments, one or more subsequent doses of the bifunctional fusion protein are administered within 7 to 28 days after the administration of the composition or vector expressing the HPV antigens. In some embodiments, one or more subsequent doses of the bifunctional fusion protein are administered at least 7, 8, 9, 10, 11, 12, 13, 14, 15, 16, 17, 18, 19, 20, 21, 22, 23, 24, 25, 26, 27, 28 or more days after administration of the composition or vector expressing the HPV antigens. In some embodiments, one of the subsequent doses of the bifunctional fusion protein is administered at 15 days after administration of the composition or vector expressing the HPV antigens.

### X. Pharmaceutical Compositions

The therapeutic compounds, including polynucleotides, fusion proteins, and viral vectors as described herein, can be integrated into pharmaceutical compositions suitable for administration. These compositions typically consist of the therapeutic compound(s) and a pharmaceutically acceptable carrier. For example, any of the therapeutic compounds described herein can make up a vaccine, such as an HPV vaccine, with or without a pharmaceutically acceptable carrier.

In some embodiments, a vaccine comprises any of the polynucleotides, fusion proteins, or viral vectors described herein. For example, a vaccine (e.g., an HPV vaccine) comprises any of the polynucleotides described herein, including a polynucleotide having the sequence of SEQ ID NO: 243. A vaccine (e.g., a HPV vaccine) comprises any of the viral vectors described herein, including a viral vector having the sequence of SEQ ID NO: 244.

In certain embodiments, the pharmaceutical composition includes the vector described herein and a carrier, with specific mention of adenovirus or adenoviral vector in some embodiments.

Proper formulation of the pharmaceutical composition is dependent upon the route of administration chosen. A summary of pharmaceutical compositions described herein is found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Any suitable carrier can be used within the context of the present disclosure, and such carriers are well known in the art. The choice of carrier will be determined, in part, by the particular use of the composition (e.g., administration to an animal) and the particular method used to administer the composition. Ideally, in the context of replication-deficient adenoviral vectors, the pharmaceutical composition preferably is free of replication-competent adenovirus. The pharmaceutical composition optionally can be sterile.

Suitable pharmaceutical compositions include aqueous and non-aqueous isotonic sterile solutions, which can contain anti-oxidants, buffers, and bacteriostats, and aqueous and non-aqueous sterile suspensions that can include suspending agents, solubilizers, thickening agents, stabilizers, and preservatives. The pharmaceutical composition can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets. Preferably, the carrier is a buffered saline solution.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, (e.g., intravenous, intradermal, subcutaneous) oral (including, inhalation), topical; (i.e., transdermal), transmucosal, or rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the therapeutic compound(s) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the therapeutic compound(s) are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The therapeutic compound(s) can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for case of administration and uniformity of dosage. Dosage unit form as used herein refers to physically discrete units suited as unitary dosages for the subject to be treated; each unit containing a predetermined quantity of therapeutic compound(s) calculated to produce the desired therapeutic effect in association with the required pharmaceutical carrier. The specification for the dosage unit forms of the disclosure are dictated by and directly dependent on the unique characteristics of the therapeutic compound(s) and the particular therapeutic effect to be achieved, and the limitations inherent in the art of compounding such a therapeutic compound(s) for the treatment of individuals.

The pharmaceutical compositions can be included in a container, pack, or dispenser together with instructions for administration.

In some embodiments, the pharmaceutical composition comprising, e.g., the vector is formulated to protect the vector from damage prior to administration. For example, the pharmaceutical composition can be formulated to reduce loss of the vector on devices used to prepare, store, or administer the vector, such as glassware, syringes, or needles. The pharmaceutical composition can be formulated to decrease the light sensitivity and/or temperature sensitivity of the vector. To this end, the pharmaceutical composition preferably comprises a pharmaceutically-acceptable liquid carrier, such as, for example, those described above, and a stabilizing agent selected from the group consisting of polysorbate 80, L-arginine, polyvinylpyrrolidone, trehalose, and combinations thereof. Use of such a pharmaceutical composition will extend the shelf life of the adenovirus or adenoviral vector, and facilitate its administration. Formulations for vector-containing compositions are further described in, for example, U.S. Pat. Nos. 6,225,289, 6,514,943, and International Patent Application Publication WO 2000/034444.

The pharmaceutical composition also can be formulated to enhance transduction efficiency. In addition, the skilled artisan will appreciate that the polynucleotide, polypeptide, vector, vaccine, or cell can be present in a composition with other therapeutic or biologically-active agents. For example, factors that control inflammation, such as ibuprofen or steroids, can be part of the composition to reduce swelling and inflammation associated with in vivo administration of the polynucleotide, polypeptide, vector, composition, vaccine, or cell. For example, in cases where an adenovirus or adenoviral vector is used to deliver an antigen-encoding nucleic acid sequence to a host, immune system stimulators or adjuvants, e.g., interleukins, lipopolysaccharide, or double-stranded RNA, may be administered to enhance or modify any immune response to the antigen. Antibiotics (e.g., microbicides and fungicides) can be present to treat existing infection and/or reduce the risk of future infection, such as infection associated with gene transfer procedures.

In certain embodiments, the pharmaceutical compositions may include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the pharmaceutical composition in an acceptable range.

In certain embodiments, the pharmaceutical composition may comprise one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical composition may be formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

In certain embodiments, the pharmaceutical composition is a liquid. In some embodiments, the composition may be lyophilized and then reconstituted before use.

In certain embodiments, pharmaceutical compositions may include one or more preservatives, for example, to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In certain embodiments, the pharmaceutical composition may include one or more antifoaming agents. Antifoaming agents can reduce foaming during processing which can result in coagulation of aqueous dispersions, bubbles in the finished film, or generally impair processing. Exemplary anti-foaming agents include silicon emulsions or sorbitan sesquoleate.

In certain embodiments, the pharmaceutical composition may include one or more antioxidants. Exemplary antioxidants include butylated hydroxytoluene (BHT), sodium ascorbate, ascorbic acid, sodium metabisulfite and tocopherol. In certain embodiments, the one or more antioxidants enhance chemical stability of the composition.

In certain embodiments, the pharmaceutical composition may include one or more stabilizing agents. Exemplary stabilizing agents include, for example: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (c) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In certain embodiments, the pharmaceutical composition may include one or more binders. Binders can impart cohesive qualities. Exemplary binders include: alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

In certain embodiments, the pharmaceutical composition may include a carrier or a pharmaceutically-compatible carrier material. These may include any commonly used excipients in pharmaceutics and should be selected on the basis of compatibility with the pharmaceutical compounds described herein. Exemplary carrier materials include binders, suspending agents, disintegration agents, filling agents, surfactants, solubilizers, stabilizers, lubricants, wetting agents, diluents, and the like. Exemplary pharmaceutically-compatible carrier materials may include acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, the pharmaceutical composition may include one or more diffusion facilitating agents, dispersing agents, and/or viscosity modulating agents, for example, to control the diffusion and homogeneity of the composition through liquid media or a granulation or blend method. In some embodiments, these agents also facilitate the effectiveness of a coating or eroding matrix. Exemplary diffusion facilitators and dispersing agents may include hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

In certain embodiments the pharmaceutical composition comprises a combination of one or more erosion facilitators with one or more diffusion facilitators.

In certain embodiments, the pharmaceutical composition may include one or more diluents. A diluent is a chemical compound that is used to dilute the substance of interest prior to delivery. Diluents can also be used to stabilize substances because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the pharmaceutical composition may include one or more filling agent. Filling agents may include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the pharmaceutical composition may include one or more lubricants or glidants. These are compounds that prevent, reduce, or inhibit adhesion or friction of materials. Exemplary lubricants may include compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

In certain embodiments, the pharmaceutical composition may include one or more plasticizers. These are compounds used to soften the microencapsulation material or film coatings to make them less brittle. Exemplary plasticizers may include polyethylene glycols such as PEG 300, PEG 400, PEG 600, PEG 1450, PEG 3350, and PEG 800, stearic acid, propylene glycol, oleic acid, triethyl cellulose and triacetin. In some embodiments, the plasticizers may also function as dispersing agents or wetting agents.

In certain embodiments, the pharmaceutical composition may include one or more solubilizers. Exemplary solubilizers may include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In certain embodiments, the pharmaceutical composition may include one or more stabilizers. Exemplary stabilizers may include any antioxidation agents, buffers, acids, preservatives and the like.

In certain embodiments, the pharmaceutical composition may include one or more suspending agents. Exemplary suspending agents may include compounds such as polyvinylpyrrolidone (e.g., polyvinylpyrrolidone K12, polyvinylpyrrolidone K17, polyvinylpyrrolidone K25, or polyvinylpyrrolidone K30), vinyl pyrrolidone/vinyl acetate copolymer (S630), polyethylene glycol (e.g., the polyethylene glycol can have a molecular weight of about 300 to about 6000, or about 3350 to about 4000, or about 7000 to about 5400), sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics (e.g., sodium carboxymethylcellulose, methylcellulose, sodium carboxymethylcellulose, hydroxypropylmethylcellulose, hydroxyethylcellulose), polysorbate-80, sodium alginate, polyethoxylated sorbitan monolaurate, polyethoxylated sorbitan monolaurate, povidone and the like.

In certain embodiments, the pharmaceutical composition may include one or more surfactants. Exemplary surfactants may include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, the surfactants can be included in the pharmaceutical composition to enhance physical stability or for other purposes.

In certain embodiments, the pharmaceutical composition may include one or more viscosity enhancing agents. Exemplary viscosity enhancing agents may include methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

In certain embodiments, the pharmaceutical composition may include one or more wetting agents. Exemplary wetting agents may include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In certain embodiments, the pharmaceutical composition may be manufactured in a conventional manner, such as by means of conventional mixing, dissolving, granulating, dragec-making, levigating, emulsifying, encapsulating, entrapping, or compression processes.

In certain embodiments, the pharmaceutical composition for administration of the vector (e.g., an adenoviral vector) described herein, may conveniently be presented in unit dosage form and be prepared by any of the methods well known in the art of pharmacy. In general, the pharmaceutical compositions may be prepared by bringing the active ingredient into association with a carrier, and then, if necessary, shaping the product into the desired formulation. In the pharmaceutical composition, the polynucleotide, polypeptide, vector, vaccine, or cell described herein is included in an amount sufficient to produce the desired effect upon the process, condition or disease sought to be treated.

In certain embodiments, the pharmaceutical composition comprises the polynucleotide, polypeptide, vector, vaccine, or cell described herein in a therapeutically-effective amount. An “effective amount” is any amount required to demonstrate a therapeutic effect in the subject. The amount may vary depending on the subject's condition, age, gender, medical history, and/or weight. The dosage may also vary depending on the condition to be treated, the anti-inflammatory agent encoded, the type of polynucleotide, polypeptide, vector, vaccine, or cell being administered, and/or the route of administration.

In some embodiments, the pharmaceutical composition comprises the vector described herein at a concentration of about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

In some embodiments, the pharmaceutical composition may be stored by freezing at a temperature of about 0° C. to about −120° C., about −10° C. to about −110° C., about −20° C. to about −100° C., about −30° C. to about −90° C., about −40° C. to about −90° C., about −50° C. to about −90° C., about −60° C. to about −90° C., about −65° C. to about −85° C., or about −70° C. to about −80° C. In some embodiments, the pharmaceutical composition may be stored by freezing at a temperature of about −60° C., about −61° C., about −62° C., about −63° C., about −64° C., about −65° C., about −66° C., about −66° C., about −67° C., about −68° C., about −69° C., about −70° C., about −71° C., about −72° C., about −73° C., about −74° C., about −75° C., about −76° C., about −77° C., about −78° C., about −79° C., about −80° C., about −81° C., about −82° C., about −83° C., about −84° C., about −85° C., about −86° C., about −87° C., about −88° C., about −89° C., or about −90° C. The pharmaceutical composition may be thawed, for example, in a water bath prior to use, avoiding prolonged exposure of the thawed composition to the water bath. The temperature of water bath used to thaw the pharmaceutical composition may be, for example, between about 30° C. to about 44° C., about 31° C. to about 43° C., about 32° C. to about 42° C., about 33° C. to about 41° C., about 34° C. to about 40° C., or about 35° C. to about 39° C. In some embodiments, the temperature of water bath used to thaw the pharmaceutical composition may be about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C. The thawed composition may be stored for up to about 15 minutes, up to about 30 minutes, up to about 45 minutes, up to about 1 hour, up to about 75 minutes, up to about 90 minutes, up to about 105 minutes, or up to about 2 hours at ambient temperature prior to administration. In some embodiments, the thawed composition will appear as a clear to slightly opalescent, colorless liquid and be substantially free of visible particulates.

### XI. Kits/Articles of Manufacture

Another aspect of the present invention is are kits and articles of manufacture for use with one or more methods described herein. Suitable kits may include a package or container that comprise the polynucleotide, polypeptide, vector, vaccine, or cell described herein, or the composition comprising the same. Suitable containers include, for example, bottles, vials, syringes, and test tubes. In some embodiments, the containers are formed from a variety of materials, such as glass or plastic. Suitable articles of manufacture may contain packaging materials. Examples of pharmaceutical packaging materials include blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions may also be included. In some embodiments, a label is on or associated with the container. In some embodiments, a label is on a container when letters, numbers or other characters forming the label are attached, molded or etched into the container itself; a label is associated with a container when it is present within a receptacle or carrier that also holds the container, e.g., as a package insert. In some embodiments, a label is used to indicate that the contents are to be used for a specific therapeutic application. The label also indicates directions for use of the contents, such as in the methods described herein.

In some embodiments, the kit contains a vial containing the composition of the present invention. In some such embodiments, the vial comprises, for example, from about 0.1 to about 20 ml of the pharmaceutical composition, from about 0.1 to about 15 ml of the pharmaceutical composition, from about 0.1 to about 10 ml of the pharmaceutical composition, from about 0.1 to about 9 ml of the pharmaceutical composition, from about 0.1 to about 8 ml of the pharmaceutical composition, from about 0.1 to about 7 ml of the pharmaceutical composition, from about 0.1 to about 6 ml of the pharmaceutical composition, from about 0.1 to about 5 ml of the pharmaceutical composition, from about 0.2 to about 5 ml of the pharmaceutical composition, from about 0.2 to about 4 ml of the pharmaceutical composition, from about 0.2 to about 3 ml of the pharmaceutical composition, from about 0.2 to about 2 ml of the pharmaceutical composition, from about 0.2 to about 1 ml of the pharmaceutical composition, from about 0.5 to about 2 ml of the pharmaceutical composition, from about 0.5 to about 1.75 ml of the pharmaceutical composition, from about 0.5 to about 1.5 ml of the pharmaceutical composition, from about 0.5 to about 1.25 ml of the pharmaceutical composition, from about 0.5 to about 1 ml of the pharmaceutical composition, from about 0.75 to about 1.25 ml of the pharmaceutical composition. In other such embodiments, the vial comprises, for example, about 0.5 ml of the pharmaceutical composition, about 0.55 ml of the pharmaceutical composition, about 0.6 ml of the pharmaceutical composition, about 0.65 ml of the pharmaceutical composition, about 0.7 ml of the pharmaceutical composition, about 0.75 ml of the pharmaceutical composition, about 0.8 ml of the pharmaceutical composition, about 0.85 ml of the pharmaceutical composition, about 0.9 ml of the pharmaceutical composition, about 0.95 ml of the pharmaceutical composition, about 1 ml of the pharmaceutical composition, about 1.05 ml of the pharmaceutical composition, about 1.1 ml of the pharmaceutical composition, about 1.15 ml of the pharmaceutical composition, or about 1.2 ml of the pharmaceutical composition.

In some embodiments, the viral vector contained within the composition may be present at a concentration of, for example, about 1×10⁹ to about 1×10¹³ particle units, about 5×10⁹ to about 5×10¹² particle units, about 1×10¹⁰ to about 1×10¹² particle units, about 1×10¹¹ to about 9×10¹¹ particle units about 1×10¹¹ to about 9×10¹¹ particle units about 1×10¹¹ to about 9×10¹¹ particle units, about 1×10¹⁰ to about 1×10¹² particle units, about 1×10¹¹ to about 9×10¹¹ particle units, about 2×10¹¹ to about 8×10¹¹ particle units, about 3×10¹¹ to about 7×10¹¹ particle units, about 4×10¹¹ to about 6×10¹¹ particle units, or about 5×10¹¹ particle units.

### XII. Methods of Evaluating Efficacy of Treatment

Regardless of the method used to introduce exogenous nucleic acids into a host cell or otherwise expose a cell to the inhibitor of the present disclosure, in order to confirm the presence of the recombinant DNA sequence in the host cell, a variety of assays can be performed. Such assays include, for example, molecular assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

### A. Immune Phenotyping

Exploratory immunologic studies may also be conducted to evaluate the study drug's effect on the immune response before and after treatment, to gain insight into potential biomarkers, and help improve the administered therapy. In one embodiments, for example, such immunologic assays may be performed at the Laboratory of Tumor Immunology and Biology (LTIB) at the NCI's Center for Cancer Research (CCR) in select participants where adequate samples are available. In certain embodiments, the samples collected from participants are blood samples. In other embodiments, the samples may be saliva samples. The immunologic assays may include: (i) analyzing PBMCs for changes in standard immune cell types (CD4 and CD8 T cells, natural killer (NK) cells, regulatory T cells (Tregs), myeloid-derived suppressor cells (MDSCs), and dendritic cells) as well as 123 immune cell subsets, using multi-color flow cytometry; (ii) analyzing PBMCs from selected subjects for function of specific immune cell subsets, including CD4 and CD8 T cells, NK cells, Tregs, and MDSCs using flow-based assays; and/or (iii) analyzing PBMCs for tumor antigen-specific immune responses using an intracellular cytokine staining assay. PBMCs will be stimulated in vitro with overlapping 15-mer peptide pools encoding the tumor-associated antigens HPV16 E6 and E7 and HPV18 E6 and E7; control peptide pools will involve the use of human leukocyte antigen peptide as a negative control and CEFT peptide mix as a positive control. CEFT is a mixture of peptides of CMV, Epstein-Barr virus, influenza, and tetanus toxin. Post-stimulation analyses of CD4 and CD8 T cells may involve the production of IFN-γ, IL-2, TNF, and the degranulation marker CD107a. If sufficient PBMCs are available, assays may also be performed for the development of T cells to other tumor-associated antigens.

### 1. Soluble Factors

In certain embodiments, Sera may be analyzed pre- and post-therapy for the following soluble factors: sCD27, sCD40 ligand using commercial ELISA kits. For example, in one embodiment, Sera may be analyzed for changes in cytokines (IFN-γ, IL-10, IL-12, IL-2, IL-4, etc.), chemokines, antibodies, tumor-associated antigens, and/or other markers using ELISA or multiplexed assays (e.g., Mesoscale, Luminex, cytokine bead array).

### 2. Anti-Vector Antibodies (AVA)

Anti-vector antibodies are one mechanism of neutralization and inefficacy of viral vectors. Longitudinal detection and titer measurement of AVA to the vaccine adenoviral vector may assist in characterizing cases of loss of efficacy.

### 3. Pharmacokinetics (“PK”)

PK measurements of drugs used in combination with the vaccine adenoviral vector of the present invention disclosed herein may be taken to collect data which will provide insight into population PKs of such drug(s) in participants receiving these novel combinations. For example, in a certain embodiment where the vaccine adenoviral vector is administered alongside bintrafusp alfa, PK measurements of bintrafusp alfa may be taken to collect data which may provide insight into population PKs of bintrafusp alfa in participants receiving such novel combination.

### B. Anti-Drug Antibody (“ADA”)

Anti-Drug Antibody development is an accepted mechanism of loss of efficacy of administered human monoclonal antibodies. In certain embodiments of the present invention, measuring titers will ensure that lack of efficacy of a certain combination drug is not due to ADA development. For example, in a certain embodiment where the vaccine adenoviral vector is administered alongside bintrafusp alfa, measuring titers may ensure that lack of efficacy of bintrafusp alfa is not due to ADA development.

### C. Tumor Tissue Analyses for Immune Markers

In other embodiments, analyses may be performed in tumor tissue pre-treatment vs. post-treatment with the vaccine adenoviral vector. In certain embodiments, archival tumor samples may be requested for pre-treatment analysis. In another embodiment, preferably tissue samples from the last 6 months may be analyzed. In yet another embodiment, for participants with lesions amenable to biopsy, two biopsies may be performed at baseline, and at Week 4-5 or Week 9 post-treatment. In certain embodiments, the study of immune infiltration as well as PD-L1 status within the tumor microenvironment pre vs. post treatment by immunohistochemistry and/or multiplex immunofluorescence may be performed.

### D. Single-Cell Proteomic Analysis

In another embodiment, tumor tissue single-cell proteomic analysis of immune and signaling pathways may be performed with the Isoplexis Single Cell Functional proteomic platform by Precigen.

### E. Immune Transcriptomic Analysis

In one embodiment, tumor tissue immune transcriptomic analysis may be performed with the Nanostring platform by Precigen.

### F. Assessment of HPV Status

In yet another embodiment, where participants have available tumor tissue (either archival or by optional biopsy), HPV testing may be performed using the Roche Cobas or Becton Dickinson HPV PCR based DNA assay, if no prior HPV testing of the tumor has been performed.

### G. Circulating Tumor DNA

In another embodiment, where sufficient plasma is available, select participant samples may be analyzed for circulating tumor DNA. Plasma DNA may be isolated with an automated purification system, and the circulating tumor/HPV DNA may be quantified with a digital droplet PCR system from Bio-Rad to obtain precise quantification.

In a further embodiment, RNA expression and T-cell receptor clonality analysis may be done on the peripheral blood, as well as archived tumor tissue or optional biopsies, to help further evaluate changes in immune response and RNA expression levels with treatment, as well as to determine tumor and infiltrating lymphocyte characteristics which may be predictive of response to treatment. In addition, such analyses may also be used to gauge resistance mechanisms and additional targets for future therapy. Coded, linked samples, may be analyzed for RNA expression levels using the Nanostring platform and T-cell receptor clonality using the ImmunoSeq platform.

### H. Salivary HPV DNA

In a certain embodiment, saliva samples may be analyzed for HPV DNA detection and quantification. Salivary HPV DNA may be quantified with digital droplet PCR with no sequencing is involved. In one embodiment, participants with p16-positive oropharyngeal cancer may provide a saliva sample by mouth rinse and gargle with 15-20 mL 0.9% NaCl for 30 seconds and spitting into the collection tube.

### XIII. Exemplary Embodiments of the Invention

The foregoing description of specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the invention will be apparent to those skilled in the art from consideration of the specification and practice of the invention disclosed herein. Some additional exemplary embodiments (“E”) of the invention include, but are not limited to:
E1. A non-naturally occurring polynucleotide encoding a polypeptide construct comprising one or more immune response-inducing human papilloma virus (HPV) peptides.
E2. The polynucleotide of E1, wherein the non-naturally occurring polynucleotide encodes a polypeptide construct comprising two or more HPV peptides.
E3. The polynucleotide of E2, wherein the two or more HPV peptides comprise one or more HPV-16 peptides.
E4. The polynucleotide of E3, wherein the polypeptide construct comprises at least one of an HPV-16 E5 peptide, an HPV-16 E6 peptide, or an HPV-16 E7 peptide.
E5. The polynucleotide of E3 or E4, wherein the HPV-16 peptide is an HPV-16 E5 peptide having the sequence of SEQ ID NO: 47.
E6. The polynucleotide of any one of E3 to E5, wherein the HPV-16 peptide is an HPV-16 E6 peptide having the sequence of SEQ ID NO: 45.
E7. The polynucleotide of any one of E3 to E6, wherein the HPV-16 peptide is an HPV-16 E7 peptide having the sequence of SEQ ID NO: 46.
E8. The polynucleotide of any one of E1 to E7, wherein the polypeptide construct comprises an HPV-18 peptide.
E9. The polynucleotide of E8, wherein the polypeptide construct comprises HPV-18 peptide comprises at least one of an HPV-18 E5 peptide, an HPV-18 E6 peptide, or an HPV-18 E7 peptide.
E10. The polynucleotide of E8 or E9, wherein the polypeptide construct comprises an HPV-18 E5 peptide having the sequence of SEQ ID NO: 50.
E11. The polynucleotide of any one of E8 to E10, wherein the HPV-18 peptide is an HPV-18 E6 peptide having the sequence of SEQ ID NO: 48.
E12. The polynucleotide of any one of E8 to E10, wherein the HPV-18 peptide comprises an HPV-18 E7 peptide having the sequence of SEQ ID NO: 49.
E13. The polynucleotide of any one of E1 to E12, wherein the polypeptide construct has the sequence of SEQ ID NO: 51.
E14. The polynucleotide of any one of E1 to E13, wherein at least one of the one or more HPV peptides is connected to an agonist peptide.
E15. The polynucleotide of E14, wherein the agonist peptide has a sequence comprising an agonist peptide sequence as shown in Table 5.
E16. The polynucleotide of E14 or E15, wherein the polypeptide construct has the sequence of SEQ ID NO: 53.
E17. The polynucleotide of any one of E1 to E16, further encoding a gene switch system for inducible control of heterologous gene expression, wherein the heterologous gene encodes a polypeptide construct as described herein.
E18. The polynucleotide of E17, wherein the gene switch system is an ecdysone receptor-based (EcR-based) gene switch system.
E19. The polynucleotide of any one of E1 to E18, wherein the polypeptide construct is for use in a vaccine.
E20. A vector comprising the polynucleotide of any one of E1 to E19.
E21. The vector of E20, wherein the vector is an adenoviral vector.
E22. The vector of E21, wherein the adenoviral vector is a gorilla adenoviral vector.
E23. A method of regulating the expression of a heterologous immune response-inducing gene in a cell, the method comprising: (a) introducing into the cell one or more polynucleotides that comprise (i) an repressible or inducible gene switch, and (ii) a heterologous immune response-inducing gene, wherein expression of the heterologous immune response-inducing gene is regulated by the gene switch, wherein said heterologous immune response-inducing gene encodes one or more HPV polypeptides; and (b) exposing said cell to a compound in an amount sufficient to repress or induce expression of the heterologous immune response-inducing gene.
E24. The method of E23, wherein the target cell is a mammalian cell.
E25. The method of E23 or E24, wherein the gene switch comprises a ligand binding domain derived from at least one of an ecdysone receptor (EcR), a ubiquitous receptor, an orphan receptor 1, an NER-1, a steroid hormone nuclear receptor 1, a retinoid X receptor interacting protein-15, a liver X receptor B, a steroid hormone receptor like protein, a liver X receptor, a liver X receptor a, a farnesoid X receptor, a receptor interacting protein 14, and a famesol receptor.
E26. An E6 peptide, wherein the E6 peptide comprises an E18A amino acid substitution and at least one of an L50G, E148A, T149A, Q150A and L151A amino acid substitution as compared to a wildtype E6 peptide.
E27. The E6 peptide of E26, wherein the E6 peptide comprises E18A, L50G, E148A, T149A, Q150A, and L151A amino acid substitutions.
E28. The E6 peptide of E26, wherein the E6 peptide has the sequence of SEQ ID NO:

45.
E29. The E6 peptide of any one of E26 to E28, wherein the E6 peptide is fused to an agonist peptide.
E30. The E6 peptide of E29, wherein the agonist peptide is fused to at least one of a C-terminus and an N-terminus of the E6 peptide.
E31. The E6 peptide of any one of E26 to E30, wherein the wildtype E6 peptide is from HPV-16.
E32. An E6 peptide, wherein the E6 peptide comprises a deletion as compared to a wildtype E6 peptide, wherein the deletion is that of the C-terminus of the wildtype E6 peptide.
E33. The E6 peptide of E32, wherein the deletion is that of the amino acids from residue position 121 to the C-terminus of the wildtype E6 peptide.
E34. The E6 peptide of E32 or E33, wherein the E6 peptide comprises at least one of an E18A and L50G substitution as compared to wildtype E6 peptide.
E35. The E6 peptide of any one of E32 to E34, wherein the wildtype E6 peptide is from HPV-18.
E36. The E6 peptide of any one of E32 to E35, wherein the E6 peptide has the sequence of SEQ ID NO: 48.
E37. An E7 peptide, wherein the E7 peptide has a deletion of the N-terminus as compared to a wildtype E7 peptide.
E38. The E7 peptide of E37, wherein the E7 peptide has the deletion of the amino acids at residue positions 1-39 of the wildtype E7 peptide.
E39. The E7 peptide of E37 or E38, wherein the E7 peptide comprises at least one of an E55A and L74R substitution as compared to wildtype E7 peptide.
E40. The E7 peptide of any one of E37 to E39, wherein the wildtype E7 peptide is from HPV-18.
E41. The E7 peptide of any one of E37 to E40, wherein the E7 peptide has the sequence of SEQ ID NO: 49.
E42. An E5 peptide, wherein the E5 peptide has a deletion of the amino acids at residue positions 41-57 of wildtype E5 peptide.
E43. The E5 peptide of E42, wherein said E5 peptide has the sequence of SEQ ID NO: 47.
E44. The E5 peptide of E42 or E43, wherein the wildtype E5 peptide is from HPV-16.
E45. An E5 peptide, wherein the E5 peptide has a deletion of the amino acids at residue positions 27-40 and/or the amino acids at residue positions 54-57 of wildtype E5 peptide.
E46. The E5 peptide of E45, wherein the E5 peptide has the sequence of SEQ ID NO: 50.
E47. The E5 peptide of E45 or E46, wherein the wildtype E5 peptide is from HPV-18. E48. A polypeptide construct comprising the peptide of any one of E26 to E47.
E49. A polypeptide construct, comprising an HPV-16 E6 peptide, having an E18A amino acid substitution and at least one of an L50G, E148A, T149A, Q150A and L151A amino acid substitution as compared to a wildtype HPV-16 E6 peptide.
E50. The polypeptide construct of E49, wherein the HPV-16 E6 peptide comprises E18A, L50G, E148A, T149A, Q150A and L151A amino acid substitutions as compared to a wildtype HPV-16 E6 peptide.
E51. The polypeptide construct of E49 or E50, wherein the HPV-16 E6 peptide has the sequence of SEQ ID NO: 45.
E52. The polypeptide construct of any one of E49 to E51, wherein the polypeptide construct further comprises an HPV-16 E7 peptide comprising at least one of an H2P, C24G, E46A and L67R amino acid substitution as compared to a wildtype HPV-16 E7 peptide.
E53. The polypeptide construct of E52, wherein the HPV-16 E7 peptide comprises said H2P, C24G, E46A and L67R amino acid substitutions as compared to a wildtype HPV-16 E7 peptide.
E54. The polypeptide construct of E53, wherein the HPV-16 E7 peptide has the sequence of SEQ ID NO: 46.
E55. The polypeptide construct of any one of E49 to E54, wherein the polypeptide construct further comprises an HPV-16 E5 peptide.
E56. The polypeptide construct of E55, wherein the HPV-16 E5 peptide has a deletion of one or more amino acids as compared to a wildtype HPV-16 E5 peptide.
E57. The polypeptide construct of E56, wherein the HPV16 E5 peptide has a deletion of the amino acids at residue positions 41-57 of the wildtype HPV-16 E5 peptide.
E58. The polypeptide construct of any one of E55 to E57, wherein the HPV-16 E5 peptide has the sequence of SEQ ID NO: 47.
E59. The polypeptide construct of any one of E49 to E58, wherein the polypeptide further comprises an HPV-18 E6 peptide.
E60. The polypeptide construct of E59, wherein the HPV-18 E6 peptide comprises an E18A and L50G substitution as compared to a wildtype HPV-18 E6 peptide.
E61. The polypeptide construct of E59 or E60, wherein the HPV-18 E6 peptide has a deletion of at least one C-terminus amino acid relative to the wildtype HPV-18 E6 peptide.
E62. The polypeptide construct of E61, wherein the deletion is of amino acids from residue position 121 to the C-terminus of the wildtype HPV-18 E6 peptide.
E63. The polypeptide construct of any one of E59 to E62, wherein the HPV-18 E6 peptide has the sequence of SEQ ID NO: 48.
E64. The polypeptide construct of any one of E49 to E63, wherein the polypeptide construct further comprises an HPV-18 E7 peptide.
E65. The polypeptide construct of E64, wherein the HPV-18 E7 peptide has an E55A and L74R substitution as compared to a wildtype HPV-18 E7 peptide.
E66. The polypeptide construct of E64 or E65, wherein the HPV-18 E7 peptide has a deletion of at least one amino acid from an N-terminus of the HPV-18 E7 peptide.
E67. The polypeptide construct of E66, wherein the deletion is that of the amino acids at residue positions 1-40 of the wildtype HPV-18 E7 peptide.
E68. The polypeptide construct of any one of E64 to E67, wherein the HPV-18 E7 peptide has the sequence of SEQ ID NO: 49.
E69. The polypeptide construct of any one of E59 to E68, wherein the polypeptide construct further comprises an HPV-18 E5 peptide.
E70. The polypeptide construct of E69, wherein the HPV-18 E5 peptide comprises a deletion of at least one amino acid as compared to a wildtype HPV-18 E5 peptide.
E71. The polypeptide construct of E70, wherein the deletion is that of the amino acids at residue positions 27-40 and/or 54-57 of wildtype HPV-18 E5 peptide.
E72. The polypeptide construct of any one of E69 to E71, wherein the HPV-18 E5 peptide has the sequence of SEQ ID NO: 50.
E73. The polypeptide construct of any one of E59 to E72, wherein the polypeptide construct has the sequence of SEQ ID NO: 51.
E74. The polypeptide construct of any one of E5973, wherein said polypeptide construct further comprises at least one agonist peptide.
E75. The polypeptide construct of E74, wherein the at least one agonist peptide has a sequence comprising an agonist peptide sequence as shown in Table 5.
E76. The polypeptide construct of E74 or E75, wherein the polypeptide construct has the sequence of SEQ ID NO: 53.
E77. A polypeptide construct comprising an ankyrin-like repeat domain and an HPV peptide.
E78. The polypeptide construct of E77, wherein the ankyrin-like repeat protein is a human ankyrin-like repeat protein.
E79. The polypeptide construct of E77 or E78, wherein the HPV peptide is linked to the ankyrin-like repeat protein by a linker.
E80. The polypeptide construct of any one of E77 to E79, comprising an HPV-16 peptide and/or an HPV-18 peptide.
E81. The polypeptide construct of any one of E77 to E80, comprising an HPV-16 E5 peptide, an HPV-16 E6 peptide, and/or an HPV-16 E7 peptide.
E82. The polypeptide construct of any one of E77 to E81, wherein said HPV peptide comprises an HPV-18 peptide, and said HPV-18 peptide comprises at least one of an E6 peptide or an E7 peptide.
E83. The polypeptide construct of any one of E77 to E82, comprising an HPV-16 E5 sequence, an HPV-16 E6 sequence, an HPV-16 E7 sequence, an HPV-18 E6 sequence or an HPV-18 E7 sequence as shown in Table 5.
E84. The polypeptide construct of any one of E77 to E83, having the sequence of SEQ ID NO: 52.
E85. The polypeptide construct of any one of E77 to E84, further comprising at least one agonist peptide.
E86. The polypeptide construct of E85, comprising three agonist peptides.
E87. The polypeptide construct of E86, having the sequence of SEQ ID NO: 54.
E88. A polypeptide construct, comprising at least two HPV amino acid sequences as shown in Table 5, connected by a KK linker.
E89. The polypeptide construct of E88, wherein the at least two HPV amino acid sequences comprise at least one of an HPV-16 peptide or an HPV-18 peptide as shown in Table 5.
E90. The polypeptide construct of E88 or E89, wherein the at least two HPV amino acid sequences comprise an HPV-16 E5 peptide, an HPV-16 E6 peptide, and/or an HPV-16 E7 peptide as shown in Table 5.
E91. The polypeptide construct of any one of E88 to E90, wherein the at least two HPV amino acid sequences comprise an HPV-18 E6 peptide and/or an HPV-18 E7 peptide as shown in Table 5.
E92. The polypeptide construct of any one of E88 to E91, wherein the at least two HPV amino acid sequences comprise each of the amino acid sequences as shown in Table 5.
E93. The polypeptide construct of E92, wherein each of the amino acid sequences is connected to another of each of the amino acid sequences by said KK linker.
E94. The polypeptide construct of any one of E88 to E93, having the sequence of SEQ ID NO: 55.
E94.1. The polypeptide construct of any one of E88 to E93, having the sequence of SEQ ID NO: 243.
E95. The polypeptide construct of any one of E48 to E94.1 for use in a vaccine.
E96. A polynucleotide encoding the polypeptide construct of any one of E58 to E95.
E97. A vector comprising the polynucleotide of E96.
E98. The vector of E97, wherein the vector is a plasmid, a viral, or a non-viral vector.
E99. The vector of E98, wherein the viral vector is an adenoviral vector.
E100. The vector of E98, wherein the adenoviral vector is a gorilla adenoviral vector.
E101. A vector, wherein the vector is an adenoviral vector and comprises a polynucleotide encoding at least one HPV peptide.
E102. A vector, wherein the vector is a gorilla adenoviral vector comprising a polynucleotide encoding at least one HPV peptide.
E103. A composition or vaccine comprising a pharmaceutically acceptable carrier and a polynucleotide in any one of E1 to E19 and E96, a polypeptide of any one of E26 to E94.1, or a vector of any one of E20 to E22 and E97 to E102.
E104. The polynucleotide, polypeptide, vector, composition or vaccine of any one of E1 to E22 and E26 to E103, wherein the polynucleotide, polypeptide, vector, composition or vaccine is capable of inducing an anti-HPV immune response.
E105. Use of a polynucleotide, polypeptide, vector, composition or vaccine in E104 for treatment of a patient that has an HPV-associated disease or disorder.
E106. The use of a polynucleotide, polypeptide, vector, composition or vaccine in E105, wherein the disease or disorder is an HPV16-associated disease or disorder.
E106.1. The use of a polynucleotide, polypeptide, vector, composition or vaccine in E105, wherein the disease or disorder is an HPV18-associated disease or disorder.
E106.2 The use of a polynucleotide, polypeptide, vector, composition or vaccine in E105, wherein the disease or disorder is an HPV45-associated disease or disorder.
E107. The use of a polynucleotide, polypeptide, vector, composition or vaccine in E105, wherein the disease or disorder is cervical cancer.
E108. The use of a polynucleotide, polypeptide, vector, composition or vaccine in 105, wherein the disease or disorder is vulvar cancer.
E109. The use of a polynucleotide, polypeptide, vector, composition or vaccine in E105, wherein the disease or disorder is anal cancer.
E110. The use of a polynucleotide, polypeptide, vector, composition or vaccine in E105, wherein the disease or disorder is penile cancer.
E111. The use of a polynucleotide, polypeptide, vector, composition or vaccine in E105, wherein the disease or disorder is a head and neck cancer.
E111.1. The use of a polynucleotide, polypeptide, vector, composition or vaccine in E105, wherein the disease or disorder is lower genital tract neoplasia.
E111.2. The use of a polynucleotide, polypeptide, vector, composition or vaccine in E105, wherein the disease or disorder is one or more of cervical, vaginal, and vulvar intraepithelial neoplasia.
E112. The use of a polynucleotide, polypeptide, vector, composition or vaccine in E105, wherein the treatment involves treating the patient with one or more additional therapies.
E113. The use of a polynucleotide, polypeptide, vector, composition or vaccine in E105, wherein the one or more additional therapies is a surgical or non-surgical therapy.
E114. The polynucleotide, polypeptide, vector, composition or vaccine in E113 for use in a method of treating a patient that has an HPV-associated disease or disorder, wherein the non-surgical therapy comprises co-administration of an additional agent.
E115. The polynucleotide, polypeptide, vector, composition or vaccine in E114 for use in a method of treating a patient that has an HPV-associated disease or disorder, wherein the additional agent is contained in the same composition that contains the polynucleotide, polypeptide, vector, composition or vaccine.
E116. The polynucleotide, polypeptide, vector, composition or vaccine in E114 for use in a method of treating a patient that has an HPV-associated disease or disorder, wherein the additional agent is a chemotherapy agent, an anti-inflammatory agent, an analgesic, a biological response modifier, a vector comprising such agents, or a cell comprising the agent or a nucleic acid encoding the same.
E117. The polynucleotide, polypeptide, vector, composition or vaccine in E114 for use in a method of treating a patient that has an HPV-associated disease or disorder, wherein the additional agent is administered at or near the same location as the polynucleotide, polypeptide, vector, composition or vaccine.
E118. The polynucleotide, polypeptide, vector, composition or vaccine in E114 for use in a method of treating a patient that has an HPV-associated disease or disorder, wherein the additional agent is administered at a different location than the polynucleotide, polypeptide, vector, composition or vaccine.
E119. The polynucleotide, polypeptide, vector, composition or vaccine in E114 for use in a method of treating a patient that has an HPV-associated disease or disorder, wherein the additional agent is administered simultaneously with the administration of the polynucleotide, polypeptide, vector, composition or vaccine.
E120. The polynucleotide, polypeptide, vector, composition or vaccine in E114 for use in a method of treating a patient that has an HPV-associated disease or disorder, wherein the additional agent is administered at a different time than administration of the polynucleotide, polypeptide, vector, composition or vaccine.
E121. The polynucleotide, polypeptide, vector, composition or vaccine in E114 for use in a method of treating a patient that has an HPV-associated disease or disorder, wherein the additional agent is selected from the group consisting of: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.
E122. The polynucleotide, polypeptide, vector, composition or vaccine in E114 for use in a method of treating a patient that has an HPV-associated disease or disorder, wherein the additional agent is selected from the group consisting of: acetaminophen, oxycodone, tramadol, and proporxyphene hydrochloride.
E123. The polynucleotide, polypeptide, vector, composition or vaccine in E114 for use in a method of treating a patient that has an HPV-associated disease or disorder, wherein the additional agent is selected from the group consisting of: molecules directed against cell surface markers (e.g., CD4, CD5, etc.).
E124. The polynucleotide, polypeptide, vector, composition or vaccine in E114 for use in a method of treating a patient that has an HPV-associated disease or disorder, wherein the additional agent is a cytokine inhibitor, such as a TNF inhibitor (e.g., etanercept (ENBREL®), adalimumab (HUMIRA®), and infliximab (REMICADE®)).
E125. The polynucleotide, polypeptide, vector, composition or vaccine in E114 for use in a method of treating a patient that has an HPV-associated disease or disorder, wherein the additional agent is a nucleotide analogs (e.g., Cidofovir).
E126. The polynucleotide, polypeptide, vector, composition or vaccine in E114 for use in a method of treating a patient that has an HPV-associated disease or disorder, wherein the additional agent is an angiogenesis inhibitor, such as Bevacizumab (AVASTIN®).
E127. The polynucleotide, polypeptide, vector, composition or vaccine in E114 for use in a method of treating a patient that has an HPV-associated disease or disorder, wherein the additional agent is a non-steroidal anti-inflammatory compound, such as COX-2-selective drugs (e.g., Celexecob (CELEBREX®)).
E128. The polynucleotide, polypeptide, vector, composition or vaccine in E114 for use in a method of treating a patient that has an HPV-associated disease or disorder, wherein the additional agent is an immune checkpoint inhibitor, such as a PD-1 inhibitors (e.g., Pembrolizumab (KEYTRUDA®), Nivolumab (OPDIVO®), and Cemiplimab (LIBTAYO®) and/or a PD-L1 inhibitor (e.g., Atezolizumab (TECENTRIQ®), Avelumah (BAVENCIO®), Durvalumab (IMFINZI®), and bintrafusp alfa).
E129. The polynucleotide, polypeptide, vector, composition or vaccine in E114 for use in a method of treating a patient that has an HPV-associated disease or disorder, wherein the additional agent is an adhesion molecule inhibitor.
E130. The polynucleotide, polypeptide, vector, composition or vaccine in E114 for use in a method of treating a patient that has an HPV-associated disease or disorder, wherein the additional agent is a monoclonal antibody.
E131. The polynucleotide, polypeptide, vector, composition or vaccine in E114 for use in a method of treating a patient that has an HPV-associated disease or disorder, wherein the additional agent is an anti-rheumatic drug, such as cyclophosphamide, cyclosporine, methotrexate, penicillamine, leflunomide, sulfasalazine, hydroxychloroquine, Gold (oral (auranofin) and intramuscular), and/or minocycline.
E132. The polynucleotide, polypeptide, vector, composition or vaccine in E114 for use in a method of treating a patient that has an HPV-associated disease or disorder, wherein the additional agent is a colony-stimulating factor, such as macrophage colony-stimulating factor, granulocyte macrophage colony-stimulating factor (GM-CSF), granulocyte colony-stimulating factor (G-CSF) or promegapoietin.
E133. The polynucleotide, polypeptide, vector, composition or vaccine in E114 for use in a method of treating a patient that has an HPV-associated disease or disorder, wherein the additional agent is a tumor necrosis factor, such as TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL)).
E134. The polynucleotide, polypeptide, vector, composition or vaccine in E114 for use in a method of treating a patient that has an HPV-associated disease or disorder, wherein the additional agent is an interleukin, such as IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, and/or IL-36, or a functional fragment or variant thereof.
E135. The polynucleotide, polypeptide, vector, composition or vaccine in E114 for use in a method of treating a patient that has an HPV-associated disease or disorder, wherein the additional agent is an interferon, such as IFN-α, IFN-β, IFN-ε, IFN-κ, and IFN-ω, interferon type II, IFN-γ, interferon type III, IFNAI, IFNA2, IFNA4, IFNA5, IFNA6, IFNA7, IFNA8, IFNAI0, IFNA13, IFNA14, IFNA16, IFNAI 7, and/or IFNA21.
E136. The polynucleotide, polypeptide, vector, composition or vaccine in E114 for use in a method of treating a patient that has an HPV-associated disease or disorder, wherein the additional agent is a chemokine selected from the group consisting of: CCLI, CCL2 (MCP-1), CCL3, CCL4, CCL5 (RANTES), CCL6, CCL7, CCL8, CCL9 (or CCLI0), CCLI 1, CCL12, CCL13, CCL14, CCL15, CCL16, CCLI 7, CCL18, CCL19, CCL20, CCL21, CCL22, CCL23, CCL24, CCL25, CCL26, CCL27, and CCL28; the CXC subfamily: CXCLI, CXCL2, CXCL3, CXCL4, CXCL5, CXCL6, CXCL7, CXCL8, CXCL9, CXCLI0, CXCLII, CXCL12, CXCL13, CXCL14, CXCL15, CXCL16, and CXCLI 7; the XC subfamily: XCLI and XCL2; and the CX3C subfamily CX3CL1.
E137. The polynucleotide, polypeptide, vector, composition or vaccine in E114 for use in a method of treating a patient that has an HPV-associated disease or disorder, wherein the additional agent is a membrane-bound cytokine, which is co-expressed with a chimeric antigen receptor (CAR).
E138. The use of a polynucleotide, polypeptide, vector, composition or vaccine in E114, wherein the additional agent is a cytokine selected from IL2, IL7, IL12, IL15, IL21, IFNγ or TNF-α, or a functional fragment or variant thereof.
E139. The polynucleotide, polypeptide, vector, composition or vaccine in E114 for use in a method of treating a patient that has an HPV-associated disease or disorder, wherein administration of the polynucleotide, polypeptide, vector, composition or vaccine in combination with the additional agent enhances the treatment of a disease or disorder and/or reduces any side-effects of treatment with the polynucleotide, polypeptide, vector, composition or vaccine alone.
E140. The polynucleotide, polypeptide, vector, composition or vaccine in E114-E139 for use in a method of treating a patient that has an HPV16-associated disease or disorder.
E140.1. The polynucleotide, polypeptide, vector, composition or vaccine in E114-E139 for use in a method of treating a patient that has an HPV18-associated disease or disorder.
E140.2 The polynucleotide, polypeptide, vector, composition or vaccine in E114-E139 for use in a method of treating a patient that has an HPV45-associated disease or disorder.
E141. A method of treating a patient that has an HPV-associated disease or disorder comprising administering the polynucleotide, polypeptide, vector, composition or vaccine in E104.
E141.1. A method of treating a patient that has an HPV-16-associated disease or disorder comprising administering the polynucleotide, polypeptide, vector, composition or vaccine in E104.
E141.2. A method of treating a patient that has an HPV-18-associated disease or disorder comprising administering the polynucleotide, polypeptide, vector, composition or vaccine in E104.
E141.3. A method of treating a patient that has an HPV-45-associated disease or disorder comprising administering the polynucleotide, polypeptide, vector, composition or vaccine in E104.
E142. The method of treatment of E141, wherein the disease or disorder is lower genital tract neoplasia (e.g., cervical, vaginal, and vulvar intraepithelial neoplasia), cervical cancer, vulvar cancer, anal cancer, penile cancer, or head and neck cancers.
E142.1. The method of treatment of E141, wherein the disease or disorder is lower genital tract neoplasia.
E142.2. The method of treatment of E141, wherein the disease or disorder is one or more of cervical, vaginal, and vulvar intraepithelial neoplasia.
E142.3. The method of treatment of E141, wherein the disease or disorder is cervical cancer.
E142.4. The method of treatment of E141, wherein the disease or disorder is vulvar cancer.
E142.5. The method of treatment of E141, wherein the disease or disorder is anal cancer.
E142.6. The method of treatment of E141, wherein the disease or disorder is penile cancer.
E142.7. The method of treatment of E141, wherein the disease or disorder is a head and neck cancer.
E143. The method of treatment of E141, further comprising treating the patient with one or more additional therapies.
E144. The method of treatment of E143, wherein the one or more additional therapies is a surgical or non-surgical therapy.
E145. The method of treatment of E144, wherein the non-surgical therapy comprises co-administration of an additional agent.
E146. The method of treatment of E145, wherein the additional agent is contained in the same composition that contains the polynucleotide, polypeptide, vector, composition or vaccine.
E147. The method of treatment of E145, wherein the additional agent is a chemotherapy agent, an anti-inflammatory agent, an analgesic, a biological response modifier, a vector comprising such agents, or a cell comprising the agent or a nucleic acid encoding the same.
E148. The method of treatment of E145, wherein the additional agent is administered at or near the same location as the polynucleotide, polypeptide, vector, composition or vaccine.
E149. The method of treatment of E145, wherein the additional agent is administered at a different location than the polynucleotide, polypeptide, vector, composition or vaccine.
E150. The method of treatment of E145, wherein the additional agent is administered simultaneous with the administration of the polynucleotide, polypeptide, vector, composition or vaccine.
E151. The method of treatment of E145, wherein the additional agent is administered at a different time than administration of the polynucleotide, polypeptide, vector, composition or vaccine.
E152. The method of treatment of E145, wherein the additional agent is selected from the group consisting of: steroids and glucocorticoids, including betamethasone, budesonide, dexamethasone, hydrocortisone acetate, hydrocortisone, hydrocortisone, methylprednisolone, prednisolone, prednisone, triamcinolone; nonsteroidal anti-inflammatory drugs (NSAIDs) including aspirin, ibuprofen, naproxen, methotrexate, sulfasalazine, leflunomide, anti-TNF medications, cyclophosphamide and mycophenolate; and sphingosine 1-phosphate receptor modulators, including fingolimod (Gilenya®), ozanimod (Zeposia®), and amiselimod. In some embodiments, NSAIDs are chosen from the group consisting of ibuprofen, naproxen, naproxen sodium, Cox-inhibitors such as VIOXX® (rofecoxib) and CELEBREX® (celecoxib), and sialylates.
E153. The method of treatment of E145, wherein the additional agent is selected from the group consisting of: acetaminophen, oxycodone, tramadol, and proporxyphene hydrochloride.
E154. The method of treatment of E145, wherein the additional agent is selected from the group consisting of: molecules directed against cell surface markers (e.g., CD4, CD5, etc.).
E155. The method of treatment of E153, wherein the additional agent is a cytokine inhibitor, such as a TNF inhibitor (e.g., etanercept (ENBREL®), adalimumab (HUMIRA®), and infliximab (REMICADE®)).
E156. The method of treatment of E145, wherein the additional agent is a nucleotide analog (e.g., Cidofovir).
E157. The method of treatment of E145, wherein the additional agent is an angiogenesis inhibitor, such as Bevacizumab (AVASTIN®).
E158. The method of treatment of E145, wherein the additional agent is a non-steroidal anti-inflammatory compound, such as COX-2-selective drugs (e.g., Celexecob (CELEBREX®)).
E159. The method of treatment of E145, wherein the additional agent is an immune checkpoint inhibitor, such as a PD-1 inhibitors (e.g., Pembrolizumab (KEYTRUDA®), Nivolumab (OPDIVO®), and Cemiplimab (LIBTAYOR) and/or a PD-L1 inhibitor (e.g., Atezolizumab (TECENTRIQ®), Avelumab (BAVENCIO®), Durvalumab (IMFINZI®), and bintrafusp alfa).
E160. The method of treatment of E145, wherein the additional agent is an adhesion molecule inhibitor.
E161. The method of treatment of E145, wherein the additional agent is a monoclonal antibody.
E162. The method of treatment of E145, wherein the additional agent is an anti-rheumatic drug, such as cyclophosphamide, cyclosporine, methotrexate, penicillamine, leflunomide, sulfasalazine, hydroxychloroquine, Gold (oral (auranofin) and intramuscular), and/or minocycline.
E163. The method of treatment of E145, wherein the additional agent is a colony-stimulating factor, such as macrophage colony-stimulating factor, granulocyte macrophage colony-stimulating factor (GM-CSF), granulocyte colony-stimulating factor (G-CSF) or promegapoietin.
E164. The method of treatment of E145, wherein the additional agent is a tumor necrosis factor, such as TNFα, lymphotoxin-alpha (LT-alpha), lymphotoxin-beta (LT-beta), T cell antigen gp39 (CD40L), CD27L, CD30L, FASL, 4-1BBL, OX40L, and TNF-related apoptosis inducing ligand (TRAIL).
E165. The method of treatment of E145, wherein the additional agent is an interleukin, such as IL-I, IL-2, IL-3, IL-4, IL-5, IL-6, IL-7, IL-8 (CXCL8), IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-35, IL-36, or a functional fragment or variant thereof.
E166. The method of treatment of E145, wherein the additional agent is an interferon, such as IFN-α, IFN-β, IFN-ε, IFN-κ, and IFN-ω, interferon type II, IFN-γ, interferon type III, IFNAI, IFNA2, IFNA4, IFNA5, IFNA6, IFNA7, IFNA8, IFNAI0, IFNA13, IFNA14, IFNA16, IFNAI 7, IFNA21, or a functional fragment or variant thereof.
E167. The method of treatment of E145, wherein the additional agent is a chemokine selected from the group consisting of: CCLI, CCL2 (MCP-1), CCL3, CCL4, CCL5 (RANTES), CCL6, CCL7, CCL8, CCL9 (or CCLI0), CCLI 1, CCL12, CCL13, CCL14, CCL15, CCL16, CCLI 7, CCL18, CCL19, CCL20, CCL21, CCL22, CCL23, CCL24, CCL25, CCL26, CCL27, and CCL28; the CXC subfamily: CXCLI, CXCL2, CXCL3, CXCL4, CXCL5, CXCL6, CXCL7, CXCL8, CXCL9, CXCLI0, CXCLII, CXCL12, CXCL13, CXCL14, CXCL15, CXCL16, and CXCLI 7; the XC subfamily: XCLI and XCL2; and the CX3C subfamily CX3CL1.
E168. The method of treatment of E145, wherein the additional agent is a membrane-bound cytokine, which is co-expressed with a chimeric antigen receptor (CAR).
E169. The method of treatment of E145, wherein the additional agent is a cytokine selected from IL2, IL7, IL12, IL15, IL21, IFNγ, TNF-α, or a functional fragment or variant thereof.
E170. The method of treatment of E145, wherein administration of the polynucleotide, polypeptide, vector, composition or vaccine in combination with the additional agent enhances the treatment of a disease or disorder and/or reduces any side-effects of treatment with the polynucleotide, polypeptide, vector, composition or vaccine alone.
E171. The method of treatment of E145, wherein malignancies caused by HPV16 are treated.
E171.1 The method of treatment of E145, wherein malignancies caused by HPV18 are treated.
E171.2 The method of treatment of E145, wherein malignancies caused by HPV16/18 are treated.
E171.3 The method of treatment of E145, wherein malignancies caused by HPV45 are treated.
E172. A method of treating a patient that has an HPV-associated disease or disorder comprising administering the vector of E104.
E172.1. A method of treating a patient that has an HPV16-associated disease or disorder comprising administering the vector of E104.
E172.2. A method of treating a patient that has an HPV18-associated disease or disorder comprising administering the vector of E104.
E172.3 A method of treating a patient that has an HPV45-associated disease or disorder comprising administering the vector of E104.
E173. The method of treatment of E172, wherein the patient is administered a dose of between about 1×10¹¹ and about 5×10¹¹ particle units (PU) of vector.
E174. The method of treatment of E172, wherein the patient is administered about 5×10¹¹ particle units (PU) of vector.
E175. The method of treatment of E172, wherein malignancies caused by HPV16 are treated.
E175.1. The method of treatment of E172, wherein malignancies caused by HPV18 are treated.
E175.2. The method of treatment of E172, wherein malignancies caused by HPV16/18 are treated.
E175.3 The method of treatment of E172, wherein malignancies caused by HPV45 are treated.
E176. The method of treatment of any one of E172 thru E175.3, wherein the patient is administered one or more doses of vector.
E176.1. The method of treatment of E176, where the doses are administered via injection.
E176.2. The method of treatment of E176, wherein the patient is administered two doses of vector.
E176.3. The method of treatment of E176, wherein the patient is administered three doses of vector.
E176.4. The method of treatment of E176, wherein the patient is administered four doses of vector.
E177. The method of treatment of E176, wherein the patient is administered more than four doses of vector.
E178. The method of treatment of E176, wherein the minimum time between administrations of the vector is 11 days.
E179. The method of treatment of E176, wherein the patient is administered escalating doses of the vector.
E180. The method of treatment of E176, wherein one or more doses of vector is 5×10¹¹ PU.
E181. The method of treatment of E176, wherein one or more doses of vector is 1×10¹¹ PU.
E182. The method of treatment of E176, wherein the volume for each injection is about 1.0 mL.
E183. The method of treatment of E176, wherein the patient is administered 5×10¹¹ PU of vector on days 1, 15, and 29.
E184. The method of treatment of E176, wherein the patient is administered 5×10¹¹ PU of vector on days 1 and 15.
E185. The method of treatment of E176, wherein the patient is administered 1×10¹¹ PU of vector on days 1, 15, and 29.
E186. The method of treatment of E176, wherein the patient is administered 1×10¹¹ PU of vector on days 1 and 15.
E187. The method of treatment of E176, wherein bintrafusp alfa is co-administered with the vector.
E188. The method of treatment of E187, wherein the dosage of bintrafusp alfa is 1,200 mg.
E189. The method of treatment of E187, wherein bintrafusp alfa is administered regardless of a patient's body weight.
E190. The method of treatment of E187, wherein bintrafusp alfa is administered intravenously.
E191. The method of treatment of E187, wherein bintrafusp alfa is administered once every 2 weeks.
E192. The method of treatment of E187, wherein bintrafusp alfa is administered no longer than a year.
E193. The method of treatment of E176, wherein a histone decetylase (“HDAC”) inhibitor is co-administered with the vector.
E194. The method of treatment of E176, wherein the dosage of HDAC inhibitor administered is 400 mg.
E195. The method of treatment of E193, wherein the HDAC inhibitor is administered orally.
E196. The method of treatment of E193, wherein the HDAC inhibitor is intravenously.
E197. The method of treatment of E193, wherein the HDAC inhibitor is vorinostat, romidepsin, or belinostat.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims.

### EXAMPLES

The following examples are included to demonstrate preferred embodiments of the invention, in addition to those embodiments disclosed earlier herein. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Antigenicity Bioinformatics Workflow for HPV Vaccine Designs

### Generating Consensus Sequences

Sample sequences for E5, E6, and E7 (for HPV16, HPV 17, HPV31, HPV 33, and HPV45, serotypes determined to have a higher predisposition for cancer) were downloaded, converted to FASTQ files, and imported into R statistical program. Individual AA sequences were read into R using the biostrings package (readAAStringSet function). Multiple sequence alignments were performed using the ClustalW algorithm in the msa R package. For each sub-group/subtype, consensus sequences were generated and output to PDF/FASTQ files using the msaPrettyPrint function.

### Predicting Binding Affinity

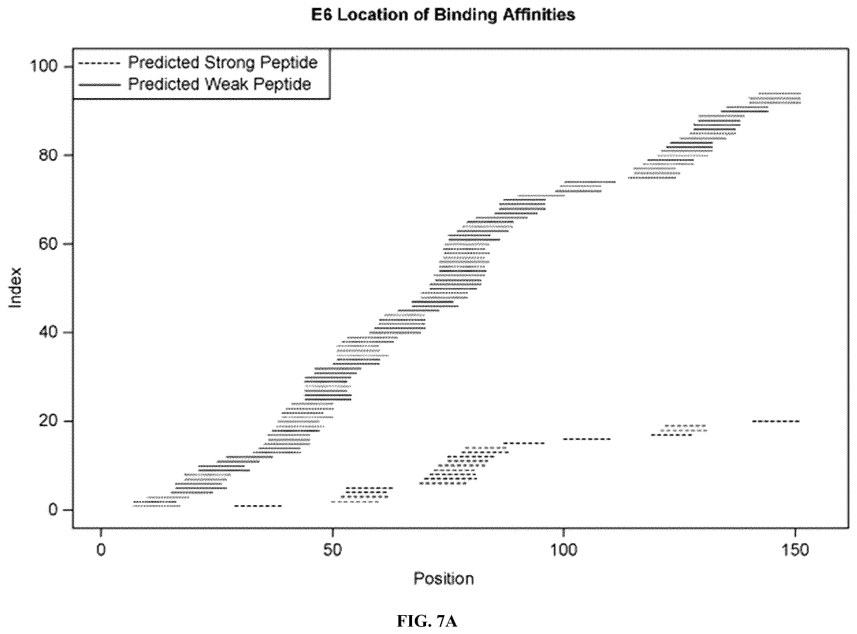

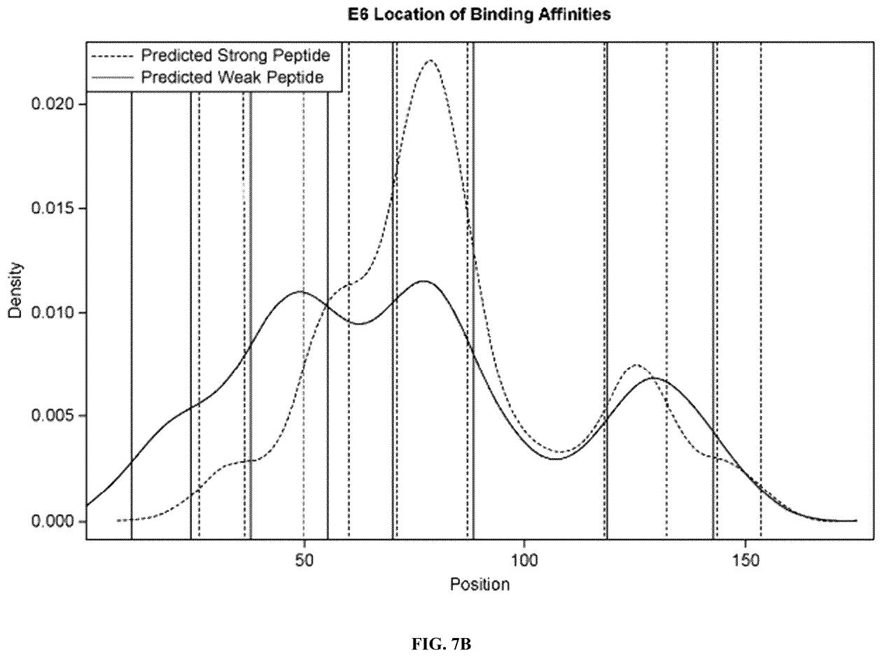

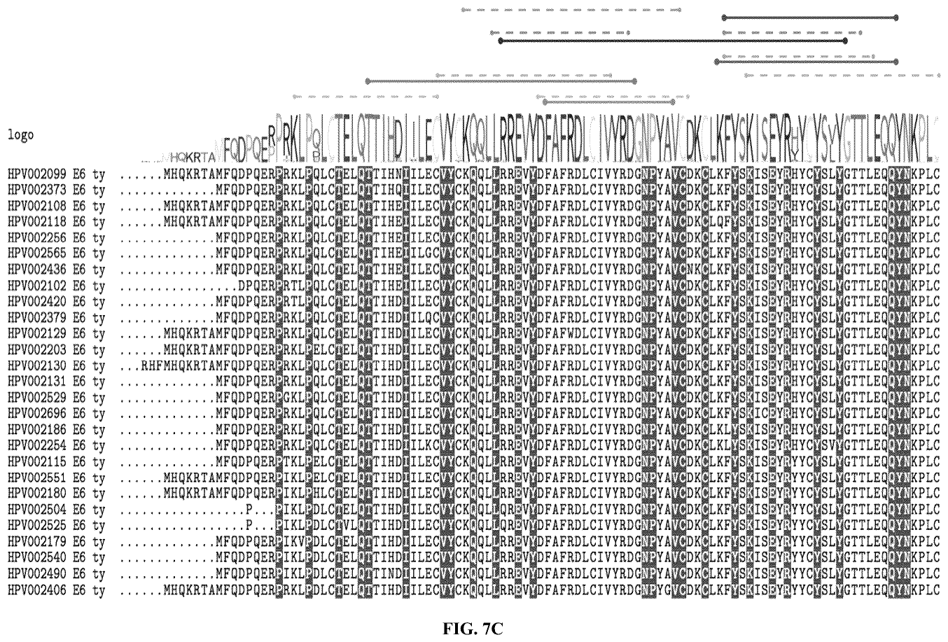

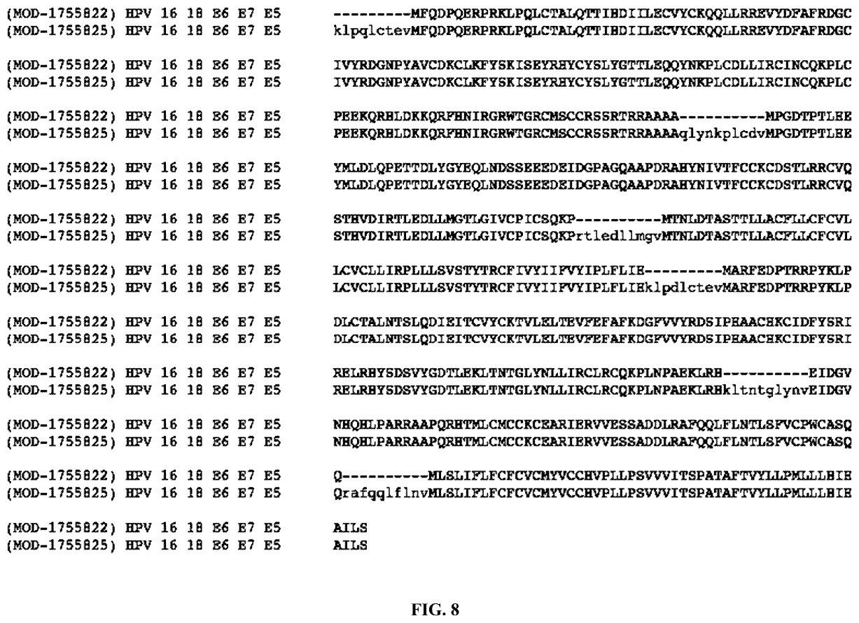

NetMHC 4.0 was applied to each consensus sequence to predict binding affinity against all major MHC-I alleles (HLA-A0101, HLA-A0201, HLA-A0301, HLA-A2402, HLA-A2601, HLA-B0702, HLA-B0801, HLA-B2705, HLA-B3901, HLA-B4001, HLA-B5801, and HLA-B1501). NetMHC 4.0 uses artificial neural networks to predict the binding affinity of peptide sequences. This analysis was performed for HPV16, HPV18, HPV31, HPV33, and HPV45. Thresholds were arbitrarily established at 0.5% (strong binders) and 2% (weak binders) ranks. Peptides with predicted binding affinity greater than 99.5% were classified as strong binders, and peptides with predicted binding affinity greater than 98% were classified as weak binders. The position of each AA within the peptide sequences were extracted and used to generate density curves (FIG. 7A and FIG. 7B). Using these density curves, first and second order differentials were calculated to determine peaks for strong and weak binders (FIG. 7C). Finally, the union of these positions was used to extract AA sequences likely to elicit a response.

### Predicting Binding Affinity in HPV16, HPV18, HPV31, HPV33, and HPV45

Previous studies have shown that HPV strains 16, 18, 31, 33, and 45 were precursors for cervical carcinoma. To identify candidate peptides with broad coverage across these strains, binding affinity was predicted within each strain. The sequences with strong/weak binding affinity predictions were extracted. Using protein blast, these sequences were aligned against the consensus sequences across all five strains. Alignments were plotted for all five serotypes and coverage was assessed.

### Example 2: HPV Antigen Designs

Naturally existing sequence variations on HPV strains could potentially hinder the development of effective HPV vaccines. To address this concern, the present vaccine design approach utilized bioinformatics and protein engineering methods to select and design antigens with broader coverage of T cell epitopes, novel mutations, and enhancer agonist peptides. Drawing on available information of extended coverage of antigen regions with CTL-specific epitopes and in silica prediction results, the designed HPV vaccine antigens can induce robust HPV-16 and HPV-18 specific responses and potentially benefit patients at high risk of HPV-derived cancers.

The new HPV vaccines include the following engineered proteins, peptides and/or modifications:
1) truncated E5 regions that fused with E6 and E7 domains of HPV16 and HPV18;
2) newly designed point mutations that can potentially abrogate p53 binding with the known PTPN13 binding mutants (Weiking et al., Cancer Gene Ther. 19 (10:667-74 (2012)). doi: 10.1038/cgt.2012.55. Epub 2012 Aug. 24);
3) a novel truncation in HPV18 E6 non-immunogenic region at the C-terminus and application of p53 binding knockout mutants; and/or
4) a novel truncation of HPV18 E7 non-immunogenic region at the N-terminus and incorporating pRb, Mi2B mutants (Weiking et al., 2012); and/or 5) agonist peptides from Tsang, et al., Vaccine 35 (19): 2605-2611 (2017) doi: 10.1016/j.vaccine.2017.03.025. Epub 2017 Apr. 4 in the newly designed HPV vaccine constructs.

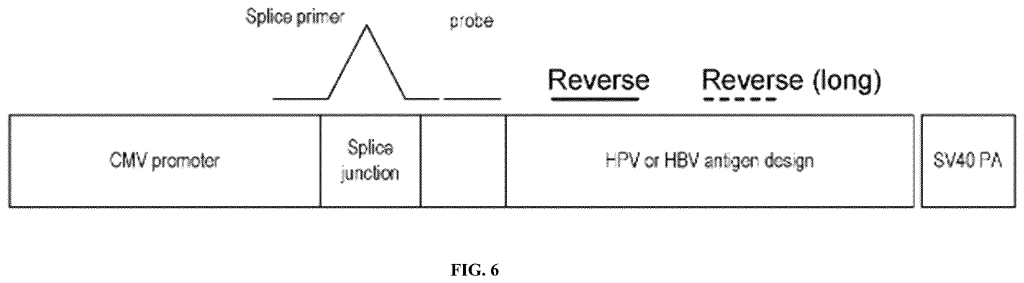

For RNA qPCR relative expression assay, 5′-TGC-CAAGAGTGACGTGTCCA-3′ (SEQ ID NO: 110) was used as a splice primer, and 5′-CCCAGGTC-CAACTGCAGCCGG-3′ (SEQ ID NO: 111) was used as a splice probe. Specific primers designed for each antigen were used as reverse primers (FIG. 6).

### HPV Design 1

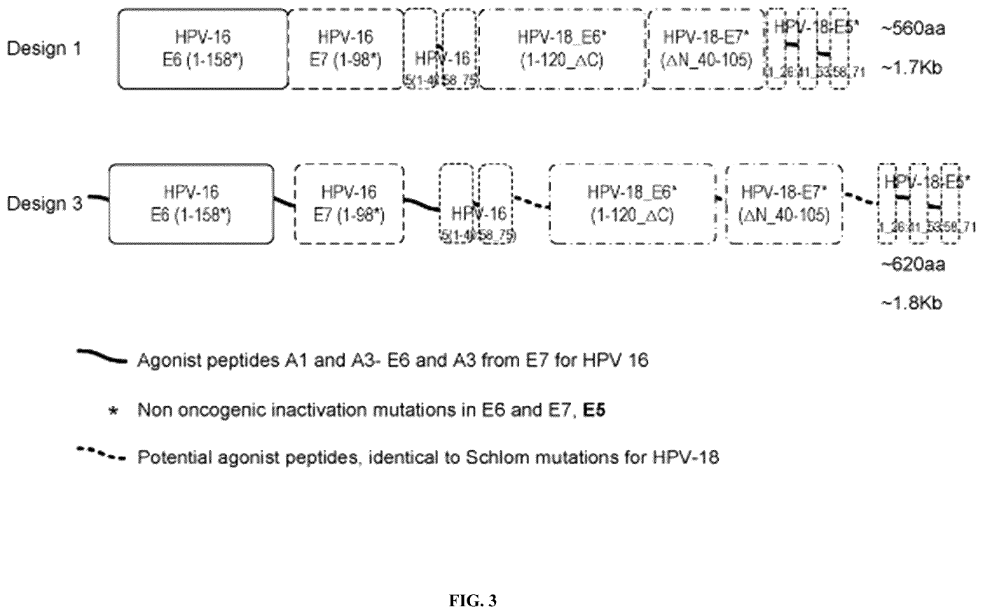

Consensus sequence information was utilized to select HPV 16/18 reference sequences for the design which included all major variants. The vaccine composition comprising different E6, E7 and E5 protein components with domain boundaries and mutation information is shown in FIG. 3. The reference sequence for E6 was obtained from the Human Papillomavirus T cell Antigen Database (Bioinformatics Core at Cancer Vaccine Center, Dana-Farber Cancer Institute). Reference sequences for E5 and E7 were obtained from the Papilloma Virus Episteme (PaVE) database.

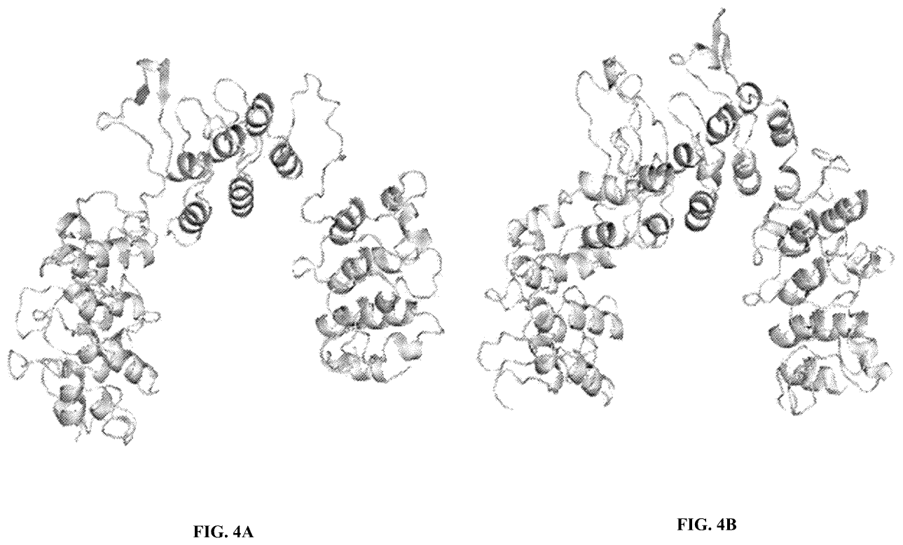

The design for HPV16 and HPV18 E5 were inspired via a combination of in silica predictions (IEDB and netMHC) and, for HPV16 also included findings of Chen et al., J Viral. 78 (3): 1333-43 (2004); whereas, the design for HPV18 included N terminal residues (1-26) and C terminal residues (41-53, 58-71) (scc, FIGS. 4A and 4B).

Inactivating mutations for HPV16 and HPV18 were introduced into E6 and E7 peptide sequences in order prevent oncogenicity (Weiking et al., 2012 October; 19 (10): 667-74. doi: 10.1038/cgt.2012.55. Epub 2012 Aug. 24). For HPV16 E6, these mutations were E18A, L50G and alanine replacement from 148-151 (ETQL (SEQ ID NO: 285) to AAAA (SEQ ID NO: 286)). For HPV16 E7, four mutations (H2P, C24G, E46A, and L67R) were included (Weiking et al., 2012). For HPV18 E6, two mutations were included (E18Aand L52G). For HPV18 E6, only the N terminus portion was predicted to contain MHC-I binding epitopes (IEBD and netMHC analysis), thus, residues starting at amino acid 121 were removed from these designs. An additional mutation was included in E6 in order to further abrogate its interaction with the p53 protein (Martinez-Zapien et al., Nature 529 (7587): 541-45 (2016). For HPV18 E7, two mutations (Weiking et al., 2012) were included (E55A and L74R) and residues from (1-40) were removed because HPV-18 E7 contained majority of the predicted MHC-I binding epitopes (IEDB and netMHC predictions) in the C terminal region.

### HPV Design 2

For designing recombinant multi-epitope proteins as HPV vaccine antigens, a total of 32 key immunogenic peptides, as listed in Table 5, were selected. These peptides included CTL specific peptides from E6 (HPV-16/-18), E7 (HPV-16/-18) and E5 (HPV16) genes. Most of these CTL epitopes were reported with multiple immunological assays, immuno-proteomics, and were included in IEDB resource and netMHC predictions. The novelty of this antigen design is to graft the CTL peptides on to a human ankyrin repeat protein scaffold for possible generation of new protein bearing T cell epitopes and or proteasome cleavage sites.

Human Ankyrin-like repeat (“ALR”) protein (PDB code 1QYM) was selected as a scaffold on to which CTL peptides were assembled randomly, enabling different types of protein linker sequences embedded between the peptides. ALR proteins have generally high expression and high stability; the ALR protein provides a scaffold for the HPV peptides and could create novel CTLs. Caution was taken by random shuffling of the peptides to prevent any reformation of E6 and E7 oncogenic proteins.

A homology model (FIG. 4A) was used to assess the overall structural feature and to compare the design against native ankyrin repeats. This model suggested that the grafted CTL peptides on an ankyrin scaffold should retain their tertiary structure. It is expected that the scaffold amino acids connecting those peptides could serve as “linkers” and potentially create new CTL epitopes and or agonist peptide epitopes.

TABLE 5
Peptides from E5 (HPV16), E6 (HPV16 and HPV18), and E7 (HPV16 and HPV18)
along with agonist peptides used to assemble multi-epitope vaccine constructs

| SEQ | | SEQ | | SEQ | |
|---|---|---|---|---|---|
| ID | | ID | | ID | |
| NO | HPV16-E6 | NO | HPV16-E7 | NO | HPV16-E5 |
| 113 | HLDKKQRFHNI | 122 | LCVQSTHVDI | 130 | YIIFVYIPL |
| 114 | RWTGRCMSCC | 123 | RTLEDLLMGT | | |
| 115 | TTLEQQYNKPLCDLL | 124 | TLGIVCPI | | |
| 116 | ISEYRHYCY | 125 | LLMGTLGIV | | |
| 117 | VYDFAFRDL | 126 | TLHEYMLDL | | |
| 118 | TIHDIILECV | 127 | AHYNIVTFCC | | |
| 119 | KLPQLCTEL | 128 | YMLDLQPETT | | |
| 120 | FAFRDLCIVY | 129 | CDSTLRLCV | | |
| 121 | LCIVYRDGNPYAVCD | | | | |
| SEQ | | SEQ | | SEQ | |
| ID | | ID | | ID | AGONIST |
| NO | HPV18-E6 | NO | HPV18-E7 | NO | PEPTIDES |
| 131 | KLTNTGLYNL | 139 | DDLRAFQQLFLNTLS | 145 | KLPQLCTEV |
| 132 | KCIDFYSRI | 140 | FQQLFLNTL | 146 | QLYNKPLCDV |
| 133 | FAFKDLFVV | 141 | QLFLNTLSFV | 147 | RTLEDLLMGV |
| 134 | NLLIRCLRC | 142 | LFLNTLSFVCPWCAS | | |
| 135 | KLPDLCTEL | 143 | TLQDIVLHL | | |
| 136 | ELTEVFEFA | 144 | SEEENDEIDGVNHQHLPARR | | |
| 137 | SLQDIEITCV | | | | |
| 138 | KTVLELTEV | | | | |

### HPV Design 3

Design 3 is similar to Design 1 with the addition of enhancer agonist peptides (FIG. 3). Overall, designs 1 and 3 contain all necessary sequences from each E5, E6, and E7 (FIGS. 4A and 4B). These were inspired via inventor-selected combinations using guidance from in silica epitope prediction analysis and available information on MHC-I binding and cytokine production following T cell activation.

For HPV16, this design includes: (1) peptides from Tsang et al., Vaccine 35 (19): 2605-2611 (2017) doi: 10.1016/j.vaccine.2017.03.025. Epub 2017 Apr. 4; (2) three peptides that exhibit better MHC-I binding and elicit more robust cytotoxic T cell lymphocyte (CTL) response comprising mutations in E6 (L19V and Q91L/L99V) and mutation in E7 (T86V); (3) two enhancer peptides fused to the inactivated N and C terminus of E6; and (4) one enhancer peptide fused to the inactivated C terminus of E7.

For HPV18, this design includes: (1) three peptides with identical mutations to mimic enhancer agonist peptides similar to peptides of HPV16 (Tsang et al., 2017) comprising the mutations in E6 (L21V and L101V); and (2) two potential mimic enhancer agonists fused to N and C terminus regions of E6 and another agonist mimic fused to the C terminal region of E7. Since HPV18 naturally had the aforementioned mutations in E6 (Q91L) and E7 (T95V) naturally, no additional modifications were needed.

### HPV Design 4

This design was based on Design 2, utilizing the same ankyrin repeat approach. However, Design 4 includes an additional three unique agonist peptides from the peptides of Tsang et al., 2017 for a total of 35 key immunogenic peptides (Table 5). The order of peptides grafted on the scaffold was again randomized in order to be different from Design 2 and for the potential to generate different CTL epitopes. A homology model of Design 4 was generated (FIG. 4B). The homology of this design was found similar to the Design 2, but indicated local structural variations.

### HPV Design 5

This design is a multi-epitope vaccine, designed by selecting all 35 key immunogenic peptides shown in Table 5. It was assembled with a charged dipeptide KK residue. Advantages of this design include potential for cleavage at “KK” residues and random modification with the “K” residue added at CTL epitopes.

### Example 3: Assessing the Predicted Binding Affinities of HPV Vaccine Constructs

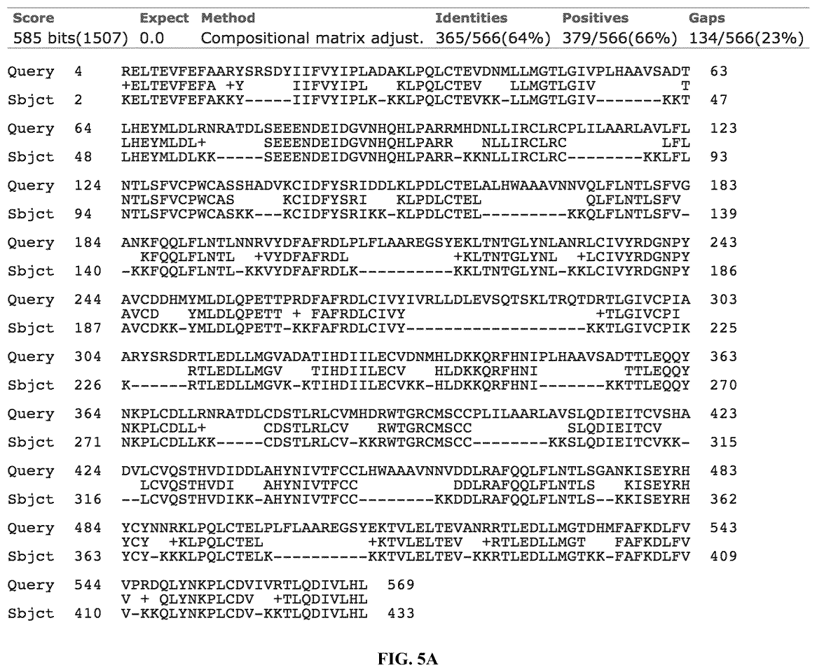

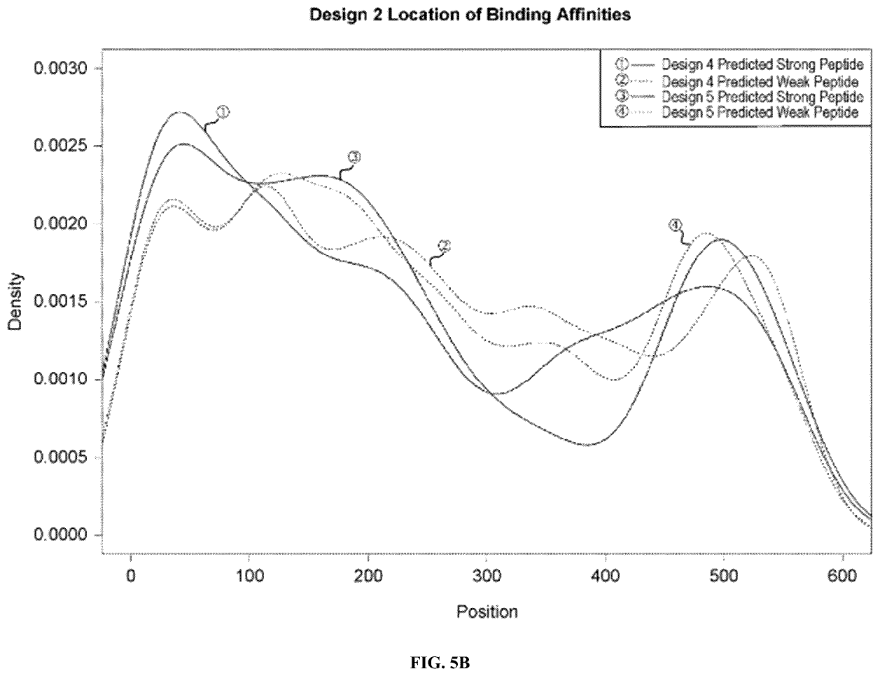

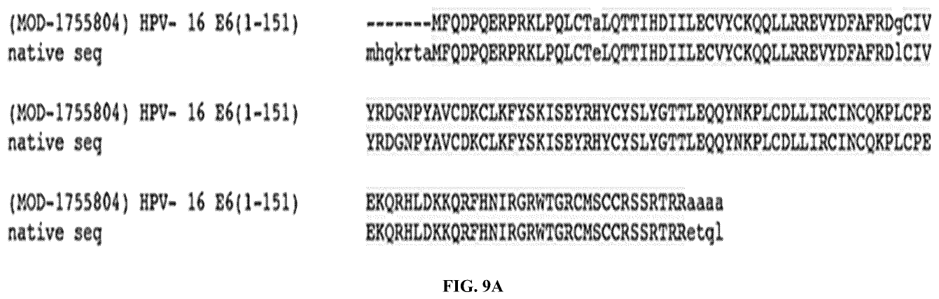

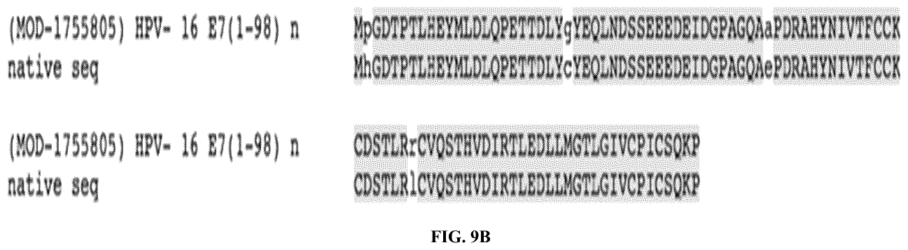

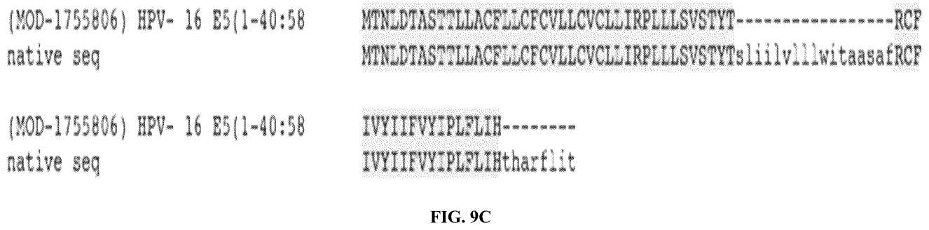

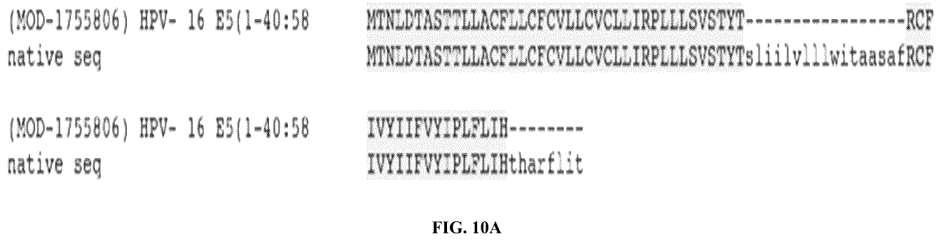

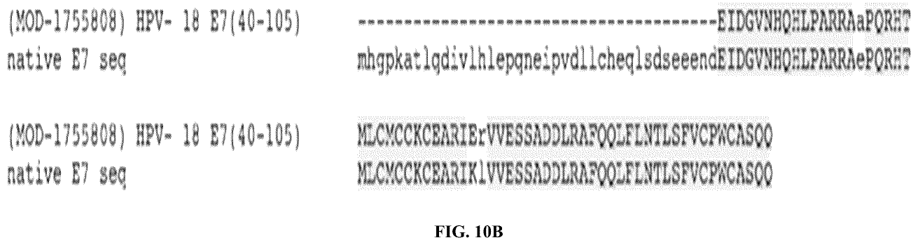

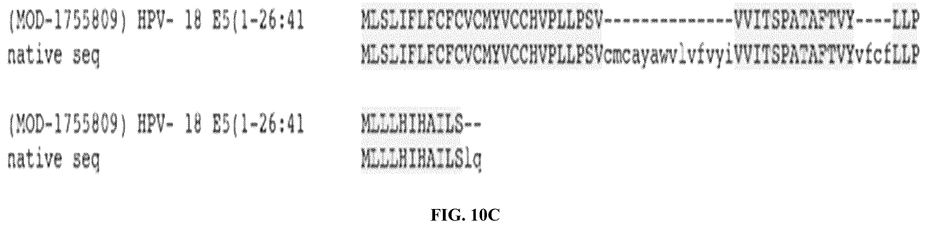

Bioinformatics predictions for the binding affinity for each design were carried out. Entire sequences were loaded into NetMHC, which was used to assess antigenicity and extent of coverage against various HPV genotypes. It should be noted that Designs 4 and 5 use the same 35 peptides. The main difference is that Design 4 peptides were grafted on Ankyrin protein and Design 5 peptides were connected by “KK” linkers (FIG. 5A). Ultimately, the binding affinity predictions on the matched regions of both designs were similar (FIG. 5B). The HPV designs were compared among themselves (Tables 5 and 6) and with reference sequences which are shown in FIG. 8, FIGS. 9A-9C, and FIGS. 10A-10C.

TABLE 6
HPV16 Design Elements (HPV Designs 1 and 3)

| MOD | HPV type | Rationale |
|---|---|---|
| 1755804 | HPV16 E6 non | Inclusion of p53 and PTPN13 binding site |
| | oncogenic | mutations [E18A, L50G, ETQL (SEQ ID NO: |
| | | 285) - AAAA (SEQ ID NO: 286)]. These |
| | | mutations do not allow E6/E7 to degrade |
| | | p53, pRb, PTPN13, or activate telomerase: |
| | | Martinez-Zapien et al., 2016; Weiking |
| | | et al., 2012 |
| 1755805 | HPV16 E7 non | Removal of Rb and Mi2B binding sites; |
| | oncogenic | prior art in Etubics E7 |
| 1755806 | HPV16 E5 (1-40, | Novel truncation of E5 with removal of |
| | 58-75) | 2 immuno-dominant regions |
| 1755814 | HPV16 E6 A1 peptide | Agonistic peptides |
| | (L19V) | |
| 1755815 | HPV16 E6 A3 peptide | Agonistic peptides |
| | (Q91L/L99V) | |
| 1755816 | HPV16 E7 A3 peptide | Agonistic peptides |
| | (T86V) | |

TABLE 7
HPV18 Design Elements (HPV Designs 1 and 3)

| HPV type | Rationale |
|---|---|
| HPV18 E6 non oncogenic | Novel C terminal truncated E6 domain (1-120) |
| | and inclusion of p53 binding sites mutations |
| | [E18A, L50G]; Martinez-Zapien et al., 2016; |
| | Weiking et al., 2012 |
| HPV18 E7 non oncogenic | Novel N terminal truncated E7 domain (40-105), |
| | and inclusion of Rb and Mi2B binding mutants |
| | [E46A, L67R]; Weiking et al., 2012 |
| HPV18 E5 (1-26:41-53:58-71) | Novel truncation of E5 with removal of 2 |
| | immuno-dominant regions |
| HPV18 E6 A1 peptide (L21V) | Agonistic peptides |
| HPV18 E6 A3 peptide (L101V) | Agonistic peptides |
| HPV18 E7 A3 peptide (T95V) | Agonistic peptides |

### Example 4: Design of the Recombinant Gorilla Adenovirus Vaccine AdV-HPV16/18

AdV-HPV16/18 is constructed on Precigen Inc.'s nonhuman primate adenoviral vector platform GC46. The GC46 gorilla adenovector was identified and isolated from nonhuman primate sources, and multiple genes (E1, E3, and E4) have been deleted to prevent viral replication. Thus, AdV-HPV16/18 is not replication competent (FIG. 11A).

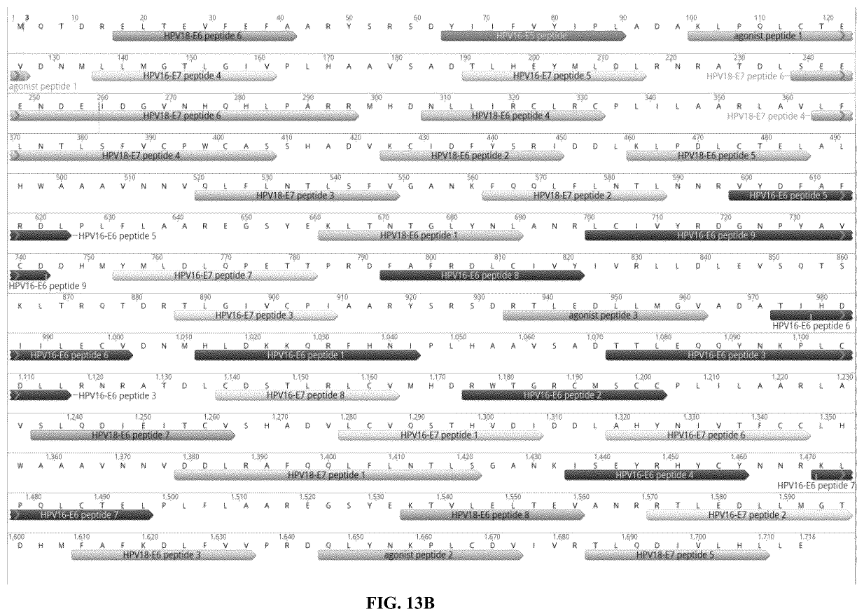

The AdV-HPV16/18 vaccine design contains 35 non-HLA-restricted CTL epitopes of HPV 16 and 18 connected by linkers, including 3 HLA-A2 agonist epitopes of HPV16 previously identified by our laboratory (28). FIG. 13B. These 35 CTL peptide sequences were constitutively expressed under control of a cytomegalovirus (CMV) promoter, and were grafted to a human ankyrin repeat protein scaffold enabling protein linker sequences embedded between the peptides wherein this scaffold retains it tertiary structure displaying the HPV epitopes. FIG. 12, FIG. 13A. Designed shuffling of the peptides prevents any reformation of E6 and E7 oncogenic protein potential and HPV protein viral function. The vaccine or the GC46 empty vector control was given as an s.c. injection of 1×10⁹ VP once per week.

### Example 5: Determining which HPV Vaccine Construct Demonstrated the Best Antitumor Efficacy In Vivo

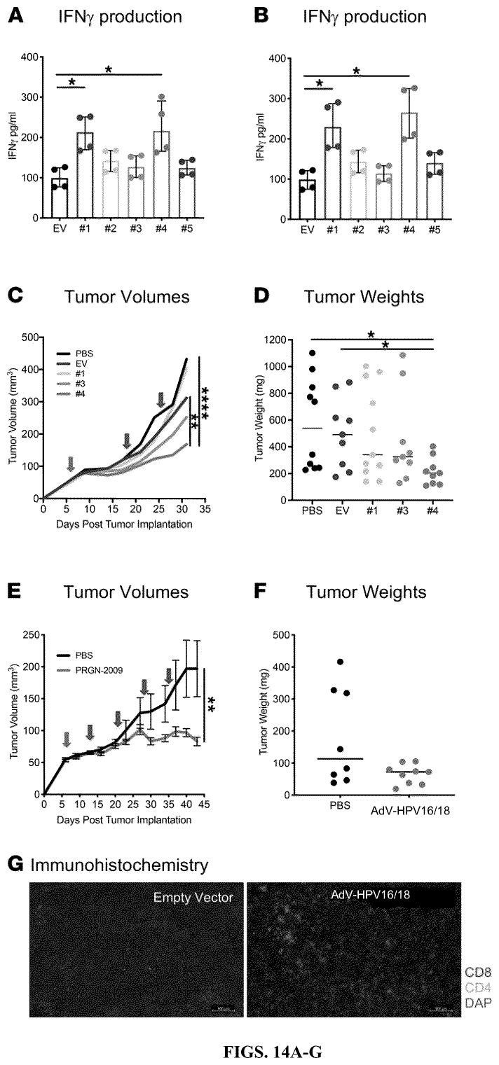

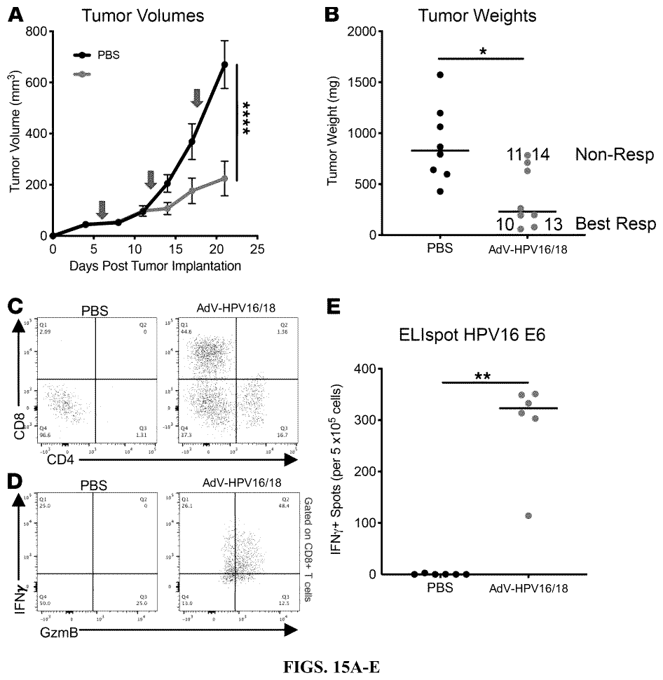

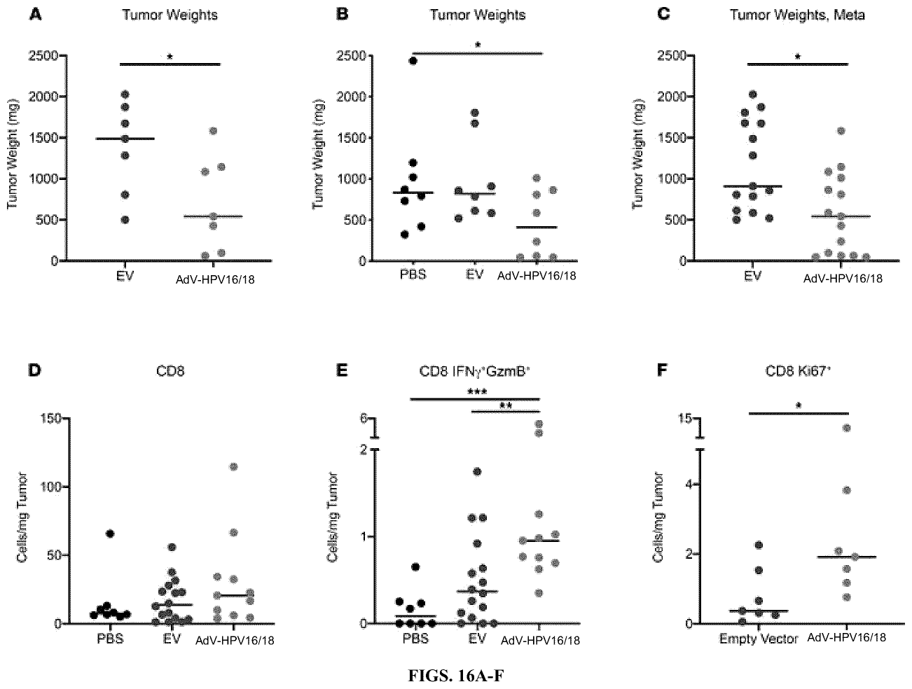

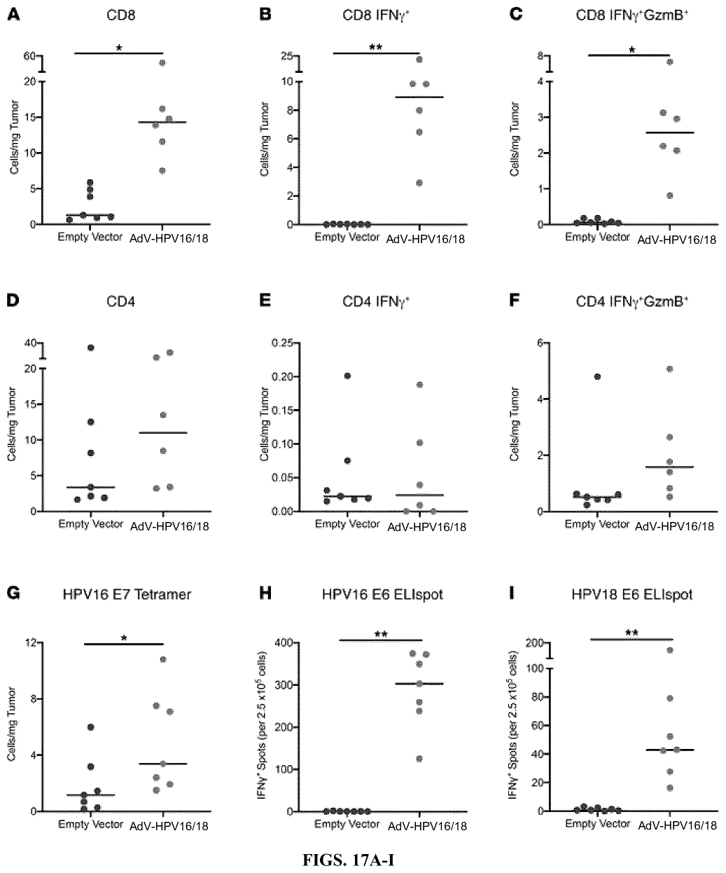

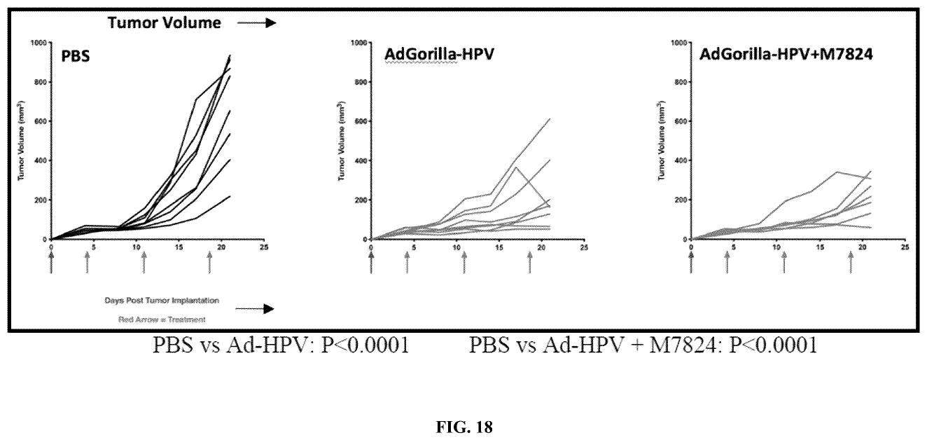

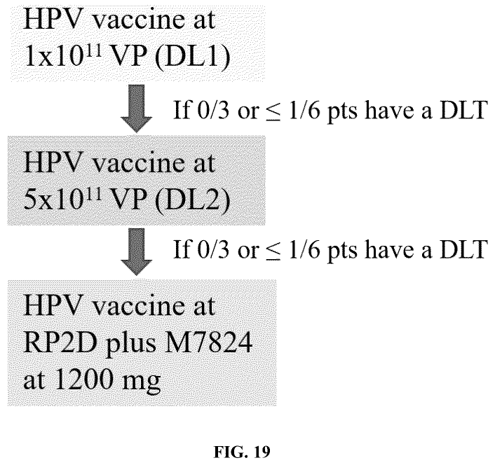

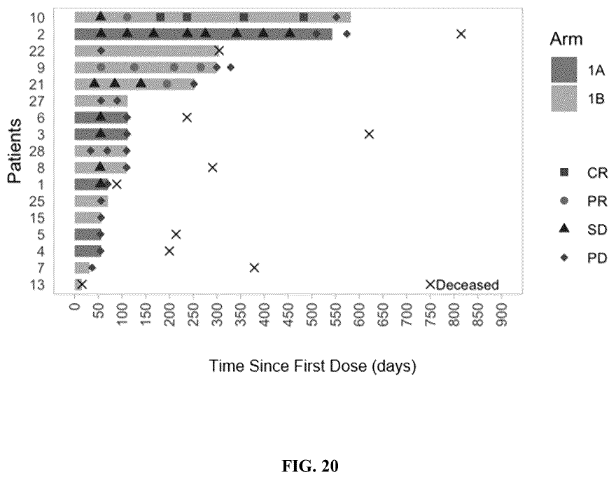

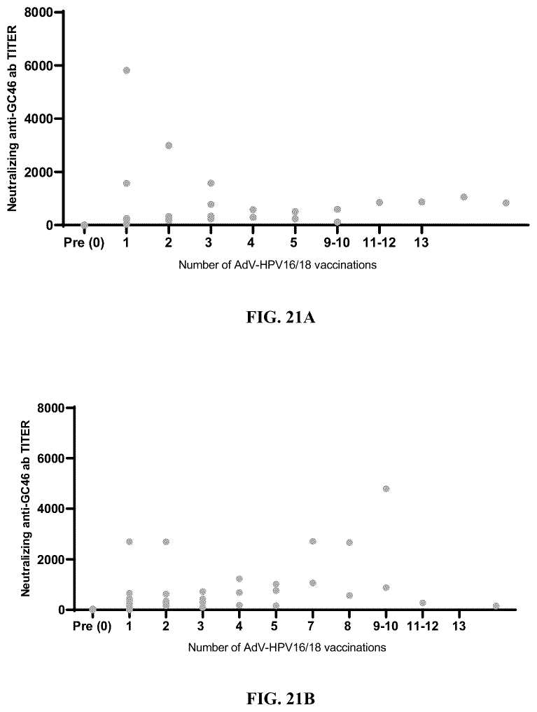

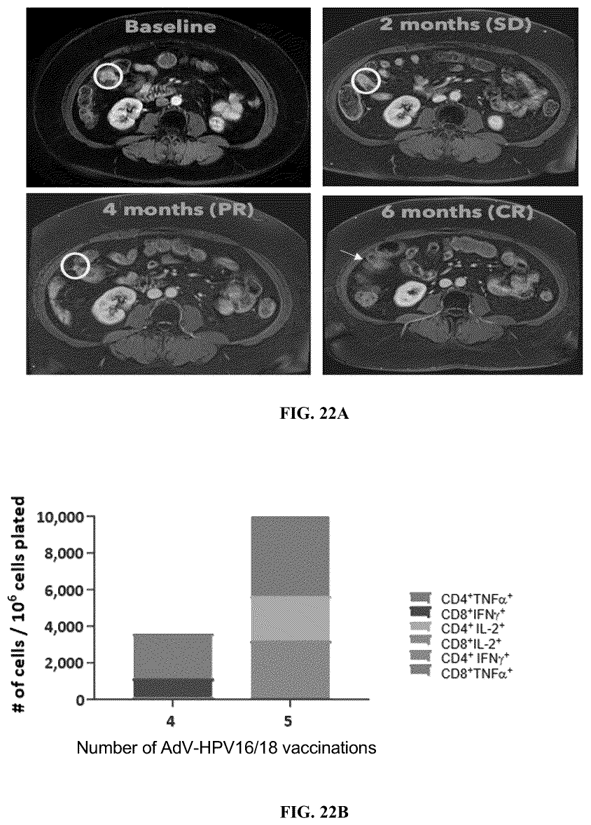

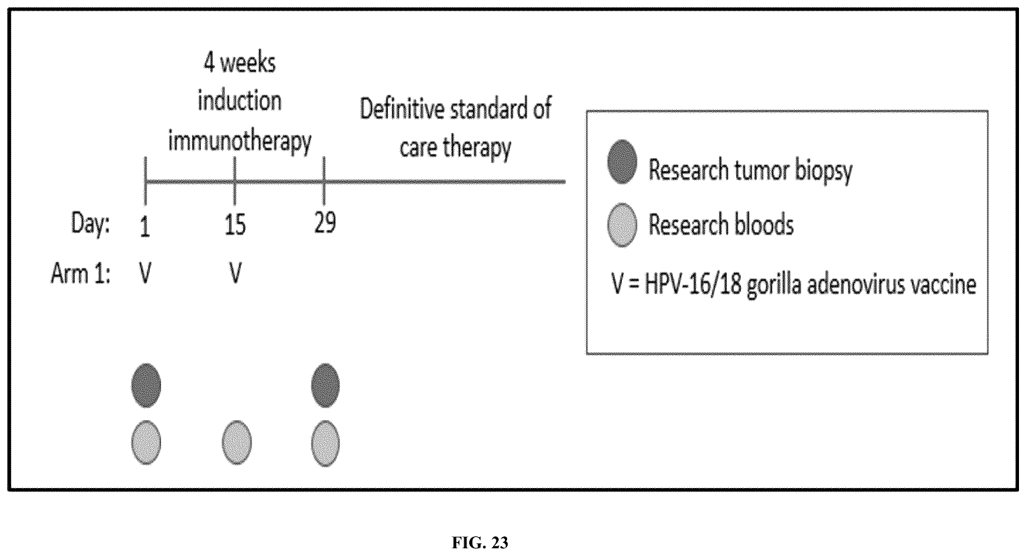

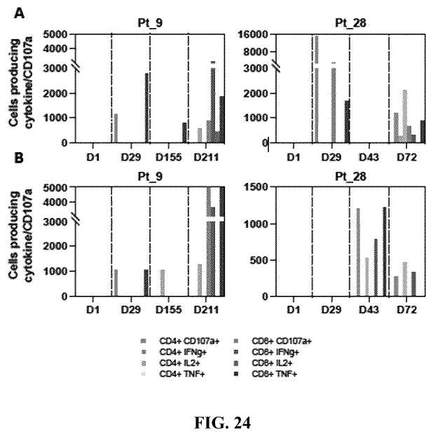

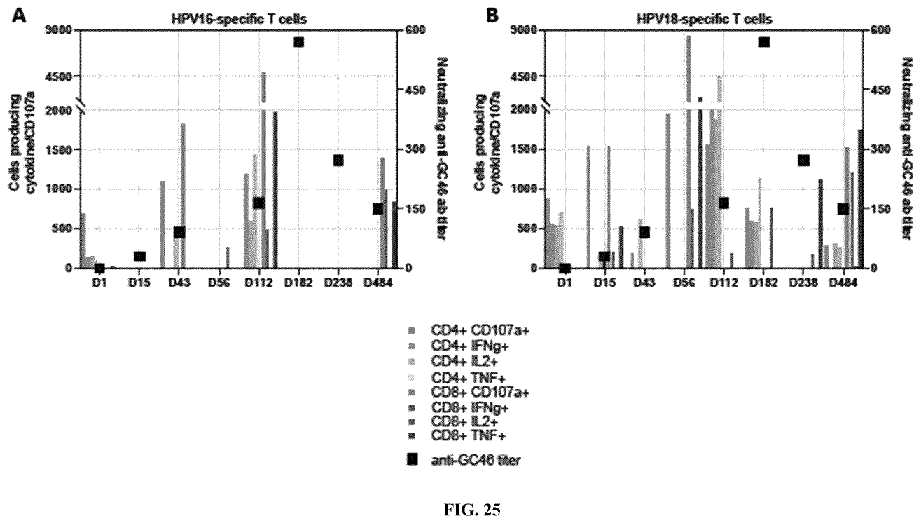

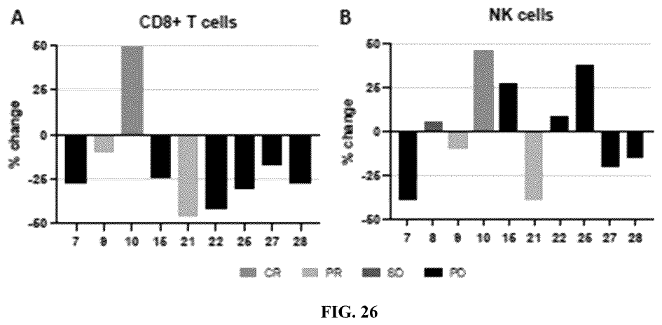

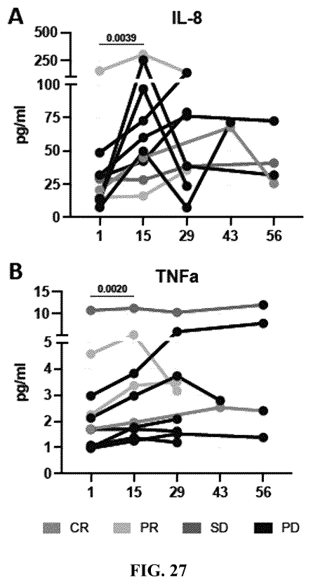

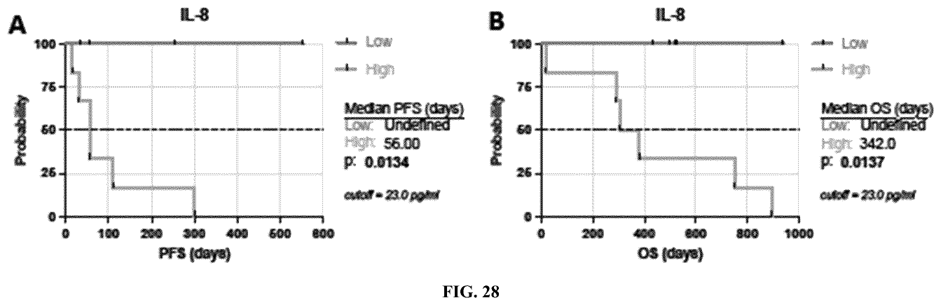

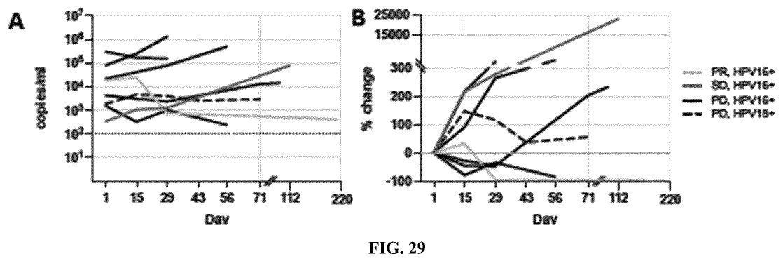

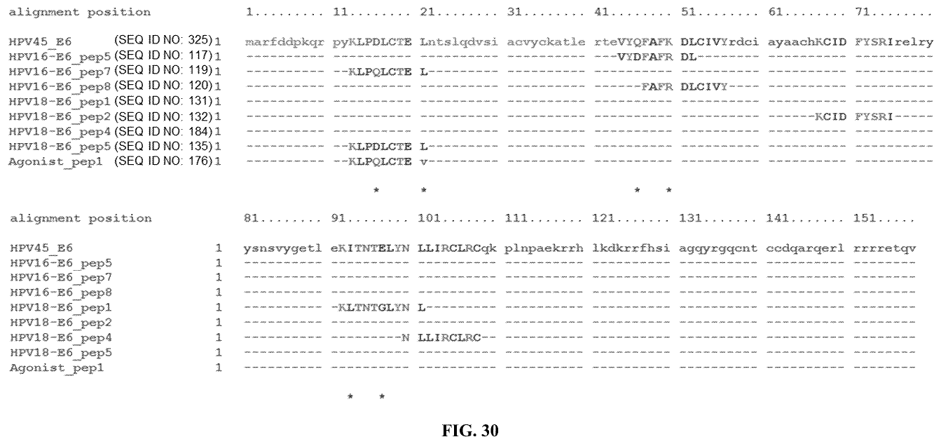

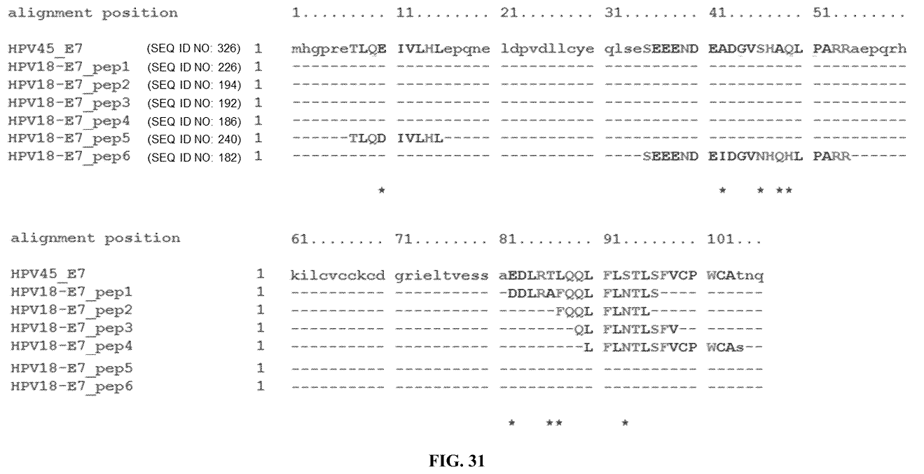

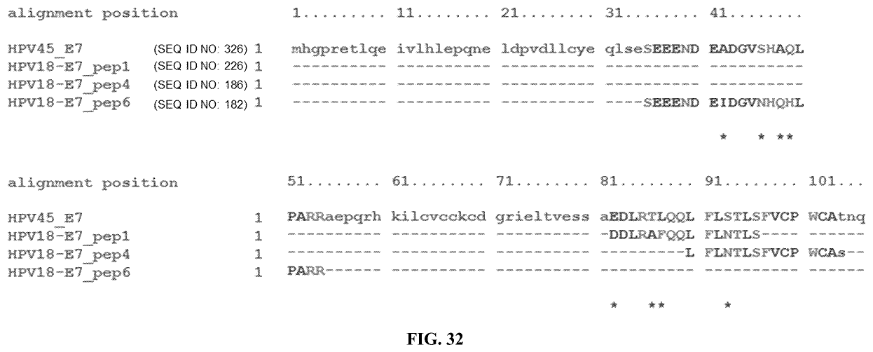

Five different HPV vaccine constructs were designed as part of the Cooperative Research and Development Agreement (CRADA) between the NCI, NIH, and Precigen Inc. Precigen Inc.'s UltraVector technology was used to optimize the vaccine design. Two constructs (nos. 1 and 3) are modular HPV16 and HPV18 fusion protein designs. Three constructs (nos. 2, 4, and 5) are multi-CTL epitopes grafted onto scaffolds or connected by linkers (FIG. 11B). Healthy-donor PBMCs were stimulated in vitro by autologous DCs infected with the 5 different HPV vaccine constructs (nos. 1-5) as described in Methods. Following these stimulations, an IFN-γ production ELISA assay using overlapping HPV16 E6 and E7 15-mer peptides was performed to detect peptide-specific T cell stimulation. The IFN-γ levels in the supernatant of PBMCs stimulated with 2 of the HPV vaccine constructs (nos. 1 and 4), were increased compared with the empty vector control and other constructs when restimulated with the HPV16 E6 15-mer peptides (FIG. 14A, P<0.05) and the HPV16 E7 15-mer peptides (FIG. 14B, P<0.05).

Cell lines used in mouse model studies. The TC-1 murine HPV16⁺ lung carcinoma cell line was a gift from T. C. Wu (Johns Hopkins University, Baltimore, Maryland, USA), and was cultured according to previous studies (42). TC-1 was derived from primary epithelial cells of C57BL/6 mice transfected with the HPV16 E6, HPV16 E7, and c-Ha-ras oncogenes (43). The SiHa human HPV+squamous cell carcinoma cell line and the THP-1 DC line were obtained from ATCC and cultured according to the manufacturer's specifications. Healthy-donor PBMCs were obtained from the NIH Blood Bank (NCT00001846), processed, and stored as previously described (44).

To determine which HPV vaccine construct demonstrated the best antitumor efficacy in vivo, NSG-β2m−/− mice bearing HPV+ cervical cancer (SiHa) were reconstituted with human HLA-A24⁺ PBMCs from a healthy donor on day 7 (FIG. 14C, blue arrow), and treated twice with weekly injections of phosphate buffered saline (PBS) control (100 μL), empty vector (1×10⁹ vector particles [VP]), or 3 HPV vaccine constructs (nos. 1, 3, or 4) (1×10⁹ VP) given s.c., as indicated by red arrows (FIG. 14C). Based on the tumor volumes for the duration of the study, mice treated with the AdV-HPV16/18 HPV vaccine construct (no. 4) displayed the lowest tumor growth rate compared with all other treatment groups (FIG. 14C). The tumor volumes of mice treated with AdV-HPV16/18 (no. 4) were significantly lower than PBS control-treated (P<0.0001) and empty vector control-treated (P<0.01) mice. At the end of study, mice treated with AdV-HPV16/18 (no. 4) also displayed the lowest tumor weights (P<0.05 versus PBS and empty vector controls, FIG. 14D). An additional experiment was performed in the same human cervical cancer model—this time, vaccinating mice 4 times on a weekly basis with AdV-HPV16/18 (no. 4). NSG-β2m−/− mice treated with AdV-HPV16/18 (no. 4) displayed significantly lower tumor volumes (P<0.01) compared with PBS control-treated mice (FIG. 14E). There was a clear trend of decreased tumor weights at the end of study with AdV-HPV16/18 treatment; however, there were no significant differences between treatment groups in this experiment (FIG. 14F). Tumors from these humanized mice were dissected at day 31 and stained by IHC. Sections from the center of the tumors showed that s.c. HPV vaccine treatment greatly upregulated CD8⁺ T cells in the tumor microenvironment (TME) compared with empty vector-treated mice (FIG. 14G).

The foregoing data suggests that the AdV-HPV16/18 HPV vaccine construct (no. 4) was the most efficacious both in vitro by IFN-γ ELISA, and in vivo, by decreased mouse tumor volumes and tumor weights at the end of the study, and it promoted increased CD8⁺ T cell infiltration.

HPV vaccine construct AdV-HPV16/18 (no. 4) was therefore used in all subsequent experiments and is referred to as AdV-HPV16/18. The higher immune and therapeutic responses seen with the AdV-HPV16/18 vaccine construct in these studies is likely due to the molecular design of the antigen, and the position of the antigen components according to Precigen's bioinformatics analyses, which created optimum presentation.

### Example 6: Treatment with the Recombinant Gorilla Adenovirus Vaccine AdV-HPV16/18 Resulted in Decreased Tumor Volumes and Induction of Antigen-Specific T Cells in the TC-1 Syngeneic Mouse Model

The syngeneic mouse model consisting of HPV⁺ TC-1 tumors has been used extensively to study various HPV vaccines and was used in further studies. C57BL/6 mice bearing s.c. TC-1 HPV16⁺ tumors were treated with 3 weekly injections of PBS control or AdV-HPV16/18 (1×10⁹ VP, s.c.). After the second injection, AdV-HPV16/18-treated mice had significantly lower tumor volumes compared with PBS control-treated mice (P<0.0001, FIG. 15A), and also displayed significantly lower tumor weights at the end of study compared with PBS control-treated mice (P<0.05, FIG. 15B).

To evaluate T cell infiltration into the TME, flow cytometry of single-cell suspensions of tumors was performed. Following AdV-HPV16/18 treatment, there was an increase in tumors in both CD8⁺ T cell infiltration (44.6% of all live cells versus 2.09% in control treated mice) and CD4⁺ T cell infiltration (16.7% versus 1.31% of all live cells) (FIG. 15C). The infiltration of multifunctional (IFN-γ⁺GzmB⁺) CD8⁺ T cells, which have previously been shown to be cytolytic, into tumor was also greatly increased following AdV-HPV16/18 treatment (48.4%) compared with PBS control (0%) (FIG. 15D). To evaluate T cell antigen specificity generated by AdV-HPV16/18 treatment, splenocytes were isolated from mice from both treatment groups for analysis using IFN-γ ELIspot. Overlapping 15-mer peptides from the HPV16 E6 protein were used as the target antigen. Only mice treated with AdV-HPV16/18 developed significant antigen-specific responses against the HPV16 E6 peptides (P<0.01) (FIG. 15E).

When comparing all AdV-HPV16/18-treated mice (n=8) to PBS-treated controls (n=8), total CD8⁺ T cells per mg of tumor increased greatly, to a 33:1 ratio. This was also seen for total CD4⁺ T cells per mg of tumor, which increased to a ratio of 13:1. As has previously been observed in multiple clinical trials, we found that the number of Tregs increased when the total CD4⁺ T cell population increased. Furthermore, the multifunctional (IFN-γ⁺GzmB⁺) CD8⁺ T cells were detected at 40-fold higher levels in AdV-HPV16/18-treated mice, and the activated (PD-1⁺) CD8⁺ T cells were seen at 70-fold higher levels; these 2 subsets were below the detection level in control mice. It should be noted (FIG. 15B) that there was a clear distinction in 2 groups of mice in antitumor effects after vaccination. Mice were thus designated as “Best Responders” or “Nonresponders” depending on their tumor weights at the end of study in an attempt to define whether differences exist between these 2 groups in terms of tumor immune infiltrate. Best Responder mice (nos. 10 and 13, FIG. 15B), displayed tumor weights below the AdV-HPV16/18 group median, and Nonresponder mice (nos. 11 and 14) had tumor weights above the group median. The multifunctional (IFN-γ⁺GzmB⁺) and activated (PD-1⁺) CD8⁺ T cells increased the most when comparing Best Responder mice to Nonresponders after AdV-HPV16/18 treatment (Table 8, middle). Multifunctional CD8⁺ T cells were detected at 7-fold higher levels in Best Responders, and activated CD8⁺ T cells were detected at 6-fold higher levels. Total CD8⁺ T cells increased to a 5:1 ratio.

When comparing AdV-HPV16/18-treated mice (Best Responders only) with PBS controls (Table 8, bottom), the previously stated trends continued to expand, with approximately 50 times more CD8⁺ T cells per mg of tumor in Best Responder mice compared with controls, and even higher ratios for multifunctional and activated CD8⁺ T cells (Table 8, bottom).

TABLE 8
Effects of AdV-HPV16/18 treatment on tumor immune cell infiltration in C57BL/6
mice bearing TC-1 (HPV 16⁺) tumors

| All ADV-HPV16/18 treated mice (n = 8) | | | | | | | |
|---|---|---|---|---|---|---|---|
| versus PBS control (n = 8) | | | | | | | |
| | ADV- | PBS | | | | | |
| Cell subsets | HPV16/18 | control | ADV-HPV16/18 /PBS ratio | | | | |
| CD8 | 10 | 0.3 | 33:1 | | | | |
| CD4 | 4 | 0.3 | 13:1 | | | | |
| Macrophages | 53 | 10 | 5:1 | | | | |
| Tregs | 12 | 1 | 12:1 | | | | |
| CD8*IFN-γ*GzmB* | 4 | <0.1 | >40:1 | | | | |
| CD8*PD-1* | 7 | <0.1 | >70:1 | | | | |
| ADV-HPV16/18 treated mice, Best Responders | | | | | | | Best |
| versus Nonresponders | | | | | | | Responder/ |
| Cell subsets | Best Responders | | | Nonresponders | | | Nonresponder |
| Mouse ID | #10 | #13 | Average | #11 | #14 | Average | ratio |
| CD8 | 22 | 10 | 16 | 4 | 3 | 3.5 | 5:1 |
| CD4 | 8 | 5 | 6 | 2 | 2 | 2 | 3:1 |
| Macrophages | 72 | 75 | 74 | 36 | 30 | 33 | 2:1 |
| Tregs | 26 | 11 | 19 | 3 | 6 | 5 | 4:1 |
| CD8*IFN-γ*GzmB* | 10 | 3 | 7 | 1 | 1 | 1 | 7:1 |
| CD8*PD-1* | 17 | 7 | 12 | 3 | 1 | 2 | 6:1 |

| ADV-HPV16/18-treated mice, | | | |
|---|---|---|---|
| Best Responders | | PBS | |
| Cell subsets | Best Responders | control | Best Responder/PBS ratio |
| CD8 | 16 | 0.3 | 53:1 |
| CD4 | 6 | 0.3 | 20:1 |
| Macrophages | 74 | 10 | 7:1 |
| Tregs | 19 | 1 | 19:1 |
| CD8*IFN-y*GzmB* | 7 | <0.1 | >70:1 |
| CD8*PD-1* | 12 | <0.1 | >120:1 |

Flow cytometry of immune cell infiltration into the tumors of C57BL/6 mice bearing TC-1 tumors and treated 3 times with weekly s.c. injections of PBS control (100 μl) or ADV-HPV16/18 (1 × 10⁹ VP) starting on day 4 after tumor implantation (FIG. 3B). The numbers of each immune cell subset per mg of tumor are shown and compared between all ADV-HPV16/18-treated mice (n = 8) versus PBS control-treated mice (n = 8) (top); ADV-HPV16/18-treatedmice. Best Responders (n = 2) versus Nonresponders (n = 2)(middle): and ADV-HPV16/18-treated Best Responders (n = 2) versus PBS control-treated mice (n = 8)(bottom).

The single-positive CD8⁺ IFN-γ⁺ T cells were increased in Best Responder AdV-HPV16/18-treated mice compared with Nonresponder AdV-HPV16/18-treated mice to a ratio of 8:1, and CD4⁺ IFN-γ⁺ T cells were increased to a ratio of 5:1. When comparing these immune cell subsets between all AdV-HPV16/18-treated mice (n=8) and PBS controls (n=8), they were undetectable in controls but were detected at low levels in vaccinated mice. Additional studies com-paring empty vector control to AdV-HPV16/18 treatment showed similar results when comparing the Best Responder and Nonresponder mice, with the highest increases observed for the activated (PD-1⁺) and multifunctional (IFN-γ⁺GzmB⁺) CD8⁺ T cells.

There was a trend toward lower numbers of myeloid-derived suppressor cells (MDSCs) (CD11b⁺Gr1⁺) in mice responding to AdV-HPV16/18 treatment. In contrast, the MDSC population trended higher for the entire AdV-HPV16/18-treated group compared with control-treated mice. In an additional experiment performed using complementary MDSC markers for granulocytic (CD11b⁺Ly6G⁺) and monocytic MDSCs (CD11b⁺Ly6C⁺), there was a trend of increase in both subsets following vaccine treatment. In the same study, markers specific for M1 and M2 macrophages were used (CD11b⁺F4/80⁺M− HC-II⁺ and CD11b⁺F4/80⁺CD206⁺, respectively). As seen in Supplemental FIG. 1C, the number of M1 tumor-associated macrophages (TAM) trended higher in AdV-HPV16/18-treated mice, and the number of M2 TAM trended lower after AdV-HPV16/18 treatment. There was a trend toward an increased M1/M2 ratio after treatment with AdV-HPV16/18 (Supplemental FIG. 1E, P=0.053). A decreased M1/M2 ratio has previously been shown to correlate with reduced survival and a poor response to chemoradiation in patients with advanced cervical cancer. Thus, the increased M1/M2 ratio observed after AdV-HPV16/18 vaccination may indicate a more beneficial TME.

Additional experiments were performed using the empty GC46 vector as a negative control in C57BL/6 mice bearing s.c. TC-1 HPV16⁺ tumors. Mice were vaccinated with 2 injections of empty vector control or AdV-HPV16/18, and AdV-HPV16/18-treated mice displayed significantly lower tumor weights at the end of study compared with empty vector control (P<0.05, FIG. 16, A-C). At the end of the studies, flow cytometry of single-cell suspensions of tumor tissue was performed, and CD8⁺ and CD4⁺ T cell subsets were evaluated. There were no changes in total CD8⁺ T cells in the tumor (FIG. 16D), but there were significant increases in multifunctional CD8⁺ T cells (CD8⁺ IFN-γ⁺GzmB⁺, FIG. 16E) and CD8⁺ T cells with a proliferative capacity (CD8⁺Ki67⁺, FIG. 16F) following AdV-HPV16/18 treatment compared with empty vector control. Certain CD4⁺ T cell subsets showed moderate increases after AdV-HPV16/18 treatment, although not to the same extent as the CD8 subsets.

Antigen-specific T cell responses were evaluated both in the tumor and in splenocytes. To evaluate antigen-specific responses in the TME of mice treated with AdV-HPV16/18, CD45+ tumor-infiltrating lymphocytes (TILs) were isolated and stimulated overnight in vitro with a mix of HPV16 E6/E7 15-mer peptides. There were not enough TILs available to assay additional HPV antigens. Flow cytometry analysis was performed and showed that AdV-HPV16/18 treatment significantly increased the amount of total CD8⁺ T cells (P<0.05), IFN-γ-producing CD8⁺ T cells (P<0.01), and IFN-γ⁺GranzymeB⁺ (IFN-γ⁺GzmB⁺) multifunctional CD8⁺ T cells (P<0.05) in the TME compared with empty vector treatment (FIGS. 17A-C). Similar results were seen with CD4⁺ T cells, but to a lesser degree (FIG. 17D-F). Antigen-specific T cell responses in the TME were further evaluated using a commercially avail-able mouse HPV16 E7 tetramer (RAHYNIVTF (SEQ ID NO: 287)). Double staining of TILs showed a significant increase (P<0.05) in CD8⁺ tetramer+ T cells in the TME of AdV-HPV16/18-treated mice compared with empty vector-treated controls (FIG. 17G).

As a measure of peripheral immune responses, splenocytes were isolated from empty vector control and AdV-HPV16/18-treated mice for analysis using IFN-γ ELIspot. Overlapping 15-mer peptides from the HPV 16 and 18 E6/E7 proteins were used as target antigens. AdV-HPV16/18-treated mice developed significant antigen-specific responses against the HPV16 E6 peptides (P<0.01, FIG. 17H) and the HPV18 E6 peptides (P<0.01, FIG. 17I). Some variations were observed within the groups, which is expected since the results are based on the stimulation of 2.5×10⁵ splenocytes per well. Little reactivity to the HPV 16 and 18 E7 peptides was seen using the ELIspot assay (Supplemental FIG. 2). Only AdV-HPV16/18-treated mice displayed IFN-γ production after stimulation. Altogether, tumor growth control, and the development of HPV antigen-specific responses induced by AdV-HPV16/18, were reproducible in multiple independent experiments using the TC-1 HPV16⁺ syngeneic mouse model.

### Example 7: AdV-HPV16/18 T Cell Depletion and Toxicology Studies

To evaluate the contribution of CD4⁺ and CD8⁺ T cells to the observed antitumor effects of vaccination with AdV-HPV16/18, depletion studies were performed. C57BL/6 mice were depleted of the CD4⁺ or CD8⁺ T cell populations using commercially obtained depleting antibodies prior to instillation of TC-1 tumors. Treatments with AdV-HPV16/18 were started on day 7. At the end of the study, AdV-HPV16/18-treated mice depleted of CD4⁺ T cells displayed larger tumor volumes than non-depleted AdV-HPV16/18-treated mice (Supplemental FIG. 3, A and C). Similar trends were also observed in CD8-depleted mice; however, this was to a lesser degree. Thus, both CD4⁺ and CD8⁺ T cells have an impact on the antitumor efficacy of the AdV-HPV16/18 vaccine.

A toxicology study was performed to further evaluate the safety and general tolerability of repeat s.c. administration of AdV-HPV16/18. AdV-HPV16/18 (1×10¹⁰ VP) s.c. was administered once a week for 3 weeks; no significant treatment-related effects in the C57BL/6 mouse were observed based on body weights; full pathological report, including organ weights and histopathology; or full blood and chemical laboratory analyses. Body weights and organ weights from AdV-HPV16/18-treated mice and control mice were in range of one another and not significantly different. Histopathology analysis was normal between AdV-HPV16/18-treated mice and controls.

### Example 8: Combination of AdV-HPV16/18 and Bintrafusp Alfa Induces Tumor Growth Inhibition in Pre-Clinical Models of HPV-Positive Malignancy

The specific combination of AdV-HPV16/18 and bintrafusp alfa was tested in wild-type C57BL/6 mice bearing TC-1 tumors (FIG. 18). The addition of bintrafusp alfa further enhanced tumor growth inhibition observed with AdV-HPV16/18 alone.

### Example 9: Treatment of Human Subjects with Recurrent or Metastatic Human Papillomavirus-Associated Cancers with Replication-Incompetent Gorilla Adenovirus Targeting HPV 16/18 in Combination with Bintrafusp Alfa

Human subjects with recurrent or metastatic human papillomavirus-associated cancers were treated with replication-incompetent gorilla adenovirus targeting HPV 16/18 in combination with bintrafusp alfa (BA).

### Preparation

For the 1×10¹¹ PU Dose, a single vial of AdV-HPV16/18 was thawed. After thawing, 0.4 ml of AdV-HPV16/18 was aseptically withdrawn from the vial and transferred into an empty sterile glass vial using an appropriately sized sterile syringe. 1.6 ml of thawed Final Formulation Buffer (FFB; manufactured by ABL) was aseptically withdrawn from a diluent vial using an appropriately sized sterile syringe and transferred into the glass vial into which the 0.4 ml of AdV-HPV16/18 had been transferred in the previous step. The contents were gently swirled without shaking to thoroughly mix. 1.0 ml of the diluted AdV-HPV16/18 was aseptically withdrawn using an appropriately sized sterile syringe and administered for the initial dose of 1×10¹¹ PU.

The top dose of 5×10¹¹ PU was obtained by aseptically withdrawing 1.0 ml of AdV-HPV16/18 from a single thawed vial using an appropriately sized sterile syringe. One ml of the undiluted AdV-HPV16/18 was administered for the top dose of 5×10¹¹ PU.

EMD Serono Research and Development Institute supplied bintrafusp alfa (BA). BA was provided in a sterile liquid formulation, packaged in USP/Ph Eur type I 50R vials with a concentration of 10 mg/mL. These vials, filled with a drug product solution allowing an extractable volume of 60 mL (600 mg/60 mL), were closed with rubber stoppers in a serum format, compliant with USP and Ph Eur standards, and sealed with an aluminum crimp seal closure. Each single-usc vial contained 600 mg of BA, formulated as 10 mg/mL of active ingredient, 6% (w/v) Trehalose, 40 mM NaCl, 5 mM Methionine, 0.05% (w/v) Tween 20, and 10 mM L-Histidine at pH 5.5. The liquid formulation was diluted directly with 0.9% sodium chloride solution for injection, and the anticipated delivery volumes were expected to be no more than 250 mL. The verified concentration range in the infusion solution was 0.16 mg/mL to 9.6 mg/mL. BA was stored and maintained at 2° C. to 8° C. until use.

### Administration.

Patients with pretreated advanced HPV-associated malignancies received AdV-HPV16/18 (5×10¹¹ PU, subcutaneous administration) on days 1, 15, and 29 (D1, D15, and D29, respectively) for 3 administrations and 1200 mg BA as an intravenous infusion every two weeks. This was followed by administration of AdV-HPV16/18 (5×10¹¹ particle units) once every 4 weeks vaccine and 1200 mg BA once every two weeks.

Safety. To first assess the safety and feasibility of treatment with AdV-HPV16/18 alone or in combination with bintrafusp alfa, a sequential study of dose-escalating AdV-HPV16/18 (two dose levels) alone or in combination with a fixed-dose of BA was implemented. Participants with recurrent/metastatic HPV-positive malignancy received two dose levels (1×10¹¹ and 5×10¹¹ PU) of AdV-HPV16/18, followed by an evaluation of the RP2D dose of AdV-HPV16/18 in combination with 1200 mg (RP2D) of BA until progression or unacceptable toxicity. This design allowed for the determination of safety and tolerability of AdV-HPV16/18 alone and in combination with BA.

The study had two arms. Dose escalation followed the rules as follows: After completion of enrollment on DL1 of Arm 1A, enrollment to DL2 of Arm 1A proceeded if 0 out of 3 or 1 out of 6 participants in DL1 (1×10¹¹ PU) of Arm 1A experienced a DLT. After completion of enrollment on DL2 (5×10¹¹ PU) of Arm 1A, enrollment to Arm 1B proceeded if □ out of 3 or 1 out of 6 participants in DL2 of Arm 1A experienced a DLT. If 2 participants in a given dose level of Arm 1A or Arm 1B experienced a DLT, accrual to that arm was halted. See FIG. 19.

There was a 4-week DLT evaluation period for each dose level.

There was a 1-week delay between the first three participants treated on a given dose level.

Results. The below tables, respectively titled “Monotherapy” and “Combination Therapy,” illustrate the patient characteristics of the participants.

Monotherapy

| Patient Information | Arm 1A (n = 6) | |
|---|---|---|
| Median age (range) | 61 | (43-70) |
| Female, n (%) | 6 | (100) |
| Tumor Types, n (%) | | |
| Cervical | 3 | (50) |
| Anal | 2 | (33) |
| Vaginal | 1 | (17) |
| HPV status, n (%) | | |
| HPV16 | 3 | (50) |
| HPV18 | — | |
| Other | 2 | (33) |
| N/A | 1 | (17) |
| Prior systemic therapies (median, range) | 2.5 | (1-3) |
| Prior anti-PD-(L)1 | 6 | (100) |
| AdV-HPV16/18 duration (months) | 3 | (1.8-17.9) |
| (median, range) | | |

Combination Therapy

| Patient Information | Arm 1B (n = 11) | |
|---|---|---|
| Median age (range) | 61 | (54-80) |
| Female, n (%) | 3 | (27) |
| Tumor Types, n (%) | | |
| OPSCC | 7 | (64) |
| Cervical | 3 | (27) |
| Anal | 1 | (9) |
| HPV status, n (%) | | |
| HPV16 | 9 | (82) |
| HPV18 | 1 | (9) |
| Other | 1 | (9) |
| N/A | — | |
| Prior systemic therapies (median, range) | 1 | (1-4) |
| Prior anti-PD-(L)1 | 10 | (91) |
| AdV-HPV16/18 treatment duration (months) | 10 | (0.5-23.0) |
| (median, range) | | |

As seen in the table of treatment-related adverse events below. AdV-HPV16/18 alone and in combination was safe and well-tolerated.

| | Arm 1A (n = 6) | | | Arm 1B (n = 11) | | |
|---|---|---|---|---|---|---|
| Treatment-related | Grade 1-2 | | Grade 3-4 | Grade 1-2 | | Grade 3-4 |
| adverse events, n (%) | (all) | | (all) | (≥10%) | | (all) |
| Injection site reactions | 4 | (66.7) | 0 | 9 | (81.8) | 0 |
| Flu-like symptoms, | 3 | (50.0) | 0 | 6 | (54.5) | 0 |
| Fatigue | 2 | (33.3) | 0 | 3 | (27.3) | 0 |
| Rash, maculopapular | 1 | (16.7) | 0 | 3 | (27.3) | 0 |

| | | | | | |
|---|---|---|---|---|---|
| Epistaxis | 0 | 0 | 3 | (27.3)* | 0 |
| Headache | 0 | 0 | 3 | (27.3) | 0 |
| Keratoacanthoma | 0 | 0 | 3 | (27.3)* | 0 |
| Fever | 0 | 0 | 2 | (18.2) | 0 |
| Lymphocyte count, | 0 | 0 | 2 | (18.2)* | 0 |
| decreased | | | | | |
| Anemia | 0 | 0 | 2 | (18.2)* | 0 |
| Oral hemorrhage | 0 | 0 | 2 | (18.2)* | 0 |

| | | | | | |
|---|---|---|---|---|---|
| Duodenal Hemorrhage | 0 | 0 | 0 | 2 | (18.2)*†‡ |
| Pharyngeal mucositis | 0 | 0 | 0 | 1 | (9.1)* |

*Attributed to BA; † both patients concurrently receiving NSAIDs; ‡1 patient died following refusal of standard supportive medical management measures (blood transfusion).

FIG. 20 schematically summarizes patient responses to AdV-HPV16/18 monotherapy (Arm 1A) and combination therapy (AdV-HPV16/18+bintrafusp alfa, Arm 1B).

AdV-HPV16/18 combination therapy resulted in a 30% ORR in patients with pretreated HPV-associated cancers, naïve or resistant to checkpoint blockade, with observed duration of responses for up to 600 days.

| | AdV- | | | |
|---|---|---|---|---|
| | HPV16/18 | | | |
| | Monotherapy | AdV-HPV16/18 Combination Therapy | | |
| | All | All | Cervical | Head and Neck |
| | (n = 10) | (n = 10) | (n = 2) | (n = 7) |
| Disease Control Rate | 50% ( 3/6) | 40% ( 4/10) | 2/2 (100%) | 2/7 (28%) |
| (DCR) at first restaging | | | | |
| Objective Response | 0% ( 0/6) | 30% ( 3/10) | 2/2 (100%) | 1/7 (14%) |
| Rate (ORR) | | | | |

Best Response by Arm

| | Arm 1A | Arm 1B |
|---|---|---|
| Evaluable patients, n | 6 | 10 |
| Best response, n | | |
| CR | — | 1^{b} |
| PR | — | 2^{a,c} |
| SD | 4^{a} | 1 |
| PD | 2 | 6 |
| ORR, % (95% CI) | — | 30 (6.7-65.3) |

^{a}1 confirmed;
^{b}immune checkpoint blockade (ICB)-resistant;
^{c}1 ICB-resistant;
1 TCR treatment-resistant;
2 patients treated beyond progression without delayed response;
CI: confidence interval

Overall, AdV-HPV16/18 treatment in generated robust HPV-specific T-cell immune response in checkpoint inhibitor refractory patients, and post-vaccination 14/16 (88%) patients in the study developed T-cell responses to HPV-16 and/or HPV-18 in the peripheral blood.

| | | |
|---|---|---|
| HPV-specific T-cell Immune Responses | Arm 1A | Arm 1B |

| | | | | |
|---|---|---|---|---|
| HPV-16, n/N (%) | ⅚ | (83) | 7/10 | (70) |
| HPV-18, n/N (%) | ⅚ | (83) | ⅞ | (88) |
| HPV-18 and/or HPV-18 n/N (%) | 6/6 | (100) | 8/10 | (80) |

N: number of patients tested

Further, as seen in FIG. 21A and FIG. 21B, repeat administrations of AdV-HPV16/18 did not lead to substantial increase in neutralizing antibody response. Neutralizing antibody data validate the ability to repeat administer Precigen's AdenoVerse therapies.

FIGS. 22A and 22B show a cervical cancer patient in the study who had a complete response to combination therapy (AdV-HPV16/18, 5×10¹¹ vp/dose, +bintrafusp alfa) starting at approximately 6 months following the start of treatment.

### Example 10: A Study of AdV-HPV16/18+Bintrafusp Alfa in HPV-Positive Oropharyngeal and Sinonasal Squamous Carcinoma Cancer Patients

Design. This study focuses on the use of AdV-HPV16/18 in treating newly diagnosed stage I (T1, T2 N1)/II/III HPV-positive oropharyngeal cancer and newly diagnosed stage II/III/IVA/IVB HPV-positive sinonasal squamous carcinoma (SNSCC). The study evaluates AdV-HPV16/18 as a monotherapy or in combination with bintrafusp alfa as a neoadjuvant or induction therapy. Where AdV-HPV16/18 is evaluated alone (Arm 2A) as a neoadjuvant or induction therapy, 20 participants with HPV-positive oropharyngeal cancer and 2 participants with HPV-SNSCC participants are enrolled. FIG. 23.

After completion of induction immunotherapy, participants are referred back to their home institution for definitive standard of care therapy. At outside institutions, standard of care treatment are performed and chosen solely by outside providers. NCI investigators have no role in treatments received at outside institutions. Although positive HPV testing is not required prior to enrolling, HPV testing is offered as an exploratory endpoint, and participants testing negative for HPV after enrolling, or whose HPV status cannot be confirmed, may be replaced with other participants for the primary efficacy analysis. Participants testing negative for HPV after enrolling or whose HPV status cannot be confirmed may continue to receive treatment on study.

### Example 11: Correlative Analyses from AdV-HPV16/18 Vaccine In-Human Study Using Peripheral Blood from 10 Patients with HPV16+, HPV18+, or HPV45+ Disease to Identify Immune Correlates Associated with Clinical Response

As previously described in Example 9, patients with pretreated advanced HPV-associated malignancies received AdV-HPV16/18 (5×10¹¹ particle units, subcutaneous administration) q2 weeks for 3 administrations and biweekly 1200 mg bintrafusp alfa (BA) followed by q4 weeks vaccine and biweekly BA (NCT04432597), with an overall response rate of 30%. Based on this study, correlative analyses was performed using peripheral blood from 10 patients with HPV16+ (n=8), HPV18+ (n=1), or HPV45+ (n=1) disease to identify immune correlates associated with clinical response.

Peripheral HPV-specific T cell responses, HPV circulating tumor DNA (ctDNA) from plasma, serum cytokines and soluble factors, and peripheral immune cell subsets were assessed to evaluate changes induced with treatment and peripheral correlates associated with clinical activity. Patients with complete response (CR, n=1), partial response (PR, n=2; 1 confirmed), or stable disease (SD, n=1) (n=4 total) were compared to patients with progressive disease (PD, n=6).

Eighty percent (8/10) of patients developed either HPV16 or HPV18-specific T-cell responses after repeat administration of AdV-HPV16/18 with BA. Eight of 10 patients had detectable HPV16 or 18 ctDNA at baseline and changes in ctDNA load with treatment generally correlated with outcomes. See FIG. 29. Early transient increases in IL-8, TNFα, and IFNγ occurred, with greater increases in IL-8 at 2 weeks and TNFα at 4 weeks in patients with PD compared to CR/PR/SD. See FIG. 27. Lower serum concentrations of IL-8 prior to start of treatment were found to be associated with prolonged survival. See FIG. 28.

The patient who achieved a durable CR had HPV45+ disease, and had a) a 50% increase in CD8+ T cell frequency at 2 weeks, b) increases in frequencies of CD8+naïve T cells expressing Ki67+ and central and effector memory T cells expressing 4-1BB, PD-1, or TIM-3, and c) considerable increases in HPV18-specific T cell responses through 484 days after treatment start, indicating durable antigen-specific T cell activity. See FIG. 25. An increase in both activated CD8+ memory T cells and mature NK cells as early as 2 weeks after the first dose of AdV-HPV16/18 was only seen in the patient who achieved CR. See FIG. 26.

The majority of patients developed HPV16/18 specific T-cell responses after repeat administration of AdV-HPV16/18 in combination with BA. See FIG. 24. Transient increases in IL-8 and TNFα 2-4 weeks after start of treatment were associated with worse outcomes, suggesting that early changes in cytokines may reflect disease course. See FIG. 27. Furthermore, the early expansion of CD8+ T cell subsets in the patient developing a CR with HPV45+ disease after receiving a vaccine targeting HPV16 and HPV18 epitopes suggest a broadening and boosting of T cell responses that enabled potent anti-tumor activity. A high degree of homology between HPV18 and HPV45 may have contributed to the expansion of HPV18 specific T cells in the patient with CR. These data highlight the importance of assessing peripheral blood in immunotherapy trials to reveal important immunological phenomena.

### Example 12: A Randomized, Study of AdV-HPV16/18+CKI in Recurrent/Metastatic Cervical Cancer Patients

Patient Eligibility. Patients are ≥18 years old, have recurrent/metastatic (r/m) cervical cancer that has failed at least 1 prior line in the metastatic setting, and have ≥1 measurable (RECIST 1.1) lesion.

Design. A Fleming 2-stage design is implemented for each arm. The null hypothesis that the true response rate (ORR) is 5% is tested against a one-sided alternative of 25%. In the first stage, 8 patients are accrued for a possible 23 total per arm. This design yields a type I error rate of one-sided alpha=0.025 and power=80% when the true response rate is 25%.

The primary objective is to determine the objective response rate following treatment with AdV-HPV16/18 (5×10¹¹ PU) in combination with CKI (cyclin-dependent kinase inhibitors) or CKI alone in patients with r/m cervical cancer. The secondary objective includes evaluating the safety and tolerability of AdV-HPV16/18 in combination with CKI or CKI alone in patients with recurrent or metastatic cervical cancer, determining progression-free survival (PFS) and overall survival (OS) following treatment with AdV-HPV16/18 when given in combination with CKI or CKI alone in patients with r/m cervical cancer, determining time to response and duration of response following treatment with AdV-HPV16/18, when given in combination with CKI in patients with r/m cervical cancer, and evaluating the potential for shedding of the AdV-HPV16/18 adenoviral vector following subcutaneous administration. The exploratory objective includes identifying alterations in immune profile and T cell markers and correlate to clinical response; evaluate the relationship between PD-L1 expression and response to the treatment with AdV-HPV16/18 when given in combination with CKI; exploratory biomarkers of mechanisms of safety and efficacy.

### Example 13: Constructing Variants of HPV Antigen Design 4—Conservative Substitutions within the Ankyrin-Like Repeat Domains

### Variant A

The design of Variant A (SEQ ID NO: 245) is based on Design 4, utilizing the same 35 key immunogenic peptides, as listed in Table 5 of Example 2. However, Variant A is constructed to have conservative substitutions within the ankyrin-like repeat domains of SEQ ID NO: 243. In designing Variant A, amino acids chemically similar (e.g., amino acids with similar charge, size, and/or hydrophobicity) to those composing the ankyrin-like repeat domains of Design 4 are selected for substitution within the ankyrin-like repeat domains and the antigen design is monitored, including, without limitation, by assessing antigenic changes, cross-reactivity, antibody response, T cell responses, and/or HPV viral fitness, to ensure that the therapeutic efficacy of Variant A does not substantially deviate from the therapeutic efficacy of Design 4.

### Variants B-E

Antigen constructs of SEQ ID NOs: 246-249 are prepared using the method described above with respect to Variant A.

### Example 14: Constructing Variants of HPV Antigen Design 4—Shuffling HPV and Agonist Peptides

### Variant F

The design of Variant F is based on Design 4, utilizing the same 35 key immunogenic peptides as Design 4. However, Variant F (SEQ ID NO: 250) comprises immunogenic peptides in a different sequential order (from amino- to carboxy-terminus) relative to Design 4. In constructing Variant F, shuffling of the immunogenic peptides is carefully monitored using a bioinformatics workflow similar to that described in Example 1, including, without limitation, assessing antigenic changes, cross-reactivity, antibody response, T cell responses, and/or HPV viral fitness, to ensure that the therapeutic efficacy of Variant F does not substantially deviate from the therapeutic efficacy of Design 4.

### Variants G-Q

Antigen constructs of SEQ ID NOs: 251-261 are prepared using the method described above with respect to Variant F.

### Example 15: Constructing Variants of HPV Antigen Design 4-Fewer HPV and Agonist Peptides

### Variants R, U, V, and W

The designs of Variant R (SEQ ID NO: 262), Variant U (SEQ ID NO: 265), Variant V (SEQ ID NO: 266), and Variant W (SEQ ID NO: 267) are similar to Design 4, but contain fewer HPV and agonist peptides. Namely, instead of having 35 immunogenic peptides like Design 4, Variant R has 34 immunogenic peptides, Variant U has 33 immunogenic peptides, Variant V has 32 immunogenic peptides, and Variant W has 31 immunogenic peptides. In constructing these variants, the antigen design is carefully monitored using a bioinformatics workflow similar to that described in Example 1, including, without limitation, assessing antigenic changes, cross-reactivity, antibody response, T cell responses, and/or HPV viral fitness, to ensure that the therapeutic efficacy of the variants does not substantially deviate from the therapeutic efficacy of Design 4.

### Example 16: Constructing Variants of HPV Antigen Design 4-Additional HPV and Agonist Peptides

### Variants S, X, Y, and Z

The designs of Variant S (SEQ ID NO: 263), Variant X (SEQ ID NO: 268), Variant Y (SEQ ID NO: 269), and Variant Z (SEQ ID NO: 270) are similar to Design 4, but contain more HPV and agonist peptides. Namely, instead of having 35 immunogenic peptides like Design 4, Variant S has 36 immunogenic peptides, Variant X has 37 immunogenic peptides, Variant Y has 38 immunogenic peptides, and Variant Z has 39 immunogenic peptides. In constructing these variants, the antigen design is carefully monitored using a bioinformatics workflow similar to that described in Example 1, including, without limitation, assessing antigenic changes, cross-reactivity, antibody response, T cell responses, and/or HPV viral fitness, to ensure that the therapeutic efficacy of the variants does not substantially deviate from the therapeutic efficacy of Design 4.

### Example 17: Constructing Variants of HPV Antigen Design 4—Combination Variants T, AA, BB, CC, and DD

The designs of Variant T (SEQ ID NO: 264), Variant AA (SEQ ID NO: 271), Variant BB (SEQ ID NO: 272), Variant CC (SEQ ID NO: 273), and Variant DD (SEQ ID NO: 274) are prepared using a combination of at least two of the methodologies set forth in Examples 13-16.

Variant T (SEQ ID NO: 264) is similar to Design 4 (SEQ ID NO: 243), but is constructed by shuffling around the 35 key immunogenic peptides listed in Table 5 of Example 2. Accordingly, while Variant T comprises the same 35 peptides as Design 4, the order of the peptides in Variant T differs from that of Design 4 from amino- to carboxy-terminus. Furthermore, relative to Design 4, Variant T comprises numerous randomized conservative amino acid substitutions within the ankyrin-like repeat domains.

Variant AA (SEQ ID NO: 271) is similar to Design 4 but has 36, as opposed to 35, immunogenic peptides and has conservative amino acid substitutions within the ankyrin-like repeat domains.

Variant BB (SEQ ID NO: 272) is similar to Design 4 but has 34, as opposed to 35, immunogenic peptides and has conservative amino acid substitutions within the ankyrin-like repeat domains.

Variant CC (SEQ ID NO: 273) is similar to Design 4, but has 34, as opposed to 35, immunogenic peptides and has conservative amino acid substitutions within the ankyrin-like repeat domains. Further, the immunogenic peptides it shares with Design 4 are shuffled in a different order.

Variant DD (SEQ ID NO: 274) is similar to Design 4, but has 36, as opposed to 35, immunogenic peptides and has conservative amino acid substitutions within the ankyrin-like repeat domains. Further, the immunogenic peptides it shares with Design 4 are shuffled in a different order.

### Example 18: Design of a Recombinant Gorilla Adenovirus Vaccine Expressing Variant(s) of HPV 16/18 Antigen Design 4

### Recombinant Gorilla Adenovirus Vaccine Expressing Variant A

Similar to AdV-HPV16/18 (i.e., HPV 16/18 antigen design 4-see Example 2), Variant A of AdV-HPV16/18—namely, a DNA sequence encoding a polypeptide sequence comprising SEQ ID NO: 245—is constructed on Precigen Inc.'s nonhuman primate adenoviral vector platform GC46. The GC46 gorilla adenovector is identified and isolated from nonhuman primate sources, and multiple genes (E1, E3, and E4) are deleted to prevent viral replication so the variant is not replication competent. The CTL peptide sequences comprising Variant A are constitutively expressed under control of a cytomegalovirus (CMV) promoter and are grafted to a human ankyrin repeat protein scaffold enabling protein linker sequences embedded between the peptides wherein this scaffold retains it tertiary structure displaying the HPV epitopes. Designed shuffling of the peptides prevents any reformation of E6 and E7 oncogenic protein potential and HPV protein viral function. The vaccine or the GC46 empty vector control is given as an s.c. injection of 1×10⁹ VP once per week.

### Recombinant Gorilla Adenovirus Vaccine Expressing Variants B-DD

Gorilla adenoviral vectors comprising the antigen constructs of each of Variants B-DD (SEQ ID NOs: 246-274) are prepared using the method described above with respect to the recombinant gorilla adenovirus vector vaccine expressing Variant A.

### Example 19: Correlative Analyses from AdV-HPV16/18 Vaccine Variants Using Peripheral Blood from Patients with HPV16+, HPV18+, or HPV45+ Disease to Identify Immune Correlates Associated with Clinical Response

### Variant A

Similar to the methodology set forth in Example 11, patients with pretreated advanced HPV-associated malignancies receive the Variant A vaccine (5×10¹¹ particle units, subcutaneous administration) q2 weeks for 3 administrations and biweekly 1200 mg bintrafusp alfa (BA) followed by q4 weeks vaccine and biweekly BA. Correlative analyses are performed using peripheral blood from patients with HPV16+, HPV18+, or HPV45+ disease to identify immune correlates associated with clinical response. Peripheral HPV-specific T cell responses, HPV circulating tumor DNA (ctDNA) from plasma, serum cytokines and soluble factors, and peripheral immune cell subsets are assessed to evaluate changes induced with treatment and peripheral correlates associated with clinical activity. The majority of patients develop Variant A-specific T-cell responses after repeat administration of Variant A in combination with BA.

### Variants B-T

Patients with pretreated advanced HPV-associated malignancies are treated with each of the Gorilla adenoviral vectors of Example 18 using the method described above with respect to the recombinant gorilla adenovirus vector vaccine expressing Variant A and similar results are observed.

### Example 20: Treatment with Variants of AdV-HPV16/18 Result in Decreased Tumor Volumes and Induction of Antigen-Specific T Cells in TC-1 Syngeneic Mouse Models

Treatment with Recombinant Gorilla Adenovirus Vaccine Expressing Variant A

C57BL/6 mice bearing s.c. TC-1 HPV16⁺ tumors are treated with 3 weekly injections of PBS control or Variant A (1×10⁹ VP, s.c.). After the second injection, Variant A-treated mice have significantly lower tumor volumes compared with PBS control-treated mice and also display significantly lower tumor weights at the end of study compared with PBS control-treated mice.

To evaluate T cell infiltration into the TME, flow cytometry of single-cell suspensions of tumors are performed. Following treatment with Variant A, there is an increase in tumors in both CD8⁺ T cell infiltration and CD4⁺ T cell infiltration. The infiltration of multifunctional (IFN-γ⁺GzmB⁺) CD8⁺ T cells, which have previously been shown to be cytolytic, into tumor are also greatly increased following treatment with Variant A compared with PBS control.

To evaluate T cell antigen specificity generated by treatment with Variant A, splenocytes are isolated from mice from both treatment groups for analysis using IFN-γ ELIspot. Overlapping 15-mer peptides from the HPV16 E6 protein are used as the target antigen. Only mice treated with Variant A develop significant antigen-specific responses against the HPV16 E6 peptides.

Total CD8⁺ T cells per mg of tumor increase greatly when all Variant A-treated mice are compared to PBS-treated controls. Total CD4⁺ T cells per mg of tumor also increase. The number of Tregs increase when the total CD4⁺ T cell population increase. Furthermore, the multifunctional (IFN-γ⁺GzmB⁺) CD8⁺ T cells and the activated (PD-1⁺) CD8⁺ T cells are detected at higher levels in Variant A-treated mice.

In mice responding to Variant A treatment, there is a trend toward lower numbers of myeloid-derived suppressor cells (MDSCs) (CD11b+Gr1⁺). In contrast, the MDSC population trend higher for the entire Variant A-treated group compared with control-treated mice.

In an additional experiment that is performed using complementary MDSC markers for granulocytic (CD11b+Ly6G+) and monocytic MDSCs (CD11b+Ly6C+), there is a trend of increase in both subsets following vaccine treatment. In the same study, markers specific for M1 and M2 macrophages are used (CD11b⁺F4/80⁺M− HC-II+ and CD11b⁺F4/80⁺CD206⁺, respectively). The number of M1 tumor-associated macrophages (TAM) trend higher in Variant A-treated mice, and the number of M2 TAM trend lower after Variant A treatment. There is a trend toward an increased M1/M2 ratio after treatment with Variant A, which likely indicates a more beneficial TME.

Additional experiments are performed using the empty GC46 vector as a negative control in C57BL/6 mice bearing s.c. TC-1 HPV16⁺ tumors. Mice are vaccinated with 2 injections of empty vector control or Variant A, and Variant A-treated mice display significantly lower tumor weights at the end of study compared with empty vector control. At the end of the studies, flow cytometry of single-cell suspensions of tumor tissue is performed, and CD8⁺ and CD4⁺ T cell subsets are evaluated. There are no changes in total CD8⁺ T cells in the tumor, but there are significant increases in multifunctional CD8⁺ T cells (CD8⁺ IFN-γ⁺GzmB⁺) and CD8⁺ T cells with a proliferative capacity (CD8⁺Ki67⁺, FIG. 16F) following Variant A treatment compared with empty vector control. Certain CD4⁺ T cell subsets will show moderate increases after Variant A treatment, although not to the same extent as the CD8 subsets.

Antigen-specific T cell responses are evaluated both in the tumor and in splenocytes. To evaluate antigen-specific responses in the TME of mice treated with Variant A, CD45⁺ tumor-infiltrating lymphocytes (TILs) are isolated and stimulated overnight in vitro with a mix of HPV16 E6/E7 15-mer peptides. Flow cytometry analysis is performed and shows that Variant A treatment significantly increases the amount of total CD8⁺ T cells, IFN-γ-producing CD8⁺ T cells, and IFN-γ⁺GranzymeB⁺ (IFN-γ⁺GzmB⁺) multifunctional CD8⁺ T cells in the TME compared with empty vector treatment. Similar results are seen with CD4⁺ T cells, but to a lesser degree. Antigen-specific T cell responses in the TME are further evaluated using a commercially available mouse HPV16 E7 tetramer (RAHYNIVTF (SEQ ID NO: 287)). Double staining of TILs show a significant increase in CD8⁺ tetramer+ T cells in the TME of Variant A-treated mice compared with empty vector-treated controls.

To measure of peripheral immune responses, splenocytes are isolated from empty vector control and Variant A-treated mice for analysis using IFN-γ ELIspot. Overlapping 15-mer peptides from the HPV 16 and 18 E6/E7 proteins are used as target antigens. Variant A-treated mice develop significant antigen-specific responses against the HPV16 E6 peptides and the HPV18 E6 peptides. Some variations are observed within the groups since the results are based on the stimulation of 2.5×10⁵ splenocytes per well. Little reactivity to the HPV 16 and 18 E7 peptides is observed using the ELIspot assay. Only Variant A-treated mice display IFN-γ production after stimulation. Altogether, tumor growth control and the development of HPV antigen-specific responses induced by Variant A are reproducible in multiple independent experiments using the TC-1 HPV16⁺ syngencic mouse model.

Treatment with Recombinant Gorilla Adenovirus Vaccine Expressing Variants B-T

C57BL/6 mice bearing s.c. TC-1 HPV16⁺ tumors are treated using each of the Gorilla adenoviral vectors of Example 18 using the method described above with respect to the recombinant gorilla adenovirus vector vaccine expressing Variant A and similar results are observed.

### Example 21: A Proposed Randomized, Open-Label Study of any One of Variants A-T+CKI in Recurrent/Metastatic Cervical Cancer Patients

Cervical cancer patients are treated with a combination of each of the gorilla adenoviral vectors of Example 18 and CKI using the method of Example 12.

### Example 22: Treating Cervical Cancer with AdV-HPV16/18 in Combination with IL-12

Patients with cervical cancer attributed to HPV 16/18 are treated with AdV-HPV16/18 (or a variant disclosed herein) as set forth in any of the foregoing Examples. Following vaccination with AdV-HPV16/18, patients receive IL-12 treatment via a proprietary payload, expression cassette and single chain IL12p70, built into a GC46 Gorilla adenovector—either separate from that of AdV-HPV16/18 or the same—that has the capability to deliver dose dependent production of bioactive IL12. The IL-12 Gorilla adenovector is administered either systemically or directly at the tumor site (i.e., intratumorally) to enhance the activation and proliferation of immune cells, particularly T cells. AdV-HPV16 primes the immune system, and IL-12 amplifies the cytotoxic potential of T cells, macrophages, and natural killer cells. This synergistic effect leads to a more potent and targeted attack on HPV16/18-infected cells. IL-12 helps create an inflammatory microenvironment within the tumor, making it less hospitable for cancer cells. This further supports the AdV-HPV16/18-induced immune response. Following treatment with AdV-HPV16/18 in combination with IL-12, cervical cancer patients demonstrate a significant reduction in HPV16/18 tumor burden and an expansion of peripheral HPV-specific T cell response. Overall, this combination therapy elicits robust and potent HPV16/18 antigen spreading and treats antigenically heterogeneous HPV16/18 tumors.

### Example 23: Treating Oropharyngeal Cancer with AdV-HPV16/18 in Combination with IL-12

Patients with oropharyngeal cancer attributed to HPV16/18 are treated with AdV-HPV 16/18 (or a variant disclosed herein) as set forth in any of the foregoing Examples. Following vaccination with AdV-HPV16/18, patients receive IL-12 treatment via a proprietary payload, expression cassette and single chain IL12p70, built into a GC46 Gorilla adenovector—either separate from that of AdV-HPV16/18 or the same—that has the capability to deliver dose dependent production of bioactive IL12. The IL-12 Gorilla adenovector is administered either systemically or directly at the tumor site to enhance the activation and proliferation of immune cells, particularly T cells. AdV-HPV16 primes the immune system, and IL-12 amplifies the cytotoxic potential of T cells, macrophages, and natural killer cells. This synergistic effect leads to a more potent and targeted attack on HPV16/18-infected cells. IL-12 helps create an inflammatory microenvironment within the tumor, making it less hospitable for cancer cells. This further supports the AdV-HPV16/18-induced immune response. Following treatment with AdV-HPV16/18 in combination with IL-12, oropharyngeal cancer patients demonstrate a significant reduction in HPV16/18 tumor burden and an expansion of peripheral HPV-specific T cell response. Overall, this combination therapy elicits robust and potent HPV16/18 antigen spreading, and treats antigenically heterogeneous HPV16/18 tumors.

### Example 24: Assessing Antigenicity of AdV-HPV16/18+IL-12 in Wild Type C57B16 Mice

AdV-HPV16/18 (or a variant disclosed herein) is administered to wild type C57B 16 mice in combination with a proprietary payload, expression cassette and single chain IL12p70, built into a GC46 Gorilla adenovector—either separate from that of AdV-HPV16/18 or the same—that has the capability to deliver dose dependent production of bioactive IL12. The treated mice demonstrate enhanced HPV-specific T cell activation and IL12 production.

### Example 25: Efficacy Study of AdV-HPV16/18+IL-12 in a SiHa Subcutaneous Xenograft Model of Cervical Cancer in Humanized Mice

Similar to the methodology set forth in Example 5, NSG-2m−/− mice bearing HPV+ cervical cancer (SiHa) are treated with AdV-HPV16/18 (or a variant disclosed herein) in combination with a proprietary payload, expression cassette and single chain IL12p70, built into a GC46 Gorilla adenovector—either separate from that of AdV-HPV16/18 or the same—that has the capability to deliver dose dependent production of bioactive IL12. Based on the tumor volumes for the duration of the study, mice treated with the AdV-HPV16/18+IL-12 display significantly lower tumor growth rates, lower tumor volumes, and lower tumor weights, compared to the PBS control and empty vector groups. Further, AdV-HPV16/18+IL-12 treatment greatly upregulates CD8+ T cells in the tumor microenvironment compared with empty vector-treated mice.

### Example 26. Clinical Trial for ADV-HPV16/18 Alone or in Combination with Anti-PD-L1/TGF-Beta Trap (M7824) for the Treatment of HPV-Associated Malignancies

A clinical trial is currently underway to investigate the safety, efficacy, and immunogenicity of ADV-HPV16/18 (HPV vaccine) alone or in combination with an anti-PD-L1/TGF-Beta Trap, M7824 (MSB0011359C), for the treatment of HPV-associated malignancies. The trial consists of two phases: Phase I for participants with recurrent/metastatic HPV-positive cancer and Phase II for participants with newly diagnosed stage I (T1, T2 N1)/II/III p16-positive oropharyngeal cancer and patients with newly diagnosed operable stage II/III/IVA/IVB/HPV-positive sinonasal squamous cell cancer (HPV-SNSCC).

In Phase I, the primary objective is to determine the safety and recommended phase II dose (RP2D) of ADV-HPV16/18 alone or in combination with M7824 administered at an RP2D of 1,200 mg every 2 weeks (q2w). A 3+3 dose escalation design will be used, evaluating ADV-HPV16/18 at two dose levels (1×10\hat{ }11 and 5×10\hat{ }11 viral particle (VP) units) as monotherapy, followed by a third dose level evaluating the RP2D dose of ADV-HPV16/18 in combination with 1200 mg (RP2D) of M7824. The combination of ADV-HPV16/18 at RP2D with 1200 mg of M7824 will be expanded to a total of 10 evaluable participants to gauge the preliminary efficacy of the combination in participants with advanced disease. There will be a 4-week dose-limiting toxicity (DLT) evaluation period for each dose level, and it is expected that up to 22 participants may enroll.

In Phase II, the primary objective for participants with newly diagnosed p16-positive oropharyngeal cancer is to determine if HPV vaccine alone (Arm 2A) is able to result in a ≥2-fold increase in cluster of differentiation 3 (CD3+) tumor-infiltrating T cells post-treatment compared with pre-treatment. Participants will receive neoadjuvant/induction immunotherapy at the National Institutes of Health (NIH) Clinical Center and then be referred back to their home institution for definitive standard of care therapy. It is expected that up to 20 participants may enroll. For participants with newly diagnosed stage II/III/IVA/IVB HPV-SNSCC, enrollment and treatment will occur similarly to participants with p16-positive oropharyngeal cancer for exploratory correlates to advise possible future trials, with up to 2 participants enrolling in this group.

The clinical trial enrolls men or women aged 18 years or older. In Phase I, participants must have cytologically or histologically confirmed locally advanced (not amenable to potentially curative local therapies) or metastatic HPV-associated malignancies, including cervical cancers, p16-positive oropharyngeal cancers, anal cancers, vulvar, vaginal, penile, and squamous cell rectal cancers, or other locally advanced or metastatic solid tumors (e.g., lung, esophagus) that are known to be HPV-positive. Prior first-line systemic therapy is required for Phase I participants.

### Example 27: A Randomized, Open-Label, Two-Arm, Phase 2 Study to Evaluate Efficacy and Safety of ADV-HPV16/18 in Combination with Pembrolizumab Versus Pembrolizumab in Patients with Recurrent or Metastatic Cervical Cancer

A randomized, two-arm, Phase 2 study is being conducted to evaluate the efficacy and safety of ADV-HPV16/18 (HPV vaccine) in combination with pembrolizumab versus pembrolizumab alone in patients with recurrent or metastatic cervical cancer who are pembrolizumab-resistant. The study aims to enroll 46 participants who meet all eligibility criteria and consent to participate. Patients will be randomized 1:1 to receive either a combination of ADV-HPV16/18 plus pembrolizumab or pembrolizumab alone.

In the experimental arm, patients will receive ADV-HPV16/18 at a dose of 5×10\hat{ }11 PU (subcutaneous injection) every 3 weeks for three administrations, followed by administrations every 6 weeks. Pembrolizumab will be administered concurrently as an intravenous infusion (400 mg) every 6 weeks. In the active comparator arm, patients will receive an intravenous infusion of pembrolizumab (400 mg) administered every 6 weeks.

The primary outcome measure is the Objective Response Rate (ORR) following treatment with ADV-HPV16/18 in combination with pembrolizumab or pembrolizumab alone. ORR will be calculated as the combination of subjects achieving a complete response or a partial response per RECIST v1.1 and presented with a 2-sided 95% confidence interval.

Secondary outcome measures include: a) Safety of ADV-HPV16/18 in combination with pembrolizumab or pembrolizumab alone, assessed through the capture of Treatment Emergent Adverse Events (TEAEs) and their severity using the Common Terminology Criteria for Adverse Events (CTCAE) v 5.0 scale; b) Progression-Free Survival (PFS) and Overall Survival (OS) following treatment, summarized using the Kaplan-Meier Product Limit estimator along with the corresponding two-sided 95% confidence intervals; c) Best Overall Responses (BOR) and Disease Control Rate (DCR) per RECIST v1.1, summarized using descriptive statistics; d) Time to Response (TTR) and Duration of Responses (DOR) following treatment, summarized using descriptive statistics; and e) Vector shedding following subcutaneous administration of ADV-HPV16/18, evaluated by collecting samples before and at specific intervals after treatment to assess the presence of adenoviral vector.

### Example 28: Phase II Trial of Immunotherapeutic HPV Vaccine ADV-HPV16/18 with Pembrolizumab Before Standard Treatment in Subjects with Newly Diagnosed HPV-Associated Oropharyngeal Cancer

### BACKGROUND

Oropharyngeal cancer (OPC) represents 2.9% of US and 0.5% of global cancer cases, corresponding to 2% of all US cancer deaths. Approximately 70% of US OPC cases are driven by human papillomavirus (HPV). Standard-of-care concurrent chemoradiotherapy for locally advanced disease, while highly active with long-term control and/or cure rates of >60%, is frequently associated with significant early and late toxicities, including impaired speech and swallow (requiring enteral nutrition in up to 20% of patients), dry mouth, dysgeusia, pharyngeal-laryngeal toxicity (25% of patients), and oral cavity toxicity (47% of patients). Therefore, attempts to de-escalate therapy to reduce toxicity while maintaining efficacy are highly desirable.

Although OPC is known to be sensitive to immune checkpoint blockade (ICB), preclinical and clinical data indicate that intact antigen detection and presentation machinery, including lymph node tissue, are required for an effective antitumor immune response. These tissues are necessarily ablated by both surgical and radiotherapeutic techniques. Thus, a neoadjuvant immunotherapy approach in OPC is rational and may both enable improved long-term disease control and facilitate treatment de-escalation and reduced morbidity.

Pembrolizumab, a humanized PD-1 blocking monoclonal antibody, has proven activity in advanced OPC and other head and neck cancers. ADV-HPV16/18 is a therapeutic HPV vaccine targeting the E6 and E7 oncoproteins of HPV 16/18 using a gorilla adenoviral vector. Preclinical and clinical data have demonstrated the generation of an HPV E6/E7-specific T cell response with a tumor response in a humanized murine model, along with increased infiltration of the tumor microenvironment by activated CD4/CD8 cells.

### Study Design:

This is a single-site, single-arm, Phase 2 study of induction Pembrolizumab and ADV-HPV16/18 in up to 20 evaluable p16+ OPC patients planned for definitive therapy (surgery or chemoRT). The primary endpoint is to determine if ADV-HPV16/18 combined with pembrolizumab in participants with p16+ OPC can result in a ≥2-fold increase in CD3+ tumor-infiltrating T cells post-treatment compared with pre-treatment levels.

Secondary objectives include determining if ADV-HPV16/18plus pembrolizumab results in significantly prolonged survival compared to the historically estimated 80% 3-year survival for p16+ OPC, assessing the 3-year overall survival and relapse-free survival rates for ADV-HPV16/18 plus pembrolizumab as neoadjuvant/induction therapy before definitive standard of care therapy in p16+ OPC participants, evaluating the safety of ADV-HPV16/18 plus pembrolizumab in p16+ OPC participants, and determining if the rate of ≥2-fold increase in tumor-infiltrating lymphocytes is significantly higher with ADV-HPV16/18 in combination with pembrolizumab compared to ADV-HPV16/18 alone (based on a previous trial at the CIO).

Exploratory objectives include determining the overall response rate (ORR) per RECIST v1.1 in subjects receiving ADV-HPV16/18 plus pembrolizumab, conducting exploratory immunologic studies to understand and improve the administered treatment (including immune cell types, clonality, subsets and their function; HPV antigen response and anti-vector antibodies; soluble factors, cytokines, chemokines, antibodies, tumor-associated antigens and immune markers), evaluating HPV status and subtype and specific immune response to ADV-HPV16/18, and assessing changes in salivary HPV DNA and salivary soluble factors.

Key inclusion criteria include newly diagnosed, pathologically confirmed Stage I-III, p16+ oropharyngeal squamous cell carcinoma planned for definitive therapy (either surgery or chemoradiotherapy), measurable disease by RECIST 1.1, age ≥18 years, ECOG≤2, and adequate hematologic, renal, and hepatic function. Patients serologically positive for HIV, HBV, or HCV are eligible if certain criteria are met. Participants must be willing to undergo two research biopsies and provide informed consent.

Key exclusion criteria include prior investigational drug, live vaccine, chemotherapy, immunotherapy, or radiotherapy exposure within 4 weeks prior to first drug administration (with some exceptions), major surgery within 28 days prior to first drug administration (with some exceptions), active/current pregnancy, active autoimmune disease with the potential to deteriorate in response to an immunostimulatory agent (with some exceptions), systemic glucocorticoids at greater than physiologic doses or other immunosuppressive agents (with some exceptions), a separate current or prior malignancy whose treatment has the potential to interfere with the safety or efficacy of the HPV WOOT regimen, prior allogeneic solid organ/tissue transplant, SpO2≤92% R/A at screening, and uncontrolled intercurrent illness deemed capable of interfering with study requirements, compliance, or planned assessments.

Assessments include screening clinical assessment, examination, blood tests; cross-sectional imaging (either PET-CT or CT-Neck/Thorax) at week 1 and CT-Neck at week 3; ENT evaluation, biopsy, saliva samples (for HPV DNA), and research bloods at week 1 and week 3; safety visit at week 4; and post-therapy follow-up (in person or remote) at month 3 and month 6.

### Example 29: A Randomized Phase II Study of Neoadjuvant Chemotherapy (NAC) with or without ADV-HPV16/18 for the Treatment of Newly Diagnosed HPV-Associated Oropharyngeal Squamous Cell Carcinoma

### BACKGROUND

Human papilloma virus (HPV)-associated oropharyngeal squamous cell carcinoma (OPSCC) is among the most common HPV-associated malignancies, with an increasing incidence. Although standard anti-cancer treatments, such as surgery followed by adjuvant post-operative radiation therapy (PORT) or concurrent chemoradiation (CRT), result in excellent oncologic control with >80% 5-year recurrence-free survival, these treatments often lead to radiation-associated long-term toxicity, including tissue fibrosis resulting in long-term swallow dysfunction and poor quality of life (QOL).

Neoadjuvant chemotherapy (NAC) followed by surgery has decades of real-world data, with clinical-to-pathologic downstaging or pathologic complete response (pCR) being observed in most patients, >90% 5-year survival, and complete avoidance of radiation treatment in >95% of patients. However, the rate of pCR, clinical-to-pathologic downstaging, and functional outcomes after NAC followed by surgery have not been studied in a formal, prospective clinical study.

A pilot correlative study of NAC with docetaxel and cisplatin (DC) in patients with newly diagnosed HPV-associated OPSCC conducted at the NIH (18DC0051) revealed induction of HPV-specific T cell immunity that associates with clinical outcome. ADV-HPV16/18 (HPV vaccine), designed to enhance HPV 16/18-specific T-cell responses, has been studied at the NIH Clinical Center (NCT04432597) for its safety and efficacy in patients with newly diagnosed HPV-associated OPSCC. Pre-clinical data suggest that chemotherapy can remodel the tumor microenvironment and enhance immunotherapy, indicating that the combination of DC and ADV-HPV16/18 may enhance anti-tumor immunity and the rate of pCR beyond that observed with DC alone.

### Objective:

The objective of this study is to determine the rate of pCR with NAC (DC) alone or in combination with ADV-HPV16/18 (DCP) in participants with newly diagnosed HPV-associated OPSCC.

### Eligibility Criteria:

Participants must have pathologically confirmed newly diagnosed surgically resectable stage I or II HPV-positive oropharyngeal squamous cell carcinoma, be 18 years of age or older, have an Eastern Cooperative Oncology Group (ECOG) performance status≤2, and have adequate organ function.

### Design:

Participants diagnosed in the community with newly diagnosed HPV-associated OPSCC will be referred to the NIH Clinical Center for neoadjuvant treatment. They will be randomized to receive either DC (Arm 1) or DCP (Arm 2) in the neoadjuvant setting. DC consists of three cycles of intravenous cisplatin plus docetaxel, administered every 21 days. ADV-HPV16/18 is 4 doses of subcutaneous vaccination administered on Day-7 of Cycle 1, and Day 11 of Cycles 1, 2, and 3. Participants will be stratified at registration for stage (I or II).

Pre- and post-treatment Positron Emission Tomography (PET)/Computed Tomography (CT) and measurement of circulating cell-free HPV DNA will be performed. Participants will return to the community to receive standard-of-care surgery, and the need for pathology-indicated, risk-stratified PORT will be determined per standard of care. Pathologic responses and follow-up to assess swallow function, QOL, hearing function, and recurrence-free survival will take place at the NIH Clinical Center.

### Example 30: Cross-Coverage Analysis of AdV-HPV16/18

Although based on HPV-16 and HPV-18 T cell immunogenic epitopes, AdV-HPV16/18 also showed good response in a patient with an HPV-45 induced tumor (see, e.g., Example 11, above). In light of this, the cross-coverage potential of AdV-HPV16/18 was analyzed in six common potentially carcinogenic HPV genotypes, including HPV-45, with bioinformatics methods. AdV-HPV16/18 epitope sequences were aligned to protein sequences of the carcinogenic HPV genotypes HPV-31, HPV-33, HPV-35, HPV-45, HPV-52, and HPV-58, using Protein BLAST in a local BLAST installation, optimized for short input sequences. AdV-HPV16/18 epitopes that yielded BLAST alignments with greater than 75% overall identity to any of the HPV proteins (calculated from percent coverage x % identity of the covered sequence) were considered for further evaluation.

MHC-I and MHC-II epitopes were predicted for the above six HPV genotypes (HPV-31, HPV-33, HPV-35, HPV-45, HPV-52, and HPV-58) in the 27 most common HLA backgrounds of each MHC class, using IEDB MHC-I and MHC-II binding prediction tools with the recommended predictions methods and epitope lengths of 8-10 AA for MHC-I, and 12-15 AA and 19-22 AA for MHC-II. AdV-HPV16/18 epitopes and IEDB predicted epitopes were compared using an in-house developed script. IEDB-predicted epitopes were considered covered by AdV-HPV16/18 if their sequence was completely contained without mismatch in HPV peptides of the respective HPV genotype that aligned to AdV-HPV16/18 epitopes with greater than 75% overall identity. Exemplary alignments between HPV45 and HPV16 and/or HPV18 epitopes are shown in FIG. 30-32.

Cross-covered MHC-I epitopes were predicted in all analyzed HPV genotypes with varying coverage by ADV-HPV16/18 epitopes. The highest cross-coverage was found in HPV-45, with about twice as many cross-covering ADV-HPV16/18 epitopes as the next extensively covered genotype. Cross-covered MHC-II epitopes were only predicted for HPV-45 and HPV-58. No MHC-II epitopes meeting the criteria of cross-coverage by AdV-HPV16/18 peptides were identified for genotypes 31, 33, 35, or 52. With the selected cross-coverage criteria, both perfect and imperfect sequence matches between AdV-HPV16/18 epitopes and predicted epitopes in the six HPV genotypes were identified. Most cross-covered predicted epitopes were imperfect matches. Exemplary predicted MCH-I epitopes and comparison with AdV-HPV16/18 epitopes are in Table 9A and all analyzed predicted MCH-I epitopes and comparison with AdV-HPV16/18 epitopes are in Table 9B.

TABLE 9A
Comparison of AdV-HPV16/18 epitopes with predicted HPV-45 MHC-I epitopes

| | | | HPV45 | HPV-45 epitope | SEQ | | |
|---|---|---|---|---|---|---|---|
| AdV-HPV16/18 | best | best | protein | aligned to AdV- | ID | Perfect | Mismatch |
| Epitope | score | rank | origin | HPV16/18 | NO: | match | position |
| HPV16-E6 | 0.493835 | 0.2 | E6 | VYqFAFkDL | 327 | no | 3, 7 |
| peptide 5 | | | | | | | |
| HPV16-E6 | 0.756432 | 0.1 | E6 | KLPdLCTEL | 135 | no | 4 |
| peptide 7 | | | | | | | |
| HPV16-E6 | 0.263645 | 0.55 | E6 | -LPdLCTEL | 328 | No | 3 |
| peptide 7 | | | | | | | |
| HPV16-E6 | 0.365002 | 0.36 | E6 | FAFKDLCI-- | 329 | no | 4 |
| peptide 8 | | | | | | | |
| HPV16-E6 | 0.260644 | 0.55 | E6 | FAFKDLCIV- | 330 | no | 4 |
| peptide 8 | | | | | | | |
| HPV16-E6 | 0.615168 | 0.16 | E6 | FAFKDLCIVY | 331 | | 4 |
| peptide 8 | | | | | | | |
| HPV16-E6 | 0.532406 | 0.15 | E6 | -AFKDLCIVY | 332 | no | 3 |
| peptide 8 | | | | | | | |
| HPV18-E6 | 0.187294 | 0.76 | E6 | KITNTeLY-- | 333 | no | 2, 6 |
| peptide 1 | | | | | | | |
| HPV18-E6 | 0.360535 | 10.55 | E6 | -iTNTeLYNL | 334 | no | 1, 5 |
| peptide 1 | | | | | | | |
| HPV18-E6 | 0.756432 | 0.1 | E6 | KLPDLCTEL | 135 | yes | - |
| peptide 5 | | | | | | | |
| HPV18-E6 | 0.263645 | 10.55 | E6 | -LPDLCTEL | 328 | yes | - |
| peptide 5 | | | | | | | |
| HPV18-E7 | 0.539952 | 0.13 | E7 | -DLRtlQQL------ | 335 | no | 4, 5 |
| peptide 1 | | | | | | | |
| HPV18-E7 | 0.238316 | 0.31 | E7 | -DLRtlQQLF----- | 336 | no | 4, 5 |
| peptide 1 | | | | | | | |
| HPV18-E7 | 0.289645 | 0.66 | E7 | -----IQQLFLsTL- | 337 | no | 1, 7 |
| peptide 1 | | | | | | | |
| HPV18-E7 | 0.89616 | 0.02 | E7 | QLFLsTLSF- | 338 | no | 5 |
| peptide 3 | | | | | | | |
| HPV18-E7 | 0.300799 | 0.49 | E7 | QLFLsTLSFV | 339 | no | 5 |
| peptide 3 | | | | | | | |
| HPV18-E7 | 0.139018 | 0.59 | E7 | -LFLsTLSF- | 340 | no | 4 |
| peptide 3 | | | | | | | |
| HPV18-E7 | 0.307591 | 0.49 | E7 | --FLsTLSFV | 341 | no | 3 |
| peptide 3 | | | | | | | |
| HPV18-E7 | 0.139018 | 0.59 | E7 | LFLsTLSF------- | 340 | No | 4 |
| peptide 4 | | | | | | | |
| HPV18-E7 | 0.307591 | 0.49 | E7 | -FLsTLSFV------ | 341 | no | 3 |
| peptide 4 | | | | | | | |
| HPV18-E7 | 0.670982 | 0.33 | E7 | --LsTLSFVCPW--- | 342 | no | 2 |
| peptide 4 | | | | | | | |
| HPV18-E7 | 0.881711 | 0.11 | E7 | ---STLSFVCPW--- | 343 | no | 1 |
| peptide 4 | | | | | | | |
| HPV18-E7 | 0.974852 | 0.02 | E7 | TLQeIVLHL | 344 | No | 4 |
| peptide 5 | | | | | | | |
| HPV18-E7 | 0.06243 | 1 | E7 | --EENDEaDGV--------- | 345 | No | 6 |
| peptide | | | | | | | |
| 6 | | | | | | | |
| HPV18-E7 | 0.141601 | 0.82 | E7 | ------EaDGVsHaq----- | 346 | no | 2, 6, 8 |
| peptide 6 | | | | | | | |
| HPV18-E7 | 0.231481 | 0.58 | E7 | ------EaDGVsHaqL---- | 347 | no | 2, 6, 8 |
| peptide 6 | | | | | | | |
| HPV18-E7 | 0.157757 | 0.88 | E7 | --------DGVsHaqL---- | 348 | no | 4, 6 |
| peptide 6 | | | | | | | |
| HPV18-E7 | 0.245522 | 0.97 | E7 | ----------GVsHaqLPAR- | 349 | no | 3, 5 |
| peptide 6 | | | | | | | |
| HPV18-E7 | 0.69893 | 0.13 | E7 | -----------VsHaqLPAR- | 350 | no | 2, 4 |
| peptide 6 | | | | | | | |
| HPV18-E7 | 0.313064 | 0.73 | E7 | -----------VsHaqLPARR | 351 | no | 2, 4 |
| peptide 6 | | | | | | | |
| HPV18-E7 | 0.163463 | 0.88 | E7 | ------------sHaqLPARR | 352 | no | 1, 3 |
| peptide 6 | | | | | | | |

MHC-I epitope predictions for all HPV-45 proteins were done with iedb.precigen.com/mhci/
Prediction method: IEDB recommended 2020.09 (NetMHCpan EL 4.1)
Epitope length range 8-10 AA, Epitope prediction in the HLA allele reference set (27 alleles)
HPV-45 epitopes are complete substrings of the corresponding aligning HPV-45 peptides
Percentile ranks <= 1 indicating strong binders are shown in bold type
best score: among 27 common HLA alleles; higher = better, maximum value = 1
best rank: among 27 common HLA alleles; best IC50 based percentile rank in background peptide set from SWISSPROT, lower = better, bolded numbers = best ranked.
Thresholds for MHC-I epitopes: A percentile rank is generated by comparing the peptide's IC50 against those of a set of random peptides from SWISSPROT database. A small numbered percentile rank indicates high affinity.
Column “HPV-45 epitope aligned to AdV-HPV16/18”: aligned to matching AdV-HPV16/18 epitope; lower case AA letters: mismatch

TABLE 9B
Comparison of PRGN-2009 epitopes with predicted HPV-45 MHC-I epitopes

| | | | | | | | | | | HPV-45 | | | |
|---|---|---|---|---|---|---|---|---|---|---|---|---|---|
| | | | | | | | | | | epitope | | HPV-45 | |
| | | | | | | | | | | in | | epitope | |
| AdV- | AdV- | SEQ | Aligning | SEQ | HPV-45 | SEQ | | | | AdV- | | aligned | |
| HPV16/18 | HPV16/18 | ID | HPV45 | ID | MHC-I | ID | best | best | HPV45 | HPV16/18 | | to AdV- | Perfect |
| Epitope | Sequence | NO: | sequence | NO: | epitope | NO: | score | rank | protein | epitope | Offset | HPV16/18 | match |
| HPV16- | VYDFAFRDL | 117 | VYQFAFK | 327 | VYQF | 353 | 9.70E−05 | 21 | E6 | VYQFAFK | 0 | VYqFAFk | no |
| E6 | | | DL | | AFKD | | | | | D- | | D- | |
| HPV16- | VYDFAFRDL | 117 | VYQFAFK | 327 | VYQF | 327 | 0.493835 | 0.2 | E6 | VYQFAFK | 0 | VYqFAFk | no |
| E6 | | | DL | | AFKD | | | | | DL | | DL | |
| | | | | | L | | | | | | | | |
| HPV16- | VYDFAFRDL | 117 | VYQFAFK | 327 | YQFA | 354 | 0.028872 | 3.2 | E6 | - | 1 | - | no |
| E6 | | | DL | | FKDL | | | | | YQFAFKD | | YqFAFkD | |
| | | | | | | | | | | L | | L | |
| HPV16- | KLPQLCTEL | 119 | KLPDLCTE | 135 | KLPDL | 355 | 0.000848 | 17 | E6 | KLPDLCT | 0 | KLPdLCT | no |
| E6 | | | L | | CTE | | | | | E− | | E− | |
| HPV16- | KLPQLCTEL | 119 | KLPDLCTE | 355 | KLPDL | 355 | 0.000848 | 17 | E6 | KLPDLCT | 0 | KLPdLCT | no |
| E6 | | | | | CTE | | | | | E− | | E− | |
| HPV16- | KLPQLCTEL | 119 | KLPDLCTE | 135 | KLPDL | 135 | 0.756432 | 0.1 | E6 | KLPDLCT | 0 | KLPdLCT | no |
| E6 | | | L | | CTEL | | | | | EL | | EL | |
| HPV16- | KLPQLCTEL | 119 | KLPDLCTE | 135 | LPDLC | 328 | 0.263645 | 0.55 | E6 | - | 1 | - | no |
| E6 | | | L | | TEL | | | | | LPDLCTE | | LPdLCTE | |
| | | | | | | | | | | L | | L | |
| HPV16- | FAFRDLCIV | 120 | FAFKDLCI | 331 | FAFK | 329 | 0.365002 | 0.36 | E6 | -FAFKDLC | 10 | -FAFkDLC | no |
| E6 | Y | | VY | | DLCI | | | | | I-- | | I-- | |
| HPV16- | FAFRDLCIV | 120 | FAFKDLCI | 331 | FAFK | 330 | 0.260644 | 0.55 | E6 | FAFKDLC | 0 | -FAFkDLC | no |
| E6 | Y | | VY | | DLCIV | | | | | IV- | | IV- | |
| HPV16- | FAFRDLCIV | 120 | FAFKDLCI | 331 | FAFK | 331 | 0.615168 | 0.16 | E6 | -FAFKDLC | 0 | -FAFkDLC | no |
| E6 | Y | | VY | | DLCIV | | | | | IVY | | IVY | |
| | | | | | Y | | | | | | | | |
| HPV16- | FAFRDLCIV | 120 | FAFKDLCI | 331 | AFKD | 356 | 0.00391 | 9.9 | E6 | -AFKDLCI | 1 | -AFkDLCI | no |
| E6 | Y | | VY | | LCIV | | | | | V- | | V- | |
| HPV16- | FAFRDLCIV | 120 | FAFKDLCI | 331 | AFKD | 332 | 0.532406 | 0.15 | E6 | - | 1 | - | no |
| E6 | Y | | VY | | LCIVY | | | | | AFKDLCI | | AFKDLCI | |
| | | | | | | | | | | VY | | VY | |
| HPV16- | FAFRDLCIV | 120 | FAFKDLCI | 331 | FKDL | 357 | 0.019934 | 3.6 | E6 | -- | 2 | -- | no |
| E6 | Y | | VY | | CIVY | | | | | FKDLCIV | | FkDLCIV | |
| | | | | | | | | | | Y | | Y | |
| HPV18- | KLTNTGLYN | 131 | KITNTELY | 358 | KITNT | 333 | 0.187294 | 0.76 | E6 | KITNTEL | 0 | KiTNTeL | no |
| E6 | L | | NL | | ELY | | | | | Y-- | | Y-- | |
| HPV18- | KLTNTGLYN | 131 | KITNTELY | 358 | KITNT | 379 | 0.001792 | 18 | E6 | KITNTEL | 0 | KITNTeL | no |
| E6 | L | | NL | | ELYN | | | | | YN- | | YN- | |
| HPV18- | KLTNTGLYN | 131 | KITNTELY | 358 | KITNT | 358 | 0.167411 | 1.5 | E6 | KITNTEL | 0 | KITNTeL | no |
| E6 | L | | NL | | ELYN | | | | | YNL | | YNL | |
| | | | | | L | | | | | | | | |
| HPV18- | KLTNTGLYN | 131 | KITNTELY | 358 | ITNTE | 380 | 0.000828 | 17 | E6 | -ITNTELY | 1 | - | no |
| E6 | L | | NL | | LYN | | | | | N- | | iTNTeLY | |
| | | | | | | | | | | | | N- | |
| HPV18- | KLTNTGLYN | 131 | KITNTELY | 358 | ITNTE | 334 | 0.360535 | 0.55 | E6 | -ITNTELY | 1 | iTNTeLY | no |
| E6 | L | | NL | | LYNL | | | | | NL | | NL | |
| HPV18- | KLTNTGLYN | 131 | KITNTELY | 358 | TNTEL | 381 | 0.004708 | 8.9 | E6 | -- | 2 | TNTeLYN | no |
| E6 | L | | NL | | YNL | | | | | TNTELYN | | L | |
| | | | | | | | | | | L | | | |
| HPV18- | KCIDFYSRI | 188 | KCIDFYSR | 188 | KCIDF | 359 | 0.047252 | 3.2 | E6 | KCIDFYS | 0 | KCIDFYS | yes |
| E6 | | | I | | YSR | | | | | R- | | R- | |
| HPV18- | KCIDFYSRI | 188 | KCIDFYSR | 188 | KCIDF | 188 | 0.064686 | 1.9 | E6 | KCIDFYS | 0 | KCIDFYS | yes |
| E6 | | | I | | YSRI | | | | | RI | | RI | |
| HPV18- | KCIDFYSRI | 188 | KCIDFYSR | 188 | CIDFY | 360 | 0.001764 | 14 | E6 | - | 1 | - | yes |
| E6 | | | I | | SRI | | | | | CIDFYSRI | | CIDFYSRIS | |
| HPV18- | NLLIRCLRC | 184 | NLLIRCLR | 184 | NLLIR | 361 | 0.112111 | 1.3 | E6 | NLLIRCL | 0 | NLLIRCL | yes |
| E6 | | | C | | CLR | | | | | R- | | R- | |
| HPV18- | NLLIRCLRC | 184 | NLLIRCLR | 184 | NLLIR | 184 | 0.03845 | 2.5 | E6 | NLLIRCL | 0 | NLLIRCL | yes |
| E6 | | | C | | CLRC | | | | | RC | | RC | |
| HPV18- | NLLIRCLRC | 184 | NLLIRCLR | 184 | LLIRC | 362 | 0.000737 | 17 | E6 | LLIRCLR | 1 | LLIRCLR | yes |
| E6 | | | C | | LRC | | | | | C | | C | |
| HPV18- | KLPDLCTEL | 135 | KLPDLCTE | 135 | KLPDL | 355 | 0.000848 | 17 | | E6KLPDLCT | 0 | KLPDLCT | yes |
| E6 | | | L | | CTE | | | | | E− | | E− | |
| HPV18- | KLPDLCTEL | 135 | KLPDLCTE | 355 | KLPDL | 355 | 0.000848 | 17 | | E6KLPDLCT | 0 | KLPDLCT | yes |
| E6 | | | | | CTE | | | | | E− | | E− | |
| HPV18- | KLPDLCTEL | 135 | KLPDLCTE | 35 | KLPDL | 135 | 0.756432 | 0.1 | E6 | KLPDLCT | 0 | KLPDLCT | yes |
| E6 | | | L | | CTEL | | | | | EL | | EL | |
| HPV18- | KLPDLCTEL | 135 | KLPDLCTE | 135 | LPDLC | 328 | 0.263645 | 0.55 | E6 | LPDLCTE | 1 | LPDLCTE | |
| E6 | | | L | | TEL | | | | | L | | L | yes |
| HPV18- | DDLRAFQQL | 139 | DDLRTLQ | 363 | DLRTL | 335 | 0.539952 | 0.13 | E7 | - | 1 | - | no |
| E7 | FLNTLS | | QLFLSTLS | | QQL | | | | | DLRTLQQ | | DLRtlQQ | |
| | | | | | | | | | | L------ | | L------ | |
| HPV18- | DDLRAFQQL | 139 | DDLRTLQ | 363 | DLRTL | 336 | 0.238316 | 0.31 | E7 | - | 1 | - | no |
| E7 | FLNTLS | | QLFLSTLS | | QQLF | | | | | DLRTLQQ | | DLRtlQQ | |
| | | | | | | | | | | LF----- | | LF----- | |
| HPV18- | DDLRAFQQL | 139 | DDLRTLQ | 363 | DLRTL | 364 | 0.061176 | 1.8 | E7 | - | 1 | - | no |
| E7 | FLNTLS | | QLFLSTLS | | QQLFL | | | | | DLRTLQQ | | DLRtlQQ | |
| | | | | | | | | | | LFL---- | | LFL---- | |
| HPV18- | DDLRAFQQL | 139 | DDLRTLQ | 363 | LRTLQ | 365 | 0.011462 | 5 | E7 | -- | 2 | -- | no |
| E7 | FLNTLS | | QLFLSTLS | | QLF | | | | | LRTLQQL | | LRtlQQLF | |
| | | | | | | | | | | F----- | | ----- | |
| HPV18- | DDLRAFQQL | 139 | DDLRTLQ | 363 | LRTLQ | 366 | 0.01347 | 4.6 | E7 | -- | 2 | -- | no |
| E7 | FLNTLS | | QLFLSTLS | | QLFL | | | | | LRTLQQL | | LRtlQQLF | |
| | | | | | | | | | | FL---- | | L---- | |
| HPV18- | DDLRAFQQL | 139 | DDLRTLQ | 363 | LRTLQ | 367 | 0.001742 | 15 | E7 | I-- | 2 | -- | no |
| E7 | FLNTLS | | QLFLSTLS | | QLFLS | | | | | LRTLQQL | | LRtlQQLF | |
| | | | | | | | | | | FLS--- | | Ls--- | |
| HPV18- | DDLRAFQQL | 139 | DDLRTLQ | 363 | RTLQ | 368 | 0.110848 | 1.6 | E7 | --- | 3 | --- | no |
| E7 | FLNTLS | | QLFLSTLS | | QLFL | | | | | RTLQQLF | | RtlQQLF | |
| | | | | | | | | | | L---- | | L---- | |
| HPV18- | DDLRAFQQL | 139 | DDLRTLQ | 363 | RTLQ | 369 | 0.095233 | 1.7 | E7 | --- | 3 | --- | no |
| E7 | FLNTLS | | QLFLSTLS | | QLFLS | | | | | RTLQQLF | | RtlQQLFL | |
| | | | | | | | | | | LS--- | | s--- | |
| HPV18- | DDLRAFQQL | 139 | DDLRTLQ | 363 | RTLQ | 370 | 0.023446 | 4.5 | E7 | --- | 3 | --- | no |
| E7 | FLNTLS | | QLFLSTLS | | QLFLS | | | | | RTLQQLF | | RtlQQLFL | |
| | | | | | T | | | | | LST-- | | sT-- | |
| HPV18- | DDLRAFQQL | 139 | DDLRTLQ | 363 | TLQQ | 371 | 0.000742 | 18 | E7 | ----TLQQ | 4 | ----tlQQ | no |
| E7 | FLNTLS | | QLFLSTLS | | LFLS | | | | | LFLS--- | | LFLs--- | |
| HPV18- | DDLRAFQQL | 139 | DDLRTLQ | 363 | TLQQ | 372 | 0.073201 | 1.7 | E7 | ----TLQQ | 4 | ----tlQQ | no |
| E7 | FLNTLS | | QLFLSTLS | | LFLST | | | | | LFLST-- | | LFLsT-- | |
| HPV18- | DDLRAFQQL | 139 | DDLRTLQ | 363 | TLQQ | 373 | 0.105004 | 1.4 | E7 | ---- | 4 | ---- | no |
| E7 | FLNTLS | | QLFLSTLS | | LFLST | | | | | TLQQLFL | | tlQQLFLs | |
| | | | | | L | | | | | STL- | | TL- | |
| HPV18- | DDLRAFQQL | 139 | DDLRTLQ | 363 | LQQLF | 374 | 0.000789 | 19 | E7 | -----LQQL | 5 | -----LQQL | no |
| E7 | FLNTLS | | QLFLSTLS | | LST | | | | | FLST-- | | FLsT-- | |
| HPV18- | DDLRAFQQL | 139 | DDLRTLQ | 363 | LQQLF | 337 | 0.289645 | 0.66 | E7 | LQQLFLS | 5 | -----LQQL | no |
| E7 | FLNTLS | | QLFLSTLS | | LSTL | | | | | TL- | | FLsTL- | |
| HPV18- | DDLRAFQQL | 139 | DDLRTLQ | 363 | LQQLF | 375 | 0.001563 | 17 | E7 | -----LQQL | 5 | -----LQQL | no |
| E7 | FLNTLS | | QLFLSTLS | | LSTLS | | | | | FLSTLS | | FLsTLS | |
| HPV18- | DDLRAFQQL | 139 | DDLRTLQ | 363 | QQLFL | 376 | 0.046092 | 2.6 | E7 | ------QQL | 6 | QQLFLsT | no |
| E7 | FLNTLS | | QLFLSTLS | | STL | | | | | FLSTL- | | L- | |
| HPV18- | DDLRAFQQL | 139 | DDLRTLQ | 363 | QQLFL | 377 | 0.004352 | 11 | E7 | ------- | 6 | ------- | no |
| E7 | FLNTLS | | QLFLSTLS | | STLS | | | | | QQLFLSTLS | | QQLFLsTLS | |
| HPV18- | DDLRAFQQL | 139 | DDLRTLQ | 363 | QLFLS | 378 | 0.00055 | 18 | E7 | | 7 | | no |
| E7 | FLNTLS | | QLFLSTLS | | TLS | | | | | QLFLSTLS | | QLFLsTLS | |
| HPV18- | FQQLFLNTL | 140 | QQLFLSTL | 376 | QQLFL | 376 | 0.046092 | 2.6 | E7 | - | 1 | QQLFLsT | no |
| E7 | | | | | STL | | | | | QQLFLST | | L | |
| | | | | | | | | | | L | | | |
| HPV18- | QLFLNTLSF | 141 | QLFLSTLS | 339 | QLFLS | 378 | 0.00055 | 18 | E7 | QLFLSTL | 0 | QLFLsTL | no |
| E7 | V | | FV | | TLS | | | | | S-- | | S-- | |
| HPV18- | QLFLNTLSF | 141 | QLFLSTLS | 339 | QLFLS | 338 | 0.89616 | 0.02 | E7 | QLFLSTL | 0 | QLFLsTL | no |
| E7 | V | | FV | | TLSF | | | | | SF- | | SF- | |
| HPV18- | QLFLNTLSF | 141 | QLFLSTLS | 339 | QLFLS | 339 | 0.300799 | 0.49 | E7 | QLFLSTL | 0 | QLFLsTL | no |
| E7 | V | | FV | | TLSFV | | | | | SFV | | SFV | |
| HPV18- | QLFLNTLSF | 141 | QLFLSTLS | 339 | LFLST | 340 | 0.139018 | 0.59 | E7 | - | 1 | LFLsTLSF | no |
| E7 | V | | FV | | LSF | | | | | LFLSTLSF | | | |
| | | | | | | | | | | - | | | |
| HPV18- | QLFLNTLSF | 141 | QLFLSTLS | 339 | LFLST | 382 | 0.022052 | 3.5 | E7 | LFLSTLSF | 1 | LFLsTLSF | no |
| E7 | V | | FV | | LSFV | | | | | V | | V | |
| HPV18- | QLFLNTLSF | 141 | QLFLSTLS | 339 | FLSTL | 341 | 0.307591 | 0.49 | E7 | -- | 2 | -- | no |
| E7 | V | | FV | | SFV | | | | | FLSTLSFV | | FLsTLSF | |
| | | | | | | | | | | | | V | |
| HPV18- | LFLNTLSFV | 142 | LFLSTLSF | 383 | LFLST | 340 | 0.139018 | 0.59 | E7 | LFLSTLSF | 0 | LFLsTLSF | no |
| E7 | CPWCAS | | VCPWCA | | LSF | | | | | | | | |
| HPV18- | LFLNTLSFV | 142 | LFLSTLSF | 383 | LFLST | 382 | 0.022052 | 3.5 | E7 | LFLSTLSF | 0 | LFLsTLSF | no |
| E7 | CPWCAS | | VCPWCA | | LSFV | | | | | V------ | | V------ | |
| HPV18- | LFLNTLSFV | 142 | LFLSTLSF | 383 | LFLST | 385 | 0.000522 | 17 | E7 | LFLSTLSF | 0 | LFLsTLSF | no |
| E7 | CPWCAS | | VCPWCA | | LSFVC | | | | | VC----- | | VC----- | |
| HPV18- | LFLNTLSFV | 142 | LFLSTLSF | 383 | FLSTL | 341 | 0.307591 | 0.49 | E7 | - | 1 | FLsTLSF | no |
| E7 | CPWCAS | | VCPWCA | | SFV | | | | | FLSTLSFV | | V------ | |
| HPV18- | LFLNTLSFV | 142 | LFLSTLSF | 383 | FLSTL | 386 | 0.049487 | 2.1 | E7 | FLSTLSFV | 1 | - | no |
| E7 | CPWCAS | | VCPWCA | | SFVC | | | | | C----- | | FLsTLSF | |
| | | | | | | | | | | | | VC----- | |
| HPV18- | LFLNTLSFV | 142 | LFLSTLSF | 383 | FLSTL | 387 | 0.002943 | 10 | E7 | - | 1 | - | no |
| E7 | CPWCAS | | VCPWCA | | SFVCP | | | | | FLSTLSFV | | FLsTLS | |
| | | | | | | | | | | CP---- | | FVCP---- | |
| HPV18- | LFLNTLSFV | 142 | LFLSTLSF | 383 | LSTLS | 388 | 0.001174 | 13 | E7 | -- | 2 | -- | no |
| E7 | CPWCAS | | VCPWCA | | FVC | | | | | LSTLSFV | | LsTLSF | |
| | | | | | | | | | | C----- | | VC----- | |
| HPV18- | LFLNTLSFV | 142 | LFLSTLSF | 383 | LSTLS | 389 | 0.001138 | 13 | E7 | -- | 2 | -- | no |
| E7 | CPWCAS | | VCPWCA | | FVCP | | | | | LSTLSFV | | LsTLSF | |
| | | | | | | | | | | CP---- | | VCP---- | |
| HPV18- | LFLNTLSFV | 142 | LFLSTLSF | 383 | LSTLS | 342 | 0.670982 | 0.33 | E7 | -- | 2 | -- | no |
| E7 | CPWCAS | | VCPWCA | | FVCP | | | | | LSTLSFV | | LsTLSFV | |
| | | | | | W | | | | | CPW--- | | CPW--- | |
| HPV18- | LFLNTLSFV | 142 | LFLSTLSF | 383 | STLSF | 384 | 0.001159 | 17 | E7 | --- | 3 | STLSFVC | No |
| E7 | CPWCAS | | VCPWCA | | VCP | | | | | STLSFVC | | P---- | |
| | | | | | | | | | | P---- | | | |
| HPV18- | LFLNTLSFV | 142 | LFLSTLSF | 383 | STLSF | 343 | 0.881711 | 0.11 | E7 | --- | 3 | STLSFVC | no |
| E7 | CPWCAS | | VCPWCA | | VCPW | | | | | STLSFVC | | PW--- | |
| | | | | | | | | | | PW--- | | | |
| HPV18- | LFLNTLSFV | 142 | LFLSTLSF | 383 | STLSF | 343 | 0.052525 | 2.8 | E7 | --- | 3 | --- | no |
| E7 | CPWCAS | | VCPWCA | | VCPW | | | | | STLSFVC | | sTLSFVC | |
| | | | | | C | | | | | PWC-- | | PWC-- | |
| HPV18- | LFLNTLSFV | 142 | LFLSTLSF | 383 | TLSFV | 390 | 0.084496 | 2.2 | E7 | ----TLSF | 4 | ----TLS | yes |
| E7 | CPWCAS | | VCPWCA | | CPW | | | | | VCPW--- | | FVCPW--- | |
| HPV18- | LFLNTLSFV | 142 | LFLSTLSF | 383 | TLSFV | 391 | 0.005172 | 6.8 | E7 | ----TLSFV | 4 | ----TLSF | yes |
| E7 | CPWCAS | | VCPWCA | | CPWC | | | | | CPWC-- | | VCPWC-- | |
| HPV18- | LFLNTLSFV | 142 | LFLSTLSF | 383 | TLSFV | 392 | 0.014523 | 4.1 | E7 | ---- | 4 | ----TLSF | yes |
| E7 | CPWCAS | | VCPWCA | | CPWC | | | | | TLSFVCP | | VCPWCA- | |
| | | | | | A | | | | | WCA- | | | |
| HPV18- | LFLNTLSFV | 142 | LFLSTLSF | 383 | LSFVC | 393 | 0.000752 | 22 | E7 | -----LSFV | 5 | -----LSF | yes |
| E7 | CPWCAS | | VCPWCA | | PWC | | | | | CPWC-- | | VCPWC-- | |
| HPV18- | LFLNTLSFV | 142 | LFLSTLSF | 383 | LSFVC | 394 | 0.005991 | 8.4 | E7 | -----LSF | 5 | -----LSF | yes |
| E7 | CPWCAS | | VCPWCA | | PWCA | | | | | VCPWCA- | | VCPWCA- | |
| HPV18- | LFLNTLSFV | 142 | LFLSTLSF | 383 | SFVCP | 395 | 0.00028 | 26 | E7 | ------SFV | 6 | ------SFV | yes |
| E7 | CPWCAS | | VCPWCA | | WCA | | | | | CPWCA- | | CPWCA- | |
| HPV18- | TLQDIVLHL | 143 | TLQEIVLH | 344 | TLQEI | 396 | 0.005456 | 7.6 | E7 | TLQEIVL | 0 | TLQeIVL | no |
| E7 | | | L | | VLH | | | | | H- | | H- | |
| HPV18- | TLQDIVLHL | 143 | TLQEIVLH | 344 | TLQEI | 344 | 0.974852 | 0.02 | E7 | TLQEIVL | 0 | TLQeIVL | no |
| E7 | | | L | | VLHL | | | | | HL | | HL | |
| HPV18- | TLQDIVLHL | 143 | TLQEIVLH | 344 | LQEIV | 397 | 0.11183 | 1.3 | E7 | -LQEIVLH | 1 | - | no |
| E7 | | | L | | LHL | | | | | L | | LQeIVLH | |
| | | | | | | | | | | | | L | |
| HPV18- | SEEENDEID | 144 | SEEENDE | 398 | SEEEN | 399 | 0.001388 | 6.5 | E7 | SEEENDEA-- | 0 | SEEENDEa- | no |
| E7 | GVNHQHLP | | ADGVSHA | | DEA | | | | | ---------- | | --------- | |
| | ARR | | QLPARR | | | | | | | | | | |
| HPV18- | SEEENDEID | 144 | SEEENDE | 398 | SEEEN | 400 | 0.000369 | 12 | E7 | SEEENDEAD | 0 | SEEENDEaD | no |
| E7 | GVNHQHLP | | ADGVSHA | | DEAD | | | | | --------- | | --------- | |
| | ARR | | QLPARR | | | | | | | -- | | -- | |
| HPV18- | SEEENDEID | 144 | SEEENDE | 398 | SEEEN | 401 | 6.60E−05 | 26 | E7 | SEEENDE | 10 | SEEENDEaDG | no |
| E7 | GVNHQHLP | | ADGVSHA | | DEAD | | | | | ADG---- | | ---------- | |
| | ARR | | QLPARR | | G | | | | | ------ | | | |
| HPV18- | SEEENDEID | 144 | SEEENDE | 398 | EEEN | 402 | 2.20E−05 | 39 | E7 | - | 1 | -EEENDEa | no |
| E7 | GVNHQHLP | | ADGVSHA | | DEAD | | | | | EEENDEA | | D------ | |
| | ARR | | QLPARR | | | | | | | D------ | | ----- | |
| | | | | | | | | | | ----- | | | |
| HPV18-E7 | SEEENDEID | 144 | SEEENDE | 398 | EEEN | 403 | 0.000108 | 22 | E7 | -EEENDEA | 1 | EEENDEa | no |
| peptide | GVNHQHLP | | ADGVSHA | | DEAD | | | | | DG----- | | DG----- | |
| 6 | ARR | | QLPARR | | G | | | | | ----- | | ----- | |
| HPV18-E7 | SEEENDEID | 144 | SEEENDE | 398 | EEEN | 404 | 0.00523 | 3.6 | E7 | -EEENDEA | 1 | - | no |
| peptide | GVNHQHLP | | ADGVSHA | | DEAD | | | | | DGV---- | | EEENDEa | |
| 6 | ARR | | QLPARR | | GV | | | | | ----- | | DGV---- | |
| | | | | | | | | | | | | ----- | |
| HPV18- | SEEENDEID | 144 | SEEENDE | 398 | EEND | 405 | 0.000402 | 12 | E7 | --EENDEA | 2 | --EENDEa | no |
| E7 | GVNHQHLP | | ADGVSHA | | EADG | | | | | DG----- | | DG----- | |
| | ARR | | QLPARR | | | | | | | ----- | | ----- | |
| HPV18- | SEEENDEID | 144 | SEEENDE | 398 | EEND | 345 | 0.06243 | 1 | E7 | --EENDEA | 2 | -EENDEa | no |
| E7 | GVNHQHLP | | ADGVSHA | | EADG | | | | | DGV---- | | DGV---- | |
| | ARR | | QLPARR | | V | | | | | ----- | | ----- | |
| HPV18- | SEEENDEID | 144 | SEEENDE | 398 | EEND | 345 | 0.000439 | 12 | E7 | --EENDEA | 2 | --EENDEa | no |
| E7 | GVNHQHLP | | ADGVSHA | | EADG | | | | | DGVS--- | | DGVs--- | |
| | ARR | | QLPARR | | VS | | | | | ----- | | ----- | |
| HPV18- | SEEENDEID | 144 | SEEENDE | 398 | ENDE | 406 | 0.000383 | 24 | E7 | ---ENDEA | 3 | ---ENDEa | no |
| E7 | GVNHQHLP | | ADGVSHA | | ADGV | | | | | DGV---- | | DGV---- | |
| | ARR | | QLPARR | | | | | | | ----- | | ----- | |
| HPV18- | SEEENDEID | 144 | SEEENDE | 398 | ENDE | 407 | 0.000168 | 27 | E7 | ---ENDEA | 3 | ---ENDEa | no |
| E7 | GVNHQHLP | | ADGVSHA | | ADGV | | | | | DGVS--- | | DGVs---- | |
| | ARR | | QLPARR | | S | | | | | ----- | | ------ | |
| HPV18-E7 | SEEENDEID | 144 | SEEENDE | 398 | ENDE | 408 | 0.007632 | 4.4 | E7 | ---ENDEAD | 3 | ---ENDEaD | no |
| peptide | GVNHQHLP | | ADGVSHA | | ADGV | | | | | GVSH--- | | GVsH--- | |
| 6 | ARR | | QLPARR | | SH | | | | | ------ | | ------ | |
| HPV18- | SEEENDEID | 144 | SEEENDE | 398 | NDEA | 409 | 3.70E−05 | 46 | E7 | ----NDEA | 4 | ----NDEaD | no |
| E7 | GVNHQHLP | | ADGVSHA | | DGVS | | | | | DGVS---- | | GVs---- | |
| | ARR | | QLPARR | | | | | | | ------ | | ------ | |
| HPV18- | SEEENDEID | 144 | SEEENDE | 398 | NDEA | 410 | 0.002927 | 7.7 | E7 | ----NDEAD | 4 | ----NDEaD | no |
| E7 | GVNHQHLP | | ADGVSHA | | DGVS | | | | | GVSH--- | | GVsH--- | |
| | ARR | | QLPARR | | H | | | | | ------ | | ------ | |
| HPV18- | SEEENDEID | 144 | SEEENDE | 398 | NDEA | 411 | 0.008188 | 2.9 | E7 | ----NDEAD | 4 | ----NDEaDG | no |
| E7 | GVNHQHLP | | ADGVSHA | | DGVS | | | | | G | | VsHa------ | |
| | ARR | | QLPARR | | HA | | | | | VSHA | | | |
| HPV18- | SEEENDEID | 144 | SEEENDE | 398 | DEAD | 412 | 0.004971 | 4 | E7 | ----- | 5 | ----- | no |
| E7 | GVNHQHLP | | ADGVSHA | | GVSH | | | | | DEADGVSH | | DEaDGVsH | |
| | ARR | | QLPARR | | | | | | | ------- | | ------- | |
| HPV18- | SEEENDEID | 144 | SEEENDE | 398 | DEAD | 413 | 0.029952 | 1.6 | E7 | ----- | 5 | ----- | no |
| E7 | GVNHQHLP | | ADGVSHA | | GVSH | | | | | DEADGV | | DEaDGVs | |
| | ARR | | QLPARR | | A | | | | | SHA------ | | Ha------ | |
| HPV18- | SEEENDEID | 144 | SEEENDE | 398 | DEAD | 414 | 0.009943 | 2.9 | E7 | -----DEAD | 5 | ----- | no |
| E7 | GVNHQHLP | | ADGVSHA | | GVSH | | | | | GVSHA | | DEaDGVs | |
| | ARR | | QLPARR | | AQ | | | | | Q----- | | Haq----- | |
| HPV18- | SEEENDEID | 144 | SEEENDE | 398 | EADG | 415 | 0.022002 | 3.8 | E7 | ------ | 6 | ------ | no |
| E7 | GVNHQHLP | | ADGVSHA | | VSHA | | | | | EADGVS | | EaDGVsH | |
| | ARR | | QLPARR | | | | | | | HA------ | | a------ | |
| HPV18- | SEEENDEID | 144 | SEEENDE | 398 | EADG | 346 | 0.141601 | 0.82 | E7 | ------ | 6 | ------ | no |
| E7 | GVNHQHLP | | ADGVSHA | | VSHA | | | | | EADGVSH | | EaDGVs | |
| | ARR | | QLPARR | | Q | | | | | AQ----- | | Haq----- | |
| HPV18- | SEEENDEID | 144 | SEEENDE | 398 | EADG | 347 | 0.231481 | 0.58 | E7 | ------ | 6 | ------ | no |
| E7 | GVNHQHLP | | ADGVSHA | | VSHA | | | | | EADGVSHA | | EaDGVsH | |
| | ARR | | QLPARR | | QL | | | | | QL---- | | aqL---- | |
| HPV18- | SEEENDEID | 144 | SEEENDE | 398 | ADGV | 416 | 0.000301 | 22 | E7 | ------- | 7 | ------- | no |
| E7 | GVNHQHLP | | ADGVSHA | | SHAQ | | | | | ADGVSHAQ | | aDGVsHaq | |
| | ARR | | QLPARR | | | | | | | ----- | | ----- | |
| HPV18- | SEEENDEID | 144 | SEEENDE | 398 | ADGV | 417 | 0.0265 | 1.9 | E7 | ------- | 7 | ------- | no |
| E7 | GVNHQHLP | | ADGVSHA | | SHAQ | | | | | ADGVSHA | | aDGVsHaq | |
| | ARR | | QLPARR | | L | | | | | QL---- | | L---- | |
| HPV18- | SEEENDEID | 144 | SEEENDE | 398 | ADGV | 418 | 0.000564 | 11 | E7 | ------- | 7 | ------- | no |
| E7 | GVNHQHLP | | ADGVSHA | | SHAQ | | | | | ADGVSHAQ | | aDGVsHaq | |
| | ARR | | QLPARR | | LP | | | | | LP--- | | LP--- | |
| HPV18- | SEEENDEID | .44 | SEEENDE | 398 | DGVS | 348 | 0.157757 | 0.88 | E7 | -------- | 8 | -------- | no |
| E7 | GVNHQHLP | | ADGVSHA | | HAQL | | | | | DGVSHAQ | | DGVsHaq | |
| | ARR | | QLPARR | | | | | | | L---- | | L---- | |
| HPV18- | SEEENDEID | 144 | SEEENDE | 398 | DGVS | 419 | 0.023822 | 3.6 | E7 | -------- | 8 | -------- | no |
| E7 | GVNHQHLP | | ADGVSHA | | HAQL | | | | | DGVSHAQL | | DGVsHaqL | |
| | ARR | | QLPARR | | P | | | | | P--- | | P--- | |
| HPV18- | SEEENDEID | 144 | SEEENDE | 398 | DGVS | 420 | 0.009755 | 5.1 | E7 | -------- | 8 | -------- | no |
| E7 | GVNHQHLP | | ADGVSHA | | HAQL | | | | | DGVSHAQL | | DGVsHaqL | |
| | ARR | | QLPARR | | PA | | | | | PA-- | | PA-- | |
| HPV18- | SEEENDEID | 144 | SEEENDE | 398 | GVSH | 421 | 0.000275 | 32 | E7 | --------- | 9 | --------- | no |
| E7 | GVNHQHLP | | ADGVSHA | | AQLP | | | | | GVSHAQLP | | GVsHaqLP | |
| | ARR | | QLPARR | | | | | | | --- | | --- | |
| HPV18- | SEEENDEID | 144 | SEEENDE | 398 | GVSH | 422 | 0.03138 | 4 | E7 | --------- | 9 | --------- | no |
| E7 | GVNHQHLP | | ADGVSHA | | AQLP | | | | | GVSHAQLPA-- | | GVsHaqLPA-- | |
| | ARR | | QLPARR | | A | | | | | | | | |
| HPV18- | SEEENDEID | .44 | SEEENDE | 398 | GVSH | 349 | 0.245522 | 0.97 | E7 | --------- | 9 | --------- | no |
| E7 | GVNHQHLP | | ADGVSHA | | AQLP | | | | | GVSHAQLPAR- | | GVsHaqLPAR- | |
| | ARR | | QLPARR | | AR | | | | | | | | |
| HPV18- | SEEENDEID | 144 | SEEENDE | 398 | VSHA | 423 | 0.004633 | 13 | E7 | ---------- | 10 | ---------- | no |
| E7 | GVNHQHLP | | ADGVSHA | | QLPA | | | | | VSHAQLPA-- | | VsHaqLPA-- | |
| | ARR | | QLPARR | | | | | | | | | | |
| HPV18- | SEEENDEID | 144 | SEEENDE | 398 | VSHA | 350 | 0.69893 | 0.13 | E7 | ---------- | 10 | ---------- | no |
| E7 | GVNHQHLP | | ADGVSHA | | QLPA | | | | | VSHAQLPAR- | | VsHaqLPAR- | |
| | ARR | | QLPARR | | R | | | | | | | | |
| HPV18- | SEEENDEID | 144 | SEEENDE | 398 | VSHA | 351 | 0.313064 | 0.73 | E7 | ---------- | 10 | ---------- | no |
| E7 | GVNHQHLP | | ADGVSHA | | QLPA | | | | | VSHAQLPARR | | VsHaqLPARR | |
| | ARR | | QLPARR | | RR | | | | | | | | |
| HPV18- | SEEENDEID | 144 | SEEENDE | 398 | SHAQ | 424 | 0.016712 | 4 | E7 | ----------- | 11 | ----------- | no |
| E7 | GVNHQHLP | | ADGVSHA | | LPAR | | | | | SHAQLPAR- | | sHaqLPAR- | |
| | ARR | | QLPARR | | | | | | | | | | |
| HPV18- | SEEENDEID | 144 | SEEENDE | 398 | SHAQ | 352 | 0.163463 | 0.88 | E7 | ----------- | 11 | ----------- | no |
| E7 | GVNHQHLP | | ADGVSHA | | LPARR | | | | | SHAQLPARR | | sHaqLPARR | |
| | ARR | | QLPARR | | | | | | | | | | |
| HPV18- | SEEENDEID | 144 | SEEENDE | 398 | HAQL | 425 | 0.055034 | 2.1 | E7 | ------------ | 12 | ------------ | no |
| E7 | GVNHQHLP | | ADGVSHA | | PARR | | | | | HAQLPARR | | HaqLPARR | |
| | ARR | | QLPARR | | | | | | | | | | |
| Agonist | KLPQLCTEV | | KLPDLCTE | 355 | KLPDL | 355 | 0.000848 | 17 | E6 | KLPDLCT | 0 | KLPdLCT | no |
| peptide | | | | | CTE | | | | | E− | | E− | |
| 1 | | | | | | | | | | | | | |

TABLE 10A
HPV-45 MHC-II epitopes with strong affinity (best percentile rank <= 10) in at
least 1 HLA background, covered by AdV-HPV16/18.

| AdV- | | best | adjusted | HPV45 | HPV-45 | | Mismatch |
|---|---|---|---|---|---|---|---|
| HPV16/18 | best | percentile | best | protein | epitope aligned | Perfect | pos. in |
| Epitope | IC50 | rank | rank | origin | to AdV-HPV16/18 ^{A} | match | core |
| HPV18-E7 | 86.9 | 2.6 | 7.71 | E7 | -DLRtlQQLFLsT-- | no | 3, 4 |
| peptide 1 | | | | | | | |
| HPV18-E7 | 43.3 | 1 | 1.56 | E7 | -DLRtlQQLFLsTL- | no | 1, 2, 8 |
| peptide 1 | | | | | | | |
| HPV18-E7 | 36.4 | 1.4 | 1.51 | E7 | -DLRtlQQLFLSTLS | no | 1, 7 |
| peptide 1 | | | | | | | |
| HPV18-E7 | 55.5 | 0.69 | 2.05 | E7 | --LRtlQQLFLsTL- | no | 1, 2, 8 |
| peptide 1 | | | | | | | |
| HPV18-E7 | 42.1 | 0.92 | 1.43 | E7 | --LRtlQQLFLSTLS | no | 1, 7 |
| peptide 1 | | | | | | | |
| HPV18-E7 | 105.4 | 1.4 | 4.15 | E7 | ---RtlQQLFLSTLS | no | 1, 7 |
| peptide 1 | | | | | | | |
| HPV18-E7 | 132.3 | 6.4 | 18.98 | E7 | -FLsTLSFVCPWC-- | no | 2 |
| peptide 4 | | | | | | | |
| HPV18-E7 | 64.3 | 7.2 | 11.23 | E7 | -FLsTLSFVCPWCA- | no | 3 |
| peptide 4 | | | | | | | |
| HPV18-E7 | 53.7 | 1.6 | 4.74 | E7 | LFLsTLSFVCPW--- | no | 3 |
| peptide 4 | | | | | | | |
| HPV18-E7 | 64.3 | 2.7 | 4.21 | E7 | LFLsTLSFVCPWC-- | no | 3 |
| peptide 4 | | | | | | | |
| HPV18-E7 | 44.3 | 3.8 | 4.09 | E7 | LFLsTLSFVCPWCA- | no | 3 |
| peptide 4 | | | | | | | |
| HPV18-E7 | 239.7 | 10 | 29.65 | E7 | --LsTLSFVCPWCA- | no | 1 |
| peptide 4 | | | | | | | |
| HPV18-E7 | 74.1 | 7.4 | 11.54 | E7 | ------- | no | 2, 4, 5 |
| peptide 6 | | | | | aDGVsHaqLPARR | | |
| HPV18-E7 | 84.1 | 5.4 | 16.01 | E7 | -------- | no | 2, 4, 5 |
| peptide 6 | | | | | DGVsHaqLPARR | | |
| HPV18-E7 | 75.3 | 9.5 | 10.23 | E7 | ------ | no | 2, 4, 5 |
| peptide 6 | | | | | EaDGVsHaqLPARR | | |

^{A} bolded text indicates the predicted 9 AA MHC-II interacting core

TABLE 10B
Comparison of AdV-HPV16/18 epitopes with predicted HPV-45 MHC-II
epitopes

| | | | | | | | | | | | HPV-45 | | HPV-45 | |
|---|---|---|---|---|---|---|---|---|---|---|---|---|---|---|
| | | SEQ | | | | | | | | | epitope | | epitope | |
| AdV- | AdV- | ID | Aligning | SEQ | HPV-45 | SEQ | | | best | HPV45 | in AdV- | | aligned | |
| HPV16/18 | HPV16/18 | | HPV45 | ID | MHC-II | ID | best | best | adjusted | protein | HPV16/18 | | to AdV- | Perfect |
| Epitope | Sequence | NO: | sequence | NO: | epitope | NO: | IC50 | rank | rank | origin | epitope | Offset | HPV16/18 | match |
| HPV18- | DDLRAF | 139 | DDLRTLQ | 363 | DLRTLQQ | 429 | 86.9 | 26 | 7.71 | E7 | DLRTLQQ | 1 | - | no |
| E7 | QQLFLN | | QLFLSTLS | | LFLST | | | | | | LFLST-- | | DLRtlQQL | |
| peptide 1 | TLS | | | | | | | | | | | | FLsT-- | |
| HPV18- | DDLRAF | 139 | DDLRTLQ | 363 | DLRTLQQ | 429 | 43.3 | 1 | 1.56 | E7 | - | 1 | -DLRtlQQL | no |
| E7 | QQLFLN | | QLFLSTLS | | LFLSTL | | | | | | DLRTLQQ | | FLsTL- | |
| peptide 1 | TLS | | | | | | | | | | LFLSTL- | | | |
| HPV18- | DDLRAF | 139 | DDLRTLQ | 363 | DLRTLQQ | 430 | 36.4 | 14 | 1.51 | E7 | DLRTLQQ | 1 | -DLRtlQQL | no |
| E7 | QQLFLN | | QLFLSTLS | | LFLSTLS | | | | | | LFLSTLS | | FLsTLS | |
| peptide 1 | TLS | | | | | | | | | | | | | |
| HPV18- | DDLRAF | 139 | DDLRTLQ | 363 | LRTLQQLF | 436 | 55.5 | 0.69 | 2.05 | E7 | -- | 2 | -- | no |
| E7 | QQLFLN | | QLFLSTLS | | LSTL | | | | | | LRTLQQL | | LRtlQQLF | |
| peptide 1 | TLS | | | | | | | | | | FLSTL- | | LsTL- | |
| HPV18- | DDLRAF | 139 | DDLRTLQ | 363 | LRTLQQLF | 437 | 42.1 | 0.92 | 1.43 | E7 | -- | 2 | -- | no |
| E7 | QQLFLN | | QLFLSTLS | | LSTLS | | | | | | LRTLQQL | | LRtlQQLF | |
| peptide 1 | TLS | | | | | | | | | | FLSTLS | | LsTLS | |
| HPV18- | DDLRAF | 139 | DDLRTLQ | 363 | RTLQQLFL | 439 | 105.4 | 14 | 4.15 | E7 | ---RTLQQLF | 3 | --- | no |
| E7 | QQLFLN | | QLFLSTLS | | STLS | | | | | | LSTLS | | RtlQQLFL | |
| peptide 1 | TLS | | | | | | | | | | | | STLS | |
| HPV18- | LFLNTL | 142 | LFLSTLSF | 383 | FLSTLSFV | 432 | 132.3 | 64 | 18.98 | E7 | FLSTLSFV | 1 | FLsTLSFV | no |
| E7 | SFVCPW | | VCPWCA | | CPWC | | | | | | CPWC-- | | CPWC-- | |
| peptide 4 | CAS | | | | | | | | | | | | | |
| HPV18- | LFLNTL | 142 | LFLSTLSF | 383 | FLSTLSFV | 433 | 64.3 | 72 | 11.23 | E7 | FLSTLSFV | 1 | FLsTLSFV | no |
| E7 | SFVCPW | | VCPWCA | | CPWCA | | | | | | CPWCA- | | CPWCA- | |
| peptide 4 | CAS | | | | | | | | | | | | | |
| HPV18- | CAS | 142 | LFLSTLSF | 383 | LFLSTLSF | 434 | 53.7 | 16 | 4.74 | E7 | LFLSTLSF | 0 | LFLsTLSF | no |
| E7 | LFLNTL | | VCPWCA | | VCPW | | | | | | VCPW--- | | VCPW--- | |
| peptide 4 | SFVCPW | | | | | | | | | | | | | |
| HPV18- | LFLNTL | 142 | LFLSTLSF | 383 | LFLSTLSF | 435 | 64.3 | 27 | 4.21 | E7 | LFLSTLSF | 0 | LFLsTLSF | no |
| E7 | SFVCPW | | VCPWCA | | VCPWC | | | | | | VCPWC-- | | VCPWC-- | |
| peptide 4 | CAS | | | | | | | | | | | | | |
| HPV18- | CAS | 142 | LFLSTLSF | 383 | LFLSTLSF | 383 | 44.3 | 38 | 4.09 | E7 | LFLSTLSF | 0 | LFLsTLSF | no |
| E7 | LFLNTL | | VCPWCA | | VCPWCA | | | | | | VCPWCA- | | VCPWCA- | |
| peptide 4 | SFVCPW | | | | | | | | | | | | | |
| HPV18- | LFLNTL | 142 | LFLSTLSF | 383 | LSTLSFVC | 438 | 239.7 | 10 | 29.65 | E7 | -- | 2 | -- | no |
| E7 | SFVCPW | | VCPWCA | | PWCA | | | | | | LSTLSFVC | | LsTLSFV | |
| peptide 4 | CAS | | | | | | | | | | PWCA- | | CPWCA- | |
| HPV18- | SEEEND | 144 | SEEENDEA | 398 | ADGVSHA | 440 | 1653.2 | 16 | 47.44 | E7 | ------- | 7 | ------- | no |
| E7 | EIDGVN | | DGVSHAQ | | QLPAR | | | | | | ADGVSHA | | aDGVsHa | |
| peptide 6 | HQHLPA | | LPARR | | | | | | | | QLPAR- | | qLPAR- | |
| | RR | | | | | | | | | | | | | |
| HPV18- | SEEEND | 144 | SEEENDEA | 398 | ADGVSHA | 426 | 74.1 | 74 | 11.54 | E7 | ------- | 7 | ------- | no |
| E7 | EIDGVN | | DGVSHAQ | | QLPARR | | | | | | ADGVSHA | | aDGVsHa | |
| peptide 6 | HQHLPA | | LPARR | | | | | | | | QLPARR | | qLPARR | |
| | RR | | | | | | | | | | | | | |
| HPV18- | SEEEND | 144 | SEEENDEA | 398 | DEADGVS | 441 | 1065.8 | 20 | 59.31 | E7 | ----- | 5 | ----- | no |
| E7 | EIDGVN | | DGVSHAQ | | HAQLP | | | | | | DEADGVS | | DEaDGVs | |
| peptide 6 | HQHLPA | | LPARR | | | | | | | | HAQLP--- | | HaqLP--- | |
| | RR | | | | | | | | | | | | | |
| HPV18- | SEEEND | 144 | SEEENDEA | 398 | DEADGVS | 442 | 1128.1 | 21 | 32.75 | E7 | ----- | 5 | ----- | no |
| E7 | EIDGVN | | DGVSHAQ | | HAQLPA | | | | | | DEADGVS | | DEaDGVs | |
| peptide 6 | HQHLPA | | LPARR | | | | | | | | HAQLPA-- | | HaqLPA-- | |
| | RR | | | | | | | | | | | | | |
| HPV18- | SEEEND | 144 | SEEENDEA | 398 | DEADGVS | 443 | 949.6 | 22 | 23.69 | E7 | ----- | 5 | ----- | no |
| E7 | EIDGVN | | DGVSHAQ | | HAQLPAR | | | | | | DEADGVS | | DEaDGVs | |
| peptide 6 | HQHLPA | | ILPARR | | | | | | | | HAQLPAR | | HaqLPAR- | |
| | RR | | | | | | | | | | | | | |
| HPV18- | SEEEND | 144 | SEEENDEA | 398 | DEADGVS | 444 | 88.3 | 12 | 12 | E7 | ----- | 5 | ----- | no |
| E7 | EIDGVN | | DGVSHAQ | | HAQLPAR | | | | | | DEADGVS | | DEaDGVs | |
| peptide 6 | HOHLPA | | LPARR | | R | | | | | | HAQLPARR | | HaqLPARR | |
| | RR | | | | | | | | | | | | | |
| HPV18- | HQHLPA | 144 | SEEENDEA | 398 | DGVSHAQ | 427 | 84.1 | 54 | 16.01 | E7 | -------- | 8 | -------- | no |
| E7 | SEEEND | | DGVSHAQ | | LPARR | | | | | | DGVSHAQ | | DGVsHaq | |
| peptide 6 | EIDGVN | | LPARR | | | | | | | | LPARR | | LPARR | |
| | RR | | | | | | | | | | | | | |
| HPV18- | SEEEND | 144 | SEEENDEA | 398 | EADGVSH | 445 | 1511.8 | 19 | 56.34 | E7 | -------- | 6 | -------- | no |
| E7 | EIDGVN | | DGVSHAQ | | AQLPA | | | | | | EADGVSH | | EaDGVsH | |
| peptide 6 | HQHLPA | | LPARR | | | | | | | | AQLPA-- | | aqLPA-- | |
| | RR | | | | | | | | | | | | | |
| HPV18- | SEEEND | 144 | SEEENDEA | 398 | EADGVSH | 446 | 1150.3 | 19 | 29.63 | E7 | -------- | 6 | -------- | no |
| E7 | EIDGVN | | DGVSHAQ | | AQLPAR | | | | | | EADGVSH | | EaDGVsH | |
| peptide 6 | HQHLPA | | LPARR | | | | | | | | AQLPAR- | | aqLPAR- | |
| | RR | | | | | | | | | | | | | |
| HPV18- | SEEEND | 144 | SEEENDEADGVSHAQ | 398 | EADGVSH | 431 | 75.3 | 95 | 10.23 | E7 | -------- | 6 | -------- | no |
| E7 | EIDGVN | | LPARR | | AQLPARR | | | | | | EADGVSH | | EaDGVsH | |
| peptide 6 | HQHLPA | | | | | | | | | | AQLPARR | | aqLPARR | |
| | RR | | | | | | | | | | | | | |
| HPV18- | HQHLPA | 144 | SEEENDEA | 398 | EEENDEA | 447 | 4253.2 | 20 | 59.31 | E7 | -EEENDEA | 1 | - | no |
| E7 | SEEEND | | DGVSHAQ | | DGVSH | | | | | | DGVSH---- | | EEENDEa | |
| peptide 6 | EIDGVN | | LPARR | | | | | | | | --- | | DGVsH---- | |
| | RR | | | | | | | | | | | | --- | |
| HPV18- | SEEEND | 144 | SEEENDEA | 398 | EEENDEA | 448 | 3661.2 | 24 | 37.42 | E7 | -EEENDEA | 1 | - | no |
| E7 | EIDGVN | | DGVSHAQ | | DGVSHA | | | | | | DGVSHA-- | | EEENDEa | |
| peptide 6 | HQHLPA | | LPARR | | | | | | | | ---- | | DGVsHa-- | |
| | RR | | | | | | | | | | | | ---- | |
| HPV18- | SEEEND | 144 | SEEENDEA | 398 | EEENDEA | 449 | 2346.3 | 28 | 30.15 | E7 | - | 1 | - | no |
| E7 | EIDGVN | | DGVSHAQ | | DGVSHAQ | | | | | | EEENDEA | | EEENDEa | |
| peptide 6 | HQHLPA | | LPARR | | | | | | | | DGVSHAQ | | DGVsHaq- | |
| | RR | | | | | | | | | | ----- | | ----- | |
| HPV18- | SEEEND | 144 | SEEENDEA | 398 | EEENDEA | 450 | 1463.3 | 33 | 33 | E7 | -EEENDEA | 1 | EEENDEa | no |
| E7 | EIDGVN | | DGVSHAQ | | DGVSHAQ | | | | | | DGVSHAQ | | DGVsHaq | |
| | HQHLPA | | ILPARR | | | | | | | | L---- | | L---- | |
| peptide 6 | RR | | | | L | | | | | | | | | |
| HPV18- | SEEEND | 144 | SEEENDEA | 398 | EEENDEA | 451 | 145.2 | 24 | 123.34 | E7 | - | 1 | - | no |
| E7 | EIDGVN | | DGVSHAQ | | DGVSHAQ | | | | | | EEENDEA | | EEENDEa | |
| peptide 6 | HOHLPA | | LPARR | | ILPARR | | | | | | DGVSHAQ | | DGVsHaq | |
| | RR | | | | | | | | | | LPARR | | LPARR | |
| HPV18- | SEEEND | 144 | SEEENDEA | 398 | EENDEAD | 452 | 4325.5 | 20 | 59.31 | E7 | -- | 2 | -- | no |
| E7 | EIDGVN | | DGVSHAQ | | GVSHA | | | | | | EENDEAD | | EENDEaD | |
| peptide 6 | HQHLPA | | LPARR | | | | | | | | GVSHA-- | | GVsHa---- | |
| | RR | | | | | | | | | | | | | |
| | | | | | | | | | | | ---- | | -- | |
| HPV18- | SEEEND | 144 | SEEENDEA | 398 | EENDEAD | 453 | 2519.2 | 24 | 37.42 | E7 | -- | 2 | -- | no |
| E7 | EIDGVN | | DGVSHAQ | | GVSHAQ | | | | | | EENDEAD | | EENDEaD | |
| peptide 6 | HQHLPA | | LPARR | | | | | | | | GVSHAQ- | | GVsHaq- | |
| | RR | | | | | | | | | | ---- | | ---- | |
| HPV18- | HQHLPA | 144 | SEEENDEA | 398 | EENDEAD | 454 | 1343.9 | 28 | 30.15 | E7 | -- | 2 | -- | no |
| E7 | SEEEND | | DGVSHAQ | | GVSHAQL | | | | | | EENDEAD | | EENDEaD | |
| peptide 6 | EIDGVN | | LPARR | | | | | | | | GVSHAQL | | GVsHaqL | |
| | RR | | | | | | | | | | ---- | | ---- | |
| HPV18- | HQHLPA | 144 | SEEENDEA | 398 | EENDEAD | 455 | 972.2 | 25 | 25 | E7 | -- | 2 | -- | no |
| E7 | SEEEND | | DGVSHAQ | | GVSHAQL | | | | | | EENDEAD | | EENDEaD | |
| peptide 6 | EIDGVN | | LPARR | | P | | | | | | GVSHAQL | | GVsHaqL | |
| | RR | | | | | | | | | | | | | |
| | | | | | | | | | | | P--- | | P--- | |
| HPV18- | SEEEND | 144 | SEEENDEA | 398 | ENDEADG | 456 | 3399.8 | 28 | 83.03 | E7 | --- | 3 | --- | no |
| E7 | EIDGVN | | DGVSHAQ | | VSHAQ | | | | | | ENDEADG | | ENDEaDG | |
| peptide 6 | HQHLPA | | LPARR | | | | | | | | VSHAQ | | VsHaq | |
| | RR | | | | | | | | | | ----- | | ----- | |
| HPV18- | SEEEND | 144 | SEEENDEA | 398 | ENDEADG | 457 | 1519 | 27 | 42.1 | E7 | --- | 3 | --- | no |
| E7 | EIDGVN | | DGVSHAQ | | VSHAQL | | | | | | ENDEADG | | ENDEaDG | |
| peptide 6 | HOHLPA | | LPARR | | | | | | | | VSHAQL | | VsHaqL | |
| | RR | | | | | | | | | | ---- | | ---- | |
| HPV18- | HQHLPA | 144 | SEEENDEA | 398 | ENDEADG | 458 | 943.6 | 25 | 26.92 | E7 | --- | 3 | --- | no |
| E7 | SEEEND | | DGVSHAQ | | VSHAQLP | | | | | | ENDEADG | | ENDEaDG | |
| peptide 6 | EIDGVN | | LPARR | | | | | | | | VSHAQLP | | VsHaqLP | |
| | RR | | | | | | | | | | --- | | --- | |
| HPV18- | SEEEND | 144 | SEEENDEA | 398 | ENDEADG | 459 | 1180.2 | 24 | 24 | E7 | --- | 3 | --- | no |
| E7 | EIDGVN | | DGVSHAQ | | VSHAQLP | | | | | | ENDEADG | | ENDEaDG | |
| peptide 6 | HOHLPA | | LPARR | | A | | | | | | VSHAQLP | | VsHaqLP | |
| | RR | | | | | | | | | | A-- | | A-- | |
| HPV18- | SEEEND | 144 | SEEENDEA | 398 | NDEADGV | 460 | 1818.6 | 28 | 83.03 | E7 | ---- | 4 | ---- | no |
| E7 | EIDGVN | | DGVSHAQ | | SHAQL | | | | | | NDEADGV | | NDEaDG | |
| peptide 6 | HQHLPA | | LPARR | | | | | | | | SHAQL---- | | VsHaqL--- | |
| | RR | | | | | | | | | | | | | |
| HPV18- | SEEEND | 144 | SEEENDEA | 398 | NDEADGV | 461 | 991.8 | 23 | 35.87 | E7 | ---- | 4 | ---- | no |
| E7 | EIDGVN | | DGVSHAQ | | SHAQLP | | | | | | NDEADGV | | NDEaDG | |
| peptide 6 | HQHLPA | | LPARR | | | | | | | | SHAQLP--- | | VsHaqLP- | |
| | RR | | | | | | | | | | | | | |
| HPV18- | SEEEND | 144 | SEEENDEA | 398 | NDEADGV | 462 | 1122.1 | 22 | 23.69 | E7 | ---- | 4 | ---- | no |
| E7 | EIDGVN | | DGVSHAQ | | SHAQLPA | | | | | | NDEADGV | | NDEaDG | |
| peptide 6 | HQHLPA | | LPARR | | | | | | | | SHAQLPA- | | VsHaqLP | |
| | RR | | | | | | | | | | - | | A-- | |
| HPV18- | SEEEND | 144 | SEEENDEA | 398 | NDEADGV | 463 | 733.2 | 24 | 24 | ET | ---- | 4 | ---- | no |
| E7 | EIDGVN | | DGVSHAQ | | SHAQLPA | | | | | | NDEADGV | | NDEaDG | |
| peptide 6 | HOHLPA | | LPARR | | R | | | | | | SHAQLPA | | VsHaqLP | |
| | RR | | | | | | | | | | R- | | AR- | |
| HPV18- | SEEEND | 144 | SEEENDEA | 398 | SEEENDEA | 464 | 4849.7 | 20 | 59.31 | E7 | SEEENDE | 0 | SEEENDE | no |
| E7 | EIDGVN | | DGVSHAQ | | DGVS | | | | | | ADGVS | | aDGVS | |
| peptide 6 | HOHLPA | | LPARR | | | | | | | | ---- | | ---- | |
| | RR | | | | | | | | | | ---- | | ---- | |
| HPV18- | SEEEND | 144 | SEEENDEA | 398 | SEEENDEA | 465 | 3873.8 | 24 | 37.42 | E7 | SEEENDE | 0 | SEEENDE | no |
| E7 | EIDGVN | | DGVSHAQ | | DGVSH | | | | | | ADGVSH-- | | aDGVsH-- | |
| peptide 6 | HOHLPA | | LPARR | | | | | | | | ----- | | ----- | |
| | RR | | | | | | | | | | | | | |
| HPV18- | SEEEND | 144 | SEEENDEA | 398 | SEEENDEA | 466 | 3616.6 | 28 | 30.15 | E7 | SEEENDE | 0 | SEEENDE | no |
| E7 | EIDGVN | | DGVSHAQ | | DGVSHA | | | | | | ADGVSHA | | aDGVsHa | |
| peptide 6 | HOHLPA | | LPARR | | | | | | | | ------ | | ------ | |
| | RR | | | | | | | | | | | | | |
| HPV18- | SEEEND | 144 | SEEENDEA | 398 | SEEENDEA | 467 | 2533.8 | 29 | 29 | E7 | SEEENDE | 0 | SEEENDE | no |
| E7 | EIDGVN | | DGVSHAQ | | DGVSHAQ | | | | | | ADGVSHA | | aDGVsHa | |
| peptide 6 | HOHLPA | | LPARR | | | | | | | | Q----- | | q----- | |
| | RR | | | | | | | | | | | | | |
| HPV18- | SEEEND | 144 | SEEENDEA | 398 | SEEENDEA | 468 | 494.1 | 21 | 123.34 | E7 | SEEENDE | 0 | SEEENDE | no |
| E7 | EIDGVN | | DGVSHAQ | | DGVSHAQ | | | | | | ADGVSHA | | aDGVsHa | |
| peptide 6 | HQHLPA | | LPARR | | LPAR | | | | | | QLPAR- | | qLPAR- | |
| | RR | | | | | | | | | | | | | |
| HPV18- | SEEEND | 144 | SEEENDEA | 398 | SEEENDEA | 398 | 144.6 | 27 | 208.74 | E7 | SEEENDE | 0 | SEEENDE | no |
| E7 | EIDGVN | | DGVSHAQ | | DGVSHAQ | | | | | | ADGVSHA | | aDGVsHa | |
| peptide 6 | HOHLPA | | LPARR | | LPARR | | | | | | QLPARR | | qLPARR | |
| | RR | | | | | | | | | | | | | |

As seen below in Table 11, epitope cross-coverage in HPV-45 is considerably better than in any of the other included HPV genotypes. In HPV-45, 14 AdV-HPV16/18 epitopes show cross-coverage with predicted MHC-I epitopes of any binding strength rank, while the next best covered HPV genotype, HPV-35, is covered by 8 ADV-HPV16/18 epitopes. In addition, 3 ADV-HPV16/18 epitopes show cross-coverage with predicted MHC-II epitopes. No MHC-II epitope matching ADV-HPV16/18 epitopes were found for HPV-35, and only 1 for HPV-58.

Next, only predicted epitopes with strong binding metrics were considered, i.e., those having a IC50 based percentile rank less than 1 for MHC-I epitopes and less than 10 for MHC-II epitopes in a fixed background of randomly-selected peptides from SWISSPROT in at least one of the included 27 HLA alleles. These thresholds are recommended by IEDB for the two MHC classes. This resulted in 10 ADV-HPV16/18 epitopes with MHC-I cross-coverage in HPV-45, and only 5 in the next best covered genotypes, HPV-31 and HPV-35. See Table 11.

TABLE 11
Predicted Cross-Coverage of ADV-HPV16/18 in Six Cancerogenic HPV Genotypes

| | | | | | Filtered for best | | Filtered for best | |
|---|---|---|---|---|---|---|---|---|
| | Unfiltered | | | | rank <= 1 | | rank <= 10 | |
| | MHC-I | | MHC-II | | MHC-I | | MHC-II | |
| | # | # ADV-HPV16/18 | # | H# ADV-PV16/18 | # | # ADV-HPV16/18 | # | # ADV-HPV16/18 |
| | pred | epitopes | pred | epitopes | pred | epitopes | pred | epitopes |
| HPV-45 | 112 | 14 | 45 | 3 | 31 | 10 | 15 | 3 |
| HPV-31 | 38 | 7 | 0 | 0 | 10 | 5 | 0 | 0 |
| HPV-33 | 14 | 5 | 0 | 0 | 5 | 4 | 0 | 0 |
| HPV-35 | 31 | 8 | 0 | 0 | 6 | 5 | 0 | 0 |
| HPV-52 | 9 | 2 | 0 | 0 | 1 | 1 | 0 | 0 |
| HPV-58 | 24 | 4 | 1 | 1 | 5 | 3 | 1 | 1 |

Table 11 legend: # pred: number of predicted epitopes in each HPV genotype that are cross-covered by ADV-HPV16/18 epitopes; # ADV-HPV16/18 epitopes: numbers of ADV-HPV16/18 epitopes with cross-coverage in the respective HPV genotype; Unfiltered: includes all predicted epitopes; Filtered for best rank: only predicted epitopes ranked in the top 1% (MHC-I) or 10% (MHC-II) IC50 based binding strength in a fixed background of randomly selected peptides from SWISSPROT, when binding to any of the included 27 HLA alleles.

Three of the ADV-HPV16/18 epitopes with coverage of strong MHC-I binding epitopes in HPV-45 also had similar coverage of other HPV genotypes. These were HPV16-E6 peptide 5 (shared with HPV-33 and HPV-58), HPV16-E6 peptide 8 (shared with HPV-31), and HPV18-E7 peptide 5 (shared with HPV-31 and HPV-35). See Table 12.

TABLE 12
Cross-Coverage of ADV-HPV16/18 Epitopes With Strongly Binding MHC-I
Epitopes in Different HPV Genotypes.

| | | Number of matching MHC-I epitopes of | | | | | |
|---|---|---|---|---|---|---|---|
| | | length 8-10 AA with best percentile | | | | | |
| ADV- | | rank <= 1 in >=1 of 27 HLA alleles | | | | | |
| HPV16/18 | ADV-HPV16/18 | HPV | HPV | HPV | HPV | HPV | HPV |
| Epitope ID | Epitope Sequence | −31 | −33 | −35 | −45 | −52 | −58 |
| HPV16-E6 | HLDKKQRFHNI (SEQ ID NO: | 2 | | | | | |
| peptide 1 | 113) | | | | | | |
| HPV16-E6 | ISEYRHYCY (SEQ ID NO: 116) | | | 2 | | | |
| peptide 4 | | | | | | | |
| HPV16-E6 | VYDFAFRDL (SEQ ID NO: | | 1 | | 1 | | 1 |
| peptide 5 | 117) | | | | | | |
| HPV16-E6 | KLPQLCTEL (SEQ ID NO: 119) | | | | 2 | | |
| peptide 7 | | | | | | | |
| HPV16-E6 | FAFRDLCIVY (SEQ ID NO: | 5 | | | 4 | | |
| peptide 8 | 120) | | | | | | |
| HPV16-E6 | LCIVYRDGNPYAVCD (SEQ | | | | | | 3 |
| peptide 9 | ID NO: 121) | | | | | | |
| HPV16-E7 | RTLEDLLMGT (SEQ ID NO: | | | 1 | | | |
| peptide 2 | 123) | | | | | | |
| HPV16-E7 | LLMGTLGIV (SEQ ID NO: 125) | 1 | 1 | 1 | | | |
| peptide 4 | | | | | | | |
| HPV16-E7 | TLHEYMLDL (SEQ ID NO: | | 1 | | | | 1 |
| peptide 5 | 126) | | | | | | |
| HPV16-E7 | YMLDLQPETT (SEQ ID NO: | 1 | | | | 1 | |
| peptide 7 | 128) | | | | | | |
| HPV16-E7 | CDSTLRLCV (SEQ ID NO: 129) | | | 1 | | | |
| peptide 8 | | | | | | | |
| HPV18-E6 | KLTNTGLYNL (SEQ ID NO: | | | | 2 | | |
| peptide 1 | 131) | | | | | | |
| HPV18-E6 | FAFKDLFVV (SEQ ID NO: | | 2 | | | | |
| peptide 3 | 133) | | | | | | |
| HPV18-E6 | KLPDLCTEL (SEQ ID NO: 135) | | | | 2 | | |
| peptide 5 | | | | | | | |
| HPV18-E7 | DDLRAFQQLFLNTLS (SEQ ID | | | | 3 | | |
| peptide 1 | NO: 139) | | | | | | |
| HPV18-E7 | QLFLNTLSFV (SEQ ID NO: | | | | 4 | | |
| peptide 3 | 141) | | | | | | |
| HPV18-E7 | LFLNTLSFVCPWCAS (SEQ ID | | | | 4 | | |
| peptide 4 | NO: 142) | | | | | | |
| HPV18-E7 | TLQDIVLHL (SEQ ID NO: 143) | 1 | | 1 | 1 | | |
| peptide 5 | | | | | | | |
| HPV18-E7 | SEEENDEIDGVNHQHLPARR | | | | 8 | | |
| peptide 6 | (SEQ ID NO: 144) | | | | | | |

Table 12 key:
Bolded and underlined numbers indicate shared coverage by an AdV-HPV16/18 epitope between HPV-45 and other genotypes.

Cross-covered HPV-45 epitopes are strong binders in several HLA backgrounds. For example, cross-covered MHC-I epitopes showed IC50 based percentile rank<=1 in certain HLA alleles (Table 13) and cross-covered MHC-II epitopes showed IC50 based percentile rank≤=10 in certain HLA alleles (Table 14).

TABLE 13
HLA alleles with MHC-I epitopes that have
IC50 based percentile rank <= 1

| | SEQ | |
|---|---|---|
| HPV-45 | ID | |
| epitope | NO: | HLA alleles |
| AFKDLCIVY | 332 | HLA-A*30:02, HLA-B*15:01 |
| DGVSHAQL | 348 | HLA-B*51:01 |
| DLRTLQQL | 335 | HLA-B*08:01 |
| DLRTLQQLF | 336 | HLA-A*26:01, HLA-B*08:01 |
| EADGVSHAQ | 346 | HLA-B*35:01 |
| EADGVSHA | 347 | HLA-A*68:02, HLA-B*35:01, HLA- |
| QL | | B*53:01 |
| EENDEADGV | 345 | HLA-B*44:02 |
| FAFKDLCI | 329 | HLA-B*51:01 |
| FAFKDLCIV | 330 | HLA-A*02:06, HLA-B*51:01 |
| FAFKDLCI | 331 | HLA-A*26:01, HLA-A*30:02, HLA- |
| VY | | B*15:01, HLA-B*35:01, |
| | | HLA-B*53:01 |
| FLSTLSFV | 341 | HLA-A*02:01, HLA-A*02:03 |
| GVSHAQLP | 349 | HLA-A*31:01 |
| AR | | |
| ITNTELYNL | 334 | HLA-A*02:06, HLA-A*32:01, HLA- |
| | | B*57:01, HLA-B*58:01 |
| KITNTELY | 333 | HLA-A*30:02 |
| KLPDLCTEL | 135 | HLA-A*02:01, HLA-A*02:01, HLA- |
| | | A*02:03, HLA-A*02:03, HLA- |
| | | A*02:06, HLA-A*02:06, HLA- |
| | | A*32:01, HLA-A*32:01 |
| LFLSTLSF | 340 | HLA-A*23:01, HLA-A*24:02 |
| LPDLCTEL | 328 | HLA-B*07:02, HLA-B*07:02, HLA- |
| | | B*51:01, HLA-B*51:01 |
| LQQLFLSTL | 337 | HLA-A*02:06, HLA-A*32:01, HLA- |
| | | B*15:01 |
| LSTLSFVC | 342 | HLA-B*57:01, HLA-B*58:01 |
| PW | | |
| QLFLSTLSF | 338 | HLA-A*23:01, HLA-A*24:02, HLA- |
| | | A*26:01, HLA-A*30:02, HLA- |
| | | A*32:01, HLA-B*08:01, HLA- |
| | | B*15:01, HLA-B*35:01 |
| QLFLSTLS | 339 | HLA-A*02:01, HLA-A*02:03 |
| FV | | |
| SHAQLPARR | 352 | HLA-A*33:01 |
| STLSFVCPW | 343 | HLA-A*32:01, HLA-B*53:01, HLA- |
| | | B*57:01, HLA-B*58:01 |
| TLQEIVLHL | 344 | HLA-A*02:01, HLA-A*02:03, HLA- |
| | | A*02:06, HLA-A*32:01, HLA- |
| | | A*68:02, HLA-B*08:01 |
| VSHAQLPAR | 350 | HLA-A*11:01, HLA-A*30:01, HLA- |
| | | A*31:01, HLA-A*33:01, HLA-A*68:01 |
| VSHAQLPA | 351 | HLA-A*31:01 |
| RR | | |
| VYQFAFKDL | 327 | HLA-A*23:01, HLA-A*24:02 |

TABLE 14
HLA alleles with MHC-II epitopes that have IC50 based percentile rank <= 10

| | SEQ ID | |
|---|---|---|
| HPV-45 epitope | NO: | HLA alleles |
| ADGVSHAQLPA | 426 | HLA-DRB5*01:01 |
| RR | | |
| DGVSHAQLPARR | 427 | HLA-DRB5*01:01 |
| DLRTLQQLFLST | 428 | HLA-DPA1*01:03/DPB1*02:01,HLA-DPA1*01:03/DPB1*04:01,HLA- |
| | | DPA1*02:01/DPB1*01:01,HLA-DPA1*02:01/DPB1*05:01,HLA- |
| | | DPA1*03:01/DPB1*04:02,HLA-DRB4*01:01 |
| DLRTLQQLFLST | 429 | HLA-DPA1*01:03/DPB1*02:01,HLA-DPA1*01:03/DPB1*04:01,HLA- |
| L | | DPA1*02:01/DPB1*01:01,HLA-DPA1*02:01/DPB1*05:01,HLA- |
| | | DPA1*03:01/DPB1*04:02,HLA-DRB4*01:01 |
| DLRTLQQLFLST | 430 | HLA-DPA1*01:03/DPB1*02:01,HLA-DPA1*01:03/DPB1*04:01,HLA- |
| ILS | | DPA1*02:01/DPB1*01:01,HLA-DPA1*02:01/DPB1*05:01,HLA- |
| | | DPA1*03:01/DPB1*04:02,HLA-DRB1*15:01,HLA-DRB4*01:01 |
| EADGVSHAQLPA | 431 | HLA-DRB5*01:01 |
| RR | | |
| FLSTLSFVCPWC | 432 | HLA-DPA1*01:03/DPB1*02:01,HLA-DPA1*01:03/DPB1*04:01,HLA- |
| | | DQA1*01:01/DQB1*05:01,HLA-DQA1*01:02/DQB1*06:02,HLA- |
| | | DRB1*07:01 |
| FLSTLSFVCPWC | 433 | HLA-DPA1*01:03/DPB1*04:01,HLA-DQA1*01:01/DQB1*05:01,HLA- |
| A | | DRB1*07:01 |
| LFLSTLSFVCPW | 434 | HLA-DPA1*01:03/DPB1*02:01,HLA-DPA1*01:03/DPB1*04:01,HLA- |
| | | DPA1*02:01/DPB1*01:01,HLA-DPA1*02:01/DPB1*05:01,HLA- |
| | | DPA1*03:01/DPB1*04:02,HLA-DQA1*01:02/DQB1*06:02,HLA- |
| | | DRB1*04:01,HLA-DRB1*07:01 |
| LFLSTLSFVCPW | 435 | HLA-DPA1*01:03/DPB1*02:01,HLA-DPA1*01:03/DPB1*04:01,HLA- |
| C | | DPA1*02:01/DPB1*05:01,HLA-DPA1*03:01/DPB1*04:02,HLA- |
| | | DRB1*07:01 |
| LFLSTLSFVCPW | 383 | HLA-DPA1*01:03/DPB1*02:01,HLA-DPA1*01:03/DPB1*04:01,HLA- |
| CA | | DPA1*02:01/DPB1*05:01,HLA-DPA1*03:01/DPB1*04:02,HLA- |
| | | DRB1*07:01 |
| LRTLQQLFLSTL | 436 | HLA-DPA1*01:03/DPB1*02:01,HLA-DPA1*01:03/DPB1*04:01,HLA- |
| | | DPA1*02:01/DPB1*01:01,HLA-DPA1*02:01/DPB1*05:01,HLA- |
| | | DPA1*02:01/DPB1*14:01,HLA-DPA1*03:01/DPB1*04:02,HLA- |
| | | DRB4*01:01 |
| LRTLQQLFLSTLS | 437 | HLA-DPA1*01:03/DPB1*02:01,HLA-DPA1*01:03/DPB1*04:01,HLA- |
| | | DPA1*02:01/DPB1*01:01,HLA-DPA1*02:01/DPB1*05:01,HLA- |
| | | DPA1*02:01/DPB1*14:01,HLA-DPA1*03:01/DPB1*04:02,HLA- |
| | | DRB1*15:01,HLA-DRB4*01:01 |
| LSTLSFVCPWCA | 438 | HLA-DQA1*01:01/DQB1*05:01,HLA-DRB1*15:01 |
| RTLQQLFLSTLS | 439 | HLA-DPA1*01:03/DPB1*02:01,HLA-DPA1*01:03/DPB1*04:01,HLA- |
| | | DPA1*02:01/DPB1*01:01,HLA-DPA1*02:01/DPB1*05:01,HLA- |
| | | DPA1*03:01/DPB1*04:02,HLA-DRB4*01:01 |

Based on the foregoing, a similarly strong T cell immune reaction to AdV-HPV16/18 as in patients with HPV-45 induced tumors is not expected in patients with tumors caused by most or, possibly, even any of the other five analyzed potentially carcinogenic HPV genotypes (HPV-31, HPV-33, HPV-35, HPV-52, and HPV-58), but is expected in patients with HPV-45.

### SEQUENCE LISTING

SEQ ID NOs: 1-147 disclosed in U.S. Ser. No. 16/978,573 are hereby incorporated by reference as if fully and expressly set forth herein as the same. SEQ ID NOs: 148-274 (some of which may be duplicative of one or more of SEQ ID NOs: 1-147) are as follows:

Key regions of AdV-HPV16/18
expression cassette

| | Loca- | | |
|---|---|---|---|
| | tion | Size | |
| Element | (bp) | (bp) | Nucleotide Sequence |
| CMV | 2943- | 365 | SEQ ID NO: 148: |
| enhancer | 3307 | | ctagttattaatagtaatcaattacggggt |
| | | | cattagttcatagcccatatatggagttcc |
| | | | gcgttacataacttacggtaaatggcccgc |
| | | | ctggctgaccgcccaacgacccccgcccat |
| | | | tgacgtcaataatgacgtatgttcccatag |
| | | | taacgccaatagggactttccattgacgtc |
| | | | aatgggtggagtatttacggtaaactgccc |
| | | | acttggcagtacatcaagtgtatcatatgc |
| | | | caagtacgccccctattgacgtcaatgacg |
| | | | gtaaatggcccgcctggcattatgcccagt |
| | | | acatgaccttatgggactttcctacttggc |
| | | | agtacatctacgtattagtcatcgctatta |
| | | | ccatg |
| CMV | 2738- | 205 | SEQ ID NO: 149: |
| promoter | 2942 | | gtgatgcggttttggcagtacatcaatggg |
| | | | cgtggatagcggtttgactcacggggattt |
| | | | ccaagtctccaccccattgacgtcaatggg |
| | | | agtttgttttggcaccaaaatcaacgggac |
| | | | tttccaaaatgtcgtaacaactccgcccca |
| | | | ttgacgcaaatgggcggtaggcgtgtacgg |
| | | | tgggaggtctatataagcagagctc |
| 2X TetO | 2696- | 42 | SEQ ID NO: 150: |
| | 2737 | | tccctatcagtgatagagatctccctatca |
| | | | gtgatagagatc |
| TSS + | 2401- | 295 | SEQ ID NO: 151: |
| 5′ UTR | 2695 | | gtcgacgagctcgtttagtgaaccgtcaga |
| with | | | tcgcctggagacgccatccacgctgttttg |
| splice | | | acctccatagaagacaccgggaccgatcca |
| unit | | | gcctccgcggccgggaacggtgcattggaa |
| | | | cgcggattccccgtgccaagagtgacgtaa |
| | | | gtaccgcctatagagtctataggcccaccc |
| | | | ccttggcttcacttgaggtgtggcaggctt |
| | | | gagatctggccatacacttgagtgacaatg |
| | | | acatccactttgcctttctctccacaggtg |
| | | | tccactcccaggtccaactgcagcc |
| Cloning | 2390- | 11 | SEQ ID NO: 152: |
| site | 2400 | | ggatccggtac |
| Kozak | 2383- | 7 | cgccacc |
| | 2389 | | |
| HPV16/18 | 664- | 1719 | SEQ ID NO: 242 |
| Antigen | 2382 | | (see below) |
| 3′ UTR | 615- | 49 | SEQ ID NO: 155: |
| | 663 | | gcggccgctctcgagtctagctagtctag |
| | | | actagctagaaagatccggg |
| SV40 | 483- | 132 | SEQ ID NO: 156: |
| poly A | 614 | | aacttgtttattgca |
| | | | gcttataatggttacaaataaagcaatagc |
| | | | atcacaaatttcacaaataaagcatttttt |
| | | | tcactgcattctagttgtggtttgtccaaa |
| | | | ctcatcaatgtatcttatcatgtctgg |

CMV-Cytomegalovirus, TetO-tetracycline operator, TSS-transcription start site, UTR-untranslated region

Location and Sequence of
AdV-HPV16/18 E4 Spacer

| Location | |
|---|---|
| (bp) | Nucleotide Sequence |
| 34040- | SEQ ID NO: 157: |
| 34317 | caattgacggccgccatagagcccac |
| | cgcatccccagcatgcctgctattg |
| | tcttcccaatcctcccccttgctgt |
| | cctgccccaccccaccccccagaat |
| | agaatgacacctactcagacaatgc |
| | gatgcaatttcctcattttattagg |
| | aaaggacagtgggagtggcaccttc |
| | cagggtcaaggaaggcacgggggag |
| | gggcaaacaacagatggctggcaac |
| | tagaaggcacagggtaccaacgcgt |
| | cgtaactataacggtcctaaggtag |
| | cg |

Exemplary HPV Antigen Design (Design 4)

| | Size | |
|---|---|---|
| Antigen region | (aa) | Amino Acid Sequence |
| Ankyrin domain | 5 | MQTDR |
| | | (SEQ ID NO: 171) |
| HPV18-E6 peptide 6 | 9 | ELTEVFEFA |
| | | (SEQ ID NO: 172) |
| Ankyrin domain | 7 | ARYSRSD |
| | | (SEQ ID NO: 173) |
| HPV16-E5 peptide | 9 | YIIFVYIPL |
| | | (SEQ ID NO: 174) |
| Ankyrin domain | 3 | ADA |
| Agonist peptide 1 | 9 | KLPQLCTEV |
| | | (SEQ ID NO: 176) |
| Ankyrin domain | 3 | DNM |
| HPV16-E7 peptide 4 | 9 | LLMGTLGIV |
| | | (SEQ ID NO: 178) |
| Ankyrin domain | 9 | PLHAAVSAD |
| | | (SEQ ID NO: 179) |
| HPV16-E7 peptide 5 | 9 | TLHEYMLDL |
| | | (SEQ ID NO: 180) |
| Ankyrin domain | 7 | RNRATDL |
| | | (SEQ ID NO: 181) |
| HPV18-E7 peptide 6 | 20 | SEEENDEIDGVNHQ |
| | | HLPARR |
| | | (SEQ ID NO: 182) |
| Ankyrin domain | 3 | MHD |
| HPV18-E6 peptide 4 | 9 | NLLIRCLRC |
| | | (SEQ ID NO: 184) |
| Ankyrin domain | 10 | PLILAARLAV |
| | | (SEQ ID NO: 185) |
| HPV18-E7 peptide 4 | 15 | LFLNTLSFVCPWCAS |
| | | (SEQ ID NO: 186) |
| Ankyrin domain | 5 | SHADV |
| | | (SEQ ID NO: 187) |
| HPV18-E6 peptide 2 | 9 | KCIDFYSRI |
| | | (SEQ ID NO: 188) |
| Ankyrin domain | 3 | DDL |
| HPV18-E6 peptide 5 | 9 | KLPDLCTEL |
| | | (SEQ ID NO: 190) |
| Ankyrin domain | 11 | ALHWAAAVNNV |
| | | (SEQ ID NO: 191) |
| HPV18-E7 peptide 3 | 10 | QLFLNTLSFV |
| | | (SEQ ID NO: 192) |
| Ankyrin domain | 4 | GANK |
| | | (SEQ ID NO: 193) |
| HPV18-E7 peptide 2 | 9 | FQQLFLNTL |
| | | (SEQ ID NO: 194) |
| Ankyrin domain | 3 | NNR |
| HPV16-E6 peptide 5 | 9 | VYDFAFRDL |
| | | (SEQ ID NO: 196) |
| Ankyrin domain | 12 | PLFLAAREGSYE |
| | | (SEQ ID NO: 197) |
| HPV18-E6 peptide 1 | 10 | KLTNTGLYNL |
| | | (SEQ ID NO: 198) |
| Ankyrin domain | 3 | ANR |
| HPV16-E6 peptide 9 | 15 | LCIVYRDGNPYAVCD |
| | | (SEQ ID NO: 200) |
| Ankyrin domain | 3 | DHM |
| HPV16-E7 peptide 7 | 10 | YMLDLQPETT |
| | | (SEQ ID NO: 202) |
| Ankyrin domain | 3 | PRD |
| HPV16-E6 peptide 8 | 10 | FAFRDLCIVY |
| | | (SEQ ID NO: 204) |
| Ankyrin domain | 21 | IVRLLDLEVSQTSKL |
| | | TRQTDR |
| | | (SEQ ID NO: 205) |
| HPV16-E7 peptide 3 | 8 | TLGIVCPI |
| | | (SEQ ID NO: 206) |
| Ankyrin domain | 8 | AARYSRSD |
| | | (SEQ ID NO: 207) |
| Agonist peptide 3 | 10 | RTLEDLLMGV |
| | | (SEQ ID NO: 208) |
| Ankyrin domain | 3 | ADA |
| HPV16-E6 peptide 6 | 10 | TIHDIILECV |
| | | (SEQ ID NO: 210) |
| Ankyrin domain | 3 | DNM |
| HPV16-E6 peptide 1 | 11 | HLDKKQRFHNI |
| | | (SEQ ID NO: 212) |
| Ankyrin domain | 9 | PLHAAVSAD |
| | | (SEQ ID NO: 213) |
| HPV16-E6 peptide 3 | 15 | TTLEQQYNKPLCDLL |
| | | (SEQ ID NO: 214) |
| Ankyrin domain | 7 | RNRATDL |
| | | (SEQ ID NO: 215) |
| HPV16-E7 peptide 8 | 9 | CDSTLRLCV |
| | | (SEQ ID NO: 216) |
| Ankyrin domain | 3 | MHD |
| HPV16-E6 peptide 2 | 10 | RWTGRCMSCC |
| | | (SEQ ID NO: 218) |
| Ankyrin domain | 10 | PLILAARLAV |
| | | (SEQ ID NO: 219) |
| HPV18-E6 peptide 7 | 10 | SLQDIEITCV |
| | | (SEQ ID NO: 220) |
| Ankyrin domain | 5 | SHADV |
| | | (SEQ ID NO: 221) |
| HPV16-E7 peptide 1 | 10 | LCVQSTHVDI |
| | | (SEQ ID NO: 222) |
| Ankyrin domain | 3 | DDL |
| HPV16-E7 peptide 6 | 10 | AHYNIVTFCC |
| | | (SEQ ID NO: 224) |
| Ankyrin domain | 10 | LHWAAAVNNV |
| | | (SEQ ID NO: 225) |
| HPV18-E7 peptide 1 | 15 | DDLRAFQQLFLNTLS |
| | | (SEQ ID NO: 226) |
| Ankyrin domain | 4 | GANK |
| | | (SEQ ID NO: 227) |
| HPV16-E6 peptide 4 | 9 | ISEYRHYCY |
| | | (SEQ ID NO: 228) |
| Ankyrin domain | 3 | NNR |
| HPV16-E6 peptide 7 | 9 | KLPQLCTEL |
| | | (SEQ ID NO: 230) |
| Ankyrin domain | 12 | PLFLAAREGSYE |
| | | (SEQ ID NO: 231) |
| HPV18-E6 peptide 8 | 9 | KTVLELTEV |
| | | (SEQ ID NO: 232) |
| Ankyrin domain | 3 | ANR |
| HPV16-E7 peptide 2 | 10 | RTLEDLLMGT |
| | | (SEQ ID NO: 234) |
| Ankyrin domain | 3 | DHM |
| HPV18-E6 peptide 3 | 9 | FAFKDLFVV |
| | | (SEQ ID NO: 236) |
| Ankyrin domain | 3 | PRD |
| Agonist peptide 2 | 10 | QLYNKPLCDV |
| | | (SEQ ID NO: 238) |
| Ankyrin domain | 3 | IVR |
| HPV18-E7 peptide 5 | 9 | TLQDIVLHL |
| | | (SEQ ID NO: 240) |
| Ankyrin domain | 2 | LE |

AdV-HPV16/18 antigen DNA open
reading frame sequence
SEQ ID NO: 242
atgcaaaccgacagagagcttaccgaggttttcgagtttgcagcc
cggtactcccggagcgactacattatttttgtctatatccctctc
gcagacgcaaagctgcctcaactgtgtacagaggttgataatatg
ctactgatgggcactctcggcatcgtgccgctccacgccgccgtg
agcgccgacaccctgcatgaatacatgctggacctgagaaatcgg
gcaaccgacttatccgaggaggagaacgacgaaattgatggagtt
aatcaccagcacctaccagcaagacgaatgcacgacaacctactg
attagatgcttgaggtgtccccttattttggcagccagactcgcc
gttctgttcctgaacaccctgagctttgtatgcccctggtgtgcc
agctcgcacgcagacgtgaaatgtattgacttctattccagaatc
gacgacctaaaactgcctgatctctgtacagagcttgcgttgcac
tgggcagccgccgtgaacaacgtgcagttgtttctgaacacctta
tccttcgtgggcgctaacaaattccagcaacttttcttgaacacc
ctaaacaacagagtttatgactttgctttcagagacctccccctg
ttccttgctgccagagagggatcatacgagaaactgacaaatacg
ggcctctacaatttggccaacagactgtgcatcgtgtacagagat
ggcaatccctacgccgtttgcgacgaccacatgtacatgctcgac
ctccagccggagacaactcccagagacttcgcttttcgggacctg
tgtatcgtgtatattgtaagactcctggacctggaagtgagccaa
acaagtaagctaaccagacaaacagacagaacacttgggatcgtg
tgtcctattgcagcccggtatagtaggtcagaccggactctggag
gacctgttaatgggggtggcggacgcaacaatccacgacattatc
ttggagtgtgttgacaatatgcacctggacaagaaacagagattc
cacaacatacccctgcacgcagccgtcagcgcggatactactctg
gagcagcaatataacaagcccctgtgcgacctcctgcggaatcgg
gcaactgatctctgtgacagtacccttcgtttgtgcgttatgcac
gacaggtggactgggagatgtatgtcttgctgtcctctcattctg
gccgcaagactggccgttagtctgcaagacatcgagatcacctgt
gtcagtcacgccgatgtgctatgcgtgcagagcacccacgttgat
attgatgacctagcacactacaacatcgttactttttgctgtcta
cactgggccgcagccgtgaataacgtggacgacctacgcgctttc
caacaactgttccttaacaccctgagcggcgcaaacaagatcagc
gagtaccgccactactgttataataaccggaagctgccacagcta
tgcacagagttgccactgttcctggcagcaagagagggatcttat
gaaaagaccgtgctggagctgacagaggtggctaatcgccgcaca
ctggaggacctgttgatgggaaccgatcacatgttcgcctttaag
gaccttttcgttgttcccagagaccagctttacaacaaacctcta
tgcgacgtaatagtgaggaccttgcaggatattgtgctccatctg
ctggaataa.
AdV-HPV16/18 antigen protein sequence
SEQ ID NO: 243
MQTDRELTEVFEFAARYSRSDYIIFVYIPLADAKLPQLCTEVDNM
LLMGTLGIVPLHAAVSADTLHEYMLDLRNRATDLSEEENDEIDGV
NHQHLPARRMHDNLLIRCLRCPLILAARLAVLFLNTLSFVCPWCA
SSHADVKCIDFYSRIDDLKLPDLCTELALHWAAAVNNVQLFLNTL
SFVGANKFQQLFLNTLNNRVYDFAFRDLPLFLAAREGSYEKLTNT
GLYNLANRLCIVYRDGNPYAVCDDHMYMLDLQPETTPRDFAFRDL
CIVYIVRLLDLEVSQTSKLTRQTDRTLGIVCPIAARYSRSDRTLE
DLLMGVADATIHDIILECVDNMHLDKKQRFHNIPLHAAVSADTTL
EQQYNKPLCDLLRNRATDLCDSTLRLCVMHDRWTGRCMSCCPLIL
AARLAVSLQDIEITCVSHADVLCVQSTHVDIDDLAHYNIVTFCCL
HWAAAVNNVDDL
AdV-HPV16/18 antigen protein sequence
SEQ ID NO: 243
RAFQQLFLNTLSGANKISEYRHYCYNNRKLPQLCTELPLFLAARE
GSYEKTVLELTEVANRRTLEDLLMGTDHMFAFKDLFVVPRDQLYN
KPLCDVIVRTLQDIVLHLLE
AdV-HPV16/18 DNA Sequence
SEQ ID NO: 244
catcatcaataatataccttattttggattgtggccaatatgata
atgaggtgggcggggagaggcggggcgggtgacgtaggacgcgcg
agtagggttgggaggtgtggcggaagtgtggcatttgcaagtggg
aggagctcacatgcaagcttccgtcgcggaaaatgtgacgttttt
gatgagcgccgcctacctccggaagtgccaattttcgcgcgcttt
tcaccggatatcgtagtaattttggggggaccatgtaagatttgg
ccattttcgcgcgaaaagtgaaacggggaagtgaaaactgaataa
tagggcgttagtcatagtgcgtaatatttaccgagggccgaggga
ctttgaccgattacgtggaggactcgcccaggtgttttttacgtg
aatttccgcgttccgggtcaaagtctccgttttattgtcaccgtc
atttgacagatcgggatcggtgatcaccgatccagacatgataag
atacattgatgagtttggacaaaccacaactagaatgcagtgaaa
aaaatgctttatttgtgaaatttgtgatgctattgctttatttgt
aaccattataagctgcaataaacaagttcccggatctttctagct
agtctagactagctagactcgagagcggccgcttattccagcaga
tggagcacaatatcctgcaaggtcctcactattacgtcgcataga
ggtttgttgtaaagctggtctctgggaacaacgaaaaggtcctta
aaggcgaacatgtgatcggttcccatcaacaggtcctccagtgtg
cggcgattagccacctctgtcagctccagcacggtcttttcataa
gatccctctcttgctgccaggaacagtggcaactctgtgcatagc
tgtggcagcttccggttattataacagtagtggcggtactcgctg
atcttgtttgcgccgctcagggtgttaaggaacagttgttggaaa
gcgcgtaggtcgtccacgttattcacggctgcggcccagtgtaga
cagcaaaaagtaacgatgttgtagtgtgctaggtcatcaatatca
acgtgggtgctctgcacgcatagcacatcggcgtgactgacacag
gtgatctcgatgtcttgcagactaacggccagtcttgcggccaga
atgagaggacagcaagacatacatctcccagtccacctgtcgtgc
ataacgcacaaacgaagggtactgtcacagagatcagttgcccga
ttccgcaggaggtcgcacaggggcttgttatattgctgctccaga
gtagtatccgcgctgacggctgcgtgcaggggtatgttgtggaat
ctctgtttcttgtccaggtgcatattgtcaacacactccaagata
atgtcgtggattgttgcgtccgccacccccattaacaggtcctcc
agagtccggtctgacctactataccgggctgcaataggacacacg
atcccaagtgttctgtctgtttgtctggttagcttacttgtttgg
ctcacttccaggtccaggagtcttacaatatacacgatacacagg
tcccgaaaagcgaagtctctgggagttgtctccggctggaggtcg
agcatgtacatgtggtcgtcgcaaacggcgtagggattgccatct
ctgtacacgatgcacagtctgttggccaaattgtagaggcccgta
tttgtcagtttctcgtatgatccctctctggcagcaaggaacagg
gggaggtctctgaaagcaaagtcataaactctgttgtttagggtg
ttcaagaaaagttgctggaatttgttagcgcccacgaaggataag
gtgttcagaaacaactgcacgttgttcacggcggctgcccagtgc
aacgcaagctctgtacagagatcaggcagttttaggtcgtcgatt
ctggaatagaagtcaatacatttcacgtctgcgtgcgagctggca
caccaggggcatacaaagctcagggtgttcaggaacagaacggcg
agtctggctgccaaaataaggggacacctcaagcatctaatcagt
aggttgtcgtgcattcgtcttgctggtaggtgctggtgattaact
ccatcaatttcgtcgttctcctcctcggataagtcggttgcccga
tttctcaggtccagcatgtattcatgcagggtgtcggcgctcacg
gcggcgtggagcggcacgatgccgagagtgcccatcagtagcata
ttatcaacctctgtacacagttg
AdV-HPV16/18 DNA Sequence
SEQ ID NO: 244
aggcagctttgcgtctgcgagagggatatagacaaaaataatgta
gtcgctccgggagtaccgggctgcaaactcgaaaacctcggtaag
ctctctgtcggtttgcatggtggcggtaccggatccggctgcagt
tggacctgggagtggacacctgtggagagaaaggcaaagtggatg
tcattgtcactcaagtgtatggccagatctcaagcctgccacacc
tcaagtgaagccaaggggggggcctatagactctataggcggtac
ttacgtcactcttggcacggggaatccgcgttccaatgcaccgtt
cccggccgcggaggctggatcggtcccggtgtcttctatggaggt
caaaacagcgtggatggcgtctccaggcgatctgacggttcacta
aacgagctcgtcgacgatctctatcactgatagggagatctctat
cactgatagggagagctctgcttatatagacctcccaccgtacac
gcctaccgcccatttgcgtcaatggggcggagttgttacgacatt
ttggaaagtcccgttgattttggtgccaaaacaaactcccattga
cgtcaatggggtggagacttggaaatccccgtgagtcaaaccgct
atccacgcccattgatgtactgccaaaaccgcatcaccatggtaa
tagcgatgactaatacgtagatgtactgccaagtaggaaagtccc
ataaggtcatgtactgggcataatgccaggcgggccatttaccgt
cattgacgtcaatagggggcgtacttggcatatgatacacttgat
gtactgccaagtgggcagtttaccgtaaatactccacccattgac
gtcaatggaaagtccctattggcgttactatgggaacatacgtca
ttattgacgtcaatgggcgggggtcgttgggggtcagccaggcgg
gccatttaccgtaagttatgtaacgcggaactccatatatgggct
atgaactaatgaccccgtaattgattactattaataactagaggc
ctgaccatctggtgctggcctgcaccagggccgagtttgggtcta
gcgatgaggataccgattgaggtgggtaaggtgggcgtggctaga
agggtggggcgtgtataaattgggggtctaagggtctctctgttt
tgtcttgcaacagccgccgccatgagcgacaccggcaacagcttt
gatggaagcatctttagcccctatctgacagtgcgcatgcctcac
tgggctggagtgcgtcagaatgtgatgggttccaacgtggatgga
cgccccgttctgccttcaaattcgtctacaatggcctacgcgacc
gtgggaggaactccgctggacgccgcgacctccgccgccgcctcc
gccgccgccgcgaccgcgcgcagcatggctacggacctttacagc
tctttggtggcgagcggcgcggcctctcgcgcgtctgctcgggat
gagaaactgaccgctctgctgcttaaactggaagacttgacccgg
gagctggctcaactgacccagcaggtctccagcttgcgtgagagc
agccttgcctccccctaatggcccataatataaataaaagccagt
ctgtttggattaagcaagtgtatgttctttatttaactctccgcg
cgcggtaagcccgggaccagcggtctcggtcgtttagggtgcggt
ggattctttccaacacgtggtacaggtggctctggatgtttagat
acatgggcatgagtccatccctggggggaggtagcaccactgcag
agcttcgtgctcgggggtggtgttgtatatgatccagtcgtagca
ggagcgctgggcgtggtgctgaaaaatgtccttaagcaagaggct
tatagctagggggaggcccttggtgtaagtgtttacaaatctgct
cagttgggaggggtgcatccggggggatataatgtgcatcttgga
ctggatttttaggttggctatgttcccacccagatcccttctggg
attcatgttgtgcaggaccaccagcacggtatatccagtacactt
gggaaatttatcgtggagcttagacgggaatgcatggaagaactt
ggagacgcccttgtggcctcccagattttccatacattcgtccat
gatgatggcaatgggcccgtgggaagctgcctgagcaaaaatgtt
tctgggatcgctcacatcgtagttatgttccagggtgaggtcatc
ataggacatctttacaaatcgggggcggagggtcccggactgggg
gatgatggtgccctcgggccccggggcgtagttcccctcacagat
ctgcatctcccaggctttcatttcagagggagggatcatatccac
ctgcggagcgatgaaaaacacagtttctggcgcaggggagattaa
ctgggatgagagcaggtttctgagcagctgtgactttccacagcc
ggtgggcccatatatcacgcctatcaccggctgcagctggtagtt
aagagagctgcagctgccgtcctcccggagcaggggggccacctc
gttcagcatatccctgacgtggatgttctccctgaccaattccgc
cagaaggcgctcgccgcccagcgaaagcagctcttgcaaggaagc
aaaatttttcagcggttttaggccgtcggccgtgggca
AdV-HPV16/18 DNA Sequence
SEQ ID NO: 244
tgtttttcagcgtctgggtcagcagttccagtctgtcccacagct
cggtgatgtgctctacggcatctcgatccagcagatctcctcgtt
tcgcgggttggggcggctttcgctgtagggcaccagccgatgggc
gtccagcggggccagagtcatgtccttccatgggcgcagggtcct
cgtcagggtggtctgggtcacggtgaaggggtgcgctccgggttg
ggcgctggccagggtgcgcttgaggctggttctgctggtgctgaa
tcgctgccgctcttcgccctgcgcgtcggccaggtagcatttgac
catggtctcgtagtcgagaccctcggcggcgtgccccttggcgcg
gagctttcccttggaggtggcgccgcacgaggggcactgcaggct
cttcagggcgtagagcttgggagcgagaaacacggactctgggga
gtaggcgtccgcgccgcaggaagcgcagaccgtctcgcattccac
cagccaagtgagctccgggcggtcagggtcaaaaaccaggttgcc
cccatgctttttgatgcgtttcttacctcggctctccatgaggcg
gtgtcccttctcggtgacgaagaggctgtccgtgtctccgtagac
cgacttcaggggcctgtcttccagcggagtgcctctgtcctcctc
gtagagaaactctgaccactctgagacgaaggcccgcgtccaggc
caggacgaaggaggccacgtgggaggggtagcggtcgttgtccac
tagcgggtccaccttctccagggtgtgcaggcacatgtccccctc
ctccgcgtccagaaaagtgattggcttgtaggtgtaggacacgtg
accgggggttcccgacgggggggtataaaaggggggggcaccctt
tcatcttcactctcttccgcatcgctgtctgcgagagccagctgc
tggggtaagtattccctctcgaaggcgggcatgacctcagcgctc
aggttgtcagtttctaaaaatgaggaggatttgatgttcacctgt
ccggaggtgatacctttgagggtacctgggtccatctggtcagaa
aacactatttttttgttgtcaagcttggtggcgaacgacccgtag
agggcgttggagagcagcttggcgatggagcgcagggtctggttt
ttgtcgcggtcggctcgctccttggccgcgatgttgagttgcacg
tactcgcgggccacgcacttccactcggggaagacggtggtgcgc
tcgtctgggatcaggcgcaccctccagcctcggttgtgcagggtg
accatgtcgacgctggtggcgacctcgccgcgcaggcgctcgttg
gtccagcagaggcggccgcccttgcgcgagcagaaggggggtagg
gggtccagctggtcctcgtttggggggtccgcgtcgatggtgaag
accccggggagcaagcgcgggtcaaagtagtcgatcttgcaagct
tgcatgtccagagcccgctgccattcgcgggcggcgagcgcgcgc
tcgtaggggttgaggggcgggccccagggcatggggtgggtgagc
gcggaggcgtacatgccgcagatgtcatacacgtacaggggttcc
ctgaggatgccgaggtaggtggggtagcagcgccccccgcggatg
ctggcgcgcacgtagtcatagagctcgtgggagggggccagcatg
ttgggcccgaggttggtgcgctgggggcgctcggcgcggaaggcg
atctgcctgaagatggcatgggagttggaggagatggtgggccgc
tggaagacgttgaagcttgcttcttgcaagcccaccgagtccctg
acgaagcaggcgtaggactcgcgcagcttgtgcaccagctcggcg
gtgacctggacgtcgagcgcgcagtagtcgagggtctcgcggatg
atgtcatacttatcctcccccttctttttccacagctcgcggttg
aggacgaactcttcgcggtctttccagtactcttggaggggaaac
ccgtccgtgtccgaacggtaagagcctagcatgtagaactggttg
acggcctggtaggggcaacagcccttctccacgggcagcgcgtag
gcctgcgccgccttgcggagggaggtgtgggtgagggcgaaagtg
tccctgaccatgactttgaggtattgatgtttgaagtctgtgtca
tcgcagccgccctgttcccacagggtgtagtccgtgcgctttttg
gagcgcgggttgggcagggagaaggtgaggtcattgaagaggatc
ttccccgctcgaggcatgaagtttctggtgatgcgaaagggccct
gggaccgaggagcggttgttgatgacctgggcggccaggacgatc
tcgtcaaagccgtttatgttgtggcccacgatgtagagctccaaa
aagcggggctggcccttgatggaggggagctttttgagttcctcg
taggtgagctcctcgggcgattccaggccgtgctcctccagggcc
cagtcttgcaagtgagggttggccgccaggaaggatcgccagagg
tcgcgggccatgagggtctgcaggcggtcgcggaaggttctgaac
tgtcgccccacggccatcttttcgggggtgatgcagta
AdV-HPV16/18 DNA Sequence
SEQ ID NO: 244
gaaggtgagggggtctttctcccaggggtcccatctgagctctcg
ggcgaggtcgcgcgcggcggcgaccagagcctcgttgccccccag
tttcatgaccagcatgaagggcacgagctgcttgccaaaggctcc
catccaagtgtaggtctctacatcgtaggtgacaaagaggcgctc
cgtgcgaggatgagagccgatcgggaagaactggatctcccgcca
ccagttggaggattggctgttgatgtggtgaaagtagaagtcccg
tctgcgggccgagcactcgtgctggcttttgtaaaagcgaccgca
gtactggcagcgctgcacgggttgtatatcttgcacgaggtgaac
ctggcgacctctgacgaggaagcgcagcgggaatctaagtccccc
gcctggggtcccgtgtggctggtggtcttctactttggttgtctg
gccgccagcatctgtctcctggagggcgatggtggagcagaccac
cacgccgcgagagccgcaggtccagatctcggcgctcggcgggcg
gagtttgatgacgacatcgcgcacattggagctgtccatggtctc
cagctcccgcggcggcaggtcagctgggagttcctggaggttcac
ctcgcagagacgggtcaaggcgcgggcagtgttgagatggtatct
gatttcaaggggcgtgttggcggcggagtcgatggcttgcaggag
gccgcagccccggggggccacgatggttccccgcggggcgcgagg
ggaggcggaagctgggggtgtgttcagaagcggtgacgcgggggg
cccccggaggtagggggggttccggccccacaggcatgggcggca
ggggcacgtcttcgccgcgcgcgggcaggggctggtgctggctcc
gaagagcgcttgcgtgcgcgacgacgcgacggttggtgtcctgta
tctgacgcctctgagtgaagaccacgggtcccgtgaccttgaacc
tgaaagagagttcgacagaatcaatctcggcatcgttgacagcgg
cctggcgcaggatctcctgcacgtcgcccgagttgtcctggtagg
cgatctctgccatgaactgctcgatctcttcttcctggagatctc
ctcgtccggcgcgctccacggtggccgccaggtcgttggagatgc
gacccatgagctgcgagaaggcgttgagcccgccctcgttccaga
cccggctgtagaccacgcccccctcggcgttgcgggcgcgcatga
ccacctgggccaggttgagctccacgtgtcgcgtgaagacggcgt
agttgcgcaggcgctggaaaaggtagttcagggtggtggcggtgt
gctcggcgacgaagaagtacatgacccagcgccgcaacgtggatt
cattgatgtcccccaaggcctccaggcgctccatggcctcgtaga
agtccacggcgaagttgaaaaactgggagttgcgagcggacacgg
tcaactcctcctccagaagacggatgagctcggcgacagtgtcgc
gcacctcgcgctcgaaggccacggggggcgcttcttcctcttcca
cctcttcttccatgatcgcttcttcttcttcctcagccgggacgg
ggggggcggcggcggcgggggaggggcgcggcggcggcggcggcg
caccgggaggcggtcgatgaagcgctcgatcatctccccccgcat
gcggcgcatggtctcggtgacggcgcggccgttctcccgggggcg
cagctcgaagacgccgcctctcatctcgccgcggggcgggcggcc
gtgaggtagcgagacggcgctgactatgcatcttaacaattgctg
tgtaggtacaccgccgagggacctgattgagtccagatccaccgg
atccgaaaacctttggaggaaagcgtctatccagtcgcagtcgca
aggtaggctgagcaccgtggcgggcgggggcgggtctggagagtt
cctggcggagatgctgctgatgatgtaattaaagtaggcggtctt
gagaaggcggatggtggacaggagcaccatgtctttgggtccggc
ctgttggatgcggaggcggtcggccatgccccaggcctcgttctg
acaccggcgcaggtctttgtagtagtcttgcatgagtctttccac
cggcacctcttctccttcctcttctccatctcgccggtggtttct
cgcgccgcccatgcgcgtgaccccaaagcccctgagcggctgcag
cagggccaggtcggcgaccacgcgctcggccaagatggcctgctg
cacctgagtgagggtcctctcgaagtcatccatgtccacgaagcg
gtggtaggcgcccgtgttgatggtgtaggtgcagttggccatgac
ggaccagttgacggtctggtgtcccggctgcgagagctccgtgta
ccgcaggcgcgagaaggcgcgggaatcgaacacgtagtcgttgca
agtccgcaccagatactggtagcccaccaggaagtgcggcggagg
ttggcgatagaggggccagcgctgggtggcgggggcgccgggcgc
caggttttccagcatgaggcggtggtatccgtagatgtacctgga
catccaggtgatg
AdV-HPV16/18 DNA Sequence
SEQ ID NO: 244
ccggcggcggtggtggtggcgcgcgcgtagtcgcggacccggttc
cagatgtttcgcaggggcgagaagtgttccatggtcggcacgctc
tggccggtgaggcgcgcgcagtcgttgacgctctatacacacaca
aaaacgaaagcgtttacagggctttcgttctgtagcctggaggaa
agtaaatgggttgggttgcggtgtgccccggttcgagaccaagct
gagctcggccggctgaagccgcagctaacgtggtattggcagtcc
cgtctcgacccaggccctgtatcctccaggatacggtcgagagcc
cttttgctttcttggccaagcgcccgtggcgcgatctgggataga
tggtcgcgatgagaggacaaaagcggctcgcttccgtagtctgga
gaaacaatcgccagggttgcgttgcggcgtaccccggttcgagcc
cctatggcggcttgaatcggccggaaccgcggctaacgagggccg
tggcagccccgtcctcaggaccccgccagccgacttctccagtta
cgggagcgagccccttttgttttttattttttagatgcatcccgt
gctgcggcagatgcgcccctcgccccggcccgatcagcagcagca
acagcaggcatgcagacccccctctcccctttccgccccggtcac
cacggccgcggcggccgtgtcgggcgcggggggcgcgctggagtc
agatgagccaccgcggcggcgacctaggcagtatctggacttgga
agagggcgagggactggcgcggctgggggcgaactctccagagcg
ccacccgcgggtgcagttgaaaagggacgcgcgcgaggcgtacct
gccgcggcagaacctgtttcgcgaccgcgggggcgaggagcccga
ggagatgcgagactgcaggttccaagcggggcgcgagctgcggcg
cgggctggacagacagcgcctgctgcgcgaggaggactttgagcc
cgacacgcagacgggcatcagccccgcgcgcgcgcacgtagccgc
ggccgacctggtgaccgcctacgagcagacggtgaaccaggagcg
caacttccaaaagagcttcaacaaccacgtgcgcacgctggtggc
gcgcgaggaggtgaccctgggtctcatgcatctgtgggacctggt
ggaggcgatcgtgcagaaccccagcagcaagcccctgaccgcgca
gctgttcctggtggtgcagcacagcagggacaacgaggccttcag
ggaggcgctgctgaacatcaccgagccggaggggcgctggctcct
ggacctgataaacatcctgcagagcatagtggtgcaggagcgcag
cctgagcctggccgagaaggtggcggccatcaactactctatgct
gagcctgggcaagttctacgcccgcaagatctacaagacccccta
cgtgcccatagacaaggaggtgaagatagacagcttctacatgcg
catggcgctgaaggtgctgaccctgagcgacgacctgggagtgta
ccgcaacgagcgcatccacaaggccgtgagcgccagccggcggcg
cgagctgagcgaccgcgagctgatgcacagtctgcagcgcgcgct
gaccggcgcgggcgagggcgacagggaggtcgagtcctacttcga
catgggggccgacctgcactggcagccgagccgccgcgccctgga
ggcggcgggggcgtacggcggccccctggcggccgatgaccagga
agaggaggactatgagctagaggagggcgagtacctggaggactg
acctggctggtggtgttttggtatagatgcaagatccgaacgtgg
cggacccggcggtccgggcggcgctgcaaagccagccgtccggca
ttaactcctctgacgactgggccgcggccatgggtcgcatcatgg
ccctgaccgcgcgcaaccccgaggctttcaggcagcagcctcagg
ccaaccggctggcggccatcttggaagcggtagtgcccgcgcgct
ccaaccccacccacgagaaggtgctggccatagtcaacgcgctgg
cggagagcagggccatccgcgcggacgaggccggactggtgtacg
atgcgctgctgcagcgggtggcgcggtacaacagcggcaacgtgc
agaccaacctggaccgcctggtgacggacgtgcgcgaggccgtgg
cgcagcgcgagcgcttgcatcaggacggtaacctgggctcgctgg
tggcgctaaacgccttcctcagcacccagccggccaacgtaccgc
gggggcaggaggactacaccaactttttgagcgcgctgcggctga
tggtgaccgaggtccctcagagcgaggtgtaccagtcggggcccg
actacttcttccagaccagcagacagggcttgcaaaccgtgaacc
tgagccaggctttcaagaacctgcgggggctgtggggagtgaagg
cgcccaccggcgaccgggctacggtgtccagcctgctaaccccca
actcgcgcctgctgctgctgctgatcgcgcccttcacggacagcg
ggagcgtctcgcgggagacctatct
AdV-HPV16/18 DNA Sequence
SEQ ID NO: 244
gggccacctgctgacgctgtaccgcgaggccatcgggcaggcgca
ggtggacgagcacaccttccaagagatcaccagcgtgagccacgc
gctggggcaggaggacacgggcagcctgcaggcgaccctgaacta
cctgctgaccaacaggcggcagaagattcccacgctgcacagcct
gacccaggaggaggagcgcatcttgcgctacgtgcagcagagcgt
gagcctgaacctgatgcgcgacggcgtgacgcccagcgtggcgct
ggacatgaccgcgcgcaacatggaaccgggcatgtacgcctccca
ccggccgttcatcaaccgcctgatggactacttgcatcgggcggc
ggccgtgaaccccgagtacttcactaatgccattctgaatcccca
ctggatgccccctccgggtttctacaacggggactttgaggtgcc
cgaggtcaacgacgggttcctctgggatgacatggatgacagtgt
gttctcacccaacccgctgcgcgccgcgtctctgcgattgaagga
gggctctgacagggaaggaccgagaagtctggcctcctccctggc
tctgggagcggtgggcgccacgggcgcggcggcgcggggcagtag
ccccttccccagcctggcagactctctgaacagcggggggtgagc
aggccccgcttgctaggcgaggaggagtatctgaacaactccctg
ctgcagcccgcgagggacaagaacgctcagcggcagcagtttccc
aacaatgggatagagagcctggtggacaagatgtccagatggaag
acgtatgcgcaggagtacaaggagtgggaggaccgccagccgcgg
cccttgccgccccctaggcagcgctggcagcggcgcgcgtccaac
cgccgctggaggcaggggcccgaggacgatgatgactctgcagat
gacagcagcgtgttggacctgggcgggagcgggaaccccttttcg
cacctgcgcccacgcctgggcaagatgttttaaaagaaaaaaaaa
aaaataaaactcaccaaggccatggcgacgagcgttggttttttg
ttcccttccttagtatgcggcgcgcggcgatgttcgaggaggggc
ctcccccctcttacgagagcgcgatggggatttctcctgcggcgc
ccctgcagcctccctacgtgcctcctcggtacctgcaacctacag
gggggagaaatagcatctgttactctgagctgcagcccctgtacg
ataccaccagactgtacctggtggacaacaagtccgcggacgtgg
cctccctgaactaccagaacgaccacagcgattttttgaccacgg
tgatccaaaacaacgacttcaccccaaccgaggccagcactcaga
ccataaacctggataacaggtcgaactggggcggcgacctgaaga
ccatcttgcacaccaacatgcccaacgtgaacgagttcatgttca
ccaactcttttaaggcgcgggtgatggtggcgcgcgagcaggggg
aggcgaagtacgagtgggtggacttcacgctgcccgagggcaact
actcagagaccatgactctcgacctgatgaacaatgcgatcgtgg
aacactatctgaaagtgggcaggcagaacggggtgaaggaaagcg
atatcggggtcaagtttgacaccagaaacttccgtctgggctggg
accccgtgaccgggctggtcatgccgggggtctacaccaacgagg
cctttcatcccgacatagtgcttctgcccggctgtggggggactt
cacccagagccggctgagcaacctgctgggcattcgcaagcggca
gcctttccaggagggtttcaagatcacctatgaggatctgaaggg
gggcaacattcccgcgctccttgatctggacgcctacgaggagag
cttgaaacccgaggagagcgctggcgacagcggcgagagtggcga
ggagcaagccggcggcggtggcggcgcgtcggtagaaaacgaaag
tacgcccgcagtggcggcggacgctgcggaggtcgagccggaggc
catgcagcaggacgcagaggagggcgcacaggagggcgcgcagaa
ggacatgaacgatggggagatcaggggagacacattcgccacccg
gggcgaagaaaaagaggcagaggcggcggcggcggcgacggcgga
ggccgaaaccgaggttgaggcagaggcagagcccgagaccgaagt
tatggaagacatgaatgatggagaacgtaggggcgacacgttcgc
cacccggggcgaagagaaggcggcggaggcagaagccgcggctga
ggaggcggctgcggctgcggccaagactgaggctgcggctaaggc
tgaggtcgaagccaatgttgcggttgaggctcaggctgaggagga
ggcggcggctgaagcagttaaggaaaaggcccaggcagagcagga
agagaaaaaacctgtcattcaacctctaaaagaagatagcaaaaa
gcgcagttacaacgtcatcgagggcagcacctttacccagtaccg
cagctggtacctggcgtacaactacggcg
AdV-HPV16/18 DNA Sequence
SEQ ID NO: 244
acccggtcaagggggtgcgctcgtggaccctgctctgcacgccgg
acgtcacctgcggctccgagcagatgtactggtcgctgccgaaca
tgatgcaagacccggtgaccttccgctccacgcggcaggttagca
acttcccggtggtgggcgccgaactgctgcccgtgcactccaaga
gtttttacaacgagcaggccgtctactcccagctgatccgccagg
ccacctctctgacccacgtgttcaatcgctttcccgagaaccaga
ttttggcgcgcccgccggcccccaccatcaccaccgtgagtgaaa
acgttcctgccctcacagatcacgggacgctaccgctgcgcaaca
gcatctcaggagtccagcgagtgaccattactgacgccagacgcc
ggacctgcccctacgtttacaaggccttgggcatagtctcgccgc
gcgtcctctccagtcgcactttttaaaacacatctacccacacgt
tccaaaatcatgtccgtactcatctcacccagcaacaacaccggc
tgggggctgcgcgcgcccagcaagatgtttggaggggcgaggaag
cgctccgaccagcaccctgtgcgcgtgcgcggccactaccgcgcg
ccctggggagcgcacaagcgcgggcgcacagggcgcaccactgtg
gacgacgtcattgactccgtagtggagcaagcgcgccactacaca
cccggcgcgccgaccgcccccgccgtgtccaccgtggaccaggcg
atcgaaagcgtggtacagggcgcgcggcactatgccaaccttaaa
agtcgccgccgccgcgtggcccgccgccatcgccggagaccccgg
gccaccgccgccgcgcgccttactaaggctctgctcaggcgcgcc
aggcgaactggccaccgggccgccatgagggccgcacggcgggct
gccgctgccgcaagcgccgtggccccgcgggcacgaaggcgcgcg
gccgccgccgccgccgccgccatttccagcttggcctcgacgcgg
cgcggtaacatatactgggtgcgcgactcggtaaccggcacgcgg
gtacccgtgcgctttcgccccccgcggaattagcacaagacaaca
tacacactgagtctcctgctgttgtgtatcccagcggcgaccgtc
agcagcggcgacatgtccaagcgcaaaattaaagaagagatgctc
caggtcatcgcgccggagatctatgggcccccgaagaaggaggag
gatgattacaagccccgcaagctaaagcgggtcaaaaagaaaaag
aaagatgatgatgatgacgaggcggtggagtttgtccgccgcatg
gcacccaggcgccccgtgcagtggaagggccggcgcgtgcagcgc
gttttgcgccccggcaccgcggtggtcttcacgcccggcgagcgc
tccacgcgcactttcaagcgggtgtacgatgaggtgtacggcgac
gaggacctgttggagcaggccaaccagcgctttggggagtttgca
tatgggaaacggccccgcgagagtctaaaagaggacctgctggcg
ctaccgctggacgagggcaatcccaccccgagtctgaagccggta
accctgcaacaggtgctgcctttgagcgcgcccagcgagcataag
cgagggttgaagcgcgaaggcggggacctggcgcccaccgtgcag
ttgatggtgcccaagcggcagaagctggaggacgtgctggagaaa
atgaaagtagagcccgggatccagcccgagatcaaggtccgcccc
atcaagcaggtggcgcccggcgtgggagtccagaccgtggacgtt
aggattcccacggaggagatggaaacccaaaccgccactccctct
tcggcggccagcgccaccaccggcaccgcttcggtagaggtgcag
acggacccctggctacccgccaccgctgttgccgccgccgccccc
cgttcgcgcgggcgcaagagaaattatccagcggccagcgcgctc
atgccccagtacgcactgcatccatccatcgcgcccacccccggc
taccgcgggtactcgtaccgcccgcgcagatcagccggcactcgc
ggccgccgccgccgtgcgaccacaaccagccgccgccgtcgccgc
cgccgccagccagtgctgacccccgtgtctgtaaggaaggtggct
cgctcggggagcacgctggtggtgcccagagcgcgctaccacccc
agcatcgtttaaagccggtctctgtatggttcttgcagatatggc
cctcacttgtcgcctccgcttcccggtgccgggataccgaggaag
aactcaccgccgcagaggcatggcgggcagcggtctccgcggcgg
ccgtcgccatcgccggcgcgcaaaaagcaggcgcatgcgcggcgg
tgtgctgcctctgctaatcccgctaatcgccgcggcgatcggtgc
cgtacccgggatcgcctccgtggccctgcaggcgtcccagaaacg
ttgactcttgcaaccttgcaagcttgcattttttggaggaaaaaa
taaaaaaagtctagactctcacgctcgcttggtcctgtgac
AdV-HPV16/18 DNA Sequence
SEQ ID NO: 244
tattttgtagaaaaaagatggaagacatcaactttgcgtcgctgg
ccccgcgtcacggctcgcgcccgttcatgggagactggacagata
tcggcaccagcaatatgagcggtggcgccttcagctggggcagtc
tgtggagcggccttaaaaattttggttccaccattaagaactatg
gcaacaaagcgtggaacagcagcacgggccagatgctgagagaca
agttgaaagagcagaacttccaggagaaggtggcgcagggcctgg
cctctggcatcagcggggtggtggacatagctaaccaggccgtgc
agaaaaagataaacagtcatctggacccccgtcctcaggtggagg
aaatgcctccagcgatggagacggtgtctcccgagggcaaaggcg
aaaagcgcccgcggcccgacagagaagagaccctggtgtcacaca
ccgaggagccgccctcttacgaggaggcagtcaaggccggcctgc
ccaccactcgccccatagcccccatggccaccggtgtggtgggcc
acaggcaacacactcccgcaacactagatctgcccccgccgtccg
agccgccgcgccagccaaaggcggcgacggtgcccgctccctcca
cttccgccgccaacagagtgcccctgcgccgcgccgcgagcggcc
cccgggcctcgcgagttagcggcaactggcagagcacactgaaca
gcatcgtgggcctgggagtgaggagtgtgaagcgccgccgttgct
actgaatgagcaagctagctaacgtgttgtatgtgtgtatgcgtc
ctatgtcgccgccagaggagctgttgagccgccggcgccgtctgc
actccagcgaatttcaagatggcgaccccatcgatgatgcctcag
tggtcgtacatgcacatctcgggccaggacgcttcggagtacctg
agccccgggctggtgcagttcgcccgcgccacagacacctacttc
aacatgagtaacaagttcaggaaccccactgtggcgcccacccac
gatgtgaccacggaccggtcgcagcgcctgacgctgcggttcatc
cccgtggatcgggaggacaccgcctactcttacaaggcgcggttc
acgctggccgtgggcgacaaccgcgtgctggacatggcctccact
tactttgacatcaggggggtgctggacaggggccccaccttcaag
ccctactcgggtactgcctacaactccctggcccccaagggcgct
cccaattcttgcgagtgggaacaagaggaaaatcaggtggtcgct
gcagatgatgaacttgaagatgaagaagcgcaagctcaagaggac
gccccagctaaaaaaattcatgtatatgcccaggcgcctcttgct
ggcgaaaagattaccaaggatggtttgcaaataggtactgaagtt
gtaggagatacatctaaggacacttttgcagacaaaacattccaa
cccgaacctcagataggcgagtctcagtggaacgaggctgatgcc
acagtagcaggaggcagagtcttgaaaaaaaccacccctatgaga
ccttgctatggatcctatgccaggcctacaaatgccaacgggggt
caaggaattatggttgccaatgaacaaggagtgttggagtctaaa
gtggagatgcaatttttttctaacactacaacccttaatgcgcgg
gatggagctggcaatcccgaaccaaaggtggtgttgtacagtgaa
gatgtccacttggaatctcctgacactcatttgtcttacaagccc
aaaaaggatgatgttaatgctaaaattatgttgggtcagcaagct
atggctaacaggcccaacctcattgcttttagagataatttcatt
ggactcatgtactacaacagcactggtaacatgggagtgctggcg
ggtcaggcctctcagttgaatgccgtggtggacctgcaggataga
aacacagaactgtcatatcagcttatgcttgattccattggggat
agatccagatacttctccatgtggaaccaggcagtggatagctat
gacccagatgtcagaatcattgaaaaccatggtgtcgaggacgag
ctacccaactactgcttccctctgggcggcataggaattactgat
acttatcaagggatcaaaaataccaatggcaatggtcagtggacc
aaagatgatcagttcgcggaccgtaatgaaataggggtgggaaac
aacttcgccatggagatcaacatccaggccaacctctggaggaac
ttcctctatgcgaacgtggggctctacctgccagacaagctcaag
tacaaccccaccaacgtggacatctctgacaaccccaacacctat
gactacatgaacaagcgtgtggtggctcccggcctggtggactgc
tttgtcaatgtgggagccaggtggtccctggactacatggacaac
gtcaaccccttcaaccaccaccgcaatgcgggtctgcgctaccgc
tccatgatcctgggcaacgggcgctacgtgcccttccacattcag
gtgccccagaagttctttgccatcaagaacctcctcctcctgccg
ggctcctacacttacgagtggaacttcaggaaggatgtcaacatg
gtcct
AdV-HPV16/18 DNA Sequence
SEQ ID NO: 244
gcagagctctctgggcaatgaccttagggtggacggggccagcat
caagtttgacagcgtcaccctctatgctaccttcttccccatggc
tcacaacaccgcctccacgctcgaggccatgctgaggaacgacac
caacgaccagtccttcaatgactacctctctggggccaacatgct
ctaccccatccccgccaaggccaccaacgtgcccatctccattcc
ctctcgcaactgggccgccttcagaggctgggcctttacccgcct
taagaccaaggaaaccccctccctgggctcgggttttgaccccta
ctttgtctactcgggatccatcccctacctggatggcaccttcta
cctcaaccacacttttaagaagatatccatcatgtatgactcctc
cgtcagctggccgggcaatgaccgcctgctcacccccaatgagtt
cgaggtcaagcgcgccgtggacggcgagggctacaacgtggccca
gtgcaacatgaccaaggactggttcctggtgcagatgctggccaa
ctacaacataggctaccagggcttctacatcccagagagctacaa
ggacaggatgtactccttcttcagaaatttccaacccatgagcag
gcaggtggtggacgagaccaaatacaaggactatcaggccattgg
catcactcaccagcacaacaactcgggattcgtgggctacctggc
tcccaccatgcgcgaggggcaggcctaccccgccaacttccccta
cccgttgataggcaagaccgcggtcgacagcgtcacccagaaaaa
gttcctctgcgaccgcaccctctggcgcatccccttctctagcaa
cttcatgtccatgggtgcgctcacggacctgggccagaacctgct
ctatgccaactccgcccatgcgctggacatgacttttgaggtgga
ccccatggacgagcccacccttctctatattgtgtttgaagtgtt
cgacgtggtcagagtgcaccagccgcaccgcggtgtcatcgagac
cgtgtacctgcgcacgcccttctcggccggcaacgccaccaccta
aggagacagcgccgccgcctgcatgacgggttccaccgagcaaga
gctcagggccatcgccagagacctgggatgcggaccctatttttt
gggcacctatgacaaacgcttcccgggcttcatctcccgagacaa
gctcgcctgcgccatcgtcaacacggccgcgcgcgagaccggggg
cgtgcactggctggcctttggctgggacccgcgctccaaaacctg
ctacctcttcgacccctttggcttctccgatcagcgcctcagaca
gatctatgagtttgagtacgaggggctgctgcgccgcagcgcgct
tgcctcctcgcccgaccgctgcatcacccttgagaagtccaccga
gaccgtgcaggggccccactcggccgcctgcggtctcttctgctg
catgtttttgcacgcctttgtgcgctggccccagagtcccatgga
tcgcaaccccaccatgaacttgctcaagggagtgcccaacgccat
gctccagagcccccaggtccagcccaccctgcgccacaaccagga
acagctctaccgcttcctggagcgccactccccctacttccgcag
tcacagcgcgcacatccggggggccacctctttctgccacttgca
acaaaacatgcaagacggaaaatgatgtacagctcgctttttaat
aaatgtaaagactgtgcactttatttatacacgggctctttctgg
ttatttattcaacaccgccgtcgccatctagaaatcgaaagggtt
ctgccgcgcgtcgccgtgcgccacgggcagagacacgttgcgata
ctggaagcggctcgcccacttgaactcgggcaccaccatgcgggg
cagtggctcctcggggaagttctcgccccacagggtgcgggtcag
ctgcagcgcgctcaggaggtcgggagccgagatcttgaagtcgca
gttggggccggaaccctgcgcgcgcgagttgcggtacacggggtt
gcagcactggaacaccagcagggccggattacgcacgctggccag
caggctctcgtcgctgatcatgtcgctgtccagatcctccgcgtt
gctcagggcgaatggggtcatcttgcagacctgcctgcccaggaa
aggcggcagcccgggcttgccgttgcagtcgcagcgcaggggcat
cagcaggtgcccgtggcccgtctgcgcctgcgggtacagcgcgcg
catgaaggcttcgatctgcctgaaagccacctgcgtcttggctcc
ctccgaaaagaacatcccacaggacttgctggagaactggttcgc
gggacagctggcatcgtgcaggcagcagcgcgcgtcggtgttggc
gatctgcaccacgttgcgaccccaccggttcttcactatcttggc
cttggaagcctgctccttcagcgcgcgctggccgttctcgctggt
cacatccatctctatcacctgctccttgttgatcatgtttgtccc
gtgcagacacttcaggtcgccctccgtctgggtgcagcggtgctc
ccacagcgcgcaaccggtgggctcccaatttttgtgggtcacccc
cgcgtaggc
AdV-HPV16/18 DNA Sequence
SEQ ID NO: 244
ctgcaggtaggcctgcaagaagcgccccatcatggccacaaaggt
cttctggctcgtaaaggtcagctgcaggccgcgatgctcttcgtt
cagccaggtcttgcagatggcggccagcgcctcggtctgctcggg
cagcatcctaaaatttgtcttcaggtcgttatccacgtggtactt
gtccatcatggcgcgcgccgcctccatgcccttctcccaggcgga
caccatgggcaggcttagggggtttatcacttccaccggcgagga
caccgtactttcgatttcttcttcctccccctcttcccggcgcgc
gcccacgctgctgcgcgctctcaccgcctgcaccaaggggtcgtc
ttcaggcaagcgccgcaccgagcgcttgccgcccttgacctgctt
aatcagcaccgggggttgctgaagcccaccatggtcagctccgcc
tgctcttcttcgtcttcgctgtctaccactatctctggggaaggg
cttctccgctctgcggcggtgcgcttcttttttttcttgggagca
gccgtgacggagtccgccacggcgacggaggtcgagggcgtgggg
ctgggggtgcgcggtaccagggcctcgtcgccctcggactcttcc
tctgactccaggcggcggcggagacgcttctttgggggcgcgcgc
gtcagcggcggcggagacggggacggggacggggacgggacgccc
tccacagggggtggtcttcgcgcagacccgcggccgcgctcgggg
gtcttttcgagctggtcttggtcccgactggccattgtatcctcc
tcctcctaggcagagagacataaggagtctatcatgcaagtcgag
aaggaggagagcttaaccaccccctctgagaccgccgatgcgccc
gccgtcgccgtcgcccccgctgccgccgacgcgcccgccacaccg
agcgacacccccgcggaccccccagccgacgcacccctgttcgag
gaagcggccgtggagcaggacccgggctttgtctcggcagaggag
gatttgcgagaggaggaggataaggagaagaagccctcagtgcca
aaagatgataaagagcaagacgagcacgacgcagatgcacaccag
ggtgaagtcgggcggggggacggagggcatgacggcgccgactac
ctagacgaagggaacgacgtgctcttgaagcacctgcatcgtcag
tgcgccatcgtttgcgacgctctgcaggagcgcagcgaagtgccc
ctcagcgtggcggaggtcagccacgcctacgagctcagcctcttc
tccccccgggtgcccccccgccgccgcgaaaacggcacatgcgag
cccaacccgcgcctcaacttctaccccgcctttgtggtgcccgag
gtcctggccacctatcacatcttctttcaaaattgcaagatcccc
ctctcgtgccgcgccaaccgtagccgcgccgataagatgctggcc
ctgcgccagggcgaccacatacctgatatcgccgctttggaagat
gtgccaaagatcttcgagggtctgggtcgcaacgagaagcgggca
gcaaactctctgcaacaggaaaacagcgaaaatgagagtcacacc
ggggtactggtggagctcgagggcgacaacgcccgcctggcggtg
gtcaagcgcagcatcgaggtcacccactttgcctaccccgcgctc
aacctgccccccaaagtcatgaacgcggccatggacgggctgatc
atgcgccgcggccggcccctcgctccagatgcaaacttgcatgag
gagaccgaggacggccagcccgtggtcagcgacgagcagctggcg
cgctggctggagaccgcggaccccgccgaactggaggagcggcgc
aagatgatgatggccgcggtgctggtcaccgtagagctggagtgt
ctgcagcgcttcttcggcgaccccgagatgcagagaaaggtcgag
gagaccctgcactacaccttccgccagggctacgtgcgccaggct
tgcaagatctccaacgtggagctcagcaacctggtgtcctacctg
ggcatcttgcatgagaaccgcctcgggcagagcgtgctgcactcc
accctgcgcggggaggcgcgccgcgactacgtgcgcgactgcgtt
tacctcttcctctgctacacctggcagacggccatgggggtctgg
cagcagtgcctggaggagcgcaacctcaaggagctggagaagctc
ctgcagcgcgcgctcaaagacctctggacgggctacaacgagcgc
tcggtggccgccgcgctggccgacctcatcttccccgagcgcctg
ctcaaaaccctccagcaggggctgcccgacttcaccagccaaagc
atgttgcaaaacttcaggaactttatcctggagcgttctggcatc
ctacccgccacctgctgcgccctgcccagcgactttgtccccctc
gtgtaccgcgagtgccccccgccgctgtggggtcactgctacctg
ttccaactggccaactacctgtcctaccacgcggacctcatggag
gactccagcggcgaggggctcatggagtgccactgccgctgcaac
ctctgcacgcccca
AdV-HPV16/18 DNA Sequence
SEQ ID NO: 244
ccgctccctggtctgcaacacccaactgctcagcgagagtcagat
tatcggtaccttcgagctacagggtccgtcctcctcagacgagaa
gtccgcggctccggggctaaaactcactccggggctgtggacttc
cgcctacctgcgcaaatttgtacctgaagactaccacgcccacga
gatcaggttttacgaagaccaatcccgcccgcccaaggcggagct
gaccgcctgcgtcatcacccagggcgagatcctaggccaattgca
agccatccaaaaagcccgccaagattttttgctgagaaagggtcg
gggggtgtatctggacccccagtcgggtgaggagctcaacccggt
tcccccgctgccgccgccgcgggaccttgcttcccaggataagca
tcgccatggctcccagaaagaagcagcagcggccgccactgccgc
caccccacatgctggaggaagaggaggaatactgggacagtcagg
cagaggaggtttcggacgaggaggagccggagacggagatggaag
agtgggaggaggacagcttagacgaggaggcttccgaagccgaag
aggcagacgcaacaccgtcaccctcggccgcagccccctcgcagg
cgcccccgaagtccgctcccagcatcagcagcaacagcagcgcta
taacctccgctcctccaccgccgcgacccacggccgaccgcagac
ccaaccgtagatgggacaccaccggaaccggggccggtaagtcct
ccgggagaggcaagcaagcgcagcgccaaggctaccgctcgtggc
gcgctcacaagaacgccatagtcgcttgcttgcaagactgcgggg
ggaacatctccttcgcccgccgcttcctgctcttccaccacggtg
tggccttcccccgtaacgtcctgcattactaccgtcatctctaca
gcccctactgcggcggcagtgagccagagacggtcggcggcggcg
gcggcgcccgtttcggcgcctaggaagacccagggcaagacttca
gccaagaaactcgcggcggccgcggcgaacgcggtcgcgggggcc
ctgcgcctgacggtgaacgaacccctgtcgacccgcgaactgaga
aaccgaatcttccccactctctatgccatcttccagcagagcaga
gggcaggatcaggaactgaaagtaaaaaacaggtctctgcgctcc
ctcacccgcagctgtctgtatcacaagagcgaagaccagcttcgg
cgcacgctggaggacgctgaggcactcttcagcaaatactgcgcg
ctcactcttaaggactagctccgcgcccttctcgaatttaggcgg
gaacgcctacgtcatcgcagcgccgccgtcatgagcaaggacatt
cccacgccatacatgtggagctatcagccgcagatgggactcgcg
ggggcgcctcccaagactactccacccgcatgaactggctcagtg
ccggcccacacatgatctcacaggttaatgatatccgcacccatc
gaaaccaaatattggtggagcaggcggcaattaccaccacgcccc
gcaataatcccaaccccagggagtggcccgcgtccctggtgtatc
aggaaattcccggccccaccaccgtactacttccgcgtgattccc
aggccgaagtccaaatgactaactcaggggcgcagctcgcgggcg
gctgtcgtcacagggtgcggcctcctcgccagggtataactcacc
tggagatccgaggcagaggtattcagctcaacgacgagtcggtga
gctcctcgctcggtctaagacctgacgggaccttccagatagccg
gagccggccgatcttccttcacgccccgccaggcgtacctgactc
tgcagagctcgtcctcggcgccgcgctcgggcggcatcgggactc
tccagttcgtgcaggagtttgtgccctcggtctacttcaacccct
tctcgggctctcccggtcgctacccggaccagttcatctcgaact
ttgacgccgcgagggactcggtggacggctacgactgaatgtcgg
gtggacccggtgcagagcaacttcgcctgaagcacctcgaccact
gccgccgccctcagtgctttgcccgctgtcagaccggtgagttcc
agtacttttccctgcccgactcgcacccggacggcccggcgcacg
gggtgcgctttttcatcccgagtcaggtgcgctctaccctaatca
gggagtttaccgcccgtcccctactggcggagttggaaaaggggc
cttctatcctaaccattgcctgcatctgctctaaccctggattgc
accaagatctttgctgtcatttgtgtgctgagtataataaaggct
gagatcagaatctactcgggctcctgtcgccatcctgtcaacgcc
accgtccaagcccggcccgatcagcccgaggtgaacctcacctgc
ggtctgcaccggcgcctgaggaaatacctagcttggtactacaac
agcactccctttgtggtttacaacagctttgaccaggacggggtc
tcactgagggataacctctcgaacctgagctactccatcaggaag
aacagcaccctcgagctacttcctcct
AdV-HPV16/18 DNA Sequence
SEQ ID NO: 244
tacctgcccgggacttaccagtgtgtcaccggtccctgcacccac
acccacctgttgatcgtaaacgactctcttccgagaacagacctc
aataactcctcttcgcagttccccagaacaggaggtgagctcagg
aaaccccgggtaaagaagggtggacaagagttaacacttgtgggg
tttctggtgtatgtgacgctggtggtggctcttttgattaaggct
tttccttccatgtctgaactctccctcttttatgaacaactcgac
tagtgctaacgggaccctacccaacgaatcgggattgaatatcgg
taaccaggttgcagtttcacttttgattaccttcatagtcctctt
cctgctagtgctgtcgcttctgtgcctgcggatcgggggctgctg
catccacgtttatatctggtgctggctgtttagaaggttcggaga
ccatcgcaggtagaataaacatgctgctgcttaccctctttgtcc
tggcgctggccgccagctgccaagccttttccgaggctgacttta
tagagccccagtgtaacgtgacttttaaagcccatgcacagcgtt
gtcatactataatcaaatgtgccaccgaacacgatgaatacctta
tccagtataaagataaatcacacaaagtggcacttgttgacatct
ggaaacccgaagaccctttggaatacaatgtgaccgttttccagg
gtgacctcttcaaaatttacaattacactttcccatttgaccaga
tgtgtgactttgtcatgtacatggaaaagcagcacaagctgtggc
ctccgactccccagggctgtgtggaaaatccaggctctttctgca
tgatctctctctgtgtaactgtgctggcactaatactcacgcttt
tgtatatcagatttaaatcaaggcaaagcttcatcgatgaaaaga
aaatgccttaaacgctttcacgcttgattgctaacaccgggtttt
tatccgcagaatgattggaatcaccctactaatcacctccctcct
tgcgattgcccatgggttggaacgaatcgaagcccctgtgggggc
caatgttaccctggtggggcctgtcggcaatgctacattaatgtg
ggaaaaatatactaaaaatcaatgggtctcttactgcactaacaa
aaacagccacaagcccagagccatctgcgatgggcaaaatctaac
cttgattgatgttcaaatgctggatgcgggctactattatgggca
gctgggtacaatgattaattactggagaccccacaaagattacat
gctccacgtagtaaagggtccccttagcagcccacccactaccac
ctctactacccccactaccaccactactcccaccaccagcactgc
cgcccagcctcctcatagcagaacaaccacttttatcaattccaa
gtcccactccccccacattgccggcgggccctccgcctcagactc
cgagaccaccgagatctgcttctgcaaatgctctgacgcctttgc
tgaggatttggaagaccacgaggaagatgagcatgacttcgcaga
tgcatgccaggcatcagaggcagaagcgctgccggtggccctcaa
acagtatgcagacccccacaccacccccaaccttcctccaccttc
ccagaagccaagtttcctgggggaaaatgaaactctgcctctctc
catactcgctctgacatctgttgctatgttgaccgctctgctggt
gcttctatgctctatatgctacctgatctgctgcagaaagaaaaa
atctcacggccatgctcaccagcccctcatgcacttcccttaccc
tccagagctgggcgaccacaaactttaagtctgcagtaactatct
gcccatcccttgtcagtcgacagcgatgagccccactaatctaac
ggcctctggacttacaacatcgtctcttaatgagaccaccgctcc
tcaagacctgtacgatggtgtctccgcgctggttaaccagtggga
tcacctgggcatatggtggctcctcataggagcagtgaccctgtg
cctaatcctggtctggatcatctgctgcatcaaaagcagaagacc
caggcggcggcccatctacaggccctttgtcatcacacctgaaga
tgatgatgacaccacttccaggctgcagaggctaaagcagctact
cttctcttttacagcatggtaaattgaatcatgcctcgcattttc
atctacttgtctctccttccactttttctgggctcttctacattg
gccgctgtgtcccacatcgaggtagactgcctcacgcccttcaca
gtctacctgcttttcggctttgtcatctgcacctttgtctgcagc
gttatcactgtagtgatctgcttcatacagtgcatcgactacgtc
tgcgtgcgggtggcttactttagacaccacccccagtatcgcaac
agggacatagcggctctcctaagacttgtttaaaatcatggccaa
attaactgtgattggtcttctgatcatctgctgcgtcctagccgc
gattgggactcaagctcctaccaccaccagcgctcccagaaagag
acatgtatcctgcagcttcaagcgtccctggaatataccccaatg
ctttactgatgaacctgaaatctctttggcttggtacttcagcgt
caccgcccttcttatcttctgcagtacggttattgcccttgccat
ctacc
AdV-HPV16/18 DNA Sequence
SEQ ID NO: 244
cttcccttgacctgggctggaatgctgtcaactctatggaatatc
ccaccttcccagaaccagacctgccagacctggttgttctaaacg
cgtttcctcctcctgctcccgttcaaaatcagtttcgccctccgt
cccccacgcccactgaggtcagctactttaatctaacaggcggag
atgactgaaaacctagacctagaaatggacggtctctgcagcgag
caacgcacactagagaggcgccggcaaaaagagctcgagcgtctt
aaacaagagctccaagacgcggtggccatacaccagtgcaaaaaa
ggtgtcttctgtctggtaaaacaggccacgctcacctatgaaaaa
acaggtgacacccaccgcctaggatacaagctgcccacacagcgc
cagaagttcgccctcatgataggcgaacaacccatcaccgtgacc
cagcactccgtggagacagaaggctgcatacacgctccctgtagg
ggcgctgactgcctctacaccttgatcaaaaccctctgcggtctc
agagacctcatcccttttaattaatcataactgtaatcaataaaa
aatcacttacttgaaatctgatagcaagcctctgtccaatttttt
cagcaacacttccttcccctcctcccaactctggtactctaggcg
cctcctagctgcaaacttcctccacagtctgaagggaatgtcaga
ttcctcctcctgtccctccgcacccacgatcttcatgttgttgca
gatgaaacgcgcgagatcgtctgacgagaccttcaaccccgtgta
cccctacgataccgagatcgctccgacttctgtccctttccttac
ccctccctttgtgtcatccgcaggaatgcaagaaaatccagctgg
ggtgctgtccctgcacttgtcagagccccttaccacccacaatgg
ggccctgactctaaaaatggggggggcctgaccctggacaaggaa
gggaatctcacttcccaaaacatcaccagtgtcgatccccctctc
aaaaaaagcaagaacaacatcagccttcagaccgccgcacccctc
gccgtcagctccggggccctaacactttttgccactcccccccta
gcggtcagtggtgacaaccttactgtgcagtctcaggcccctctc
actttggaagactcaaaactaactctggccaccaaaggaccccta
actgtgtccgaaggcaaacttgtcctagaaacagaggctcccctg
catgcaagtgacagcagcagcctgggccttagcgttacggcccca
cttagcattaacaatgacagcctaggactagacatgcaagcgccc
attagctctcgagatggaaaactggctctaacagtggcggccccc
ctaactgtggtcgagggtatcaatgctttggcagtagccacaggt
aagggtattgggctaaatgaaaccaacacacacctgcaggcaaaa
ctggtcgcacccctaggctttgataccaacggcaacattaagcta
agcgttgcaggaggcatgaggctaaacaataacacactgatacta
gatgtaaactacccatttgaggctcaaggccaactgagcctaaga
gtgggctcgggcccactatatgtagattctagtagtcataaccta
accattagatgccttaggggattgtatataacatcttctaacaac
caaaacggtctagaagccaacattaaactaacaagaggccttgtg
tatgacggaaatgccatagcagttaatgttggcaaagggctggaa
tacagccctactgacacaacagaaaaacctatacagactaaaata
ggtctaggcatggagtatgataccgagggagccatgatgacaaaa
ctaggctctggactaagctttgacaattcaggagccattgtagtg
ggaaacaaaaatgatgacaggcttactttgtggaccacaccggac
ccatcgcccaactgtcagatctactctgaaaaagatgctaaacta
accttggtactgactaaatgtggcagtcaggttgtaggcacagta
tctattgccgctcttaaaggtagcctcgtgccaatcactagtgca
atcagtgtggttcaggtatacctaaggtttgatgaaaatggggta
ctaatgagtaactcttcacttaatggcgaatactggaattttaga
aacggagactcaactaatggcacaccatatacaaacgcagtgggt
ttcatgcctaatctactggcctatcctaaaggtcaaactacaact
gcaaaaagtaacattgtcagccaggtctacatgaatggggacgat
actaaacccatgacatttacaatcaacttcaatggccttagtgaa
acaggggatacccctgttagtaaatattccatgacattctcatgg
aggtggccaaatggaagctacatagggcacaattttgtaacaaac
tcctttaccttctcctacatcgcccaagaataaagaaagcacaga
gatgcttgtttttgatttcaaaattgtgtgcttttatttattttc
agcttacagtatttccagtagtcattcaaataaagcttaatcaaa
ctgcatgagaacccttccacatagcttaaattagcaccagtgcaa
atggagaaaacaattgacggccgccatagagcccaccgcatcccc
agcatgcctgctat
AdV-HPV16/18 DNA Sequence
SEQ ID NO: 244
tgtcttcccaatcctcccccttgctgtcctgccccaccccacccc
ccagaatagaatgacacctactcagacaatgcgatgcaatttcct
cattttattaggaaaggacagtgggagtggcaccttccagggtca
aggaaggcacgggggaggggcaaacaacagatggctggcaactag
aaggcacagggtaccaacgcgtcgtaactataacggtcctaaggt
agcgaaaagcactctcacagcaccagcactaatcagagtgtgaag
agggccaagtgccgaacgagtatatataggaataaaaaatgacgt
aaatgtgtaaaggtcagaaaacgcccagaaaaatacacagaccaa
cgcccgaaacgaaaacccgcgaaaaaatacccagaacttcctcaa
caaccgccacttccgctttctcacggtacgtcacttccgcaagaa
aagcaaaactacatttcccacatgtgtaaaaacgaaaccccgccc
cttgtaaccgcccacaacttacatcatcaaaacgtaaactcctac
gtcacccgccccgcctctccccgcccacctcattatcatattggc
cacaatccaaaataaggtatattattgatgatg

AdV-HPV16/18 Antigen Protein Variants

| Variant | Amino Acid Sequence |
|---|---|
| A | SEQ ID NO: 245: |
| | IQTNRELTEVFEFAPRYSHSDYIIFVYIPL |
| | GDAKLPQLCTEVDDLLLMGTLGIVPLHAPM |
| | SADTLHEYMLDLHNRATDLSEEENDEIDGV |
| | NHQHLPARRIHDNLLIRCLRCPLILAARLA |
| | LLFLNTLSFVCPWCASSHADMKCIDFYSRI |
| | DNLKLPDLCTELALHWTAAVNNLQLFLNTL |
| | SFVPANKFQQLFLNTLNDRVYDFAFRDLPL |
| | FLAAKEGSYEKLTNTGLYNLASRLCIVYRD |
| | GNPYAVCDNHMYMLDLQPETTPRQFAFRDL |
| | CIVYIVRFLNLEVSQTSKFTRQTERTLGIV |
| | CPIAAHYSKSDRTLEDLLMGVANPTIHDII |
| | LECVQNMHLDKKQRFHNIPLHAAYSADTTL |
| | EQQYNKPLCDLLRSRATDLCDSTLRLCVVH |
| | DRWTGRCMSCCPLILAPRLAVSLQDIEITC |
| | VSHADILCVQSTHVDIDDIAHYNIVTFCCL |
| | HWGATMSNVDDLRAFQQLFLNTLSGPSKIS |
| | EYRHYCYDNRKLPQLCTELPLFLGAKEPSY |
| | EKTVLELTEVADRRTLEDLLMGTQHMFAFK |
| | DLFVVPRNQLYNKPLCDVIIHTLQDIVLHL |
| | FE |
| B | SEQ ID NO: 246: |
| | MQTDHELTEVFEFAARYGRGDYIIFVYIPL |
| | AQAKLPQLCTEVNNMLLMGTLGIVPLRAPV |
| | SADTLHEYMLDLRNRPTNLSEEENDEIDGV |
| | NHQHLPARRMRDNLLIRCLRCPLILAPRLA |
| | +10VLFLNTLSFVCPWCASSRAIVKCIDFY |
| | SRINDLKLPDLCTELALHWAGAVNNIQLFL |
| | NTLSFVGPNKFQQLFLNTLDNRVYDFAFRD |
| | LPLFLATREGSYEKLTNTGLYNLANQLCIV |
| | YRDGNPYAVCDNQMYMLDLQPETTPHDFAF |
| | RDLCIVYVIRLLELEVSQSTKLTRQTQRTL |
| | GIVCPIAAHYSRSQRTLEDLLMGVPDPTIH |
| | DIILECVENMHLDKKQRFHNIPLHTAVSAN |
| | TTLEQQYNKPLCDLLHNRATILCDSTLRLC |
| | VMRDRWTGRCMSCCPLILPARLPVSLQDIE |
| | ITCVSQADVLCVQSTHVDIDNLAHYNIVTF |
| | CCLQWAAAINNVDDLRAFQQLFLNTLSPTN |
| | KISEYRHYCYNNHKLPQLCTELPLFLPARE |
| | PSYEKTVLELTEVTNRRTLEDLLMGTNHMF |
| | AFKDLFVVAREQLYNKPLCDVMVRTLQDIV |
| | LHLLQ |
| C | SEQ ID NO: 247: |
| | METDRELTEVFEFAARFSRSDYIIFVYIPL |
| | ADAKLPQLCTEVDSMLLMGTLGIVPLHAAM |
| | SADTLHEYMLDLRNHATDLSEEENDEIDGV |
| | NHQHLPARRMHDNLLIRCLRCPIILAPRLA |
| | VLFLNTLSFVCPWCASSHADVKCIDFYSRI |
| | DDLKLPDLCTELALHWAAAVNSVQLFLNTL |
| | SFVGASKFQQLFLNTLNNRVYDFAFRDLPL |
| | FLAAREGSFEKLTNTGLYNLANRLCIVYRD |
| | GNPYAVCDDHMYMLDLQPETTPRDFAFRDL |
| | CIVYIVRILDLEVSETSKLTHQTDRTLGIV |
| | CPIAARFSRSDRTLEDLLMGVADATIHDII |
| | LECVDNMHLDKKQRFHNIPLHAPVSPDTTL |
| | EQQYNKPLCDLLRNHATDLCDSTLRLCVMH |
| | DRWTGRCMSCCPILIAARLAVSLQDIEITC |
| | VSHADVLCVQSTHVDIDDLAHYNIVTFCCI |
| | HWAAAVNNVDDLRAFQQLFLNTLSGASKIS |
| | EYRHYCYNNRKLPQLCTELPIFIAAREGSY |
| | EKTVLELTEVANRRTLEDLLMGTDHMFAFK |
| | DLFVVPRDQLYNKPLCDVIVRTLQDIVLHL |
| | LE |
| D | SEQ ID NO: 248: |
| | MQTNRELTEVFEFAARYSESDYIIFVYIPL |
| | ANAKLPQLCTEVQNMLLMGTLGIVPLKAAV |
| | SAQTLHEYMLDLKNRATNLSEEENDEIDGV |
| | NHQHLPARRLHDNLLIRCLRCPLILAAELA |
| | VLFLNTLSFVCPWCASSKADVKCIDFYSRI |
| | DDMKLPDLCTELALQWAAAINNIQLFLNTL |
| | SFVGANRFQQLFLNTLNDRVYDFAFRDLPI |
| | FLAAREGTYEKLTNTGLYNLGNRLCIVYRD |
| | GNPYAVCDDHIYMLDLQPETTARDFAFRDL |
| | CIVYIMRLLDIEVTQTSKLSRQPDRTLGIV |
| | CPIAAKYSRSNRTLEDLLMGVANATIHDII |
| | LECVDDMHLDKKQRFHNIPLQAAVSANTTL |
| | EQQYNKPLCDLLRNRATNLCDSTLRLCVMH |
| | NRWTGRCMSCCPLILAAELAVSLQDIEITC |
| | VSHANVLCVQSTHVDIDQLAHYNIVTFCCL |
| | QWAAAMNNMDDLRAFQQLFLNTLSAANKIS |
| | EYRHYCYNNKKLPQLCTELPFLLAAEEGSY |
| | EKTVLELTEVANHRTLEDLLMGTDQMFAFK |
| | DLFVVPRNQLYNKPLCDVIVHTLQDIVLHL |
| | LD |
| E | SEQ ID NO: 249: |
| | MKTDRELTEVFEFAAKYSRSDYIIFVYIPL |
| | AEAKLPQLCTEVENMLLMGTLGIVPLHAGV |
| | SADTLHEYMLDLRNRTTDLSEEENDEIDGV |
| | NHQHLPARRMHENLLIRCLRCPLILAAHLA |
| | VLFLNTLSFVCPWCASSHSDVKCIDFYSRI |
| | DDIKLPDLCTELALHWAAAMNNVQLFLNTL |
| | SFVGAQKFQQLFLNTLNSRVYDFAFRDLPL |
| | FLAAHEGSYEKLTNTGLYNLASRLCIVYRD |
| | GNPYAVCDDHVYMLDLQPETTPKDFAFRDL |
| | CIVYIVRLLDLEVSHTSKLTRQTDRTLGIV |
| | CPIAAKYSRSDRTLEDLLMGVAEATIHDII |
| | LECVDNIHLDKKQRFHNIPLHAGVSADTTL |
| | EQQYNKPLCDLLRNRTTDLCDSTLRLCVMH |
| | NRWTGRCMSCCPLILAAHLAVSLQDIEITC |
| | VSHPDVLCVQSTHVDIDDIAHYNIVTFCCL |
| | HWAAAINNVDDLRAFQQLFLNTLSGAQKIS |
| | EYRHYCYNDRKLPQLCTELPLFLAAEEGSY |
| | EKTVLELTEVASRRTLEDLLMGTDHVFAFK |
| | DLFVVPKDQLYNKPLCDVIVKTLQDIVLHL |
| | LN |
| F | SEQ ID NO: 250: |
| | MQTDRYIIFVYIPLARYSRSDELTEVFEFA |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| | SEQ ID NO: 251: |
| G | MQTDRELTEVFEFAARYSRSDYIIFVYIPL |
| | ADAKLPQLCTEVDNMLLMGTLGIVPLHAAV |
| | SADSEEENDEIDGVNHQHLPARRRNRATDL |
| | TLHEYMLDLMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| H | SEQ ID NO: 252: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDLFLNTLSFVCPWCASPLI |
| | LAARLAVNLLIRCLRCSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| I | SEQ ID NO: 253: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDTLQDIVLHLIVRQLYNKPLCDV |
| | LE |
| J | SEQ ID NO: 254: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKLPDLCTEL |
| | KCIDFYSRIDDLKCIDFYSRIKLPDLCTEL |
| | ALHWAAAVNNVQLFLNTLSFVGANKFQQLF |
| | LNTLNNRVYDFAFRDLPLFLAAREGSYEKL |
| | TNTGLYNLANRLCIVYRDGNPYAVCDDHMY |
| | MLDLQPETTPRDFAFRDLCIVYIVRLLDLE |
| | VSQTSKLTRQTDRTLGIVCPIAARYSRSDR |
| | TLEDLLMGVADATIHDIILECVDNMHLDKK |
| | QRFHNIPLHAAVSADTTLEQQYNKPLCDLL |
| | RNRATDLCDSTLRLCVMHDRWTGRCMSCCP |
| | LILAARLAVSLQDIEITCVSHADVLCVQST |
| | HVDIDDLAHYNIVTFCCLHWAAAVNNVDDL |
| | RAFQQLFLNTLSGANKISEYRHYCYNNRKL |
| | PQLCTELPLFLAAREGSYEKTVLELTEVAN |
| | RRTLEDLLMGTDHMFAFKDLFVVPRDQLYN |
| | KPLCDVIVRTLQDIVLHLLE |
| K | SEQ ID NO: 255: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLQLFLNTLSFVALHWAAAVNNVKLPDLC |
| | TELGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| L | SEQ ID NO: 256: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVFQQLFLN |
| | TLGANKQLFLNTLSFVNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| M | SEQ ID NO: 257: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKVYDFAFRDLNNRFQQLFLNTLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| N | SEQ ID NO: 258: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRKLTNTGLYNLP |
| | LFLAAREGSYEVYDFAFRDLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| O | SEQ ID NO: 259: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYELCIVYRDGNPYAVCDANRKL |
| | TNTGLYNLDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| P | SEQ ID NO: 260: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRYMLDLQP |
| | ETTDHMLCIVYRDGNPYAVCDPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| Q | SEQ ID NO: 261: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMFAFRDLCIVYPRDYMLDLQ |
| | PETTIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LE |
| R | SEQ ID NO: 262: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKNNRVYDFAFRDLPLFLAAREGSY |
| | EKLTNTGLYNLANRLCIVYRDGNPYAVCDD |
| | HMYMLDLQPETTPRDFAFRDLCIVYIVRLL |
| | DLEVSQTSKLTRQTDRTLGIVCPIAARYSR |
| | SDRTLEDLLMGVADATIHDIILECVDNMHL |
| | DKKQRFHNIPLHAAVSADTTLEQQYNKPLC |
| | DLLRNRATDLCDSTLRLCVMHDRWTGRCMS |
| | CCPLILAARLAVSLQDIEITCVSHADVLCV |
| | QSTHVDIDDLAHYNIVTFCCLHWAAAVNNV |
| | DDLRAFQQLFLNTLSGANKISEYRHYCYNN |
| | RKLPQLCTELPLFLAAREGSYEKTVLELTE |
| | VANRRTLEDLLMGTDHMFAFKDLFVVPRDQ |
| | LYNKPLCDVIVRTLQDIVLHLLE |
| S | SEQ ID NO: 263: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LEYLQYGVYRFADA |
| T | SEQ ID NO: 264: |
| | MQTDRYIIFVYIPLARYSRSDELTEVFEFA |
| | GDAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | FE |
| U | SEQ ID NO: 265: |
| | MQTDRELTEVFEFAARYSRSDADAKLPQLC |
| | TEVDNMLLMGTLGIVPLHAAVSADTLHEYM |
| | LDLRNRATDLSEEENDEIDGVNHQHLPARR |
| | MHDNLLIRCLRCPLILAARLAVLFLNTLSF |
| | VCPWCASSHADVKCIDFYSRIDDLKLPDLC |
| | TELALHWAAAVNNVQLFLNTLSFVGANKNN |
| | RVYDFAFRDLPLFLAAREGSYEKLTNTGLY |
| | NLANRLCIVYRDGNPYAVCDDHMYMLDLQP |
| | ETTPRDFAFRDLCIVYIVRLLDLEVSQTSK |
| | LTRQTDRTLGIVCPIAARYSRSDRTLEDLL |
| | MGVADATIHDIILECVDNMHLDKKQRFHNI |
| | PLHAAVSADTTLEQQYNKPLCDLLRNRATD |
| | LCDSTLRLCVMHDRWTGRCMSCCPLILAAR |
| | LAVSLQDIEITCVSHADVLCVQSTHVDIDD |
| | LAHYNIVTFCCLHWAAAVNNVDDLRAFQQL |
| | FLNTLSGANKISEYRHYCYNNRKLPQLCTE |
| | LPLFLAAREGSYEKTVLELTEVANRRTLED |
| | LLMGTDHMFAFKDLFVVPRDQLYNKPLCDV |
| | IVRTLQDIVLHLLE |
| V | SEQ ID NO: 266: |
| | MQTDRELTEVFEFAARYSRSDADAKLPQLC |
| | TEVDNMLLMGTLGIVPLHAAVSADTLHEYM |
| | LDLRNRATDLSEEENDEIDGVNHQHLPARR |
| | MHDNLLIRCLRCPLILAARLAVLFLNTLSF |
| | VCPWCASSHADVKCIDFYSRIDDLKLPDLC |
| | TELALHWAAAVNNVQLFLNTLSFVGANKNN |
| | RVYDFAFRDLPLFLAAREGSYEKLTNTGLY |
| | NLANRLCIVYRDGNPYAVCDDHMYMLDLQP |
| | ETTPRDFAFRDLCIVYIVRLLDLEVSQTSK |
| | LTRQTDRTLGIVCPIAARYSRSDRTLEDLL |
| | MGVADATIHDIILECVDNMHLDKKQRFHNI |
| | PLHAAVSADTTLEQQYNKPLCDLLRNRATD |
| | LMHDRWTGRCMSCCPLILAARLAVSLQDIE |
| | ITCVSHADVLCVQSTHVDIDDLAHYNIVTF |
| | CCLHWAAAVNNVDDLRAFQQLFLNTLSGAN |
| | KISEYRHYCYNNRKLPQLCTELPLFLAARE |
| | GSYEKTVLELTEVANRRTLEDLLMGTDHMF |
| | AFKDLFVVPRDQLYNKPLCDVIVRTLQDIV |
| | LHLLE |
| W | SEQ ID NO: 267: |
| | MQTDRELTEVFEFAARYSRSDADAKLPQLC |
| | TEVDNMLLMGTLGIVPLHAAVSADTLHEYM |
| | LDLRNRATDLSEEENDEIDGVNHQHLPARR |
| | MHDNLLIRCLRCPLILAARLAVLFLNTLSF |
| | VCPWCASSHADVKCIDFYSRIDDLKLPDLC |
| | TELALHWAAAVNNVQLFLNTLSFVGANKNN |
| | RVYDFAFRDLPLFLAAREGSYEKLTNTGLY |
| | NLANRLCIVYRDGNPYAVCDDHMYMLDLQP |
| | ETTPRDFAFRDLCIVYIVRLLDLEVSQTSK |
| | LTRQTDRTLGIVCPIAARYSRSDRTLEDLL |
| | MGVADATIHDIILECVDNMHLDKKQRFHNI |
| | PLHAAVSADTTLEQQYNKPLCDLLRNRATD |
| | LMHDRWTGRCMSCCPLILAARLAVSLQDIE |
| | ITCVSHADVLCVQSTHVDIDDLAHYNIVTF |
| | CCLHWAAAVNNVDDLRAFQQLFLNTLSGAN |
| | KNNRKLPQLCTELPLFLAAREGSYEKTVLE |
| | LTEVANRRTLEDLLMGTDHMFAFKDLFVVP |
| | RDQLYNKPLCDVIVRTLQDIVLHLLE |
| X | SEQ ID NO: 268: |
| | MQTDRELTEVFEFAARYSRSDYIIFVYIPL |
| | ADAKLPQLCTEVDNMLLMGTLGIVPLHAAV |
| | SADTLHEYMLDLRNRATDLSEEENDEIDGV |
| | NHQHLPARRMHDNLLIRCLRCPLILAARLA |
| | VLFLNTLSFVCPWCASSHADVKCIDFYSRI |
| | DDLKLPDLCTELALHWAAAVNNVQLFLNTL |
| | SFVGANKFQQLFLNTLNNRVYDFAFRDLPL |
| | FLAAREGSYEKLTNTGLYNLANRLCIVYRD |
| | GNPYAVCDDHMYMLDLQPETTPRDFAFRDL |
| | CIVYIVRLLDLEVSQTSKLTRQTDRTLGIV |
| | CPIAARYSRSDRTLEDLLMGVADATIHDII |
| | LECVDNMHLDKKQRFHNIPLHAAVSADTTL |
| | EQQYNKPLCDLLRNRATDLCDSTLRLCVMH |
| | DRWTGRCMSCCPLILAARLAVSLQDIEITC |
| | VSHADVLCVQSTHVDIDDLAHYNIVTFCCL |
| | HWAAAVNNVDDLRAFQQLFLNTLSGANKIS |
| | EYRHYCYNNRKLPQLCTELPLFLAAREGSY |
| | EKTVLELTEVANRRTLEDLLMGTDHMFAFK |
| | DLFVVPRDQLYNKPLCDVIVRTLQDIVLHL |
| | LEYLQYGVYRFADAQLLRREVYVIVR |
| Y | SEQ ID NO: 269: |
| | ARYSRSDTLGIVCPICSADAMQTDRELTEV |
| | FEFAARYSRSDYIIFVYIPLADAKLPQLCT |
| | EVDNMLLMGTLGIVPLHAAVSADTLHEYML |
| | DLRNRATDLSEEENDEIDGVNHQHLPARRM |
| | HDNLLIRCLRCPLILAARLAVLFLNTLSFV |
| | CPWCASSHADVKCIDFYSRIDDLKLPDLCT |
| | ELALHWAAAVNNVQLFLNTLSFVGANKFQQ |
| | LFLNTLNNRVYDFAFRDLPLFLAAREGSYE |
| | KLTNTGLYNLANRLCIVYRDGNPYAVCDDH |
| | MYMLDLQPETTPRDFAFRDLCIVYIVRLLD |
| | LEVSQTSKLTRQTDRTLGIVCPIAARYSRS |
| | DRTLEDLLMGVADATIHDIILECVDNMHLD |
| | KKQRFHNIPLHAAVSADTTLEQQYNKPLCD |
| | LLRNRATDLCDSTLRLCVMHDRWTGRCMSC |
| | CPLILAARLAVSLQDIEITCVSHADVLCVQ |
| | STHVDIDDLAHYNIVTFCCLHWAAAVNNVD |
| | DLRAFQQLFLNTLSGANKISEYRHYCYNNR |
| | KLPQLCTELPLFLAAREGSYEKTVLELTEV |
| | ANRRTLEDLLMGTDHMFAFKDLFVVPRDQL |
| | YNKPLCDVIVRTLQDIVLHLLEYLQYGVYR |
| | FADAQLLRREVYVIVR |
| Z | SEQ ID NO: 270: |
| | DDLKLPDLCTELARYSRSDTLGIVCPICSA |
| | DAMQTDRELTEVFEFAARYSRSDYIIFVYI |
| | PLADAKLPQLCTEVDNMLLMGTLGIVPLHA |
| | AVSADTLHEYMLDLRNRATDLSEEENDEID |
| | GVNHQHLPARRMHDNLLIRCLRCPLILAAR |
| | LA+10VLFLNTLSFVCPWCASSHADVKCID |
| | FYSRIDDLKLPDLCTELALHWAAAVNNVQL |
| | FLNTLSFVGANKFQQLFLNTLNNRVYDFAF |
| | RDLPLFLAAREGSYEKLTNTGLYNLANRLC |
| | IVYRDGNPYAVCDDHMYMLDLQPETTPRDF |
| | AFRDLCIVYIVRLLDLEVSQTSKLTRQTDR |
| | TLGIVCPIAARYSRSDRTLEDLLMGVADAT |
| | IHDIILECVDNMHLDKKQRFHNIPLHAAVS |
| | ADTTLEQQYNKPLCDLLRNRATDLCDSTLR |
| | LCVMHDRWTGRCMSCCPLILAARLAVSLQD |
| | IEITCVSHADVLCVQSTHVDIDDLAHYNIV |
| | TFCCLHWAAAVNNVDDLRAFQQLFLNTLSG |
| | ANKISEYRHYCYNNRKLPQLCTELPLFLAA |
| | REGSYEKTVLELTEVANRRTLEDLLMGTDH |
| | MFAFKDLFVVPRDQLYNKPLCDVIVRTLQD |
| | IVLHLLEYLQYGVYRFADAQLLRREVYVIV |
| | R |
| AA | SEQ ID NO: 271: |
| | IQTNRELTEVFEFAPRYSHSDYIIFVYIPL |
| | GDAKLPQLCTEVDDLLLMGTLGIVPLHAPM |
| | SADTLHEYMLDLHNRATDLSEEENDEIDGV |
| | NHQHLPARRIHDNLLIRCLRCPLILAARLA |
| | LLFLNTLSFVCPWCASSHADMKCIDFYSRI |
| | DNLKLPDLCTELALHWTAAVNNLQLFLNTL |
| | SFVPANKFQQLFLNTLNDRVYDFAFRDLPL |
| | FLAAKEGSYEKLTNTGLYNLASRLCIVYRD |
| | GNPYAVCDNHMYMLDLQPETTPRQFAFRDL |
| | CIVYIVRFLNLEVSQTSKFTRQTERTLGIV |
| | CPIAAHYSKSDRTLEDLLMGVANPTIHDII |
| | LECVQNMHLDKKQRFHNIPLHAAYSADTTL |
| | EQQYNKPLCDLLRSRATDLCDSTLRLCVVH |
| | DRWTGRCMSCCPLILAPRLAVSLQDIEITC |
| | VSHADILCVQSTHVDIDDIAHYNIVTFCCL |
| | HWGATMSNVDDLRAFQQLFLNTLSGPSKIS |
| | EYRHYCYDNRKLPQLCTELPLFLGAKEPSY |
| | EKTVLELTEVADRRTLEDLLMGTQHMFAFK |
| | DLFVVPRNQLYNKPLCDVIIHTLQDIVLHL |
| | FEYLQYGVYRFADA |
| BB | SEQ ID NO: 272: |
| | MKTDRELTEVFEFAAKYSRSDYIIFVYIPL |
| | AEAKLPQLCTEVENMLLMGTLGIVPLHAGV |
| | SADTLHEYMLDLRNRTTDLSEEENDEIDGV |
| | NHQHLPARRMHENLLIRCLRCPLILAAHLA |
| | VLFLNTLSFVCPWCASSHSDVKCIDFYSRI |
| | DDIKLPDLCTELALHWAAAMNNVQLFLNTL |
| | SFVGAQKFQQLFLNTLNSRVYDFAFRDLPL |
| | FLAAHEGSYEKLTNTGLYNLASRLCIVYRD |
| | GNPYAVCDDHVYMLDLQPETTPKDFAFRDL |
| | CIVYIVRLLDLEVSHTSKLTRQTDRTLGIV |
| | CPIAAKYSRSDRTLEDLLMGVAEADNIHLD |
| | KKQRFHNIPLHAGVSADTTLEQQYNKPLCD |
| | LLRNRTTDLCDSTLRLCVMHNRWTGRCMSC |
| | CPLILAAHLAVSLQDIEITCVSHPDVLCVQ |
| | STHVDIDDIAHYNIVTFCCLHWAAAINNVD |
| | DLRAFQQLFLNTLSGAQKISEYRHYCYNDR |
| | KLPQLCTELPLFLAAEEGSYEKTVLELTEV |
| | ASRRTLEDLLMGTDHVFAFKDLFVVPKDQL |
| | YNKPLCDVIVKTLQDIVLHLLN |
| CC | SEQ ID NO: 273: |
| | MKTDRELTEVFEFAAKYSRSDYIIFVYIPL |
| | AEALLMGTLGIVENMKLPQLCTEVPLHAGV |
| | SADTLHEYMLDLRNRTTDLSEEENDEIDGV |
| | NHQHLPARRMHENLLIRCLRCPLILAAHLA |
| | VLFLNTLSFVCPWCASSHSDVKCIDFYSRI |
| | DDIKLPDLCTELALHWAAAMNNVQLFLNTL |
| | SFVGAQKVYDFAFRDLNSRFQQLFLNTLPL |
| | FLAAHEGSYEKLTNTGLYNLASRLCIVYRD |
| | GNPYAVCDDHVYMLDLQPETTPKDFAFRDL |
| | CIVYIVRLLDLEVSHTSKLTRQTDRTLGIV |
| | CPIAAKYSRSDRTLEDLLMGVAEADNIHLD |
| | KKQRFHNIPLHAGVSADTTLEQQYNKPLCD |
| | LLRNRTTDLCDSTLRLCVMHNSLQDIEITC |
| | VPLILAAHLAVRWTGRCMSCCSHPDVLCVQ |
| | STHVDIDDIAHYNIVTFCCLHWAAAINNVD |
| | DLRAFQQLFLNTLSGAQKISEYRHYCYNDR |
| | KLPQLCTELPLFLAAEEGSYEKTVLELTEV |
| | ASRRTLEDLLMGTDHVFAFKDLFVVPKDQL |
| | YNKPLCDVIVKTLQDIVLHLLN |
| DD | SEQ ID NO: 274: |
| | IQTNRYIIFVYIPLPRYSHSDELTEVFEFA |
| | GDAKLPQLCTEVDDLLLMGTLGIVPLHAPM |
| | SADTLHEYMLDLHNRATDLSEEENDEIDGV |
| | NHQHLPARRIHDNLLIRCLRCPLILAARLA |
| | LLFLNTLSFVCPWCASSHADMKCIDFYSRI |
| | DNLKLPDLCTELALHWTAAVNNLQLFLNTL |
| | SFVPANKFQQLFLNTLNDRVYDFAFRDLPL |
| | FLAAKEGSYEKLTNTGLYNLASRLCIVYRD |
| | GNPYAVCDNHMYMLDLQPETTPRQFAFRDL |
| | CIVYIVRFLNLEVSQTSKFTRQTERTLGIV |
| | CPIAAHYSKSDRTLEDLLMGVANPTIHDII |
| | LECVQNMHLDKKQRFHNIPLHAAYSADCDS |
| | TLRLCVRSRATDLTTLEQQYNKPLCDLLVH |
| | DRWTGRCMSCCPLILAPRLAVSLQDIEITC |
| | VSHADILCVQSTHVDIDDIAHYNIVTFCCL |
| | HWGATMSNVDDLRAFQQLFLNTLSGPSKIS |
| | EYRHYCYDNRKLPQLCTELPLFLGAKEPSY |
| | EKTVLELTEVADRRTLEDLLMGTQHMFAFK |
| | DLFVVPRNQLYNKPLCDVIIHTLQDIVLHL |
| | FEYLQYGVYRFADA |

1. (canceled)

2. The method of claim 34, wherein the fusion protein comprises:
(a) an HPV-16 E5 peptide comprising the amino acid sequence of SEQ ID NO: 130;
(b) an HPV-16 E6 peptide comprising the amino acid sequence of any one of SEQ ID NOs: 113-121;
(c) an HPV-18 E6 peptide comprising the amino acid sequence of any one of SEQ ID NOs: 131-138;
(d) an HPV-16 E7 peptide comprising the amino acid sequence of any one of SEQ ID NOs: 122-129; and/or
(e) an HPV-18 E7 peptide comprising the amino acid sequence of any one of SEQ ID NOs: 139-144.

3-7. (canceled)

8. The method of claim 34, wherein the fusion protein further comprises an agonist peptide comprising the amino acid sequence of any one of SEQ ID NOs: 145-147.

9. The method of claim 34, wherein the fusion protein further comprises an ankyrin-like repeat domain.

10-11. (canceled)

12. The method of claim 34, wherein the fusion protein comprises an amino acid sequence having at least 90% identity with any one of SEQ ID NOs: 51-53 and 243.

13. The method of claim 34, wherein the fusion protein comprises an amino acid sequence having at least 95% identity with any one of SEQ ID NOs: 51-53 and 243.

14-16. (canceled)

17. The method of claim 34, wherein the fusion protein comprises the amino acid sequence of SEQ ID NO: 243 or a conservatively-substituted variant thereof.

18. The method of claim 34, wherein the fusion protein comprises the amino acid sequence of SEQ ID NO: 243.

19-23. (canceled)

24. The method of claim 34, comprising administering a vector comprising the polynucleotide.

25-26. (canceled)

27. The method of claim 24, wherein the vector is an adenoviral vector is deficient in one or more elements selected from an E1-E4 region and an L1-L5 region.

28. (canceled)

29. The method of claim 24, wherein the vector is a gorilla adenoviral vector.

30. The method of claim 24, wherein the vector is a GC46 gorilla adenoviral vector.

31. (canceled)

32. A method of inducing an anti-HPV immune response in a subject in need thereof, the method comprising administering to the subject a therapeutically effective amount of a polynucleotide encoding a fusion protein, the fusion protein comprising:
(a) an HPV-16 E5 peptide or an HPV-18 E5 peptide; and
(b) an HPV-16 E6 peptide, an HPV-16 E7 peptide, an HPV-18 E6 peptide, or an HPV-18 E7 peptide.

33. (canceled)

34. A method of treating an HPV-associated disease or disorder in a subject in need thereof, the method comprising administering to the subject a therapeutically effective amount of a polynucleotide encoding a fusion protein, the fusion protein comprising:
(a) an HPV-16 E5 peptide or an HPV-18 E5 peptide; and
(b) an HPV-16 E6 peptide, an HPV-16 E7 peptide, an HPV-18 E6 peptide, or an HPV-18 E7 peptide.

35. The method of claim 34, wherein the HPV-associated disease or disorder is an HPV-16 associated disease or disorder, an HPV-18 associated disease or disorder, or an HPV-45 associated disease or disorder.

36. The method of claim 34, wherein the HPV-associated disease or disorder is an HPV-associated cancer.

37. The method of claim 36, wherein the HPV-associated cancer is a lower genital tract neoplasia, a cervical cancer, a vulvar cancer, an anal cancer, a penile cancer, or a head and neck cancer.

38. The method of claim 29, wherein the therapeutically effective amount comprises about 0.1×10⁹ to about 10×10¹² particle units.

39. (canceled)

40. The method of claim 34, further comprising administering: an anti-inflammatory agent; an analgesic; a biological response modifier; a cytokine; an interferon; an interleukin; a CAR-T cell; a bifunctional protein; a colony-stimulating factor; a tumor necrosis factor; a surface active agent; a small molecule enzyme inhibitor; a chemotherapy agent; and/or an anti-metastatic agent.

41. The method of claim 34, further comprising administering a biological response modifier and/or a chemotherapy agent.

42. The method of claim 34, further comprising administering an immune checkpoint inhibitor.

43-50. (canceled)


SEQUENCE LISTING (324 sequences)

SEQ ID NO: 1 - DNA, 30 bp, synthetic construct
  source 1..30: mol_type = other DNA
agctctttggtggcgagcggcgcggcctct

SEQ ID NO: 2 - DNA, 439 bp, synthetic construct
  source 1..439: mol_type = other DNA
aacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgc

SEQ ID NO: 3 - DNA, 34 bp, synthetic construct
  source 1..34: mol_type = other DNA
actgaggctgcggctaaggctgaggtcgaagcca

SEQ ID NO: 4 - DNA, 45 bp, synthetic construct
  source 1..45: mol_type = other DNA
ataggtgtggatgccacacaggcgggagataaccctatatatgct

SEQ ID NO: 5 - DNA, 30 bp, synthetic construct
  source 1..30: mol_type = other DNA
gtagcaggccccctagctgtggccaatggc

SEQ ID NO: 6 - DNA, 399 bp, synthetic construct
  source 1..399: mol_type = other DNA
atgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccc

SEQ ID NO: 7 - DNA, 3168 bp, synthetic construct
  source 1..3168: mol_type = other DNA
atggacagctccaatgtgcgcgatgtcgtcatcaaactccgcccgccgagcgccgagatctggacctgcggctctcgcggcgtggtggtctgctccaccatcgccctccaggagacagatgctggcggccagacaaccaaagtagaagaccaccagccacacgggaccccaggcgggggacttagattcccgctgcgcttcctcgtcagaggtcgccaggttcacctcgtgcaagatatacaacccgtgcagcgctgccagtactgcggtcgcttttacaaaagccagcacgagtgctcggcccgcagacgggacttctactttcaccacatcaacagccaatcctccaactggtggcgggagatccagttcttcccgatcggctctcatcctcgcacggagcgcctctttgtcacctacgatgtagagacctacacttggatgggagcctttggcaagcagctcgtgcccttcatgctggtcatgaaactggggggcgacgaggctctggtcgccgccgcgcgcgacctcgcccgagagctcagatgggacccctgggagaaagaccccctcaccttctactgcatcacccccgaaaagatggccgtggggcgacagttcagaaccttccgcgaccgcctgcagaccctcatggcccgcgacctctggcgatccttcctggcggccaaccctcacttgcaagactgggccctggaggagcacggcctggaatcgcccgaggagctcacctacgaggaactcaaaaagctcccctccatcaagggccagccccgctttttggagctctacatcgtgggccacaacataaacggctttgacgagatcgtcctggccgcccaggtcatcaacaaccgctcctcggtcccagggccctttcgcatcaccagaaacttcatgcctcgagcggggaagatcctcttcaatgacctcaccttctccctgcccaacccgcgctccaaaaagcgcacggactacaccctgtgggaacagggcggctgcgatgacacagacttcaaacatcaatacctcaaagtcatggtcagggacactttcgccctcacccacacctccctccgcaaggcggcgcaggcctacgcgctgcccgtggagaagggctgttgcccctaccaggccgtcaaccagttctacatgctaggctcttaccgttcggacacggacgggtttcccctccaagagtactggaaagaccgcgaagagttcgtcctcaaccgcgagctgtggaaaaagaagggggaggataagtatgacatcatccgcgagaccctcgactactgcgcgctcgacgtccaggtcaccgccgagctggtgcacaagctgcgcgagtcctacgcctccttcgtcagggactcggtgggcttgcaagaagcaagcttcaacgtcttccagcggcccaccatctcctccaactcccatgccatcttcaggcagatcgccttccgcgccgagcgcccccagcgcaccaacctcgggcccaacatgctggccccctcccacgagctctatgactacgtgcgcgccagcatccgcggggggcgctgctaccccacctacctcggcatcctcagggaacccctgtacgtgtatgacatctgcggcatgtacgcctccgcgctcacccaccccatgccctggggcccgcccctcaacccctacgagcgcgcgctcgccgcccgcgaatggcagcgggctctggacatgcaagcttgcaagatcgactactttgacccgcgcttgctccccggggtcttcaccatcgacgcggaccccccaaacgaggaccagctggaccccctaccccccttctgctcgcgcaagggcggccgcctctgctggaccaacgagcgcctgcgcggcgaggtcgccaccagcgtcgacatggtcaccctgcacaaccgaggctggagggtgcgcctaatcccagacgagcgcaccaccgtcttccccgagtggaagtgcgtggcccgcgagtacgtgcaactcaacatcgcggccaaggagcgagccgaccgcgacaaaaaccagaccctgcgctccatcgccaagctgctctccaacgccctctacgggtcgttcgccaccaagcttgacaacaaaaaaatagtgttttctgaccagatggacccaggtaccctcaaaggtatcacctccggacaggtgaacatcaaatcctcctcatttttagaaactgacaacctgagcgctgaggtcatgcccgccttcgagagggaatacttaccccagcagctggccctcgcagacagcgatgcggaagagagtgaagatgaaagggcgcccacccccttttatacccccccgtcgggaacccccggtcacgtgtcctacacctacaagccaatcacttttctggacgcggaggagggggacatgtgcctgcacaccctggagaaggtggacccgctagtggacaacgaccgctacccctcccacgtggcctccttcgtcctggcctggacgcgggccttcgtctcagagtggtcagagtttctctacgaggaggacagaggcactccgctggaagacaggcccctgaagtcggtctacggggacacggacagcctcttcgtcaccgagaagggacaccgcctcatggagagccgaggtaagaaacgcatcaaaaagcatgggggcaacctggtttttgaccctgaccgcccggagctcacttggctggtggaatgcgagacggtctgcgcttcctgcggcgcggacgcctactccccagagtccgtgtttctcgctcccaagctctacgccctgaagagcctgcagtgcccctcgtgcggcgccacctccaagggaaagctccgcgccaaggggcacgccgccgagggtctcgactacgagaccatggtcaaatgctacctggccgacgcgcagggcgaagagcggcagcgattcagcaccagcagaaccagcctcaagcgcaccctggccagcgcccaacccggagcgcaccccttcaccgtgacccagaccaccctgacgaggaccctgcgcccatggaaggacatgactctggccccgctggacgcccatcggctggtgccctacagcgaaagccgccccaacccgcgaaacgaggagatctgctggatcgagatgccg

SEQ ID NO: 8 - DNA, 1974 bp, synthetic construct
  source 1..1974: mol_type = other DNA
atgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttt

SEQ ID NO: 9 - DNA, 2877 bp, synthetic construct
  source 1..2877: mol_type = other DNA
atggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacc

SEQ ID NO: 10 - DNA, 1749 bp, synthetic construct
  source 1..1749: mol_type = other DNA
atgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaa

SEQ ID NO: 11 - protein, 10 aa, synthetic construct
SSLVASGAAS

SEQ ID NO: 12 - protein, 147 aa, synthetic construct
KHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIA

SEQ ID NO: 13 - protein, 15 aa, synthetic construct
KTEAAAKAEVEANVA

SEQ ID NO: 14 - protein, 15 aa, synthetic construct
IGVDATQAGDNPIYA

SEQ ID NO: 15 - protein, 15 aa, synthetic construct
LNVAGPLAVANGINA

SEQ ID NO: 16 - protein, 133 aa, synthetic construct
MSDTGNSFDGSIFSPYLTVRMPHWAGVRQNVMGSNVDGRPVLPSNSSTMAYATVGGTPLDAATSAAASAAAATARSMATDLYSSLVASGAASRASARDEKLTALLLKLEDLTRELGQLTQQVSSLRESSLASP

SEQ ID NO: 17 - protein, 1056 aa, synthetic construct
MDSSNVRDVVIKLRPPSAEIWTCGSRGVVVCSTIALQETDAGGQTTKVEDHQPHGTPGGGLRFPLRFLVRGRQVHLVQDIQPVQRCQYCGRFYKSQHECSARRRDFYFHHINSQSSNWWREIQFFPIGSHPRTERLFVTYDVETYTWMGAFGKQLVPFMLVMKLGGDEALVAAARDLARELRWDPWEKDPLTFYCITPEKMAVGRQFRTFRDRLQTLMARDLWRSFLAANPHLQDWALEEHGLESPEELTYEELKKLPSIKGQPRFLELYIVGHNINGFDEIVLAAQVINNRSSVPGPFRITRNFMPRAGKILFNDLTFSLPNPRSKKRTDYTLWEQGGCDDTDFKHQYLKVMVRDTFALTHTSLRKAAQAYALPVEKGCCPYQAVNQFYMLGSYRSDTDGFPLQEYWKDREEFVLNRELWKKKGEDKYDIIRETLDYCALDVQVTAELVHKLRESYASFVRDSVGLQEASFNVFQRPTISSNSHAIFRQIAFRAERPQRTNLGPNMLAPSHELYDYVRASIRGGRCYPTYLGILREPLYVYDICGMYASALTHPMPWGPPLNPYERALAAREWQRALDMQACKIDYFDPRLLPGVFTIDADPPNEDQLDPLPPFCSRKGGRLCWTNERLRGEVATSVDMVTLHNRGWRVRLIPDERTTVFPEWKCVAREYVQLNIAAKERADRDKNQTLRSIAKLLSNALYGSFATKLDNKKIVFSDQMDPGTLKGITSGQVNIKSSSFLETDNLSAEVMPAFEREYLPQQLALADSDAEESEDERAPTPFYTPPSGTPGHVSYTYKPITFLDAEEGDMCLHTLEKVDPLVDNDRYPSHVASFVLAWTRAFVSEWSEFLYEEDRGTPLEDRPLKSVYGDTDSLFVTEKGHRLMESRGKKRIKKHGGNLVFDPDRPELTWLVECETVCASCGADAYSPESVFLAPKLYALKSLQCPSCGATSKGKLRAKGHAAEGLDYETMVKCYLADAQGEERQRFSTSRTSLKRTLASAQPGAHPFTVTQTTLTRTLRPWKDMTLAPLDAHRLVPYSESRPNPRNEEICWIEMP

SEQ ID NO: 18 - protein, 658 aa, synthetic construct
MRRAAMFEEGPPPSYESAMGISPAAPLQPPYVPPRYLQPTGGRNSICYSELQPLYDTTRLYLVDNKSADVASLNYQNDHSDFLTTVIQNNDFTPTEASTQTINLDNRSNWGGDLKTILHTNMPNVNEFMFTNSFKARVMVAREQGEAKYEWVDFTLPEGNYSETMTLDLMNNAIVEHYLKVGRQNGVKESDIGVKFDTRNFRLGWDPVTGLVMPGVYTNEAFHPDIVLLPGCGVDFTQSRLSNLLGIRKRQPFQEGFKITYEDLKGGNIPALLDLDAYEESLKPEESAGDSGESGEEQAGGGGGASVENESTPAVAADAAEVEPEAMQQDAEEGAQEGAQKDMNDGEIRGDTFATRGEEKEAEAAAAATAEAETEVEAEAEPETEVMEDMNDGERRGDTFATRGEEKAAEAEAAAEEAAAAAAKTEAAAKAEVEANVAVEAQAEEEAAAEAVKEKAQAEQEEKKPVIQPLKEDSKKRSYNVIEGSTFTQYRSWYLAYNYGDPVKGVRSWTLLCTPDVTCGSEQMYWSLPNMMQDPVTFRSTRQVSNFPVVGAELLPVHSKSFYNEQAVYSQLIRQATSLTHVFNRFPENQILARPPAPTITTVSENVPALTDHGTLPLRNSISGVQRVTITDARRRTCPYVYKALGIVSPRVLSSRTF

SEQ ID NO: 19 - protein, 959 aa, synthetic construct
MATPSMMPQWSYMHISGQDASEYLSPGLVQFARATDTYFNMSNKFRNPTVAPTHDVTTDRSQRLTLRFIPVDREDTAYSYKARFTLAVGDNRVLDMASTYFDIRGVLDRGPTFKPYSGTAYNSLAPKGAPNSCEWEQDEPAQAAIAEDEEELEEEQAQDEQAPTKKTHVYAQAPLSGEKITKDGLQIGVDATQAGDNPIYADKTFQPEPQIGESQWNEADATVAGGRVLKKTTPMRPCYGSYAKPTNANGGQGIMVANDQGALESKVEMQFFSTTTSLNVREGENNLQPKVVLYSEDVNLESPDTHLSYKPKKDDTNSKIMLGQQAMPNRPNLIAFRDNFIGLMYYNSTGNMGVLAGQASQLNAVVDLQDRNTELSYQLMLDSIGDRSRYFSMWNQAVDSYDPDVRIIENHGVEDELPNYCFPLGGIGITDTYQCIKPTAAANNTTWSKDEEFSDRNEIGVGNNFAMEINIQANLWRNFLYANVGLYLPDKLKYNPTNVDISDNPNTYDYMNKRVVAPGLVDCFVNVGARWSLDYMDNVNPFNHHRNAGLRYRSMILGNGRYVPFHIQVPQKFFAIKNLLLLPGSYTYEWNFRKDVNMVLQSSLGNDLRVDGASIKFDSVTLYATFFPMAHNTASTLEAMLRNDTNDQSFNDYLSGANMLYPIPAKATNVPISIPSRNWAAFRGWAFTRLKTKETPSLGSGFDPYFVYSGSIPYLDGTFYLNHTFKKISIMYDSSVSWPGNDRLLTPNEFEVKRAVDGEGYNVAQCNMTKDWFLVQMLANYNIGYQGFYIPESYKDRMYSFFRNFQPMSRQVVDETKYKDYQAIGITHQHNNSGFVGYLAPTMREGQAYPANFPYPLIGKTAVDSVTQKKFLCDRTLWRIPFSSNFMSMGALTDLGQNLLYANSAHALDMTFEVDPMDEPTLLYIVFEVFDVVRVHQPHRGVIETVYLRTPFSAGNATT

SEQ ID NO: 20 - protein, 583 aa, synthetic construct
MKRARSSDETFNPVYPYDTEIAPTSVPFLTPPFVSSAGMQENPAGVLSLHLSEPLTTHNGALTLKMGGGLTLDKEGNLTSQNITSVDPPLKKSKNNISLQTAAPLAVSSGALTLFATPPLAVSGDNLTVQSQAPLTLEDSKLTLATKGPLTVSEGKLVLETEAPLHASDSSSLGLSVTAPLSINNDSLGLDLQAPIVSQNGKLALNVAGPLAVANGINALTVGTGKGIGLNETSTHLQAKLVAPLGFDTNGNIKLSVAGGMRLNNDTLILDVNYPFEAQGQLSLRVGQGPLYVDSSSHNLTIRCLRGLYITSSNNQTGLEANIKLTKGLVYDGNAIAVNVGQGLQYSTTATSEGVYPIQSKIGLGMEYDTNGAMMTKLGSGLSFDNSGAIVVGNKNDDRLTLWTTPDPSPNCRIYSEKDTKLTLVLTKCGSQILGTVSALAVRGSLAPITNASSIVQIFLRFDENGLLMSNSSLDGDYWNYRNGDSTNSTPYTNAVGFMPNLAAYPKGQATAAKSSIVSQVYMDGDTTKPITLKINFNGIDETTENTPVSKYSMTFSWSWPTASYIGHTFATNSFTFSYIAQE

SEQ ID NO: 21 - DNA, 32728 bp, synthetic construct
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtc

SEQ ID NO: 22 - DNA, 32728 bp, synthetic construct
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaactt

SEQ ID NO: 23 - DNA, 32728 bp, synthetic construct
  source 1..32728: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaac

SEQ ID NO: 24 - DNA, 32728 bp, synthetic construct
  source 1..32728: mol_type = other DNA
ataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagcgcgtaatatttaccgagggccgagggactttgaccgattacgtggaggactcgcccaggtgttttttacgtgaatttccgcgttccgggtcaaagtctccgtttttattgtcaccgtcatttgacgcggagggtatttaaacccgctgcgctcctcaagaggccactcttgagtgccagcgagaagagttttctcctctgctccgcttcggtgatcgaaaaatgagacacatagcctgcactccgggtcttttgtccggtcgggcggcggccgagcttttggacgctttgatcaatgatgtcctaagcgatgattttccgtctactacccactttagcccacctactcttcacgaactgtacgatctggatgtactggtggatgtgaacgatcccaacgaggaggcggtttctgcgttttttcccgagtctgcgctgttggccgctcaggagggatttgacctacacactccgccgcctattttagagtctccgctgccggagcccagtggtataccttatatgcctgaactgcttcccgaagtggtagacctgacctgccacgagcctggctttccgcccagcgacgatgagggtgagccttttgttttagactttgctgagatacctgggcacggttgcaggtcttgtgcatatcatcagagggttaccggagaccccgaggttaagtgttcgctgtgctatatgaggatgacctcttcctttatctacagtaagtttttgtctaggtgggcttttgggtaggtgggttttgtgtcagaacaggtgtaaacgttgcttgtgttttttgtacctgtaggtccggtgtccgagccagacccggagcccgaccgcgatcccgagccggatcccgagcctcctcgcaggacaaggaaactaccttccattctgtgcaagtctcagacacctgtaaggaccagcgaggcagacagcaccgactctggcacttctacctctccccctgaaattcacccagtggttcctctgggtatacataaacctgttgctgttaaagtttgcgggcgacgccctgcagtacagtgcattgaggacttgcttcacgatcccgaggaacctttggacttgagccttaaacgccctaggcaataaaccccacctaagtaataaaccccacctaagtaataaaccctgccgcccttggttattgagatgacgcccaatgtttgcttttgaatgacttcatgtgtgtaataaaagtgagtgtgatcataggtctcttgtttgtctgggcggggcttaagggtatataagtctcttggggctaaacttggttacacttgaccccaatggaggcgtgggggtgcttggaggagtttgcggacgtgcgccgtttgctggacgagagctctagcaatacctatactatttggaggtatctgtggggctctactcaggccaagttggtttccagaattaagcaggattacaagtgcgattttgaagagctttttagttcctgcggtgagcttttgcaatccttgaatctgggccatcaggctattttccaggaaaaggttctctcgactttggatttttccactcccgggcgcaccgccgcttgtgtggcttttgtgtcttttgtgcaagataaatggagcgaggagacccacctgagtcacggctacgtactggatttcatggcgatggctctttggagggctcacaacaaatggaagattcagaaggaactgtacggttccgccctacgtcgtccacttctgtcgcgacaggggctgaggtttcccgaccatcggcagcatcagaatctggaagacgagtcggaggagcgagcggaggagaagatcagcttgagagccggcctggaccctcctcaggaggaatgaatctcccgcaggtggttgacctgtttccagaactgagacgggtcctgactatcagggaggatggtcagtttgtgaagaagtttaagagggatcggggtgagggagatgatgaggcggctagcaatttagcttttagtctgatgactcgccaccgaccggaatgtattacctatcagcagattaaggagagttgtgccaacgagctggatcttttgggtcagaagtatagcatagaacagcttaccacttactggcttcagcctggggatgattgggaagaggcgatcagggtgtatgcaaaggtggccctgcggcccgattgcaagtataagattactaagttggttaatattagaaactgctgctatatttctgggaacggggccgaagtggagatagatactcaggacagggtggcttttaggtgttgcatgataaacatgtggcccgggatactggggatggatggggtggtattcatgaatgtgaggtttacgggccccaactttaatggcacggtgttcatgggcaacaccaacttgctcctgcatggtgcgagtttctatgggtttaataacacctgtatagaggcctggaccgatgtaaaggttcgaggttgttccttttatagctgttggaaggcggtggtgtgtcgccctaaaagcaggggttctgtgaaaaaatgcttgtttgaaaggtgcaccttaggcatcctctctgagggcaactccagggtgcgccataatgtggcttcgaactgcggttgcttcatgcaagtgaagggggtgagcgttatcaagcataactcggtgtgtggaaactgcgaggatcgcgcctcccagatgctgacctgctttgatggcaactgtcacctgttgaagaccattcatataagcagccaccccagaaaggcctggcccgtgtttgagcataacatcttgacccgctgctccttgcatctgggggtcaggaggggtatgttcctgccttaccagtgtaactttagccacactaaaatcctgctggaacccgagtgcatgaccaaggtcagcctgaatggtgtgtttgatgtgactctgaaaatctggaaggtgctgaggtatgatgagaccaggaccaggtgccgaccctgcgagtgcggcggcaagcacatgagaaatcagcctgtgatgttggatgtgaccgaggagcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctaga

SEQ ID NO: 25 - DNA, 32728 bp, synthetic construct
  source 1..32728: mol_type = other DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacgcttaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctgggtcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtgcacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacgaatcgggggcggagggtcccggactgggggatgatggtaccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagcctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtccccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcgccctttcatcttcactctcttccgcatcgctgtctgcgagggccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggattaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaaggaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgtcgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgtgagaaggcgttgagcccgccctcgttccagacccggctgtagaccacgcccccctcggcgtcgcgagcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgttgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgagcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggtcttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtaaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgtttatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgaggagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcacccagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgtcgtggccccgcgggcacgaaggcgcgcggccgctgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgtgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaataaaaaaaagtctagactctcacgctcgcttggtcctgtgactattttgtagaaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagatgaaccagctcaggcagcaatagctgaagatgaagaagaacttgaagaagaacaagctcaggacgaacaggcgcccactaagaaaacccatgtatacgcccaggcacctctttctggtgaaaaaattactaaggatggtttgcaaataggtgtggatgccacacaggcgggagataaccctatatatgctgataaaacattccaacccgaacctcagataggtgagtctcagtggaacgaggctgatgccacagtagcaggaggcagagtcttaaaaaagaccacccctatgagaccttgctatggatcctatgccaaacctactaatgccaatggcggtcaagggatcatggtggccaatgatcagggagcgcttgaatctaaagttgagatgcaatttttctccaccacaacgtctcttaatgtaagggaaggtgaaaacaatcttcagccaaaagtagtgctatacagcgaagatgttaacttggaatcccctgacactcatttgtcttacaaacctaaaaaggatgacaccaactctaaaatcatgttgggtcagcaagccatgcccaacagacccaacctcattgcttttagggacaactttattggacttatgtactacaacagcacaggcaacatgggagtgctggcaggacaggcctcccagctaaacgctgtggtagacttgcaagacagaaacacagagctgtcataccaactgatgcttgattccattggagacagatcaagatacttttccatgtggaaccaggcagtggacagctatgacccagatgtcagaatcattgaaaaccatggggttgaagatgagctgcccaactattgctttcccctgggcggtattggaattacagacacataccagtgcataaaaccaaccgcagctgctaataacactacatggtctaaggatgaagaatttagtgatcgcaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaaaaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaagaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttaaactcgggcaccaccatgcggggcagtggttcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattatgcacgctggccagcaggctctcgtcgctgatcatgtcgctgtccagatcctccgcgttgctcagggcgaacggggtcatcttgcagacctgcctgcccaggaaaggcggcagcccgggcttgccgttgcagtcgcagcgcaggggcatcagcaggtgcccgcggcccgactgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtaccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagcgccgcctgctcttcttcgtcttcgctgtctaccactatctctggggaagggcttctccgctctgcggcggcgcgcttcttttttttcttgggagcggccgtgatggagtccgccacggcgacggaggtcgagggcgtggggctgggggtgcgcggtaccagggcctcgtcgccctcggactcttcctctgactccaggcggcggcggagtcgcttctttgggggcgcgcgcgtcagcggcggcggagacggggacggggacggggacgggacgccctccacagggggtggtcttcgcgcagacccgcggccgcgctcgggggtcttctcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggacccccccgccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccattgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtacccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtaccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctaaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgtggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagatctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagactttttgctgaagaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgaggaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcacagctcgcgggcggctgtcgtcacagggtgcggcctcctcgccagggtataactcacctggagatccgaggcagaggtattcagctcaacgacgagtcggtgagctcctcgctcggtctcagacctgacgggaccttccagatagccggagccggccgatcttccttcacgccccgccaggcgtacctgactctgcaaagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacgagagttaacacttgtggggtttctggtgtatgtgacgctggtggtggctcttttgattaaggcttttccttccatgtctgaactctccctcttcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaatgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcattgatgaaaagaaaatgccttaatcgctttcacgcttgattgctaacaccgggtttttatccgcagaatgattggaatcaccctactaatcacctccctccttgcgattgcccatgggttggaacgaatcgaagtccctgtgggggccaatgttaccctggtggggcctgtcggcaatgctacattaatgtgggaaaaatatactaaaaatcaatgggtctcttactgcactaacaaaaatagccacaagcccagagccatctgcgatgggcaaaatctaaccttgattgatgttcaattgctggatgcgggctactattatgggcagctgggtacaatgattaattactggagaccccacagagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgaaaccaccgagatctgcttctgcaaatgctctgacgccattgcccaggatttggaagatcacgaggaagatgagcatgacttcgcagatgcatgccaggcatcagagccagaagcgctgccggtggccctcaaacagtatgcagacccccacaccacccccgaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccaaaagttcgccctcatgataggcgaacaacccatcaccgtgacccagcactccgtggagacagaaggctgcatacatgctccctgtaggggcgctgactgcctctacaccttgatcaaaaccctctgcggtctcagagaccttatccctttcaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagatctgcaggcacccattgtctctcaaaatggaaaactggctctaaatgtagcaggccccctagctgtggccaatggcattaatgctttgacagtaggcacaggcaaaggtattggtctaaatgaaaccagcactcacttgcaagcaaagttggtcgcccccctaggctttgataccaatggcaacattaagctaagcgttgcaggaggcatgagactaaataatgacacacttatactagatgtaaactacccatttgaagctcaaggccaactaagtctaagagtgggccagggtccgctgtatgtagattctagcagccataacctgaccattagatgccttagaggattatacataacatcgtctaataaccaaaccggtctagaggccaacataaaactaacaaaaggccttgtctatgatggaaatgccatagcagtcaatgttggtcaaggattgcaatacagcactactgccacatcggaaggtgtgtatcctatacagtctaagataggtttgggaatggaatatgataccaacggagccatgatgacaaaactaggctctggactaagctttgacaattcaggagccattgtagtgggaaacaaaaatgatgacaggcttactctgtggactacaccagacccatctcctaactgtagaatttattctgaaaaagatactaaactaaccttggtgctgactaagtgtggcagccaaatcctaggcacagtatctgcccttgctgtcagaggcagccttgcgcccatcactaatgcatccagcatagtccaaatatttctaagatttgatgaaaatggactattgatgagcaactcatcgctagacggtgattactggaattacagaaatggggactccactaatagcacaccatatacaaatgcagtaggctttatgcctaatctagcagcctatcctaaaggtcaggctacagctgcaaaaagcagtattgtaagccaggtatacatggatggtgacactactaaacctataacactaaaaataaacttcaatggcattgatgaaacaacagaaaatacccctgttagtaaatattccatgacattctcatggagctggcccaccgcaagctacataggccacacttttgcaacaaactcttttactttctcctacatcgcccaagaataaagaaagcacagagatgcttgttttgatttcaaaattgtgtgcttttatttattttcagcttacagtatttccagtagtcattcgaataaagcttaatcaaactgcatgagaacccttccacatagcttaaattagcaccagtgcaaatggagaaaagcctcgaggtcgttgcgcggccgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatcgcgatccggcccgaggctgtagccgacgatggtgcgccaggagagttgttgattcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgcgaaatattcccgtgcaccttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgcaagtccgcatcttcatgacgaccaaagccagtaaagtagaacggtttgtggttaatcaggaactgttcgcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtg

SEQ ID NO: 26 - DNA, 114 bp, synthetic construct
  source 1..114: mol_type = other DNA
acattttgacacccccataatatttttccagaattaacagtataaattgcatctcttgttcaagagttccctatcactctctttaatcactactcacagtaacctcaactcctg

SEQ ID NO: 27 - DNA, 55 bp, synthetic construct
  source 1..55: mol_type = other DNA
tcaagagttccctatcactctctttaatcactactcacagtaacctcaactcctg

SEQ ID NO: 28 - DNA, 380 bp, synthetic construct
  source 1..380: mol_type = other DNA
tgatatcttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 29 - DNA, 373 bp, synthetic construct
  source 1..373: mol_type = other DNA
ttttctgagttacttttgtatccccacccccttaaagaaaggaggaaaaactgtttcatacagaaggcgttaattgcatgaattagagctatcacctaagtgtgggctaatgtaacaaagagggatttcacctacatccattcagtcagtctttgggggtttaaagaaattccaaagagtcatcagaagaggaaaaatgaaggtaatgttttttcagactggtaaagtctttgaaaatatgtgtaatatgtaaaacattttgacacccccataatatttttccagaattaacagtataaattgcatctcttgttcaagagttccctatcactctctttaatcactactcacagtaacctcaactcctgccaca

SEQ ID NO: 30 - DNA, 161 bp, synthetic construct
  source 1..161: mol_type = other DNA
aattggtcccatcgaagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 31 - DNA, 201 bp, synthetic construct
  source 1..201: mol_type = other DNA
aattggtcccatcgaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 32 - DNA, 268 bp, synthetic construct
  source 1..268: mol_type = other DNA
aattggtcccatcgaagagggatttcacctacataagagggatttcacctacataagagggatttcacctacataattggtaagagggatttcacctacataagagggatttcacctacataagagggatttcacctacataattggtcccgggacattttgacacccccataatatttttccagaattaacagtataaattgcatctcttgttcaagagttccctatcactctctttaatcactactcacagtaacctcaactcctg

SEQ ID NO: 33 - DNA, 177 bp, synthetic construct
  source 1..177: mol_type = other DNA
aattggtcccatcgaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 34 - DNA, 256 bp, synthetic construct
  source 1..256: mol_type = other DNA
tgatatcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 35 - DNA, 249 bp, synthetic construct
  source 1..249: mol_type = other DNA
aattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 36 - DNA, 358 bp, synthetic construct
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctccttaatcactactcacagtaacctcaactcctg

SEQ ID NO: 37 - DNA, 374 bp, synthetic construct
  source 1..374: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctgaattccatg

SEQ ID NO: 38 - DNA, 358 bp, synthetic construct
  source 1..358: mol_type = other DNA
gaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 39 - DNA, 365 bp, synthetic construct
  source 1..365: mol_type = other DNA
tgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctg

SEQ ID NO: 40 - DNA, 244 bp, synthetic construct
  source 1..244: mol_type = other DNA
gagcgtgcgtgaggctccggtgcccgtcagtgggcagagcgcacatcgcccacagtccccgagaagttggggggagggggtcggcgattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 41 - DNA, 236 bp, synthetic construct
  source 1..236: mol_type = other DNA
gcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 42 - DNA, 1266 bp, synthetic construct
  source 1..1266: mol_type = other DNA
gagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgag

SEQ ID NO: 43 - DNA, 571 bp, synthetic construct
  source 1..571: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacg

SEQ ID NO: 44 - DNA, 81 bp, synthetic construct
  source 1..81: mol_type = other DNA
aggtctatataagcagagctcgtttagtgaaccctcattctggagacggatcccgagccgagtgttttgacctccatagaa

SEQ ID NO: 45 - protein, 151 aa, synthetic construct
MFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAA

SEQ ID NO: 46 - protein, 98 aa, synthetic construct
MPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKP

SEQ ID NO: 47 - protein, 58 aa, synthetic construct
MTNLDTASTTLLACFLLCFCVLLCVCLLIRPLLLSVSTYTRCFIVYIIFVYIPLFLIH

SEQ ID NO: 48 - protein, 120 aa, synthetic construct
MARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRH

SEQ ID NO: 49 - protein, 66 aa, synthetic construct
EIDGVNHQHLPARRAAPQRHTMLCMCCKCEARIERVVESSADDLRAFQQLFLNTLSFVCPWCASQQ

SEQ ID NO: 50 - protein, 53 aa, synthetic construct
MLSLIFLFCFCVCMYVCCHVPLLPSVVVITSPATAFTVYLLPMLLLHIHAILS

SEQ ID NO: 51 - protein, 547 aa, synthetic construct
MMFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPMTNLDTASTTLLACFLLCFCVLLCVCLLIRPLLLSVSTYTRCFIVYIIFVYIPLFLIHMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHEIDGVNHQHLPARRAAPQRHTMLCMCCKCEARIERVVESSADDLRAFQQLFLNTLSFVCPWCASQQMLSLIFLFCFCVCMYVCCHVPLLPSVVVITSPATAFTVYLLPMLLLHIHAILS

SEQ ID NO: 52 - protein, 563 aa, synthetic construct
MQTDRTGETALHLAARYSRSDHLDKKQRFHNIADARWTGRCMSCCDNMTTLEQQYNKPLCDLLPLHAAVSADISEYRHYCYRNRATDLVYDFAFRDLMHDTIHDIILECVPLILAARLAVKLPQLCTELSHADVFAFRDLCIVYDDLLCIVYRDGNPYAVCDALHWAAAVNNVLCVQSTHVDIGANKRTLEDLLMGTNNRTLGIVCPIPLFLAAREGSYELLMGTLGIVANRTLHEYMLDLDHMAHYNIVTFCCPRDYMLDLQPETTIVRLLDLEVSQTSKLTRQTDRTGETALHLAARYSRSDCDSTLRLCVADAKLTNTGLYNLDNMKCIDFYSRIPLHAAVSADFAFKDLFVVRNRATDLNLLIRCLRCMHDKLPDLCTELPLILAARLAVELTEVFEFASHADVSLQDIEITCVDDLKTVLELTEVLHWAAAVNNVDDLRAFQQLFLNTLSGANKFQQLFLNTLNNRQLFLNTLSFVPLFLAAREGSYELFLNTLSFVCPWCASANRTLQDIVLHLDHMSEEENDEIDGVNHQHLPARRPRDYIIFVYIPLIVRLLDLE

SEQ ID NO: 53 - protein, 605 aa, synthetic construct
MKLPQLCTEVMFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAQLYNKPLCDVMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPRTLEDLLMGVMTNLDTASTTLLACFLLCFCVLLCVCLLIRPLLLSVSTYTRCFIVYIIFVYIPLFLIHKLPDLCTEVMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHKLTNTGLYNVEIDGVNHQHLPARRAAPQRHTMLCMCCKCEARIERVVESSADDLRAFQQLFLNTLSFVCPWCASQQRAFQQLFLNVMLSLIFLFCFCVCMYVCCHVPLLPSVVVITSPATAFTVYLLPMLLLHIHAILS

SEQ ID NO: 54 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 55 - protein, 436 aa, synthetic construct
MKKELTEVFEFAKKYIIFVYIPLKKKLPQLCTEVKKLLMGTLGIVKKTLHEYMLDLKKSEEENDEIDGVNHQHLPARRKKNLLIRCLRCKKLFLNTLSFVCPWCASKKKCIDFYSRIKKKLPDLCTELKKQLFLNTLSFVKKFQQLFLNTLKKVYDFAFRDLKKKLTNTGLYNLKKLCIVYRDGNPYAVCDKKYMLDLQPETTKKFAFRDLCIVYKKTLGIVCPIKKRTLEDLLMGVKKTIHDIILECVKKHLDKKQRFHNIKKTTLEQQYNKPLCDLLKKCDSTLRLCVKKRWTGRCMSCCKKSLQDIEITCVKKLCVQSTHVDIKKAHYNIVTFCCKKDDLRAFQQLFLNTLSKKISEYRHYCYKKKLPQLCTELKKKTVLELTEVKKRTLEDLLMGTKKFAFKDLFVVKKQLYNKPLCDVKKTLQDIVLHLKK

SEQ ID NO: 56 - DNA, 6380 bp, synthetic construct
  source 1..6380: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgatgttccaggatcctcaagaacggcctcggaaactacctcagctgtgtacagccttacagacaacaatacatgatataatcctggagtgcgtctactgtaaacagcaactgttgcgtcgcgaggtctatgatttcgccttcagggatggttgtattgtatacagagacggcaatccgtatgcagtgtgtgataagtgtctcaagttctatagcaaaatcagtgaataccggcactattgctatagcctgtatggaactacactagagcagcagtataacaagccactctgtgacctgctaattagatgtatcaactgccagaagcctctgtgccccgaagagaagcagcggcaccttgacaagaagcagcggtttcataatatcagagggaggtggaccgggcggtgtatgtcctgctgccgcagcagcaggacccgtcgggccgcggcggcaatgccaggggacacgcccacactccacgagtacatgctcgaccttcagccggaaaccaccgatctctacggctatgaacagttgaacgacagctccgaagaggaagatgagatcgacggcccagcaggacaggcggcccctgacagagcacactacaatattgttaccttctgctgcaagtgcgacagcacgctgcgccgatgcgtacaatctacgcatgtggacattaggacccttgaggatctgcttatggggacactcggaattgtttgtcccatatgcagtcagaaacctatgaccaacctagacaccgcgagcacgactcttctggcttgcttcctgctatgtttttgcgtgctgctttgtgtttgcctactcattcggcccctgctgctgtctgtgagcacgtacacgaggtgcttcattgtctatattatcttcgtttacataccgctgttcctgattcacatggcaagatttgaagatcccacgcggcggccatacaaactccctgatctctgcacggcactgaacacttcgttgcaggacatagaaatcacttgcgtgtactgcaagactgtgctagagctgaccgaggtgtttgagtttgccttcaaggacggttttgtggtgtatcgggatagcatcccccatgccgcctgccataaatgtattgatttctattcgagaattagagaactcaggcattattccgactccgtatacggcgacaccctagaaaaactgaccaacaccggcttgtataacctcctcatccgctgcctccgatgccagaagccacttaacccagccgagaaattacgccacgagatcgacggggtcaaccatcagcacctgccggccagacgagccgcccctcagcgccataccatgctgtgcatgtgttgcaagtgcgaagccaggatcgaaagggtggtggaatctagcgctgacgacttaagggcattccaacaactctttcttaacaccctgagcttcgtgtgtccttggtgtgcttcccaacagatgcttagtctgatttttctgttttgcttttgcgtgtgtatgtatgtctgctgccacgtgcctctcctaccatctgtagtggtgattacatcccccgcgactgccttcaccgtgtacctgttgccaatgctcctccttcacatacacgccatcctgagctaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 57 - DNA, 6428 bp, synthetic construct
  source 1..6428: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgcagactgatagaaccggcgaaaccgctctacaccttgccgcccgttatagtcgaagcgaccacctcgataagaaacagcgattccacaacatcgcagacgcaagatggaccgggcggtgtatgtcctgctgtgacaatatgacaacactggaacagcagtataacaagcccctgtgtgacctgttgcccctgcacgccgccgtgtcagccgacatatccgagtatcggcattactgctaccggaaccgtgccaccgacctcgtgtacgactttgcatttagagacctgatgcacgataccatccatgacatcatcctggaatgtgtcccactgatcctggctgctcggcttgcagtgaagctaccccagctatgcactgagctatcacacgcagacgtgttcgccttcagggatctgtgcattgtttacgatgacctgttgtgtattgtgtacagagatggcaacccttatgcagtgtgtgacgccctgcattgggctgccgccgtgaataatgtcctttgtgtccagagcacccacgtcgatataggcgctaataagcggacactggaggacctgttgatggggactaataatagaaccctgggaatcgtgtgccccattccattattcctggccgcccgcgagggatcttatgaactcctgatgggcacactaggcatcgtggccaataggacattgcacgagtatatgctagacttagaccacatggctcactataacattgtcacattttgttgcccccgcgattacatgcttgatttacagcccgaaacaaccatcgtgagactgctcgatctggaggtatctcagacaagtaagctcacaagacaaacagacagaaccggcgagactgctttgcatctggctgcacgatattccaggtccgactgtgatagcaccctgcggttatgtgtggcagacgcaaaacttaccaataccggcctatataacctcgacaacatgaagtgcatcgacttctacagcagaatccccctccatgccgccgtgagcgccgattttgccttcaaggacctgttcgtggtgagaaaccgagccaccgacctgaacctgttgattcggtgtttacgctgtatgcacgacaagctgcccgatttgtgtactgagctgcctctcattctggccgcacggctcgcagttgaactaaccgaagtgttcgagttcgcctcccacgccgacgtgtccctacaggacatcgagatcacatgcgtagacgatctgaaaaccgttctcgaactcacagaagtgcttcattgggccgccgcagtcaacaacgtggacgatctccgggcattccagcaactgttcctgaatacactgtctggcgctaacaagtttcagcagttatttttgaacacacttaataaccgccagctattcttgaataccctgtcattcgtgccgctgttcttggctgcgcgcgaggggtcctacgagctgtttctgaatacgctctccttcgtgtgtccctggtgcgcctccgctaataggaccctgcaagacatcgtacttcacctggatcacatgtcagaggaggagaacgatgagatcgacggtgtcaaccaccagcacctccccgcccggaggccccgagattacatcatttttgtatatattcccttaatcgtaagattactcgacctggagtaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 58 - DNA, 6554 bp, synthetic construct
  source 1..6554: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgaagctaccgcagctctgtacagaagtgatgttccaggatcctcaagaacggcctcggaaactacctcagctgtgtacagccttacagacaacaatacatgatataatcctggagtgcgtctactgtaaacagcaactgttgcgtcgcgaggtctatgatttcgccttcagggatggttgtattgtatacagagacggcaatccgtatgcagtgtgtgataagtgtctcaagttctatagcaaaatcagtgaataccggcactattgctatagcctgtatggaactacactagagcagcagtataacaagccactctgtgacctgctaattagatgtatcaactgccagaagcctctgtgccccgaagagaagcagcggcaccttgacaagaagcagcggtttcataatatcagagggaggtggaccgggcggtgtatgtcctgctgccgcagcagcaggacccgtcgggccgcggcggcacagctgtacaacaaaccactgtgtgacgtgatgccaggggacacgcccacactccacgagtacatgctcgaccttcagccggaaaccaccgatctctacggctatgaacagttgaacgacagctccgaagaggaagatgagatcgacggcccagcaggacaggcggcccctgacagagcacactacaatattgttaccttctgctgcaagtgcgacagcacgctgcgccgatgcgtacaatctacgcatgtggacattaggacccttgaggatctgcttatggggacactcggaattgtttgtcccatatgcagtcagaaacctcggacactggaagatttgctcatgggcgttatgaccaacctagacaccgcgagcacgactcttctggcttgcttcctgctatgtttttgcgtgctgctttgtgtttgcctactcattcggcccctgctgctgtctgtgagcacgtacacgaggtgcttcattgtctatattatcttcgtttacataccgctgttcctgattcacaagctgcccgatttgtgcacagaggtgatggcaagatttgaagatcccacgcggcggccatacaaactccctgatctctgcacggcactgaacacttcgttgcaggacatagaaatcacttgcgtgtactgcaagactgtgctagagctgaccgaggtgtttgagtttgccttcaaggacggttttgtggtgtatcgggatagcatcccccatgccgcctgccataaatgtattgatttctattcgagaattagagaactcaggcattattccgactccgtatacggcgacaccctagaaaaactgaccaacaccggcttgtataacctcctcatccgctgcctccgatgccagaagccacttaacccagccgagaaattacgccacaagctgacgaacaccggtttgtacaatgtggagatcgacggggtcaaccatcagcacctgccggccagacgagccgcccctcagcgccataccatgctgtgcatgtgttgcaagtgcgaagccaggatcgaaagggtggtggaatctagcgctgacgacttaagggcattccaacaactctttcttaacaccctgagcttcgtgtgtccttggtgtgcttcccaacagcgcgccttccaacaattgttcctcaatgtaatgcttagtctgatttttctgttttgcttttgcgtgtgtatgtatgtctgctgccacgtgcctctcctaccatctgtagtggtgattacatcccccgcgactgccttcaccgtgtacctgttgccaatgctcctccttcacatacacgccatcctgagctaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 59 - DNA, 6455 bp, synthetic construct
  source 1..6455: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgcaaaccgacagagagcttaccgaggttttcgagtttgcagcccggtactcccggagcgactacattatttttgtctatatccctctcgcagacgcaaagctgcctcaactgtgtacagaggttgataatatgctactgatgggcactctcggcatcgtgccgctccacgccgccgtgagcgccgacaccctgcatgaatacatgctggacctgagaaatcgggcaaccgacttatccgaggaggagaacgacgaaattgatggagttaatcaccagcacctaccagcaagacgaatgcacgacaacctactgattagatgcttgaggtgtccccttattttggcagccagactcgccgttctgttcctgaacaccctgagctttgtatgcccctggtgtgccagctcgcacgcagacgtgaaatgtattgacttctattccagaatcgacgacctaaaactgcctgatctctgtacagagcttgcgttgcactgggcagccgccgtgaacaacgtgcagttgtttctgaacaccttatccttcgtgggcgctaacaaattccagcaacttttcttgaacaccctaaacaacagagtttatgactttgctttcagagacctccccctgttccttgctgccagagagggatcatacgagaaactgacaaatacgggcctctacaatttggccaacagactgtgcatcgtgtacagagatggcaatccctacgccgtttgcgacgaccacatgtacatgctcgacctccagccggagacaactcccagagacttcgcttttcgggacctgtgtatcgtgtatattgtaagactcctggacctggaagtgagccaaacaagtaagctaaccagacaaacagacagaacacttgggatcgtgtgtcctattgcagcccggtatagtaggtcagaccggactctggaggacctgttaatgggggtggcggacgcaacaatccacgacattatcttggagtgtgttgacaatatgcacctggacaagaaacagagattccacaacatacccctgcacgcagccgtcagcgcggatactactctggagcagcaatataacaagcccctgtgcgacctcctgcggaatcgggcaactgatctctgtgacagtacccttcgtttgtgcgttatgcacgacaggtggactgggagatgtatgtcttgctgtcctctcattctggccgcaagactggccgttagtctgcaagacatcgagatcacctgtgtcagtcacgccgatgtgctatgcgtgcagagcacccacgttgatattgatgacctagcacactacaacatcgttactttttgctgtctacactgggccgcagccgtgaataacgtggacgacctacgcgctttccaacaactgttccttaacaccctgagcggcgcaaacaagatcagcgagtaccgccactactgttataataaccggaagctgccacagctatgcacagagttgccactgttcctggcagcaagagagggatcttatgaaaagaccgtgctggagctgacagaggtggctaatcgccgcacactggaggacctgttgatgggaaccgatcacatgttcgcctttaaggaccttttcgttgttcccagagaccagctttacaacaaacctctatgcgacgtaatagtgaggaccttgcaggatattgtgctccatctgctggaataagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 60 - DNA, 6047 bp, synthetic construct
  source 1..6047: mol_type = other DNA
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagccggatccggtaccgccaccatgaagaaagaattgaccgaagtatttgaatttgccaagaagtacatcatctttgtttacatacccctgaagaagaaattaccccagctgtgtacagaggtgaagaagttattgatgggaacactcggaattgtgaaaaagaccctgcacgaatatatgctagatttgaagaagtctgaggaggagaacgacgagatcgacggggtgaaccaccagcacctccccgccaggagaaagaagaatcttctcatacggtgtctgcggtgcaaaaagctgttcctgaacaccctgtcgttcgtatgtccctggtgcgcttccaagaagaagtgcatcgacttttactctcggatcaagaagaagctgcccgatctgtgtactgagctgaagaagcaactgttcctgaacacgctgtcgtttgtgaagaagtttcagcagctgttcctgaacactctaaagaaggtgtacgattttgctttcagagacctgaaaaagaagttgacaaatacggggctgtataatctgaagaaactctgtatcgtttacagggacggtaacccttacgctgtgtgtgacaaaaagtacatgctagacctccagcccgaaacgaccaagaagttcgcgttcagagacctgtgtatcgtctacaagaaaacactaggcatcgtgtgtcccattaagaaacggactcttgaggacctattgatgggtgtgaaaaagacaattcatgacattatcttggagtgtgtgaagaaacatctcgataagaaacaaagattccataacatcaaaaagacaaccctggaacagcagtataacaagcctctttgcgatctgctaaagaaatgtgactccaccttgaggctgtgcgtaaagaagcgatggaccggaagatgtatgtcttgttgtaaaaagagtctccaagatattgagattacttgcgtgaaaaagttgtgcgttcagagtacacatgttgatattaagaaagcccattataatatcgtgacattttgctgtaagaaggatgacctgagagcttttcagcagctgtttctgaatacactgagcaagaagatcagcgagtaccgacactattgttacaagaaaaagctgccccagctgtgtaccgaattgaagaagaaaaccgtcctagagcttaccgaagtgaagaagcgtaccctggaggacctgttaatgggcacgaagaagtttgcattcaaggacctgtttgtcgtgaaaaagcagctgtacaataagcccctgtgcgacgttaaaaagacactccaagacatcgtcttacacctgaaaaagtaagcggccgctctcgagtctagctagtctagactagctagaaagatccgggaacttgtttattgcagcttataatggttacaaataaagcaatagcatcacaaatttcacaaataaagcatttttttcactgcattctagttgtggtttgtccaaactcatcaatgtatcttatcatgtctggatcggtgatcaccgatcccgatctgtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 61 - DNA, 10410 bp, synthetic construct
  source 1..10410: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctctatcgataatgcaggtcggagtactgtcctccgagcggagtactgtcctccgagcggagtactgtcctccgagcggagtactgtcctccgagcggagtactgtcctccgagcggagtactgtcctccgagcggagactcttcgaaggaagaggggcggggtcgatcgaccccgcccctcttccttcgaaggaagaggggcggggtcgaagacctagagggtatataatgggtgccttagctggtgtgtgagctcatcttcctgtagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttgggcgagctcgaattcattgatcccccgggctgcaggaattcgatatcaagctcgggatccgaattccgccccccccccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaacgtacgtcgacatcgagaacttgtttattgcagcttataatggttacaaataaagcaatagcatcacaaatttcacaaataaagcatttttttcactgcattctagttgtggtttgtccaaactcatcaatgtatcttatcatgtctgggcgcgccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaggcggcctctagagcggccgccaccgcggggagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 62 - DNA, 10040 bp, synthetic construct
  source 1..10040: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggagatcacgcgtgccaccatgggtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtctcgagggcggcggagagggcagaggaagtcttctaacatgcggtgacgtggaggagaatcccggccctaggatgggtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 63 - DNA, 9949 bp, synthetic construct
  source 1..9949: mol_type = other DNA
ttcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctgggtcaagtcttccagtttaagcagcagagcggtcagtttctcatcccgagcagacgcgcgagaggccgcgccgctcgccaccaaagagctgtaaaggtccgtagccatgctgcgcgcggtcgcggcggcggcggaggcggcggcggaggtcgcggcgtccagcggagttcctcccacggtcgcgtaggccattgtagacgaatttgaaggcagaacggggcgtccatccacgttggaacccatcacattctgacgcactccagcccagtgaggcatgcgcactgtcagataggggctaaagatgcttccatcaaagctgttgccggtgtcgctcatggcggcggctgttgcaagacaaaacagagagacccttagacccccaatttatacacgccccacccttctagccacgcccaccttacccacctcaatcggtatcctcatcgctagacccaaactcggccctggtgcaggccagcaccagatggtcaggcctgcaggtacgtagccgcaataaaatatctttattttcattacatctgtgtgttggttttttgtgtgaatccatagtactaacatacgctctccatcaaaacaaaacgaaacaaaacaaactagcaaaataggctgtccccagtgcaagtccaggtgccagaacatttctctatccataatgcaggggtaccggaaggaagaggggcggggtcgatcgaccccgcccctcttccttcgaaggaagaggggcggggtccaattgcggagtactgtcctccgagcggagtactgtcctccgagcggagtactgtcctccgagcggagtactgtcctccgagcggagtactgtcctccgagcggagtactgtcctccgagcggagagtccccggggacctagagggtatataatgggtgccttagctggtgtgtgacctcatcttcctgtacgcccctgcaggcagccgctaaatccaaggtaaggtcagaagagctagcgccaccatgtgtcaccagcagttggtcatctcttggttcagcctggtttttctggcatctcccctcgtggccatctgggaactgaagaaagatgtttatgtcgtagaattggattggtatcccgacgcccctggagaaatggtggtcctgacatgtgacacccctgaagaagatggtatcacctggaccttggaccagagcagtgaggtcttaggctctggcaagaccctgaccatccaagtcaaagagtttggagatgctggccagtacacctgtcacaaaggaggcgaggttctaagccattcgctcctgctgcttcacaaaaaggaagatggaatttggtccactgacattctgaaggaccagaaagaacccaagaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactcagcgccgagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagcccctgaagaacagcagacaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgctccggtggcggtggcggcggatctagaaacctccccgtggccactccagacccaggaatgttcccatgccttcaccacagccagaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagctgctgatggaccccaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaatcagagcagtgactattgatagagtgatgagctatctgaatgcttcctaaatcgatttatttatcggcataaataatttttttgaagaagtaatactatttttctttttttttgtaaataaatgggttaagggatgtaacattgtttgttgtttggtgggggttggggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctgggtacgtgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgagctagcgccgccaccatgggccctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtggggaattcgagatgcctgtggacaggatcctggaggcagagcttgctgtggaacagaagagtgaccagggcgttgagggtcctgggggaaccgggggtagcggcagcagcccaaatgaccctgtgactaacatctgtcaggcagctgacaaacagctattcacgcttgttgagtgggcgaagaggatcccacacttttcctccttgcctctggatgatcaggtcatattgctgcgggcaggctggaatgaactcctcattgcctccttttcacaccgatccattgatgttcgagatggcatcctccttgccacaggtcttcacgtgcaccgcaactcagcccattcagcaggagtaggagccatctttgatcgggtgctgacagagctagtgtccaaaatgcgtgacatgaggatggacaagacagagcttggctgcctgagggcaatcattctgtttaatccagaggtgaggggtttgaaatccgcccaggaagttgaacttctacgtgaaaaagtatatgccgctttggaagaatatactagaacaacacatcccgatgaaccaggaagatttgcaaaacttttgcttcgtctgccttctttacgttccataggccttaagtgtttggagcatttgtttttctttcgccttattggagatgttccaattgatacgttcctgatggagatgcttgaatcaccttctgattcataatctagcctagcccccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatctctaggcgccaccatgaagctactgtcttctatcgaacaagcatgcgatatttgccgacttaaaaagctcaagtgctccaaagaaaaaccgaagtgcgccaagtgtctgaagaacaactgggagtgtcgctactctcccaaaaccaaaaggtctccgctgactagggcacatctgacagaagtggaatcaaggctagaaagactggaacagctatttctactgatttttcctcgagaagaccttgacatgattttgaaaatggattctttacaggatataaaagcattgttaacaggattatttgtacaagataatgtgaataaagatgccgtcacagatagattggcttcagtggagactgatatgcctctaacattgagacagcatagaataagtgcgacatcatcatcggaagagagtagtaacaaaggtcaaagacagttgactgtatcgccggaattcccggggatccggcctgagtgcgtagtacccgagactcagtgcgccatgaagcggaaagagaagaaagcacagaaggagaaggacaaactgcctgtcagcacgacgacggtggacgaccacatgccgcccattatgcagtgtgaacctccacctcctgaagcagcaaggattcacgaagtggtcccaaggtttctctccgacaagctgttggtgacaaaccggcagaaaaacatcccccagttgacagccaaccagcagttccttatcgccaggctcatctggtaccaggacgggtacgagcagccttctgatgaagatttgaagaggattacgcagacgtggcagcaagcggacgatgaaaacgaagagtcggacactcccttccgccagatcacagagatgactatcctcacggtccaacttatcgtggagttcgcgaagggattgccagggttcgccaagatctcgcagcctgatcaaattacgctgcttaaggcttgctcaagtgaggtaatgatgctccgagtcgcgcgacgatacgatgcggcctcagacagtattctgttcgcgaacaaccaagcgtacactcgcgacaactaccgcaaggctggcatggccgaggtcatcgaggatctactgcacttctgccggtgcatgtactctatggcgttggacaacatccattacgcgctgctcacggctgtcgtcatcttttctgaccggccagggttggagcagccgcaactggtggaagagatccagcggtactacctgaatacgctccgcatctatatcctgaaccagctgagcgggtcggcgcgttcgtccgtcatatacggcaagatcctctcaatcctctctgagctacgcacgctcggcatgcaaaactccaacatgtgcatctccctcaagctcaagaacagaaagctgccgcctttcctcgaggagatctgggatgtggcggacatgtcgcacacccaaccgccgcctatcctcgagtcccccacgaatctctaaatcgattacgctcctctactctttgagacatcactggcctataataaatgggttaatttatgtaacaaaattgccttggcttgttaactttattagacattctgatgtttgcattgtgtaaatactgttgtattggaaaagcgtgccaagatggattattgtaattcagtgtcttttttagtagcgtcacgtgccaaacactgttagtcacagagggcatgagacagcctgtgctggaacagctcagttcatagggctatggagatggggagaaaggggcgcttctgtcagagacaagctgtggtctgggaaggccttagcactaaaagcaccacaatgagaagcaaccgccagaagcagggcccgcaggcctttgttccagctgcaaagagaaaggaaaaagtggggaataagagttggggctgcggagggggtggggagcattgtgcaggttccgtacttgaacagaaagcagggaccaacacaaggaagggcgcgccaccggtcaaatgacggtgacaataaaacggagactttgacccggaacgcggaaattcacgtaaaaaacacctgggcgagtcctccacgtaatcggtcaaagtccctcggccctcggtaaatattacgcactatgactaacgccctattattcagttttcacttccccgtttcacttttcgcgcgaaaatggccaaatcttacatggtcccgcccaaaattactacgatatccggtgaaaagcgcgcgaaaattggcacttccggaggtaggcggcgctcatcaaaaacgtcacattttccgcgacggaagcttgcatgtgagctcctcccacttgcaaatgccacacttccgccacacctcccaaccctactcgcgcgtcctacgtcacccgccccgcctctccccgcccacctcattatcatattggccacaatccaaaataaggtatattattgatgatggtttaaacgcccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaataca

SEQ ID NO: 64 - DNA, 54 bp, synthetic construct
  source 1..54: mol_type = other DNA
ggcagcacctccggcagcggcaagcctggcagcggcgagggcagcaccaagggc

SEQ ID NO: 65 - DNA, 78 bp, synthetic construct
  source 1..78: mol_type = other DNA
tctggcggaggatctggaggaggcggatctggaggaggaggcagtggaggcggaggatctggcggaggatctctgcag

SEQ ID NO: 66 - skipped

SEQ ID NO: 67 - DNA, 12 bp, synthetic construct
  source 1..12: mol_type = other DNA
agtggcagcggc

SEQ ID NO: 68 - DNA, 45 bp, synthetic construct
  source 1..45: mol_type = other DNA
ggtggcggtggctcgggcggtggtgggtcgggtggcggcggatct

SEQ ID NO: 69 - DNA, 12 bp, synthetic construct
  source 1..12: mol_type = other DNA
cgtgcaaagcgt

SEQ ID NO: 70 - DNA, 84 bp, synthetic construct
  source 1..84: mol_type = other DNA
agagccaagagggcaccggtgaaacagactttgaattttgaccttctgaagttggcaggagacgttgagtccaaccctgggccc

SEQ ID NO: 71 - DNA, 54 bp, synthetic construct
  source 1..54: mol_type = other DNA
gagggcagaggaagtctgctaacatgcggtgacgtcgaggagaatcctggacct

SEQ ID NO: 72 - DNA, 75 bp, synthetic construct
  source 1..75: mol_type = other DNA
agagctaagaggggaagcggagagggcagaggaagtctgctaacatgcggtgacgtcgaggagaatcctggacct

SEQ ID NO: 73 - DNA, 78 bp, synthetic construct
  source 1..78: mol_type = other DNA
agggccaagaggagtggcagcggcgagggcagaggaagtcttctaacatgcggtgacgtggaggagaatcccggccct

SEQ ID NO: 74 - DNA, 57 bp, synthetic construct
  source 1..57: mol_type = other DNA
gcaacgaacttctctctcctaaaacaggctggtgatgtggaggagaatcctggtcca

SEQ ID NO: 75 - DNA, 66 bp, synthetic construct
  source 1..66: mol_type = other DNA
ggaagcggagctactaacttcagcctgctgaagcaggctggagacgtggaggagaaccctggacct

SEQ ID NO: 76 - DNA, 60 bp, synthetic construct
  source 1..60: mol_type = other DNA
cagtgtactaattatgctctcttgaaattggctggagatgttgagagcaaccctggacct

SEQ ID NO: 77 - DNA, 66 bp, synthetic construct
  source 1..66: mol_type = other DNA
gtcaaacagaccctaaactttgatctgctaaaactggccggggatgtggaaagtaatcccggcccc

SEQ ID NO: 78 - DNA, 93 bp, synthetic construct
  source 1..93: mol_type = other DNA
cgtgcaaagcgtgcaccggtgaaacagggaagcggagctactaacttcagcctgctgaagcaggctggagacgtggaggagaaccctggacct

SEQ ID NO: 79 - DNA, 24 bp, synthetic construct
  source 1..24: mol_type = other DNA
gcaccggtgaaacagggaagcgga

SEQ ID NO: 80 - DNA, 15 bp, synthetic construct
  source 1..15: mol_type = other DNA
gcaccggtgaaacag

SEQ ID NO: 81 - protein, 18 aa, synthetic construct
GSTSGSGKPGSGEGSTKG

SEQ ID NO: 82 - protein, 26 aa, synthetic construct
SGGGSGGGGSGGGGSGGGGSGGGSLQ

SEQ ID NO: 83 - skipped

SEQ ID NO: 84 - protein, 4 aa, synthetic construct
SGSG

SEQ ID NO: 85 - protein, 15 aa, synthetic construct
GGGGSGGGGSGGGGS

SEQ ID NO: 86 - protein, 4 aa, synthetic construct
RAKR

SEQ ID NO: 87 - protein, 28 aa, synthetic construct
RAKRAPVKQTLNFDLLKLAGDVESNPGP

SEQ ID NO: 88 - protein, 18 aa, synthetic construct
EGRGSLLTCGDVEENPGP

SEQ ID NO: 89 - protein, 25 aa, synthetic construct
RAKRGSGEGRGSLLTCGDVEENPGP

SEQ ID NO: 90 - protein, 26 aa, synthetic construct
RAKRSGSGEGRGSLLTCGDVEENPGP

SEQ ID NO: 91 - protein, 19 aa, synthetic construct
ATNFSLLKQAGDVEENPGP

SEQ ID NO: 92 - protein, 22 aa, synthetic construct
GSGATNFSLLKQAGDVEENPGP

SEQ ID NO: 93 - protein, 20 aa, synthetic construct
QCTNYALLKLAGDVESNPGP

SEQ ID NO: 94 - protein, 22 aa, synthetic construct
VKQTLNFDLLKLAGDVESNPGP

SEQ ID NO: 95 - protein, 31 aa, synthetic construct
RAKRAPVKQGSGATNFSLLKQAGDVEENPGP

SEQ ID NO: 96 - protein, 8 aa, synthetic construct
APVKQGSG

SEQ ID NO: 97 - protein, 5 aa, synthetic construct
APVKQ

SEQ ID NO: 98 - protein, 154 aa, synthetic construct
MAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSA

SEQ ID NO: 99 - protein, 454 aa, synthetic construct
GPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 100 - protein, 226 aa, synthetic construct
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYI

SEQ ID NO: 101 - protein, 180 aa, synthetic construct
MQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQ

SEQ ID NO: 102 - protein, 226 aa, synthetic construct; identical to SEQ ID NO: 100

SEQ ID NO: 103 - protein, 180 aa, synthetic construct; identical to SEQ ID NO: 101

SEQ ID NO: 104 - protein, 154 aa, synthetic construct; identical to SEQ ID NO: 98

SEQ ID NO: 105 - protein, 454 aa, synthetic construct; identical to SEQ ID NO: 99

SEQ ID NO: 106 - protein, 1014 aa, synthetic construct
MENITSGFLGPLLVLQAGFFLLTRILTIPQSLDSWWTSLSFLGGTTVCLGQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYIMQLFHLCLIISCSCPTVQASKLCLGWLWDMDIDPYKEFGASVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQMAARLCCQLDPARDVLCLRPVGAESRGRPFSGPLGALSSSSPPAVPTDHGAHLSLRGLPVCAFSSAGPCALRFTSARRMETTVNAHQFLPKVLHKRTLGLSAMSTTDLEAYFKDCLFKDWEELGEELRLKVFVLGGCRHKLVCAPAPCNFFTSAGPCAEHGEHHIRIPRTPARVTGGVFLVDKNPHNTAESRLVVDFSQFSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAICSVVRRAFPHCLAFSGAKSVQHLESLFTAVTNFLLSLGIHLNPNKTKRWGYSLNFMGYVIGSWGSLPQDHIRHKIKECFRKLPVHRPIDWKVCQRIVGLLGFAAPFTQCGYPALMPLYACIQSKQAFTFSPTYKAFLCKQYLNLYPVARQRPGLCQVFADATPTGWGLVMGHQRMRGTFSSRKYTSFPWLLGCAANWILRGTSFVYVPSALNPADDPSRGRLGPCRPLLHLPFRPTTGRTSLYADSPSVPSHLPDRVHFASPLHVAWRPP

SEQ ID NO: 107 - protein, 1021 aa, synthetic construct
SVELLSFLPSDFFPSVRDLLDTATALYRDALESPEHCTPHHTALRHVCLCWGDLMNLATWVGTNLEDQASRDLVVSYVNTNMGLKFRQLLWFHISCLTFGRDLVLEYLVSFGVWIRTPPAYRPSNAPILSTLPETTVVRQRGRTIVLHKRTLGLMGQNLSTSNPLGFFPDHQLDPAFRANTNNPDWDFNPNKDTWPDANKVGAGAFGLGFTPPHGGLLGWSPQAQGIMQTLPANPPPASTNRQSGRQPTPLLPKVLHKRTLMPLSYQHFRKLLLLDNEAGPLEEELPRLADEDLNRRVAEDLNLGNLNVSIPWTHKVGNFTGLYSSSVPVFNPHWKTPSFPNIHLHQDIIKKCEQFVGPLTVNEKRRLKLIMPARFYPNFTKYLPLDKGIKPYYPEHLVNHYFHTRHYLHTLWKAGILYKRVSTHSASFCGSPYSWEQELQHGAESFHQQSSGILSRPSVGSSLQSKHQQSRLGLQSQQGHLARRQQGRSWSIRTRVHPTARRPSGVEPSGSGHNANLASKSASCLYQSTVRTAAYPAVSTSENHSSSGHAVELHNLPPNSARSQSERPVSPCWWLQFRNSKPCSDYCLSHIVNLLEDWGPCHKRTLGLSAMSPPLRTTHPQAMQWNSTTFHQTLQDPRVRGLYLPAGGSSSGTVNPVPTTASPTLSTSSRIGDPALNQFLPKVLHKRSRGNYRVSWPKFAVPNLQSLTNLLSSNLCWLSLDVSAAFYHLPLHPAAMPHLLVGSSGLSRYVARLSSNSRIINHQHGTLQNLHDSCSRNLYVSLLLLYKTFGWKLHLYSHPIILGFRKIPMGVGLSPFLLAQFTSAITVNAHQFLPKQNSQSPTSNHSPTSCPPTCVGYRWMCLRRFIIFLFILLLCLIFLLVLLDYQGMLPVCPLIPGSSTTSTGPCRTCTTPAQGTSMYPSCCCTKPSDGNCTCIPIPSSWAFGKFLWEWASARFSWLSLLVPFVQWFVGLSPTVWLSVIWMMWYWGPSLYNTLSPFLPLLPIFFYLWVYILSAMSTTDL

SEQ ID NO: 108 - protein, 532 aa, synthetic construct
MQTDRTGETALHLAARYSRSDFLPSDFFPSVADAGVWIRTPPADNMEYLVSFGVWPLHAAVSADCWGELMTLRNRATDLGGPNLDNILMHDILRSFIPLLPLILAARLAVSTLPETTVVRRSHADVFLGGPPVCLDDLFLLTRILTIALHWAAAVNNVLTFGRETVLEYGANKWLSLLVPFVNNRFLKQQYMNLPLFLAAREGSYEDLLDTASALYANRFLSKQYMDLDHMTVSTKLCKIPRDLWFHISCLTFIVRLLDLEVSQTSKLTRQTDRTGETALHLAARYSRSDLTTVPAASLLAADAGLSRYVARLDNMKLHLYSHPIPLHAAVSADGLSPTVWLSVRNRATDLFLLSLGIHLMHDSLYADSPSVPLILAARLAVHKRTLGLSAMSHADVTLCIPHVAVDDLLLLKATLCIALHWAAAVNNVQFLPKVLHKRGANKALMPLYACINNRTVNAHQFLPKPLFLAAREGSYELPKVLHKRTLANRVLHKRTLGLDHMLSAMSTTDLPRDLLVPFVQWFVIVRLLDLE

SEQ ID NO: 109 - protein, 368 aa, synthetic construct
KKFLPSDFFPSVKKCWGELMTLKKGVWIRTPPAKKSTLPETTVVRRKKLTFGRETVLEYKKDLLDTASALYKKLWFHISCLTFKKEYLVSFGVWKKGGPNLDNILKKLTTVPAASLLAKKILRSFIPLLKKFLGGPPVCLKKFLLTRILTIKKWLSLLVPFVKKGLSPTVWLSVKKLLVPFVQWFVKKFLKQQYMNLKKFLSKQYMDLKKTVSTKLCKIKKGLSRYVARLKKKLHLYSHPIKKFLLSLGIHLKKSLYADSPSVKKALMPLYACIKKLLLKATLCIKKTLCIPHVAVKKVLHKRTLGLKKLPKVLHKRTLKKHKRTLGLSAMKKQFLPKVLHKRKKTVNAHQFLPKKKLSAMSTTDLKK

SEQ ID NO: 110 - DNA, 20 bp, synthetic construct
  source 1..20: mol_type = other DNA
tgccaagagtgacgtgtcca

SEQ ID NO: 111 - DNA, 21 bp, synthetic construct
  source 1..21: mol_type = other DNA
cccaggtccaactgcagccgg

SEQ ID NO: 112 - DNA, 12 bp, synthetic construct
  source 1..12: mol_type = other DNA
agagctaagagg

SEQ ID NO: 113 - protein, 11 aa, synthetic construct
HLDKKQRFHNI

SEQ ID NO: 114 - protein, 10 aa, synthetic construct
RWTGRCMSCC

SEQ ID NO: 115 - protein, 15 aa, synthetic construct
TTLEQQYNKPLCDLL

SEQ ID NO: 116 - protein, 9 aa, synthetic construct
ISEYRHYCY

SEQ ID NO: 117 - protein, 9 aa, synthetic construct
VYDFAFRDL

SEQ ID NO: 118 - protein, 10 aa, synthetic construct
TIHDIILECV

SEQ ID NO: 119 - protein, 9 aa, synthetic construct
KLPQLCTEL

SEQ ID NO: 120 - protein, 10 aa, synthetic construct
FAFRDLCIVY

SEQ ID NO: 121 - protein, 15 aa, synthetic construct
LCIVYRDGNPYAVCD

SEQ ID NO: 122 - protein, 10 aa, synthetic construct
LCVQSTHVDI

SEQ ID NO: 123 - protein, 10 aa, synthetic construct
RTLEDLLMGT

SEQ ID NO: 124 - protein, 8 aa, synthetic construct
TLGIVCPI

SEQ ID NO: 125 - protein, 9 aa, synthetic construct
LLMGTLGIV

SEQ ID NO: 126 - protein, 9 aa, synthetic construct
TLHEYMLDL

SEQ ID NO: 127 - protein, 10 aa, synthetic construct
AHYNIVTFCC

SEQ ID NO: 128 - protein, 10 aa, synthetic construct
YMLDLQPETT

SEQ ID NO: 129 - protein, 9 aa, synthetic construct
CDSTLRLCV

SEQ ID NO: 130 - protein, 9 aa, synthetic construct
YIIFVYIPL

SEQ ID NO: 131 - protein, 10 aa, synthetic construct
KLTNTGLYNL

SEQ ID NO: 132 - protein, 9 aa, synthetic construct
KCIDFYSRI

SEQ ID NO: 133 - protein, 9 aa, synthetic construct
FAFKDLFVV

SEQ ID NO: 134 - protein, 9 aa, synthetic construct
NLLIRCLRC

SEQ ID NO: 135 - protein, 9 aa, synthetic construct
KLPDLCTEL

SEQ ID NO: 136 - protein, 9 aa, synthetic construct
ELTEVFEFA

SEQ ID NO: 137 - protein, 10 aa, synthetic construct
SLQDIEITCV

SEQ ID NO: 138 - protein, 9 aa, synthetic construct
KTVLELTEV

SEQ ID NO: 139 - protein, 15 aa, synthetic construct
DDLRAFQQLFLNTLS

SEQ ID NO: 140 - protein, 9 aa, synthetic construct
FQQLFLNTL

SEQ ID NO: 141 - protein, 10 aa, synthetic construct
QLFLNTLSFV

SEQ ID NO: 142 - protein, 15 aa, synthetic construct
LFLNTLSFVCPWCAS

SEQ ID NO: 143 - protein, 9 aa, synthetic construct
TLQDIVLHL

SEQ ID NO: 144 - protein, 20 aa, synthetic construct
SEEENDEIDGVNHQHLPARR

SEQ ID NO: 145 - protein, 9 aa, synthetic construct
KLPQLCTEV

SEQ ID NO: 146 - protein, 10 aa, synthetic construct
QLYNKPLCDV

SEQ ID NO: 147 - protein, 10 aa, synthetic construct
RTLEDLLMGV

SEQ ID NO: 148 - DNA, 365 bp, synthetic construct
  source 1..365: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 149 - DNA, 205 bp, synthetic construct
  source 1..205: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 150 - DNA, 42 bp, synthetic construct
  source 1..42: mol_type = other DNA
tccctatcagtgatagagatctccctatcagtgatagagatc

SEQ ID NO: 151 - DNA, 295 bp, synthetic construct
  source 1..295: mol_type = other DNA
gtcgacgagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcacttgaggtgtggcaggcttgagatctggccatacacttgagtgacaatgacatccactttgcctttctctccacaggtgtccactcccaggtccaactgcagcc

SEQ ID NO: 152 - DNA, 11 bp, synthetic construct
  source 1..11: mol_type = other DNA
ggatccggtac

SEQ ID NO: 153-154 - skipped (2 entries)

SEQ ID NO: 155 - DNA, 49 bp, synthetic construct
  source 1..49: mol_type = other DNA
gcggccgctctcgagtctagctagtctagactagctagaaagatccggg

SEQ ID NO: 156 - DNA, 132 bp, synthetic construct
  source 1..132: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 157 - DNA, 278 bp, synthetic construct
  source 1..278: mol_type = other DNA
caattgacggccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagggtaccaacgcgtcgtaactataacggtcctaaggtagcg

SEQ ID NO: 158-170 - skipped (13 entries)

SEQ ID NO: 171 - protein, 5 aa, Alphapapillomavirus sp.
MQTDR

SEQ ID NO: 172 - protein, 9 aa, Alphapapillomavirus sp.
ELTEVFEFA

SEQ ID NO: 173 - protein, 7 aa, Alphapapillomavirus sp.
ARYSRSD

SEQ ID NO: 174 - protein, 9 aa, Alphapapillomavirus sp.
YIIFVYIPL

SEQ ID NO: 175 - skipped

SEQ ID NO: 176 - protein, 9 aa, Alphapapillomavirus sp.
KLPQLCTEV

SEQ ID NO: 177 - skipped

SEQ ID NO: 178 - protein, 9 aa, Alphapapillomavirus sp.
LLMGTLGIV

SEQ ID NO: 179 - protein, 9 aa, Alphapapillomavirus sp.
PLHAAVSAD

SEQ ID NO: 180 - protein, 9 aa, Alphapapillomavirus sp.
TLHEYMLDL

SEQ ID NO: 181 - protein, 7 aa, Alphapapillomavirus sp.
RNRATDL

SEQ ID NO: 182 - protein, 20 aa, Alphapapillomavirus sp.
SEEENDEIDGVNHQHLPARR

SEQ ID NO: 183 - skipped

SEQ ID NO: 184 - protein, 9 aa, Alphapapillomavirus sp.
NLLIRCLRC

SEQ ID NO: 185 - protein, 10 aa, Alphapapillomavirus sp.
PLILAARLAV

SEQ ID NO: 186 - protein, 15 aa, Alphapapillomavirus sp.
LFLNTLSFVCPWCAS

SEQ ID NO: 187 - protein, 5 aa, Alphapapillomavirus sp.
SHADV

SEQ ID NO: 188 - protein, 9 aa, Alphapapillomavirus sp.
KCIDFYSRI

SEQ ID NO: 189 - skipped

SEQ ID NO: 190 - protein, 9 aa, Alphapapillomavirus sp.
KLPDLCTEL

SEQ ID NO: 191 - protein, 11 aa, Alphapapillomavirus sp.
ALHWAAAVNNV

SEQ ID NO: 192 - protein, 10 aa, Alphapapillomavirus sp.
QLFLNTLSFV

SEQ ID NO: 193 - protein, 4 aa, Alphapapillomavirus sp.
GANK

SEQ ID NO: 194 - protein, 9 aa, Alphapapillomavirus sp.
FQQLFLNTL

SEQ ID NO: 195 - skipped

SEQ ID NO: 196 - protein, 9 aa, Alphapapillomavirus sp.
VYDFAFRDL

SEQ ID NO: 197 - protein, 12 aa, Alphapapillomavirus sp.
PLFLAAREGSYE

SEQ ID NO: 198 - protein, 10 aa, Alphapapillomavirus sp.
KLTNTGLYNL

SEQ ID NO: 199 - skipped

SEQ ID NO: 200 - protein, 15 aa, Alphapapillomavirus sp.
LCIVYRDGNPYAVCD

SEQ ID NO: 201 - skipped

SEQ ID NO: 202 - protein, 10 aa, Alphapapillomavirus sp.
YMLDLQPETT

SEQ ID NO: 203 - skipped

SEQ ID NO: 204 - protein, 10 aa, Alphapapillomavirus sp.
FAFRDLCIVY

SEQ ID NO: 205 - protein, 21 aa, Alphapapillomavirus sp.
IVRLLDLEVSQTSKLTRQTDR

SEQ ID NO: 206 - protein, 8 aa, Alphapapillomavirus sp.
TLGIVCPI

SEQ ID NO: 207 - protein, 8 aa, Alphapapillomavirus sp.
AARYSRSD

SEQ ID NO: 208 - protein, 10 aa, Alphapapillomavirus sp.
RTLEDLLMGV

SEQ ID NO: 209 - skipped

SEQ ID NO: 210 - protein, 10 aa, Alphapapillomavirus sp.
TIHDIILECV

SEQ ID NO: 211 - skipped

SEQ ID NO: 212 - protein, 11 aa, Alphapapillomavirus sp.
HLDKKQRFHNI

SEQ ID NO: 213 - protein, 9 aa, Alphapapillomavirus sp.
PLHAAVSAD

SEQ ID NO: 214 - protein, 15 aa, Alphapapillomavirus sp.
TTLEQQYNKPLCDLL

SEQ ID NO: 215 - protein, 7 aa, Alphapapillomavirus sp.
RNRATDL

SEQ ID NO: 216 - protein, 9 aa, Alphapapillomavirus sp.
CDSTLRLCV

SEQ ID NO: 217 - skipped

SEQ ID NO: 218 - protein, 10 aa, Alphapapillomavirus sp.
RWTGRCMSCC

SEQ ID NO: 219 - protein, 10 aa, Alphapapillomavirus sp.
PLILAARLAV

SEQ ID NO: 220 - protein, 10 aa, Alphapapillomavirus sp.
SLQDIEITCV

SEQ ID NO: 221 - protein, 5 aa, Alphapapillomavirus sp.
SHADV

SEQ ID NO: 222 - protein, 10 aa, Alphapapillomavirus sp.
LCVQSTHVDI

SEQ ID NO: 223 - skipped

SEQ ID NO: 224 - protein, 10 aa, Alphapapillomavirus sp.
AHYNIVTFCC

SEQ ID NO: 225 - protein, 10 aa, Alphapapillomavirus sp.
LHWAAAVNNV

SEQ ID NO: 226 - protein, 15 aa, Alphapapillomavirus sp.
DDLRAFQQLFLNTLS

SEQ ID NO: 227 - protein, 4 aa, Alphapapillomavirus sp.
GANK

SEQ ID NO: 228 - protein, 9 aa, Alphapapillomavirus sp.
ISEYRHYCY

SEQ ID NO: 229 - skipped

SEQ ID NO: 230 - protein, 9 aa, Alphapapillomavirus sp.
KLPQLCTEL

SEQ ID NO: 231 - protein, 12 aa, Alphapapillomavirus sp.
PLFLAAREGSYE

SEQ ID NO: 232 - protein, 9 aa, Alphapapillomavirus sp.
KTVLELTEV

SEQ ID NO: 233 - skipped

SEQ ID NO: 234 - protein, 10 aa, Alphapapillomavirus sp.
RTLEDLLMGT

SEQ ID NO: 235 - skipped

SEQ ID NO: 236 - protein, 9 aa, Alphapapillomavirus sp.
FAFKDLFVV

SEQ ID NO: 237 - skipped

SEQ ID NO: 238 - protein, 10 aa, Alphapapillomavirus sp.
QLYNKPLCDV

SEQ ID NO: 239 - skipped

SEQ ID NO: 240 - protein, 9 aa, Alphapapillomavirus sp.
TLQDIVLHL

SEQ ID NO: 241 - skipped

SEQ ID NO: 242 - DNA, 1719 bp, synthetic construct
  source 1..1719: mol_type = other DNA
atgcaaaccgacagagagcttaccgaggttttcgagtttgcagcccggtactcccggagcgactacattatttttgtctatatccctctcgcagacgcaaagctgcctcaactgtgtacagaggttgataatatgctactgatgggcactctcggcatcgtgccgctccacgccgccgtgagcgccgacaccctgcatgaatacatgctggacctgagaaatcgggcaaccgacttatccgaggaggagaacgacgaaattgatggagttaatcaccagcacctaccagcaagacgaatgcacgacaacctactgattagatgcttgaggtgtccccttattttggcagccagactcgccgttctgttcctgaacaccctgagctttgtatgcccctggtgtgccagctcgcacgcagacgtgaaatgtattgacttctattccagaatcgacgacctaaaactgcctgatctctgtacagagcttgcgttgcactgggcagccgccgtgaacaacgtgcagttgtttctgaacaccttatccttcgtgggcgctaacaaattccagcaacttttcttgaacaccctaaacaacagagtttatgactttgctttcagagacctccccctgttccttgctgccagagagggatcatacgagaaactgacaaatacgggcctctacaatttggccaacagactgtgcatcgtgtacagagatggcaatccctacgccgtttgcgacgaccacatgtacatgctcgacctccagccggagacaactcccagagacttcgcttttcgggacctgtgtatcgtgtatattgtaagactcctggacctggaagtgagccaaacaagtaagctaaccagacaaacagacagaacacttgggatcgtgtgtcctattgcagcccggtatagtaggtcagaccggactctggaggacctgttaatgggggtggcggacgcaacaatccacgacattatcttggagtgtgttgacaatatgcacctggacaagaaacagagattccacaacatacccctgcacgcagccgtcagcgcggatactactctggagcagcaatataacaagcccctgtgcgacctcctgcggaatcgggcaactgatctctgtgacagtacccttcgtttgtgcgttatgcacgacaggtggactgggagatgtatgtcttgctgtcctctcattctggccgcaagactggccgttagtctgcaagacatcgagatcacctgtgtcagtcacgccgatgtgctatgcgtgcagagcacccacgttgatattgatgacctagcacactacaacatcgttactttttgctgtctacactgggccgcagccgtgaataacgtggacgacctacgcgctttccaacaactgttccttaacaccctgagcggcgcaaacaagatcagcgagtaccgccactactgttataataaccggaagctgccacagctatgcacagagttgccactgttcctggcagcaagagagggatcttatgaaaagaccgtgctggagctgacagaggtggctaatcgccgcacactggaggacctgttgatgggaaccgatcacatgttcgcctttaaggaccttttcgttgttcccagagaccagctttacaacaaacctctatgcgacgtaatagtgaggaccttgcaggatattgtgctccatctgctggaataa

SEQ ID NO: 243 - protein, 572 aa, unidentified; identical to SEQ ID NO: 54

SEQ ID NO: 244 - DNA, 34706 bp, unidentified
  source 1..34706: mol_type = unassigned DNA
catcatcaataatataccttattttggattgtggccaatatgataatgaggtgggcggggagaggcggggcgggtgacgtaggacgcgcgagtagggttgggaggtgtggcggaagtgtggcatttgcaagtgggaggagctcacatgcaagcttccgtcgcggaaaatgtgacgtttttgatgagcgccgcctacctccggaagtgccaattttcgcgcgcttttcaccggatatcgtagtaattttgggcgggaccatgtaagatttggccattttcgcgcgaaaagtgaaacggggaagtgaaaactgaataatagggcgttagtcatagtgcgtaatatttaccgagggccgagggactttgaccgattacgtggaggactcgcccaggtgttttttacgtgaatttccgcgttccgggtcaaagtctccgttttattgtcaccgtcatttgacagatcgggatcggtgatcaccgatccagacatgataagatacattgatgagtttggacaaaccacaactagaatgcagtgaaaaaaatgctttatttgtgaaatttgtgatgctattgctttatttgtaaccattataagctgcaataaacaagttcccggatctttctagctagtctagactagctagactcgagagcggccgcttattccagcagatggagcacaatatcctgcaaggtcctcactattacgtcgcatagaggtttgttgtaaagctggtctctgggaacaacgaaaaggtccttaaaggcgaacatgtgatcggttcccatcaacaggtcctccagtgtgcggcgattagccacctctgtcagctccagcacggtcttttcataagatccctctcttgctgccaggaacagtggcaactctgtgcatagctgtggcagcttccggttattataacagtagtggcggtactcgctgatcttgtttgcgccgctcagggtgttaaggaacagttgttggaaagcgcgtaggtcgtccacgttattcacggctgcggcccagtgtagacagcaaaaagtaacgatgttgtagtgtgctaggtcatcaatatcaacgtgggtgctctgcacgcatagcacatcggcgtgactgacacaggtgatctcgatgtcttgcagactaacggccagtcttgcggccagaatgagaggacagcaagacatacatctcccagtccacctgtcgtgcataacgcacaaacgaagggtactgtcacagagatcagttgcccgattccgcaggaggtcgcacaggggcttgttatattgctgctccagagtagtatccgcgctgacggctgcgtgcaggggtatgttgtggaatctctgtttcttgtccaggtgcatattgtcaacacactccaagataatgtcgtggattgttgcgtccgccacccccattaacaggtcctccagagtccggtctgacctactataccgggctgcaataggacacacgatcccaagtgttctgtctgtttgtctggttagcttacttgtttggctcacttccaggtccaggagtcttacaatatacacgatacacaggtcccgaaaagcgaagtctctgggagttgtctccggctggaggtcgagcatgtacatgtggtcgtcgcaaacggcgtagggattgccatctctgtacacgatgcacagtctgttggccaaattgtagaggcccgtatttgtcagtttctcgtatgatccctctctggcagcaaggaacagggggaggtctctgaaagcaaagtcataaactctgttgtttagggtgttcaagaaaagttgctggaatttgttagcgcccacgaaggataaggtgttcagaaacaactgcacgttgttcacggcggctgcccagtgcaacgcaagctctgtacagagatcaggcagttttaggtcgtcgattctggaatagaagtcaatacatttcacgtctgcgtgcgagctggcacaccaggggcatacaaagctcagggtgttcaggaacagaacggcgagtctggctgccaaaataaggggacacctcaagcatctaatcagtaggttgtcgtgcattcgtcttgctggtaggtgctggtgattaactccatcaatttcgtcgttctcctcctcggataagtcggttgcccgatttctcaggtccagcatgtattcatgcagggtgtcggcgctcacggcggcgtggagcggcacgatgccgagagtgcccatcagtagcatattatcaacctctgtacacagttgaggcagctttgcgtctgcgagagggatatagacaaaaataatgtagtcgctccgggagtaccgggctgcaaactcgaaaacctcggtaagctctctgtcggtttgcatggtggcggtaccggatccggctgcagttggacctgggagtggacacctgtggagagaaaggcaaagtggatgtcattgtcactcaagtgtatggccagatctcaagcctgccacacctcaagtgaagccaagggggtgggcctatagactctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctcgtcgacgatctctatcactgatagggagatctctatcactgatagggagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagaggcctgaccatctggtgctggcctgcaccagggccgagtttgggtctagcgatgaggataccgattgaggtgggtaaggtgggcgtggctagaagggtggggcgtgtataaattgggggtctaagggtctctctgttttgtcttgcaacagccgccgccatgagcgacaccggcaacagctttgatggaagcatctttagcccctatctgacagtgcgcatgcctcactgggctggagtgcgtcagaatgtgatgggttccaacgtggatggacgccccgttctgccttcaaattcgtctacaatggcctacgcgaccgtgggaggaactccgctggacgccgcgacctccgccgccgcctccgccgccgccgcgaccgcgcgcagcatggctacggacctttacagctctttggtggcgagcggcgcggcctctcgcgcgtctgctcgggatgagaaactgaccgctctgctgcttaaactggaagacttgacccgggagctggctcaactgacccagcaggtctccagcttgcgtgagagcagccttgcctccccctaatggcccataatataaataaaagccagtctgtttggattaagcaagtgtatgttctttatttaactctccgcgcgcggtaagcccgggaccagcggtctcggtcgtttagggtgcggtggattctttccaacacgtggtacaggtggctctggatgtttagatacatgggcatgagtccatccctggggtggaggtagcaccactgcagagcttcgtgctcgggggtggtgttgtatatgatccagtcgtagcaggagcgctgggcgtggtgctgaaaaatgtccttaagcaagaggcttatagctagggggaggcccttggtgtaagtgtttacaaatctgctcagttgggaggggtgcatccggggggatataatgtgcatcttggactggatttttaggttggctatgttcccacccagatcccttctgggattcatgttgtgcaggaccaccagcacggtatatccagtacacttgggaaatttatcgtggagcttagacgggaatgcatggaagaacttggagacgcccttgtggcctcccagattttccatacattcgtccatgatgatggcaatgggcccgtgggaagctgcctgagcaaaaatgtttctgggatcgctcacatcgtagttatgttccagggtgaggtcatcataggacatctttacaaatcgggggcggagggtcccggactgggggatgatggtgccctcgggccccggggcgtagttcccctcacagatctgcatctcccaggctttcatttcagagggagggatcatatccacctgcggagcgatgaaaaacacagtttctggcgcaggggagattaactgggatgagagcaggtttctgagcagctgtgactttccacagccggtgggcccatatatcacgcctatcaccggctgcagctggtagttaagagagctgcagctgccgtcctcccggagcaggggggccacctcgttcagcatatccctgacgtggatgttctccctgaccaattccgccagaaggcgctcgccgcccagcgaaagcagctcttgcaaggaagcaaaatttttcagcggttttaggccgtcggccgtgggcatgtttttcagcgtctgggtcagcagttccagtctgtcccacagctcggtgatgtgctctacggcatctcgatccagcagatctcctcgtttcgcgggttggggcggctttcgctgtagggcaccagccgatgggcgtccagcggggccagagtcatgtccttccatgggcgcagggtcctcgtcagggtggtctgggtcacggtgaaggggtgcgctccgggttgggcgctggccagggtgcgcttgaggctggttctgctggtgctgaatcgctgccgctcttcgccctgcgcgtcggccaggtagcatttgaccatggtctcgtagtcgagaccctcggcggcgtgccccttggcgcggagctttcccttggaggtggcgccgcacgaggggcactgcaggctcttcagggcgtagagcttgggagcgagaaacacggactctggggagtaggcgtccgcgccgcaggaagcgcagaccgtctcgcattccaccagccaagtgagctccgggcggtcagggtcaaaaaccaggttgcccccatgctttttgatgcgtttcttacctcggctctccatgaggcggtgtcccttctcggtgacgaagaggctgtccgtgtctccgtagaccgacttcaggggcctgtcttccagcggagtgcctctgtcctcctcgtagagaaactctgaccactctgagacgaaggcccgcgtccaggccaggacgaaggaggccacgtgggaggggtagcggtcgttgtccactagcgggtccaccttctccagggtgtgcaggcacatgtccccctcctccgcgtccagaaaagtgattggcttgtaggtgtaggacacgtgaccgggggttcccgacgggggggtataaaagggggtgggcaccctttcatcttcactctcttccgcatcgctgtctgcgagagccagctgctggggtaagtattccctctcgaaggcgggcatgacctcagcgctcaggttgtcagtttctaaaaatgaggaggatttgatgttcacctgtccggaggtgatacctttgagggtacctgggtccatctggtcagaaaacactatttttttgttgtcaagcttggtggcgaacgacccgtagagggcgttggagagcagcttggcgatggagcgcagggtctggtttttgtcgcggtcggctcgctccttggccgcgatgttgagttgcacgtactcgcgggccacgcacttccactcggggaagacggtggtgcgctcgtctgggatcaggcgcaccctccagcctcggttgtgcagggtgaccatgtcgacgctggtggcgacctcgccgcgcaggcgctcgttggtccagcagaggcggccgcccttgcgcgagcagaaggggggtagggggtccagctggtcctcgtttggggggtccgcgtcgatggtgaagaccccggggagcaagcgcgggtcaaagtagtcgatcttgcaagcttgcatgtccagagcccgctgccattcgcgggcggcgagcgcgcgctcgtaggggttgaggggcgggccccagggcatggggtgggtgagcgcggaggcgtacatgccgcagatgtcatacacgtacaggggttccctgaggatgccgaggtaggtggggtagcagcgccccccgcggatgctggcgcgcacgtagtcatagagctcgtgggagggggccagcatgttgggcccgaggttggtgcgctgggggcgctcggcgcggaaggcgatctgcctgaagatggcatgggagttggaggagatggtgggccgctggaagacgttgaagcttgcttcttgcaagcccaccgagtccctgacgaagcaggcgtaggactcgcgcagcttgtgcaccagctcggcggtgacctggacgtcgagcgcgcagtagtcgagggtctcgcggatgatgtcatacttatcctcccccttctttttccacagctcgcggttgaggacgaactcttcgcggtctttccagtactcttggaggggaaacccgtccgtgtccgaacggtaagagcctagcatgtagaactggttgacggcctggtaggggcaacagcccttctccacgggcagcgcgtaggcctgcgccgccttgcggagggaggtgtgggtgagggcgaaagtgtccctgaccatgactttgaggtattgatgtttgaagtctgtgtcatcgcagccgccctgttcccacagggtgtagtccgtgcgctttttggagcgcgggttgggcagggagaaggtgaggtcattgaagaggatcttccccgctcgaggcatgaagtttctggtgatgcgaaagggccctgggaccgaggagcggttgttgatgacctgggcggccaggacgatctcgtcaaagccgtttatgttgtggcccacgatgtagagctccaaaaagcggggctggcccttgatggaggggagctttttgagttcctcgtaggtgagctcctcgggcgattccaggccgtgctcctccagggcccagtcttgcaagtgagggttggccgccaggaaggatcgccagaggtcgcgggccatgagggtctgcaggcggtcgcggaaggttctgaactgtcgccccacggccatcttttcgggggtgatgcagtagaaggtgagggggtctttctcccaggggtcccatctgagctctcgggcgaggtcgcgcgcggcggcgaccagagcctcgttgccccccagtttcatgaccagcatgaagggcacgagctgcttgccaaaggctcccatccaagtgtaggtctctacatcgtaggtgacaaagaggcgctccgtgcgaggatgagagccgatcgggaagaactggatctcccgccaccagttggaggattggctgttgatgtggtgaaagtagaagtcccgtctgcgggccgagcactcgtgctggcttttgtaaaagcgaccgcagtactggcagcgctgcacgggttgtatatcttgcacgaggtgaacctggcgacctctgacgaggaagcgcagcgggaatctaagtcccccgcctggggtcccgtgtggctggtggtcttctactttggttgtctggccgccagcatctgtctcctggagggcgatggtggagcagaccaccacgccgcgagagccgcaggtccagatctcggcgctcggcgggcggagtttgatgacgacatcgcgcacattggagctgtccatggtctccagctcccgcggcggcaggtcagctgggagttcctggaggttcacctcgcagagacgggtcaaggcgcgggcagtgttgagatggtatctgatttcaaggggcgtgttggcggcggagtcgatggcttgcaggaggccgcagccccggggggccacgatggttccccgcggggcgcgaggggaggcggaagctgggggtgtgttcagaagcggtgacgcgggcgggcccccggaggtagggggggttccggccccacaggcatgggcggcaggggcacgtcttcgccgcgcgcgggcaggggctggtgctggctccgaagagcgcttgcgtgcgcgacgacgcgacggttggtgtcctgtatctgacgcctctgagtgaagaccacgggtcccgtgaccttgaacctgaaagagagttcgacagaatcaatctcggcatcgttgacagcggcctggcgcaggatctcctgcacgtcgcccgagttgtcctggtaggcgatctctgccatgaactgctcgatctcttcttcctggagatctcctcgtccggcgcgctccacggtggccgccaggtcgttggagatgcgacccatgagctgcgagaaggcgttgagcccgccctcgttccagacccggctgtagaccacgcccccctcggcgttgcgggcgcgcatgaccacctgggccaggttgagctccacgtgtcgcgtgaagacggcgtagttgcgcaggcgctggaaaaggtagttcagggtggtggcggtgtgctcggcgacgaagaagtacatgacccagcgccgcaacgtggattcattgatgtcccccaaggcctccaggcgctccatggcctcgtagaagtccacggcgaagttgaaaaactgggagttgcgagcggacacggtcaactcctcctccagaagacggatgagctcggcgacagtgtcgcgcacctcgcgctcgaaggccacggggggcgcttcttcctcttccacctcttcttccatgatcgcttcttcttcttcctcagccgggacgggagggggcggcggcggcgggggaggggcgcggcggcggcggcggcgcaccgggaggcggtcgatgaagcgctcgatcatctccccccgcatgcggcgcatggtctcggtgacggcgcggccgttctcccgggggcgcagctcgaagacgccgcctctcatctcgccgcggggcgggcggccgtgaggtagcgagacggcgctgactatgcatcttaacaattgctgtgtaggtacaccgccgagggacctgattgagtccagatccaccggatccgaaaacctttggaggaaagcgtctatccagtcgcagtcgcaaggtaggctgagcaccgtggcgggcgggggcgggtctggagagttcctggcggagatgctgctgatgatgtaattaaagtaggcggtcttgagaaggcggatggtggacaggagcaccatgtctttgggtccggcctgttggatgcggaggcggtcggccatgccccaggcctcgttctgacaccggcgcaggtctttgtagtagtcttgcatgagtctttccaccggcacctcttctccttcctcttctccatctcgccggtggtttctcgcgccgcccatgcgcgtgaccccaaagcccctgagcggctgcagcagggccaggtcggcgaccacgcgctcggccaagatggcctgctgcacctgagtgagggtcctctcgaagtcatccatgtccacgaagcggtggtaggcgcccgtgttgatggtgtaggtgcagttggccatgacggaccagttgacggtctggtgtcccggctgcgagagctccgtgtaccgcaggcgcgagaaggcgcgggaatcgaacacgtagtcgttgcaagtccgcaccagatactggtagcccaccaggaagtgcggcggaggttggcgatagaggggccagcgctgggtggcgggggcgccgggcgccaggttttccagcatgaggcggtggtatccgtagatgtacctggacatccaggtgatgccggcggcggtggtggtggcgcgcgcgtagtcgcggacccggttccagatgtttcgcaggggcgagaagtgttccatggtcggcacgctctggccggtgaggcgcgcgcagtcgttgacgctctatacacacacaaaaacgaaagcgtttacagggctttcgttctgtagcctggaggaaagtaaatgggttgggttgcggtgtgccccggttcgagaccaagctgagctcggccggctgaagccgcagctaacgtggtattggcagtcccgtctcgacccaggccctgtatcctccaggatacggtcgagagcccttttgctttcttggccaagcgcccgtggcgcgatctgggatagatggtcgcgatgagaggacaaaagcggctcgcttccgtagtctggagaaacaatcgccagggttgcgttgcggcgtaccccggttcgagcccctatggcggcttgaatcggccggaaccgcggctaacgagggccgtggcagccccgtcctcaggaccccgccagccgacttctccagttacgggagcgagccccttttgttttttattttttagatgcatcccgtgctgcggcagatgcgcccctcgccccggcccgatcagcagcagcaacagcaggcatgcagacccccctctcccctttccgccccggtcaccacggccgcggcggccgtgtcgggcgcggggggcgcgctggagtcagatgagccaccgcggcggcgacctaggcagtatctggacttggaagagggcgagggactggcgcggctgggggcgaactctccagagcgccacccgcgggtgcagttgaaaagggacgcgcgcgaggcgtacctgccgcggcagaacctgtttcgcgaccgcgggggcgaggagcccgaggagatgcgagactgcaggttccaagcggggcgcgagctgcggcgcgggctggacagacagcgcctgctgcgcgaggaggactttgagcccgacacgcagacgggcatcagccccgcgcgcgcgcacgtagccgcggccgacctggtgaccgcctacgagcagacggtgaaccaggagcgcaacttccaaaagagcttcaacaaccacgtgcgcacgctggtggcgcgcgaggaggtgaccctgggtctcatgcatctgtgggacctggtggaggcgatcgtgcagaaccccagcagcaagcccctgaccgcgcagctgttcctggtggtgcagcacagcagggacaacgaggccttcagggaggcgctgctgaacatcaccgagccggaggggcgctggctcctggacctgataaacatcctgcagagcatagtggtgcaggagcgcagcctgagcctggccgagaaggtggcggccatcaactactctatgctgagcctgggcaagttctacgcccgcaagatctacaagaccccctacgtgcccatagacaaggaggtgaagatagacagcttctacatgcgcatggcgctgaaggtgctgaccctgagcgacgacctgggagtgtaccgcaacgagcgcatccacaaggccgtgagcgccagccggcggcgcgagctgagcgaccgcgagctgatgcacagtctgcagcgcgcgctgaccggcgcgggcgagggcgacagggaggtcgagtcctacttcgacatgggggccgacctgcactggcagccgagccgccgcgccctggaggcggcgggggcgtacggcggccccctggcggccgatgaccaggaagaggaggactatgagctagaggagggcgagtacctggaggactgacctggctggtggtgttttggtatagatgcaagatccgaacgtggcggacccggcggtccgggcggcgctgcaaagccagccgtccggcattaactcctctgacgactgggccgcggccatgggtcgcatcatggccctgaccgcgcgcaaccccgaggctttcaggcagcagcctcaggccaaccggctggcggccatcttggaagcggtagtgcccgcgcgctccaaccccacccacgagaaggtgctggccatagtcaacgcgctggcggagagcagggccatccgcgcggacgaggccggactggtgtacgatgcgctgctgcagcgggtggcgcggtacaacagcggcaacgtgcagaccaacctggaccgcctggtgacggacgtgcgcgaggccgtggcgcagcgcgagcgcttgcatcaggacggtaacctgggctcgctggtggcgctaaacgccttcctcagcacccagccggccaacgtaccgcgggggcaggaggactacaccaactttttgagcgcgctgcggctgatggtgaccgaggtccctcagagcgaggtgtaccagtcggggcccgactacttcttccagaccagcagacagggcttgcaaaccgtgaacctgagccaggctttcaagaacctgcgggggctgtggggagtgaaggcgcccaccggcgaccgggctacggtgtccagcctgctaacccccaactcgcgcctgctgctgctgctgatcgcgcccttcacggacagcgggagcgtctcgcgggagacctatctgggccacctgctgacgctgtaccgcgaggccatcgggcaggcgcaggtggacgagcacaccttccaagagatcaccagcgtgagccacgcgctggggcaggaggacacgggcagcctgcaggcgaccctgaactacctgctgaccaacaggcggcagaagattcccacgctgcacagcctgacccaggaggaggagcgcatcttgcgctacgtgcagcagagcgtgagcctgaacctgatgcgcgacggcgtgacgcccagcgtggcgctggacatgaccgcgcgcaacatggaaccgggcatgtacgcctcccaccggccgttcatcaaccgcctgatggactacttgcatcgggcggcggccgtgaaccccgagtacttcactaatgccattctgaatccccactggatgccccctccgggtttctacaacggggactttgaggtgcccgaggtcaacgacgggttcctctgggatgacatggatgacagtgtgttctcacccaacccgctgcgcgccgcgtctctgcgattgaaggagggctctgacagggaaggaccgagaagtctggcctcctccctggctctgggagcggtgggcgccacgggcgcggcggcgcggggcagtagccccttccccagcctggcagactctctgaacagcgggcgggtgagcaggccccgcttgctaggcgaggaggagtatctgaacaactccctgctgcagcccgcgagggacaagaacgctcagcggcagcagtttcccaacaatgggatagagagcctggtggacaagatgtccagatggaagacgtatgcgcaggagtacaaggagtgggaggaccgccagccgcggcccttgccgccccctaggcagcgctggcagcggcgcgcgtccaaccgccgctggaggcaggggcccgaggacgatgatgactctgcagatgacagcagcgtgttggacctgggcgggagcgggaaccccttttcgcacctgcgcccacgcctgggcaagatgttttaaaagaaaaaaaaaaaaataaaactcaccaaggccatggcgacgagcgttggttttttgttcccttccttagtatgcggcgcgcggcgatgttcgaggaggggcctcccccctcttacgagagcgcgatggggatttctcctgcggcgcccctgcagcctccctacgtgcctcctcggtacctgcaacctacaggggggagaaatagcatctgttactctgagctgcagcccctgtacgataccaccagactgtacctggtggacaacaagtccgcggacgtggcctccctgaactaccagaacgaccacagcgattttttgaccacggtgatccaaaacaacgacttcaccccaaccgaggccagcactcagaccataaacctggataacaggtcgaactggggcggcgacctgaagaccatcttgcacaccaacatgcccaacgtgaacgagttcatgttcaccaactcttttaaggcgcgggtgatggtggcgcgcgagcagggggaggcgaagtacgagtgggtggacttcacgctgcccgagggcaactactcagagaccatgactctcgacctgatgaacaatgcgatcgtggaacactatctgaaagtgggcaggcagaacggggtgaaggaaagcgatatcggggtcaagtttgacaccagaaacttccgtctgggctgggaccccgtgaccgggctggtcatgccgggggtctacaccaacgaggcctttcatcccgacatagtgcttctgcccggctgtggggtggacttcacccagagccggctgagcaacctgctgggcattcgcaagcggcagcctttccaggagggtttcaagatcacctatgaggatctgaaggggggcaacattcccgcgctccttgatctggacgcctacgaggagagcttgaaacccgaggagagcgctggcgacagcggcgagagtggcgaggagcaagccggcggcggtggcggcgcgtcggtagaaaacgaaagtacgcccgcagtggcggcggacgctgcggaggtcgagccggaggccatgcagcaggacgcagaggagggcgcacaggagggcgcgcagaaggacatgaacgatggggagatcaggggagacacattcgccacccggggcgaagaaaaagaggcagaggcggcggcggcggcgacggcggaggccgaaaccgaggttgaggcagaggcagagcccgagaccgaagttatggaagacatgaatgatggagaacgtaggggcgacacgttcgccacccggggcgaagagaaggcggcggaggcagaagccgcggctgaggaggcggctgcggctgcggccaagactgaggctgcggctaaggctgaggtcgaagccaatgttgcggttgaggctcaggctgaggaggaggcggcggctgaagcagttaaggaaaaggcccaggcagagcaggaagagaaaaaacctgtcattcaacctctaaaagaagatagcaaaaagcgcagttacaacgtcatcgagggcagcacctttacccagtaccgcagctggtacctggcgtacaactacggcgacccggtcaagggggtgcgctcgtggaccctgctctgcacgccggacgtcacctgcggctccgagcagatgtactggtcgctgccgaacatgatgcaagacccggtgaccttccgctccacgcggcaggttagcaacttcccggtggtgggcgccgaactgctgcccgtgcactccaagagtttttacaacgagcaggccgtctactcccagctgatccgccaggccacctctctgacccacgtgttcaatcgctttcccgagaaccagattttggcgcgcccgccggcccccaccatcaccaccgtgagtgaaaacgttcctgccctcacagatcacgggacgctaccgctgcgcaacagcatctcaggagtccagcgagtgaccattactgacgccagacgccggacctgcccctacgtttacaaggccttgggcatagtctcgccgcgcgtcctctccagtcgcactttttaaaacacatctacccacacgttccaaaatcatgtccgtactcatctcacccagcaacaacaccggctgggggctgcgcgcgcccagcaagatgtttggaggggcgaggaagcgctccgaccagcaccctgtgcgcgtgcgcggccactaccgcgcgccctggggagcgcacaagcgcgggcgcacagggcgcaccactgtggacgacgtcattgactccgtagtggagcaagcgcgccactacacacccggcgcgccgaccgcccccgccgtgtccaccgtggaccaggcgatcgaaagcgtggtacagggcgcgcggcactatgccaaccttaaaagtcgccgccgccgcgtggcccgccgccatcgccggagaccccgggccaccgccgccgcgcgccttactaaggctctgctcaggcgcgccaggcgaactggccaccgggccgccatgagggccgcacggcgggctgccgctgccgcaagcgccgtggccccgcgggcacgaaggcgcgcggccgccgccgccgccgccgccatttccagcttggcctcgacgcggcgcggtaacatatactgggtgcgcgactcggtaaccggcacgcgggtacccgtgcgctttcgccccccgcggaattagcacaagacaacatacacactgagtctcctgctgttgtgtatcccagcggcgaccgtcagcagcggcgacatgtccaagcgcaaaattaaagaagagatgctccaggtcatcgcgccggagatctatgggcccccgaagaaggaggaggatgattacaagccccgcaagctaaagcgggtcaaaaagaaaaagaaagatgatgatgatgacgaggcggtggagtttgtccgccgcatggcacccaggcgccccgtgcagtggaagggccggcgcgtgcagcgcgttttgcgccccggcaccgcggtggtcttcacgcccggcgagcgctccacgcgcactttcaagcgggtgtacgatgaggtgtacggcgacgaggacctgttggagcaggccaaccagcgctttggggagtttgcatatgggaaacggccccgcgagagtctaaaagaggacctgctggcgctaccgctggacgagggcaatcccaccccgagtctgaagccggtaaccctgcaacaggtgctgcctttgagcgcgcccagcgagcataagcgagggttgaagcgcgaaggcggggacctggcgcccaccgtgcagttgatggtgcccaagcggcagaagctggaggacgtgctggagaaaatgaaagtagagcccgggatccagcccgagatcaaggtccgccccatcaagcaggtggcgcccggcgtgggagtccagaccgtggacgttaggattcccacggaggagatggaaacccaaaccgccactccctcttcggcggccagcgccaccaccggcaccgcttcggtagaggtgcagacggacccctggctacccgccaccgctgttgccgccgccgccccccgttcgcgcgggcgcaagagaaattatccagcggccagcgcgctcatgccccagtacgcactgcatccatccatcgcgcccacccccggctaccgcgggtactcgtaccgcccgcgcagatcagccggcactcgcggccgccgccgccgtgcgaccacaaccagccgccgccgtcgccgccgccgccagccagtgctgacccccgtgtctgtaaggaaggtggctcgctcggggagcacgctggtggtgcccagagcgcgctaccaccccagcatcgtttaaagccggtctctgtatggttcttgcagatatggccctcacttgtcgcctccgcttcccggtgccgggataccgaggaagaactcaccgccgcagaggcatggcgggcagcggtctccgcggcggccgtcgccatcgccggcgcgcaaaaagcaggcgcatgcgcggcggtgtgctgcctctgctaatcccgctaatcgccgcggcgatcggtgccgtacccgggatcgcctccgtggccctgcaggcgtcccagaaacgttgactcttgcaaccttgcaagcttgcattttttggaggaaaaaataaaaaaagtctagactctcacgctcgcttggtcctgtgactattttgtagaaaaaagatggaagacatcaactttgcgtcgctggccccgcgtcacggctcgcgcccgttcatgggagactggacagatatcggcaccagcaatatgagcggtggcgccttcagctggggcagtctgtggagcggccttaaaaattttggttccaccattaagaactatggcaacaaagcgtggaacagcagcacgggccagatgctgagagacaagttgaaagagcagaacttccaggagaaggtggcgcagggcctggcctctggcatcagcggggtggtggacatagctaaccaggccgtgcagaaaaagataaacagtcatctggacccccgtcctcaggtggaggaaatgcctccagcgatggagacggtgtctcccgagggcaaaggcgaaaagcgcccgcggcccgacagagaagagaccctggtgtcacacaccgaggagccgccctcttacgaggaggcagtcaaggccggcctgcccaccactcgccccatagcccccatggccaccggtgtggtgggccacaggcaacacactcccgcaacactagatctgcccccgccgtccgagccgccgcgccagccaaaggcggcgacggtgcccgctccctccacttccgccgccaacagagtgcccctgcgccgcgccgcgagcggcccccgggcctcgcgagttagcggcaactggcagagcacactgaacagcatcgtgggcctgggagtgaggagtgtgaagcgccgccgttgctactgaatgagcaagctagctaacgtgttgtatgtgtgtatgcgtcctatgtcgccgccagaggagctgttgagccgccggcgccgtctgcactccagcgaatttcaagatggcgaccccatcgatgatgcctcagtggtcgtacatgcacatctcgggccaggacgcttcggagtacctgagccccgggctggtgcagttcgcccgcgccacagacacctacttcaacatgagtaacaagttcaggaaccccactgtggcgcccacccacgatgtgaccacggaccggtcgcagcgcctgacgctgcggttcatccccgtggatcgggaggacaccgcctactcttacaaggcgcggttcacgctggccgtgggcgacaaccgcgtgctggacatggcctccacttactttgacatcaggggggtgctggacaggggccccaccttcaagccctactcgggtactgcctacaactccctggcccccaagggcgctcccaattcttgcgagtgggaacaagaggaaaatcaggtggtcgctgcagatgatgaacttgaagatgaagaagcgcaagctcaagaggacgccccagctaaaaaaattcatgtatatgcccaggcgcctcttgctggcgaaaagattaccaaggatggtttgcaaataggtactgaagttgtaggagatacatctaaggacacttttgcagacaaaacattccaacccgaacctcagataggcgagtctcagtggaacgaggctgatgccacagtagcaggaggcagagtcttgaaaaaaaccacccctatgagaccttgctatggatcctatgccaggcctacaaatgccaacgggggtcaaggaattatggttgccaatgaacaaggagtgttggagtctaaagtggagatgcaatttttttctaacactacaacccttaatgcgcgggatggagctggcaatcccgaaccaaaggtggtgttgtacagtgaagatgtccacttggaatctcctgacactcatttgtcttacaagcccaaaaaggatgatgttaatgctaaaattatgttgggtcagcaagctatggctaacaggcccaacctcattgcttttagagataatttcattggactcatgtactacaacagcactggtaacatgggagtgctggcgggtcaggcctctcagttgaatgccgtggtggacctgcaggatagaaacacagaactgtcatatcagcttatgcttgattccattggggatagatccagatacttctccatgtggaaccaggcagtggatagctatgacccagatgtcagaatcattgaaaaccatggtgtcgaggacgagctacccaactactgcttccctctgggcggcataggaattactgatacttatcaagggatcaaaaataccaatggcaatggtcagtggaccaaagatgatcagttcgcggaccgtaatgaaataggggtgggaaacaacttcgccatggagatcaacatccaggccaacctctggaggaacttcctctatgcgaacgtggggctctacctgccagacaagctcaagtacaaccccaccaacgtggacatctctgacaaccccaacacctatgactacatgaacaagcgtgtggtggctcccggcctggtggactgctttgtcaatgtgggagccaggtggtccctggactacatggacaacgtcaaccccttcaaccaccaccgcaatgcgggtctgcgctaccgctccatgatcctgggcaacgggcgctacgtgcccttccacattcaggtgccccagaagttctttgccatcaagaacctcctcctcctgccgggctcctacacttacgagtggaacttcaggaaggatgtcaacatggtcctgcagagctctctgggcaatgaccttagggtggacggggccagcatcaagtttgacagcgtcaccctctatgctaccttcttccccatggctcacaacaccgcctccacgctcgaggccatgctgaggaacgacaccaacgaccagtccttcaatgactacctctctggggccaacatgctctaccccatccccgccaaggccaccaacgtgcccatctccattccctctcgcaactgggccgccttcagaggctgggcctttacccgccttaagaccaaggaaaccccctccctgggctcgggttttgacccctactttgtctactcgggatccatcccctacctggatggcaccttctacctcaaccacacttttaagaagatatccatcatgtatgactcctccgtcagctggccgggcaatgaccgcctgctcacccccaatgagttcgaggtcaagcgcgccgtggacggcgagggctacaacgtggcccagtgcaacatgaccaaggactggttcctggtgcagatgctggccaactacaacataggctaccagggcttctacatcccagagagctacaaggacaggatgtactccttcttcagaaatttccaacccatgagcaggcaggtggtggacgagaccaaatacaaggactatcaggccattggcatcactcaccagcacaacaactcgggattcgtgggctacctggctcccaccatgcgcgaggggcaggcctaccccgccaacttcccctacccgttgataggcaagaccgcggtcgacagcgtcacccagaaaaagttcctctgcgaccgcaccctctggcgcatccccttctctagcaacttcatgtccatgggtgcgctcacggacctgggccagaacctgctctatgccaactccgcccatgcgctggacatgacttttgaggtggaccccatggacgagcccacccttctctatattgtgtttgaagtgttcgacgtggtcagagtgcaccagccgcaccgcggtgtcatcgagaccgtgtacctgcgcacgcccttctcggccggcaacgccaccacctaaggagacagcgccgccgcctgcatgacgggttccaccgagcaagagctcagggccatcgccagagacctgggatgcggaccctattttttgggcacctatgacaaacgcttcccgggcttcatctcccgagacaagctcgcctgcgccatcgtcaacacggccgcgcgcgagaccgggggcgtgcactggctggcctttggctgggacccgcgctccaaaacctgctacctcttcgacccctttggcttctccgatcagcgcctcagacagatctatgagtttgagtacgaggggctgctgcgccgcagcgcgcttgcctcctcgcccgaccgctgcatcacccttgagaagtccaccgagaccgtgcaggggccccactcggccgcctgcggtctcttctgctgcatgtttttgcacgcctttgtgcgctggccccagagtcccatggatcgcaaccccaccatgaacttgctcaagggagtgcccaacgccatgctccagagcccccaggtccagcccaccctgcgccacaaccaggaacagctctaccgcttcctggagcgccactccccctacttccgcagtcacagcgcgcacatccggggggccacctctttctgccacttgcaacaaaacatgcaagacggaaaatgatgtacagctcgctttttaataaatgtaaagactgtgcactttatttatacacgggctctttctggttatttattcaacaccgccgtcgccatctagaaatcgaaagggttctgccgcgcgtcgccgtgcgccacgggcagagacacgttgcgatactggaagcggctcgcccacttgaactcgggcaccaccatgcggggcagtggctcctcggggaagttctcgccccacagggtgcgggtcagctgcagcgcgctcaggaggtcgggagccgagatcttgaagtcgcagttggggccggaaccctgcgcgcgcgagttgcggtacacggggttgcagcactggaacaccagcagggccggattacgcacgctggccagcaggctctcgtcgctgatcatgtcgctgtccagatcctccgcgttgctcagggcgaatggggtcatcttgcagacctgcctgcccaggaaaggcggcagcccgggcttgccgttgcagtcgcagcgcaggggcatcagcaggtgcccgtggcccgtctgcgcctgcgggtacagcgcgcgcatgaaggcttcgatctgcctgaaagccacctgcgtcttggctccctccgaaaagaacatcccacaggacttgctggagaactggttcgcgggacagctggcatcgtgcaggcagcagcgcgcgtcggtgttggcgatctgcaccacgttgcgaccccaccggttcttcactatcttggccttggaagcctgctccttcagcgcgcgctggccgttctcgctggtcacatccatctctatcacctgctccttgttgatcatgtttgtcccgtgcagacacttcaggtcgccctccgtctgggtgcagcggtgctcccacagcgcgcaaccggtgggctcccaatttttgtgggtcacccccgcgtaggcctgcaggtaggcctgcaagaagcgccccatcatggccacaaaggtcttctggctcgtaaaggtcagctgcaggccgcgatgctcttcgttcagccaggtcttgcagatggcggccagcgcctcggtctgctcgggcagcatcctaaaatttgtcttcaggtcgttatccacgtggtacttgtccatcatggcgcgcgccgcctccatgcccttctcccaggcggacaccatgggcaggcttagggggtttatcacttccaccggcgaggacaccgtactttcgatttcttcttcctccccctcttcccggcgcgcgcccacgctgctgcgcgctctcaccgcctgcaccaaggggtcgtcttcaggcaagcgccgcaccgagcgcttgccgcccttgacctgcttaatcagcaccggcgggttgctgaagcccaccatggtcagctccgcctgctcttcttcgtcttcgctgtctaccactatctctggggaagggcttctccgctctgcggcggtgcgcttcttttttttcttgggagcagccgtgacggagtccgccacggcgacggaggtcgagggcgtggggctgggggtgcgcggtaccagggcctcgtcgccctcggactcttcctctgactccaggcggcggcggagacgcttctttgggggcgcgcgcgtcagcggcggcggagacggggacggggacggggacgggacgccctccacagggggtggtcttcgcgcagacccgcggccgcgctcgggggtcttttcgagctggtcttggtcccgactggccattgtatcctcctcctcctaggcagagagacataaggagtctatcatgcaagtcgagaaggaggagagcttaaccaccccctctgagaccgccgatgcgcccgccgtcgccgtcgcccccgctgccgccgacgcgcccgccacaccgagcgacacccccgcggaccccccagccgacgcacccctgttcgaggaagcggccgtggagcaggacccgggctttgtctcggcagaggaggatttgcgagaggaggaggataaggagaagaagccctcagtgccaaaagatgataaagagcaagacgagcacgacgcagatgcacaccagggtgaagtcgggcggggggacggagggcatgacggcgccgactacctagacgaagggaacgacgtgctcttgaagcacctgcatcgtcagtgcgccatcgtttgcgacgctctgcaggagcgcagcgaagtgcccctcagcgtggcggaggtcagccacgcctacgagctcagcctcttctccccccgggtgcccccccgccgccgcgaaaacggcacatgcgagcccaacccgcgcctcaacttctaccccgcctttgtggtgcccgaggtcctggccacctatcacatcttctttcaaaattgcaagatccccctctcgtgccgcgccaaccgtagccgcgccgataagatgctggccctgcgccagggcgaccacatacctgatatcgccgctttggaagatgtgccaaagatcttcgagggtctgggtcgcaacgagaagcgggcagcaaactctctgcaacaggaaaacagcgaaaatgagagtcacaccggggtactggtggagctcgagggcgacaacgcccgcctggcggtggtcaagcgcagcatcgaggtcacccactttgcctaccccgcgctcaacctgccccccaaagtcatgaacgcggccatggacgggctgatcatgcgccgcggccggcccctcgctccagatgcaaacttgcatgaggagaccgaggacggccagcccgtggtcagcgacgagcagctggcgcgctggctggagaccgcggaccccgccgaactggaggagcggcgcaagatgatgatggccgcggtgctggtcaccgtagagctggagtgtctgcagcgcttcttcggcgaccccgagatgcagagaaaggtcgaggagaccctgcactacaccttccgccagggctacgtgcgccaggcttgcaagatctccaacgtggagctcagcaacctggtgtcctacctgggcatcttgcatgagaaccgcctcgggcagagcgtgctgcactccaccctgcgcggggaggcgcgccgcgactacgtgcgcgactgcgtttacctcttcctctgctacacctggcagacggccatgggggtctggcagcagtgcctggaggagcgcaacctcaaggagctggagaagctcctgcagcgcgcgctcaaagacctctggacgggctacaacgagcgctcggtggccgccgcgctggccgacctcatcttccccgagcgcctgctcaaaaccctccagcaggggctgcccgacttcaccagccaaagcatgttgcaaaacttcaggaactttatcctggagcgttctggcatcctacccgccacctgctgcgccctgcccagcgactttgtccccctcgtgtaccgcgagtgccccccgccgctgtggggtcactgctacctgttccaactggccaactacctgtcctaccacgcggacctcatggaggactccagcggcgaggggctcatggagtgccactgccgctgcaacctctgcacgccccaccgctccctggtctgcaacacccaactgctcagcgagagtcagattatcggtaccttcgagctacagggtccgtcctcctcagacgagaagtccgcggctccggggctaaaactcactccggggctgtggacttccgcctacctgcgcaaatttgtacctgaagactaccacgcccacgagatcaggttttacgaagaccaatcccgcccgcccaaggcggagctgaccgcctgcgtcatcacccagggcgagatcctaggccaattgcaagccatccaaaaagcccgccaagattttttgctgagaaagggtcggggggtgtatctggacccccagtcgggtgaggagctcaacccggttcccccgctgccgccgccgcgggaccttgcttcccaggataagcatcgccatggctcccagaaagaagcagcagcggccgccactgccgccaccccacatgctggaggaagaggaggaatactgggacagtcaggcagaggaggtttcggacgaggaggagccggagacggagatggaagagtgggaggaggacagcttagacgaggaggcttccgaagccgaagaggcagacgcaacaccgtcaccctcggccgcagccccctcgcaggcgcccccgaagtccgctcccagcatcagcagcaacagcagcgctataacctccgctcctccaccgccgcgacccacggccgaccgcagacccaaccgtagatgggacaccaccggaaccggggccggtaagtcctccgggagaggcaagcaagcgcagcgccaaggctaccgctcgtggcgcgctcacaagaacgccatagtcgcttgcttgcaagactgcggggggaacatctccttcgcccgccgcttcctgctcttccaccacggtgtggccttcccccgtaacgtcctgcattactaccgtcatctctacagcccctactgcggcggcagtgagccagagacggtcggcggcggcggcggcgcccgtttcggcgcctaggaagacccagggcaagacttcagccaagaaactcgcggcggccgcggcgaacgcggtcgcgggggccctgcgcctgacggtgaacgaacccctgtcgacccgcgaactgagaaaccgaatcttccccactctctatgccatcttccagcagagcagagggcaggatcaggaactgaaagtaaaaaacaggtctctgcgctccctcacccgcagctgtctgtatcacaagagcgaagaccagcttcggcgcacgctggaggacgctgaggcactcttcagcaaatactgcgcgctcactcttaaggactagctccgcgcccttctcgaatttaggcgggaacgcctacgtcatcgcagcgccgccgtcatgagcaaggacattcccacgccatacatgtggagctatcagccgcagatgggactcgcggcgggcgcctcccaagactactccacccgcatgaactggctcagtgccggcccacacatgatctcacaggttaatgatatccgcacccatcgaaaccaaatattggtggagcaggcggcaattaccaccacgccccgcaataatcccaaccccagggagtggcccgcgtccctggtgtatcaggaaattcccggccccaccaccgtactacttccgcgtgattcccaggccgaagtccaaatgactaactcaggggcgcagctcgcgggcggctgtcgtcacagggtgcggcctcctcgccagggtataactcacctggagatccgaggcagaggtattcagctcaacgacgagtcggtgagctcctcgctcggtctaagacctgacgggaccttccagatagccggagccggccgatcttccttcacgccccgccaggcgtacctgactctgcagagctcgtcctcggcgccgcgctcgggcggcatcgggactctccagttcgtgcaggagtttgtgccctcggtctacttcaaccccttctcgggctctcccggtcgctacccggaccagttcatctcgaactttgacgccgcgagggactcggtggacggctacgactgaatgtcgggtggacccggtgcagagcaacttcgcctgaagcacctcgaccactgccgccgccctcagtgctttgcccgctgtcagaccggtgagttccagtacttttccctgcccgactcgcacccggacggcccggcgcacggggtgcgctttttcatcccgagtcaggtgcgctctaccctaatcagggagtttaccgcccgtcccctactggcggagttggaaaaggggccttctatcctaaccattgcctgcatctgctctaaccctggattgcaccaagatctttgctgtcatttgtgtgctgagtataataaaggctgagatcagaatctactcgggctcctgtcgccatcctgtcaacgccaccgtccaagcccggcccgatcagcccgaggtgaacctcacctgcggtctgcaccggcgcctgaggaaatacctagcttggtactacaacagcactccctttgtggtttacaacagctttgaccaggacggggtctcactgagggataacctctcgaacctgagctactccatcaggaagaacagcaccctcgagctacttcctccttacctgcccgggacttaccagtgtgtcaccggtccctgcacccacacccacctgttgatcgtaaacgactctcttccgagaacagacctcaataactcctcttcgcagttccccagaacaggaggtgagctcaggaaaccccgggtaaagaagggtggacaagagttaacacttgtggggtttctggtgtatgtgacgctggtggtggctcttttgattaaggcttttccttccatgtctgaactctccctcttttatgaacaactcgactagtgctaacgggaccctacccaacgaatcgggattgaatatcggtaaccaggttgcagtttcacttttgattaccttcatagtcctcttcctgctagtgctgtcgcttctgtgcctgcggatcgggggctgctgcatccacgtttatatctggtgctggctgtttagaaggttcggagaccatcgcaggtagaataaacatgctgctgcttaccctctttgtcctggcgctggccgccagctgccaagccttttccgaggctgactttatagagccccagtgtaacgtgacttttaaagcccatgcacagcgttgtcatactataatcaaatgtgccaccgaacacgatgaataccttatccagtataaagataaatcacacaaagtggcacttgttgacatctggaaacccgaagaccctttggaatacaatgtgaccgttttccagggtgacctcttcaaaatttacaattacactttcccatttgaccagatgtgtgactttgtcatgtacatggaaaagcagcacaagctgtggcctccgactccccagggctgtgtggaaaatccaggctctttctgcatgatctctctctgtgtaactgtgctggcactaatactcacgcttttgtatatcagatttaaatcaaggcaaagcttcatcgatgaaaagaaaatgccttaaacgctttcacgcttgattgctaacaccgggtttttatccgcagaatgattggaatcaccctactaatcacctccctccttgcgattgcccatgggttggaacgaatcgaagcccctgtgggggccaatgttaccctggtggggcctgtcggcaatgctacattaatgtgggaaaaatatactaaaaatcaatgggtctcttactgcactaacaaaaacagccacaagcccagagccatctgcgatgggcaaaatctaaccttgattgatgttcaaatgctggatgcgggctactattatgggcagctgggtacaatgattaattactggagaccccacaaagattacatgctccacgtagtaaagggtccccttagcagcccacccactaccacctctactacccccactaccaccactactcccaccaccagcactgccgcccagcctcctcatagcagaacaaccacttttatcaattccaagtcccactccccccacattgccggcgggccctccgcctcagactccgagaccaccgagatctgcttctgcaaatgctctgacgcctttgctgaggatttggaagaccacgaggaagatgagcatgacttcgcagatgcatgccaggcatcagaggcagaagcgctgccggtggccctcaaacagtatgcagacccccacaccacccccaaccttcctccaccttcccagaagccaagtttcctgggggaaaatgaaactctgcctctctccatactcgctctgacatctgttgctatgttgaccgctctgctggtgcttctatgctctatatgctacctgatctgctgcagaaagaaaaaatctcacggccatgctcaccagcccctcatgcacttcccttaccctccagagctgggcgaccacaaactttaagtctgcagtaactatctgcccatcccttgtcagtcgacagcgatgagccccactaatctaacggcctctggacttacaacatcgtctcttaatgagaccaccgctcctcaagacctgtacgatggtgtctccgcgctggttaaccagtgggatcacctgggcatatggtggctcctcataggagcagtgaccctgtgcctaatcctggtctggatcatctgctgcatcaaaagcagaagacccaggcggcggcccatctacaggccctttgtcatcacacctgaagatgatgatgacaccacttccaggctgcagaggctaaagcagctactcttctcttttacagcatggtaaattgaatcatgcctcgcattttcatctacttgtctctccttccactttttctgggctcttctacattggccgctgtgtcccacatcgaggtagactgcctcacgcccttcacagtctacctgcttttcggctttgtcatctgcacctttgtctgcagcgttatcactgtagtgatctgcttcatacagtgcatcgactacgtctgcgtgcgggtggcttactttagacaccacccccagtatcgcaacagggacatagcggctctcctaagacttgtttaaaatcatggccaaattaactgtgattggtcttctgatcatctgctgcgtcctagccgcgattgggactcaagctcctaccaccaccagcgctcccagaaagagacatgtatcctgcagcttcaagcgtccctggaatataccccaatgctttactgatgaacctgaaatctctttggcttggtacttcagcgtcaccgcccttcttatcttctgcagtacggttattgcccttgccatctacccttcccttgacctgggctggaatgctgtcaactctatggaatatcccaccttcccagaaccagacctgccagacctggttgttctaaacgcgtttcctcctcctgctcccgttcaaaatcagtttcgccctccgtcccccacgcccactgaggtcagctactttaatctaacaggcggagatgactgaaaacctagacctagaaatggacggtctctgcagcgagcaacgcacactagagaggcgccggcaaaaagagctcgagcgtcttaaacaagagctccaagacgcggtggccatacaccagtgcaaaaaaggtgtcttctgtctggtaaaacaggccacgctcacctatgaaaaaacaggtgacacccaccgcctaggatacaagctgcccacacagcgccagaagttcgccctcatgataggcgaacaacccatcaccgtgacccagcactccgtggagacagaaggctgcatacacgctccctgtaggggcgctgactgcctctacaccttgatcaaaaccctctgcggtctcagagacctcatcccttttaattaatcataactgtaatcaataaaaaatcacttacttgaaatctgatagcaagcctctgtccaattttttcagcaacacttccttcccctcctcccaactctggtactctaggcgcctcctagctgcaaacttcctccacagtctgaagggaatgtcagattcctcctcctgtccctccgcacccacgatcttcatgttgttgcagatgaaacgcgcgagatcgtctgacgagaccttcaaccccgtgtacccctacgataccgagatcgctccgacttctgtccctttccttacccctccctttgtgtcatccgcaggaatgcaagaaaatccagctggggtgctgtccctgcacttgtcagagccccttaccacccacaatggggccctgactctaaaaatggggggcggcctgaccctggacaaggaagggaatctcacttcccaaaacatcaccagtgtcgatccccctctcaaaaaaagcaagaacaacatcagccttcagaccgccgcacccctcgccgtcagctccggggccctaacactttttgccactccccccctagcggtcagtggtgacaaccttactgtgcagtctcaggcccctctcactttggaagactcaaaactaactctggccaccaaaggacccctaactgtgtccgaaggcaaacttgtcctagaaacagaggctcccctgcatgcaagtgacagcagcagcctgggccttagcgttacggccccacttagcattaacaatgacagcctaggactagacatgcaagcgcccattagctctcgagatggaaaactggctctaacagtggcggcccccctaactgtggtcgagggtatcaatgctttggcagtagccacaggtaagggtattgggctaaatgaaaccaacacacacctgcaggcaaaactggtcgcacccctaggctttgataccaacggcaacattaagctaagcgttgcaggaggcatgaggctaaacaataacacactgatactagatgtaaactacccatttgaggctcaaggccaactgagcctaagagtgggctcgggcccactatatgtagattctagtagtcataacctaaccattagatgccttaggggattgtatataacatcttctaacaaccaaaacggtctagaagccaacattaaactaacaagaggccttgtgtatgacggaaatgccatagcagttaatgttggcaaagggctggaatacagccctactgacacaacagaaaaacctatacagactaaaataggtctaggcatggagtatgataccgagggagccatgatgacaaaactaggctctggactaagctttgacaattcaggagccattgtagtgggaaacaaaaatgatgacaggcttactttgtggaccacaccggacccatcgcccaactgtcagatctactctgaaaaagatgctaaactaaccttggtactgactaaatgtggcagtcaggttgtaggcacagtatctattgccgctcttaaaggtagcctcgtgccaatcactagtgcaatcagtgtggttcaggtatacctaaggtttgatgaaaatggggtactaatgagtaactcttcacttaatggcgaatactggaattttagaaacggagactcaactaatggcacaccatatacaaacgcagtgggtttcatgcctaatctactggcctatcctaaaggtcaaactacaactgcaaaaagtaacattgtcagccaggtctacatgaatggggacgatactaaacccatgacatttacaatcaacttcaatggccttagtgaaacaggggatacccctgttagtaaatattccatgacattctcatggaggtggccaaatggaagctacatagggcacaattttgtaacaaactcctttaccttctcctacatcgcccaagaataaagaaagcacagagatgcttgtttttgatttcaaaattgtgtgcttttatttattttcagcttacagtatttccagtagtcattcaaataaagcttaatcaaactgcatgagaacccttccacatagcttaaattagcaccagtgcaaatggagaaaacaattgacggccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagggtaccaacgcgtcgtaactataacggtcctaaggtagcgaaaagcactctcacagcaccagcactaatcagagtgtgaagagggccaagtgccgaacgagtatatataggaataaaaaatgacgtaaatgtgtaaaggtcagaaaacgcccagaaaaatacacagaccaacgcccgaaacgaaaacccgcgaaaaaatacccagaacttcctcaacaaccgccacttccgctttctcacggtacgtcacttccgcaagaaaagcaaaactacatttcccacatgtgtaaaaacgaaaccccgccccttgtaaccgcccacaacttacatcatcaaaacgtaaactcctacgtcacccgccccgcctctccccgcccacctcattatcatattggccacaatccaaaataaggtatattattgatgatg

SEQ ID NO: 245 - protein, 572 aa, synthetic construct
IQTNRELTEVFEFAPRYSHSDYIIFVYIPLGDAKLPQLCTEVDDLLLMGTLGIVPLHAPMSADTLHEYMLDLHNRATDLSEEENDEIDGVNHQHLPARRIHDNLLIRCLRCPLILAARLALLFLNTLSFVCPWCASSHADMKCIDFYSRIDNLKLPDLCTELALHWTAAVNNLQLFLNTLSFVPANKFQQLFLNTLNDRVYDFAFRDLPLFLAAKEGSYEKLTNTGLYNLASRLCIVYRDGNPYAVCDNHMYMLDLQPETTPRQFAFRDLCIVYIVRFLNLEVSQTSKFTRQTERTLGIVCPIAAHYSKSDRTLEDLLMGVANPTIHDIILECVQNMHLDKKQRFHNIPLHAAYSADTTLEQQYNKPLCDLLRSRATDLCDSTLRLCVVHDRWTGRCMSCCPLILAPRLAVSLQDIEITCVSHADILCVQSTHVDIDDIAHYNIVTFCCLHWGATMSNVDDLRAFQQLFLNTLSGPSKISEYRHYCYDNRKLPQLCTELPLFLGAKEPSYEKTVLELTEVADRRTLEDLLMGTQHMFAFKDLFVVPRNQLYNKPLCDVIIHTLQDIVLHLFE

SEQ ID NO: 246 - protein, 572 aa, synthetic construct
MQTDHELTEVFEFAARYGRGDYIIFVYIPLAQAKLPQLCTEVNNMLLMGTLGIVPLRAPVSADTLHEYMLDLRNRPTNLSEEENDEIDGVNHQHLPARRMRDNLLIRCLRCPLILAPRLAVLFLNTLSFVCPWCASSRAIVKCIDFYSRINDLKLPDLCTELALHWAGAVNNIQLFLNTLSFVGPNKFQQLFLNTLDNRVYDFAFRDLPLFLATREGSYEKLTNTGLYNLANQLCIVYRDGNPYAVCDNQMYMLDLQPETTPHDFAFRDLCIVYVIRLLELEVSQSTKLTRQTQRTLGIVCPIAAHYSRSQRTLEDLLMGVPDPTIHDIILECVENMHLDKKQRFHNIPLHTAVSANTTLEQQYNKPLCDLLHNRATILCDSTLRLCVMRDRWTGRCMSCCPLILPARLPVSLQDIEITCVSQADVLCVQSTHVDIDNLAHYNIVTFCCLQWAAAINNVDDLRAFQQLFLNTLSPTNKISEYRHYCYNNHKLPQLCTELPLFLPAREPSYEKTVLELTEVTNRRTLEDLLMGTNHMFAFKDLFVVAREQLYNKPLCDVMVRTLQDIVLHLLQ

SEQ ID NO: 247 - protein, 572 aa, synthetic construct
METDRELTEVFEFAARFSRSDYIIFVYIPLADAKLPQLCTEVDSMLLMGTLGIVPLHAAMSADTLHEYMLDLRNHATDLSEEENDEIDGVNHQHLPARRMHDNLLIRCLRCPIILAPRLAVLFLNTLSFVCPWCASSHADVKCIDFYSRIDDLKLPDLCTELALHWAAAVNSVQLFLNTLSFVGASKFQQLFLNTLNNRVYDFAFRDLPLFLAAREGSFEKLTNTGLYNLANRLCIVYRDGNPYAVCDDHMYMLDLQPETTPRDFAFRDLCIVYIVRILDLEVSETSKLTHQTDRTLGIVCPIAARFSRSDRTLEDLLMGVADATIHDIILECVDNMHLDKKQRFHNIPLHAPVSPDTTLEQQYNKPLCDLLRNHATDLCDSTLRLCVMHDRWTGRCMSCCPILIAARLAVSLQDIEITCVSHADVLCVQSTHVDIDDLAHYNIVTFCCIHWAAAVNNVDDLRAFQQLFLNTLSGASKISEYRHYCYNNRKLPQLCTELPIFIAAREGSYEKTVLELTEVANRRTLEDLLMGTDHMFAFKDLFVVPRDQLYNKPLCDVIVRTLQDIVLHLLE

SEQ ID NO: 248 - protein, 572 aa, synthetic construct
MQTNRELTEVFEFAARYSESDYIIFVYIPLANAKLPQLCTEVQNMLLMGTLGIVPLKAAVSAQTLHEYMLDLKNRATNLSEEENDEIDGVNHQHLPARRLHDNLLIRCLRCPLILAAELAVLFLNTLSFVCPWCASSKADVKCIDFYSRIDDMKLPDLCTELALQWAAAINNIQLFLNTLSFVGANRFQQLFLNTLNDRVYDFAFRDLPIFLAAREGTYEKLTNTGLYNLGNRLCIVYRDGNPYAVCDDHIYMLDLQPETTARDFAFRDLCIVYIMRLLDIEVTQTSKLSRQPDRTLGIVCPIAAKYSRSNRTLEDLLMGVANATIHDIILECVDDMHLDKKQRFHNIPLQAAVSANTTLEQQYNKPLCDLLRNRATNLCDSTLRLCVMHNRWTGRCMSCCPLILAAELAVSLQDIEITCVSHANVLCVQSTHVDIDQLAHYNIVTFCCLQWAAAMNNMDDLRAFQQLFLNTLSAANKISEYRHYCYNNKKLPQLCTELPFLLAAEEGSYEKTVLELTEVANHRTLEDLLMGTDQMFAFKDLFVVPRNQLYNKPLCDVIVHTLQDIVLHLLD

SEQ ID NO: 249 - protein, 572 aa, synthetic construct
MKTDRELTEVFEFAAKYSRSDYIIFVYIPLAEAKLPQLCTEVENMLLMGTLGIVPLHAGVSADTLHEYMLDLRNRTTDLSEEENDEIDGVNHQHLPARRMHENLLIRCLRCPLILAAHLAVLFLNTLSFVCPWCASSHSDVKCIDFYSRIDDIKLPDLCTELALHWAAAMNNVQLFLNTLSFVGAQKFQQLFLNTLNSRVYDFAFRDLPLFLAAHEGSYEKLTNTGLYNLASRLCIVYRDGNPYAVCDDHVYMLDLQPETTPKDFAFRDLCIVYIVRLLDLEVSHTSKLTRQTDRTLGIVCPIAAKYSRSDRTLEDLLMGVAEATIHDIILECVDNIHLDKKQRFHNIPLHAGVSADTTLEQQYNKPLCDLLRNRTTDLCDSTLRLCVMHNRWTGRCMSCCPLILAAHLAVSLQDIEITCVSHPDVLCVQSTHVDIDDIAHYNIVTFCCLHWAAAINNVDDLRAFQQLFLNTLSGAQKISEYRHYCYNDRKLPQLCTELPLFLAAEEGSYEKTVLELTEVASRRTLEDLLMGTDHVFAFKDLFVVPKDQLYNKPLCDVIVKTLQDIVLHLLN

SEQ ID NO: 250 - protein, 572 aa, synthetic construct
MQTDRYIIFVYIPLARYSRSDELTEVFEFAADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 251 - protein, 572 aa, synthetic construct
MQTDRELTEVFEFAARYSRSDYIIFVYIPLADAKLPQLCTEVDNMLLMGTLGIVPLHAAVSADSEEENDEIDGVNHQHLPARRRNRATDLTLHEYMLDLMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 252 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDLFLNTLSFVCPWCASPLILAARLAVNLLIRCLRCSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 253 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDTLQDIVLHLIVRQLYNKPLCDVLE

SEQ ID NO: 254 - protein, 590 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKLPDLCTELKCIDFYSRIDDLKCIDFYSRIKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 255 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLQLFLNTLSFVALHWAAAVNNVKLPDLCTELGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 256 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVFQQLFLNTLGANKQLFLNTLSFVNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 257 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKVYDFAFRDLNNRFQQLFLNTLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 258 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRKLTNTGLYNLPLFLAAREGSYEVYDFAFRDLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 259 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYELCIVYRDGNPYAVCDANRKLTNTGLYNLDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 260 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRYMLDLQPETTDHMLCIVYRDGNPYAVCDPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 261 - protein, 572 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMFAFRDLCIVYPRDYMLDLQPETTIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 262 - protein, 563 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 263 - protein, 584 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLEYLQYGVYRFADA

SEQ ID NO: 264 - protein, 572 aa, synthetic construct
MQTDRYIIFVYIPLARYSRSDELTEVFEFAGDAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLFE

SEQ ID NO: 265 - protein, 554 aa, synthetic construct
MQTDRELTEVFEFAARYSRSDADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 266 - protein, 545 aa, synthetic construct
MQTDRELTEVFEFAARYSRSDADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 267 - protein, 536 aa, synthetic construct
MQTDRELTEVFEFAARYSRSDADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLMHDRWTGRCMSCCPLILAARLAVSLQDIEITCVSHADVLCVQSTHVDIDDLAHYNIVTFCCLHWAAAVNNVDDLRAFQQLFLNTLSGANKNNRKLPQLCTELPLFLAAREGSYEKTVLELTEVANRRTLEDLLMGTDHMFAFKDLFVVPRDQLYNKPLCDVIVRTLQDIVLHLLE

SEQ ID NO: 268 - protein, 596 aa, synthetic construct
MQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLEYLQYGVYRFADAQLLRREVYVIVR

SEQ ID NO: 269 - protein, 616 aa, synthetic construct
ARYSRSDTLGIVCPICSADAMQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLEYLQYGVYRFADAQLLRREVYVIVR

SEQ ID NO: 270 - protein, 628 aa, synthetic construct
DDLKLPDLCTELARYSRSDTLGIVCPICSADAMQTDRELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHLLEYLQYGVYRFADAQLLRREVYVIVR

SEQ ID NO: 271 - protein, 584 aa, synthetic construct
IQTNRELTEVFEFAPRYSHSDYIIFVYIPLGDAKLPQLCTEVDDLLLMGTLGIVPLHAPMSADTLHEYMLDLHNRATDLSEEENDEIDGVNHQHLPARRIHDNLLIRCLRCPLILAARLALLFLNTLSFVCPWCASSHADMKCIDFYSRIDNLKLPDLCTELALHWTAAVNNLQLFLNTLSFVPANKFQQLFLNTLNDRVYDFAFRDLPLFLAAKEGSYEKLTNTGLYNLASRLCIVYRDGNPYAVCDNHMYMLDLQPETTPRQFAFRDLCIVYIVRFLNLEVSQTSKFTRQTERTLGIVCPIAAHYSKSDRTLEDLLMGVANPTIHDIILECVQNMHLDKKQRFHNIPLHAAYSADTTLEQQYNKPLCDLLRSRATDLCDSTLRLCVVHDRWTGRCMSCCPLILAPRLAVSLQDIEITCVSHADILCVQSTHVDIDDIAHYNIVTFCCLHWGATMSNVDDLRAFQQLFLNTLSGPSKISEYRHYCYDNRKLPQLCTELPLFLGAKEPSYEKTVLELTEVADRRTLEDLLMGTQHMFAFKDLFVVPRNQLYNKPLCDVIIHTLQDIVLHLFEYLQYGVYRFADA

SEQ ID NO: 272 - protein, 562 aa, synthetic construct
MKTDRELTEVFEFAAKYSRSDYIIFVYIPLAEAKLPQLCTEVENMLLMGTLGIVPLHAGVSADTLHEYMLDLRNRTTDLSEEENDEIDGVNHQHLPARRMHENLLIRCLRCPLILAAHLAVLFLNTLSFVCPWCASSHSDVKCIDFYSRIDDIKLPDLCTELALHWAAAMNNVQLFLNTLSFVGAQKFQQLFLNTLNSRVYDFAFRDLPLFLAAHEGSYEKLTNTGLYNLASRLCIVYRDGNPYAVCDDHVYMLDLQPETTPKDFAFRDLCIVYIVRLLDLEVSHTSKLTRQTDRTLGIVCPIAAKYSRSDRTLEDLLMGVAEADNIHLDKKQRFHNIPLHAGVSADTTLEQQYNKPLCDLLRNRTTDLCDSTLRLCVMHNRWTGRCMSCCPLILAAHLAVSLQDIEITCVSHPDVLCVQSTHVDIDDIAHYNIVTFCCLHWAAAINNVDDLRAFQQLFLNTLSGAQKISEYRHYCYNDRKLPQLCTELPLFLAAEEGSYEKTVLELTEVASRRTLEDLLMGTDHVFAFKDLFVVPKDQLYNKPLCDVIVKTLQDIVLHLLN

SEQ ID NO: 273 - protein, 562 aa, synthetic construct
MKTDRELTEVFEFAAKYSRSDYIIFVYIPLAEALLMGTLGIVENMKLPQLCTEVPLHAGVSADTLHEYMLDLRNRTTDLSEEENDEIDGVNHQHLPARRMHENLLIRCLRCPLILAAHLAVLFLNTLSFVCPWCASSHSDVKCIDFYSRIDDIKLPDLCTELALHWAAAMNNVQLFLNTLSFVGAQKVYDFAFRDLNSRFQQLFLNTLPLFLAAHEGSYEKLTNTGLYNLASRLCIVYRDGNPYAVCDDHVYMLDLQPETTPKDFAFRDLCIVYIVRLLDLEVSHTSKLTRQTDRTLGIVCPIAAKYSRSDRTLEDLLMGVAEADNIHLDKKQRFHNIPLHAGVSADTTLEQQYNKPLCDLLRNRTTDLCDSTLRLCVMHNSLQDIEITCVPLILAAHLAVRWTGRCMSCCSHPDVLCVQSTHVDIDDIAHYNIVTFCCLHWAAAINNVDDLRAFQQLFLNTLSGAQKISEYRHYCYNDRKLPQLCTELPLFLAAEEGSYEKTVLELTEVASRRTLEDLLMGTDHVFAFKDLFVVPKDQLYNKPLCDVIVKTLQDIVLHLLN

SEQ ID NO: 274 - protein, 584 aa, synthetic construct
IQTNRYIIFVYIPLPRYSHSDELTEVFEFAGDAKLPQLCTEVDDLLLMGTLGIVPLHAPMSADTLHEYMLDLHNRATDLSEEENDEIDGVNHQHLPARRIHDNLLIRCLRCPLILAARLALLFLNTLSFVCPWCASSHADMKCIDFYSRIDNLKLPDLCTELALHWTAAVNNLQLFLNTLSFVPANKFQQLFLNTLNDRVYDFAFRDLPLFLAAKEGSYEKLTNTGLYNLASRLCIVYRDGNPYAVCDNHMYMLDLQPETTPRQFAFRDLCIVYIVRFLNLEVSQTSKFTRQTERTLGIVCPIAAHYSKSDRTLEDLLMGVANPTIHDIILECVQNMHLDKKQRFHNIPLHAAYSADCDSTLRLCVRSRATDLTTLEQQYNKPLCDLLVHDRWTGRCMSCCPLILAPRLAVSLQDIEITCVSHADILCVQSTHVDIDDIAHYNIVTFCCLHWGATMSNVDDLRAFQQLFLNTLSGPSKISEYRHYCYDNRKLPQLCTELPLFLGAKEPSYEKTVLELTEVADRRTLEDLLMGTQHMFAFKDLFVVPRNQLYNKPLCDVIIHTLQDIVLHLFEYLQYGVYRFADA

SEQ ID NO: 275 - DNA, 15 bp, synthetic construct
  source 1..15: mol_type = other DNA
  misc_difference 8: note = a, c, t or g
rrgktcantgamcyy

SEQ ID NO: 276 - DNA, 13 bp, synthetic construct
  source 1..13: mol_type = other DNA
  misc_difference 7: note = a, c, t or g
aggtcanaggtca

SEQ ID NO: 277 - DNA, 15 bp, synthetic construct
  source 1..15: mol_type = other DNA
gggttgaatgaattt

SEQ ID NO: 278 - protein, 8 aa, synthetic construct
  VARIANT 2: note = V or I
  VARIANT 4: note = Any amino acid
DXEXNPGP

SEQ ID NO: 279 - protein, 30 aa, synthetic construct
  VARIANT 1..30: note = This sequence may encompass 0-15 GS repeating units
GSGSGSGSGSGSGSGSGSGSGSGSGSGSGS

SEQ ID NO: 280 - protein, 30 aa, synthetic construct
  VARIANT 1..30: note = This sequence may encompass 0-15 SG repeating units
SGSGSGSGSGSGSGSGSGSGSGSGSGSGSG

SEQ ID NO: 281 - protein, 45 aa, synthetic construct
  VARIANT 1..45: note = This sequence may encompass 0-15 GSG repeating units
GSGGSGGSGGSGGSGGSGGSGGSGGSGGSGGSGGSGGSGGSGGSG

SEQ ID NO: 282 - protein, 60 aa, synthetic construct
  VARIANT 1..60: note = This sequence may encompass 0-15 SGSG repeating units
SGSGSGSGSGSGSGSGSGSGSGSGSGSGSGSGSGSGSGSGSGSGSGSGSGSGSGSGSGSG

SEQ ID NO: 283 - protein, 5 aa, synthetic construct
GGGGS

SEQ ID NO: 284 - protein, 27 aa, synthetic construct
  VARIANT 2..26: note = This region may encompass 2-5 EAAAK units
AEAAAKEAAAKEAAAKEAAAKEAAAKA

SEQ ID NO: 285 - protein, 4 aa, Alphapapillomavirus sp.
ETQL

SEQ ID NO: 286 - protein, 4 aa, synthetic construct
AAAA

SEQ ID NO: 287 - protein, 9 aa, synthetic construct
RAHYNIVTF

SEQ ID NO: 288 - protein, 104 aa, unidentified
MHQKRTAMFQDPQERPRKLPQLCTELQTTIHNIILECVYCKQQLLRREVYDFAFRDLCIVYRDGNPYAVCDKCLKFYSKISEYRHYCYSLYGTTLEQQYNKPLC

SEQ ID NO: 289 - protein, 97 aa, unidentified
MFQDPQERPRKLPQLCTELQTTIHQIILECVYCKQQLLRREVYDFAFRDLCIVYRDGNPYAVCDKCLKFYSKISEYRHYCYSLYGTTLEQQYNKPLC

SEQ ID NO: 290 - protein, 104 aa, unidentified
MHQKRTAMFQDPQERPRKLPQLCTELQTTIHEIILECVYCKQQLLRREVYDFAFRDLCIVYRDGNPYAVCDKCLKFYSKISEYRHYCYSLYGTTLEQQYNKPLC

SEQ ID NO: 291 - protein, 104 aa, unidentified
MHQKRTAMFQDPQERPRKLPQLCTELQTTIHEIILECVYCKQQLLRREVYDFAFRDLCIVYRDGNPYAVCDKCLQFYSKISEYRHYCYSLYGTTLEQQYNKPLC

SEQ ID NO: 292 - protein, 97 aa, unidentified
MFQDPQERPRKLPQLCTELQTTIHEIILECVYCKQQLLRREVYDFAFRDLCIVYRDGNPYAVCDKCLKFYSKISEYRHYCYSLYGTTLEQQYNKPLC

SEQ ID NO: 293 - protein, 97 aa, unidentified
MFQDPQERPRKLPQLCTELQTTIHEIILGCVYCKQQLLRREVYDFAFRDLCIVYRDGNPYAVCDKCLKFYSKISEYRHYCYSLYGTTLEQQYNKPLC

SEQ ID NO: 294 - protein, 97 aa, unidentified
MFQDPQERPRKLPQLCTELQTTIHEIILECVYCKQQLLRREVYDFAFRDLCIVYRDGNPYAVCNKCLKFYSKISEYRHYCYSLYGTTLEQQYNKPLC

SEQ ID NO: 295 - protein, 94 aa, unidentified
DPQERPRTLPQLCTELQTTIHEIILECVYCKQQLLRREVYDFAFRDLCIVYRDGNPYAVCDKCLKFYSKISEYRHYCYSLYGTTLEQQYNKPLC

SEQ ID NO: 296 - protein, 97 aa, unidentified
MFQDPQERPRTLPQLCTELQTTIHDIILECVYCKQQLLRREVYDFAFRDLCIVYRDGNPYAVCDKCLKFYSKISEYRHYCYSLYGTTLEQQYNKPLC

SEQ ID NO: 297 - protein, 97 aa, unidentified
MFQDPQERPRKLPQLCTELQTTIHDIILQCVYCKQQLLRREVYDFAFRDLCIVYRDGNPYAVCDKCLKFYSKISEYRHYCYSLYGTTLEQQYNKPLC

SEQ ID NO: 298 - protein, 104 aa, unidentified
MHQKRTAMFQDPQERPRKLPQLCTELQTTIHDIILECVYCKQQLLRREVYDFAFWDLCIVYRDGNPYAVCDKCLKFYSKISEYRHYCYSLYGTTLEQQYNKPLC

SEQ ID NO: 299 - protein, 104 aa, unidentified
MHQKRTAMFQDPQERPRKLPELCTELQTTIHDIILECVYCKQQLLRREVYDFAFRDLCIVYRDGNPYAVCDKCLKFYSKISEYRHYCYSLYGTTLEQQYNKPLC

SEQ ID NO: 300 - protein, 107 aa, unidentified
RHFMHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLC

SEQ ID NO: 301 - protein, 97 aa, unidentified
MFQDPQERPRKLPQLCTELQTTIHDIILECVYCKQQLLRREVYDFAFRDLCIVYRDGNPYAVCDKCLKFYSKISEYRHYCYSLYGTTLEQQYNKPLC

SEQ ID NO: 302 - protein, 97 aa, unidentified
MFQDPQERPGKLPQLCTELQTTIHDIILECVYCKQQLLRREVYDFAFRDLCIVYRDGNPYAVCDKCLKFYSKISEYRHYCYSLYGTTLEQQYNKPLC

SEQ ID NO: 303 - protein, 97 aa, unidentified
MFQDPQERPRKLPQLCTELQTTIHDIILECVYCKQQLLRREVYDFAFRDLCIVYRDGNPYAVCDKCLKFYSKICEYRHYCYSLYGTTLEQQYNKPLC

SEQ ID NO: 304 - protein, 97 aa, unidentified
MFQDPQERPRKLPQLCTELQTTIHDIILECVYCKQQLLRREVYDFAFRDLCIVYRDGNPYAVCDKCLKLYSKISEYRHYCYSLYGTTLEQQYNKPLC

SEQ ID NO: 305 - protein, 97 aa, unidentified
MFQDPQERPRKLPQLCTELQTTIHDIILKCVYCKQQLLRREVYDFAFRDLCIVYRDGNPYAVCDKCLKLYSKISEYRHYCYSVYGTTLEQQYNKPLC

SEQ ID NO: 306 - protein, 97 aa, unidentified
MFQDPQERPTKLPELCTELQTTIHDIILECVYCKQQLLRREVYDFAFRDLCIVYRDGNPYAVCDKCLKFYSKISEYRYYCYSLYGTTLEQQYNKPLC

SEQ ID NO: 307 - protein, 104 aa, unidentified
MHQKRTAMFQDPQERPIKLPELCTELQTTIHDIILECVYCKQQLLRREVYDFAFRDLCIVYRDGNPYAVCDKCLKFYSKISEYRYYCYSLYGTTLEQQYNKPLC

SEQ ID NO: 308 - protein, 104 aa, unidentified
MHQKRTAMFQDPQERPIKLPHLCTELQTTIHDIILECVYCKQQLLRREVYDFAFRDLCIVYRDGNPYAVCDKCLKFYSKISEYRYYCYSLYGTTLEQQYNKPLC

SEQ ID NO: 309 - protein, 90 aa, unidentified
PPIKLPDLCTELQTTIHDIILECVYCKQQLLQREVYDFAFRDLCIVYRDGNPYAVCDKCLKFYSKISEYRYYCYSLYGTTLEQQYNKPLC

SEQ ID NO: 310 - protein, 90 aa, unidentified
PPIKLPDLCTVLQTTIHDIILECVYCKQQLLQREVYDFAFRDLCIVYRDGNPYAVCDKCLKFYSKISEYRYYCYSLYGTTLEQQYNKPLC

SEQ ID NO: 311 - protein, 97 aa, unidentified
MFQDPQERPIKVPDLCTELQTTIHDIILECVYCKQQLLRREVYDFAFRDLCIVYRDGNPYAVCDKCLKFYSKISEYRYYCYSLYGTTLEQQYNKPLC

SEQ ID NO: 312 - protein, 97 aa, unidentified
MFQDPQERPIKLPDLCTELQTTIHDIILECVYCKQQLLRREVYDFAFRDLCIVYRDGNPYAVCDKCLKFYSKISEYRYYCYSLYGTTLEQQYNKPLC

SEQ ID NO: 313 - protein, 97 aa, unidentified
MFQDPQERPIKLPDLCTELQTTINDIILECVYCKQQLLRREVYDFAFRDLCIVYRDGNPYAVCDKCLKFYSKISEYRYYCYSLYGTTLEQQYNKPLC

SEQ ID NO: 314 - protein, 104 aa, unidentified
MHQKRTAMFQDPQERPRKLPDLCTELQTTIHDIILECVYCKQQLLRREVYDFAFRDLCIVYRDGNPYGVCDKCLKFYSKISEYRYYCYSLYGTTLEQQYNKPLC

SEQ ID NO: 315 - protein, 158 aa, Alphapapillomavirus sp.
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 316 - protein, 98 aa, Alphapapillomavirus sp.
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 317 - protein, 83 aa, Alphapapillomavirus sp.
MTNLDTASTTLLACFLLCFCVLLCVCLLIRPLLLSVSTYTSLIILVLLLWITAASAFRCFIVYIIFVYIPLFLIHTHARFLIT

SEQ ID NO: 318 - protein, 158 aa, Alphapapillomavirus sp.
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 319 - protein, 105 aa, Alphapapillomavirus sp.
MHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 320 - protein, 73 aa, Alphapapillomavirus sp.
MLSLIFLFCFCVCMYVCCHVPLLPSVCMCAYAWVLVFVYIVVITSPATAFTVYVFCFLLPMLLLHIHAILSLQ

SEQ ID NO: 321 - protein, 566 aa, synthetic construct
RELTEVFEFAARYSRSDYIIFVYIPLADAKLPQLCTEVDNMLLMGTLGIVPLHAAVSADTLHEYMLDLRNRATDLSEEENDEIDGVNHQHLPARRMHDNLLIRCLRCPLILAARLAVLFLNTLSFVCPWCASSHADVKCIDFYSRIDDLKLPDLCTELALHWAAAVNNVQLFLNTLSFVGANKFQQLFLNTLNNRVYDFAFRDLPLFLAAREGSYEKLTNTGLYNLANRLCIVYRDGNPYAVCDDHMYMLDLQPETTPRDFAFRDLCIVYIVRLLDLEVSQTSKLTRQTDRTLGIVCPIAARYSRSDRTLEDLLMGVADATIHDIILECVDNMHLDKKQRFHNIPLHAAVSADTTLEQQYNKPLCDLLRNRATDLCDSTLRLCVMHDRWTGRCMSCCPLILAARLAVSLQDIEITCVSHADVLCVQSTHVDIDDLAHYNIVTFCCLHWAAAVNNVDDLRAFQQLFLNTLSGANKISEYRHYCYNNRKLPQLCTELPLFLAAREGSYEKTVLELTEVANRRTLEDLLMGTDHMFAFKDLFVVPRDQLYNKPLCDVIVRTLQDIVLHL

SEQ ID NO: 322 - protein, 432 aa, synthetic construct
KELTEVFEFAKKYIIFVYIPLKKKLPQLCTEVKKLLMGTLGIVKKTLHEYMLDLKKSEEENDEIDGVNHQHLPARRKKNLLIRCLRCKKLFLNTLSFVCPWCASKKKCIDFYSRIKKKLPDLCTELKKQLFLNTLSFVKKFQQLFLNTLKKVYDFAFRDLKKKLTNTGLYNLKKLCIVYRDGNPYAVCDKKYMLDLQPETTKKFAFRDLCIVYKKTLGIVCPIKKRTLEDLLMGVKKTIHDIILECVKKHLDKKQRFHNIKKTTLEQQYNKPLCDLLKKCDSTLRLCVKKRWTGRCMSCCKKSLQDIEITCVKKLCVQSTHVDIKKAHYNIVTFCCKKDDLRAFQQLFLNTLSKKISEYRHYCYKKKLPQLCTELKKKTVLELTEVKKRTLEDLLMGTKKFAFKDLFVVKKQLYNKPLCDVKKTLQDIVLHL

SEQ ID NO: 323 - protein, 546 aa, synthetic construct
MFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPMTNLDTASTTLLACFLLCFCVLLCVCLLIRPLLLSVSTYTRCFIVYIIFVYIPLFLIHMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHEIDGVNHQHLPARRAAPQRHTMLCMCCKCEARIERVVESSADDLRAFQQLFLNTLSFVCPWCASQQMLSLIFLFCFCVCMYVCCHVPLLPSVVVITSPATAFTVYLLPMLLLHIHAILS

SEQ ID NO: 324 - protein, 604 aa, synthetic construct
KLPQLCTEVMFQDPQERPRKLPQLCTALQTTIHDIILECVYCKQQLLRREVYDFAFRDGCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRAAAAQLYNKPLCDVMPGDTPTLHEYMLDLQPETTDLYGYEQLNDSSEEEDEIDGPAGQAAPDRAHYNIVTFCCKCDSTLRRCVQSTHVDIRTLEDLLMGTLGIVCPICSQKPRTLEDLLMGVMTNLDTASTTLLACFLLCFCVLLCVCLLIRPLLLSVSTYTRCFIVYIIFVYIPLFLIHKLPDLCTEVMARFEDPTRRPYKLPDLCTALNTSLQDIEITCVYCKTVLELTEVFEFAFKDGFVVYRDSIPHAACHKCIDFYSRIRELRHYSDSVYGDTLEKLTNTGLYNLLIRCLRCQKPLNPAEKLRHKLTNTGLYNVEIDGVNHQHLPARRAAPQRHTMLCMCCKCEARIERVVESSADDLRAFQQLFLNTLSFVCPWCASQQRAFQQLFLNVMLSLIFLFCFCVCMYVCCHVPLLPSVVVITSPATAFTVYLLPMLLLHIHAILS